(19) United States
(12) Patent Application Publication
(10) Pub. No.: US 2024/0139190 A1
(43) Pub. Date: May 2, 2024

(54) NOVEL ANTIPARASITIC COMPOUNDS AND METHODS

(71) Applicant: The Board of Regents of The University of Texas System, Austin, TX (US)

(72) Inventors: Dawn M. WETZEL, Dallas, TX (US); Imran ULLAH, Dallas, TX (US); Laela M. BOOSHEHRI, Dallas, TX (US); Joseph READY, Dallas, TX (US); Suraksha GAHALAWAT, Dallas, TX (US); Bin HU, Dallas, TX (US); Yesu ADDEPALLI, Dallas, TX (US)

(21) Appl. No.: 17/768,529

(22) PCT Filed: Oct. 19, 2020
(86) PCT No.: PCT/US20/56360

§ 371 Date: Apr. 13, 2022

Related U.S. Application Data
(60) Provisional application No. 62/923,241, filed on Oct. 18, 2019.

(51) Int. Cl. A61K 31/513; A61K 31/506; A61K 31/519; A61K 31/5377; A61P 33/02

(52) U.S. Cl. CPC A61K 31/513; A61K 31/506; A61K 31/519; A61K 31/5377; A61P 33/02

(57) ABSTRACT

Novel compounds for treating or inhibiting leishmaniasis and other parasitic protozoan diseases are disclosed herein. The compounds bind to Leishmania tubulin, induce parasite microtubule polymerization, stall Leishmania cell division, and have broad antiparasitic activity.

This application claims the benefit of priority of U.S. Provisional Patent Application No. 62/923,241, filed Oct. 18, 2019, which is hereby incorporated by reference in its entirety.

### FIELD OF THE INVENTION

This invention relates to the fields of organic chemistry and antiparasitic drugs.

### BACKGROUND

Neglected tropical diseases caused by parasitic trypanosomatids afflict 27 million people worldwide. The three parasitic trypanosomatids all share a unique mitochondrial structure, possess flagella, and share similar subcellular organization. Human leishmaniasis is endemic in nearly 100 countries, and 350 million people worldwide are at risk for this disfiguring (cutaneous (CL) or mucocutaneous) or lethal (visceral (VL)) disease. Leishmaniasis is caused by obligate intracellular single-celled parasites of the Leishmania genus, which have two life cycle stages. The fast-growing promastigote, which lives in sandflies, transforms into the slow-growing amastigote inside the human phagocytic cell and causes the disease leishmaniasis. Disease severity is dependent on both the parasite species and the host immune response. Chagas disease is caused by Trypanosoma cruzi, an intracellular parasite that invades the heart, gut, and smooth muscle. Chagas disease is the most common cause of non-ischemic cardiomyopathy in Central and South America; it also results in megaesophagus and megacolon, and either complication can be fatal. In sub-Saharan Africa, human African trypanosomiasis (HAT) leads to sleep-wake cycle disturbances, neurological complications, and eventual death if it is not treated. HAT is caused by T. brucei, an extracellular pathogen that divides in the bloodstream but can invade the CNS. There are two forms that infect humans: Trypanosoma brucei gambiense, Trypanosoma brucei rhodesiense, and one that infects cattle: Trypanosoma brucei brucei. Although these diseases generally afflict the developing world, leishmaniasis and Chagas disease are spreading into previously non-endemic areas, including the southern United States.

Apicomplexan parasites such as Plasmodium, the causative agent of malaria, result in more than 200 million infections and 800,000 deaths per year in tropical and subtropical regions. Infection by five Plasmodium species results in human disease: P. falciparum, P. vivax, P. ovale, P. malariae, and P. knowlesi. After a mosquito bites a human host, the parasites initially invade the liver and then enter replicative stages in erythrocytes. Disease manifestations are caused by this blood stage. Some parasites differentiate into gametocytes, which then are ingested by mosquitoes and allow the disease to spread. Other medically-important apicomplexans include Toxoplasma (toxoplasmosis, which affects immunocompromised hosts) and Cryptosporidium (cryptosporidiosis, which is a significant cause of diarrheal disease worldwide)

Therapy for nearly all protozoan infections, including current treatments for leishmaniasis, depends on poorly effective, centuries-old, toxic drugs that are simultaneously expensive and difficult to administer. Injected pentavalent antimony compounds are used in the developing world to treat leishmaniasis, but they cause renal, liver, and cardiac toxicity. The only oral drug for leishmaniasis, miltefosine, is so teratogenic that women of childbearing age must avoid pregnancy for 5 months after therapy. Recent Infectious Disease Society of America leishmaniasis guidelines state that injected amphotericin, which targets parasite membrane sterols, is the treatment of choice for visceral disease in the US; however, amphotericin is associated with a high risk of severe renal and cardiac toxicity, particularly during long courses such as those needed for leishmaniasis. Standard treatment for the early stages of HAT is pentamidine for T. brucei gambiense and suramin for T. brucei rhodesiense. Later CNS infections are traditionally treated with melarsoprol, which causes fatal encephalopathy in up to 10% of treated patients; although eflornithine is an alternative, it cannot be used in T. brucei rhodesiense infection. Chagas disease is treated with nifurtimox and benznidazole, which cause significant side effects and are of negligible benefit in chronic disease. All new drug combinations for malaria therapy depend on artemisins, to which resistance has already emerged. There is a single drug available to treat toxoplasmosis and another single drug used to treat cryptosporidiosis. Therefore, there is a clear need for new drugs with novel modes of action that circumvent resistance mechanisms. In particular, a drug that could be used to treat all protozoal infections, or at least all trypanosomatid and apicomplexan infections, would be ideal.

### SUMMARY

The inventors have found novel compositions for treating disorders caused by trypanosomatid parasites, including leishmaniasis, African trypanosomiasis, and Chagas disease. These compositions also kill the apicomplexan parasite Plasmodium falciparum, the causative agent for the most severe human form of malaria. The compositions disclosed herein may serve as alternatives to the extremely toxic anti-parasitic drugs that are currently in use. Experiments show that the compositions selectively disrupt Leishmania and other protozoal tubulin dynamics. Specifically, the compositions were found to promote leishmanial tubulin polymerization in a concentration-dependent manner. Due to tubulin's conservation across the protozoa and the compositions' activities against multiple parasites, there is significant potential for this scaffold to allow development of broad-spectrum antiparasitic agents to treat a multitude of devastating protozoal infections, including leishmaniasis, African trypanosomiasis, Chagas disease, malaria, toxoplasmosis, and cryptosporidiosis.

The present disclosure provides a method for treating or inhibiting a parasitic disease in a subject comprising administering to the subject a composition comprising a compound of the formula below:

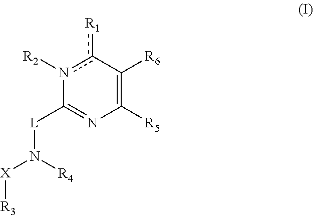

where R₁ is alkyl, oxygen, alkoxy, substituted or unsubstituted amine, or substituted or unsubstituted benzoate; R₂ is H, alkyl, or substituted or unsubstituted benzyl; L is a substituted or unsubstituted aryl or substituted or unsubstituted heteroaryl, including but not limited to phenyl, pyrazole, imidazole, and pyridyl, wherein L optionally includes a carbonyl moiety linking L to the amine group; X is H, CO, SO₂, or CONH; when X is not H, R₃ is alkyl, substituted or unsubstituted cycloalkyl, substituted or unsubstituted heterocyclyl, substituted or unsubstituted aryl, or substituted or unsubstituted heteroaryl; R₄ is H, alkyl, substituted or unsubstituted aryl, substituted or unsubstituted heteroaryl, substituted or unsubstituted heterocyclyl, substituted or unsubstituted cycloalkyl, or substituted or unsubstituted benzyl; R₅ and R₆ are each independently selected from H, halide, linear, cyclic or branched alkyl, alkoxy, substituted or unsubstituted aryl, substituted or unsubstituted heteroaryl, substituted or unsubstituted benzyloxy, substituted or unsubstituted benzoate, and substituted or unsubstituted arylsulfonate, or join to form a carbocyclic or heterocyclic ring; or a pharmaceutically-acceptable salt, or prodrug thereof. In some aspects, when R₁ is alkyl, alkoxy, amine, substituted amine or benzoate, the bond between the R₁-bearing carbon and the vicinal amine is a double bond and there is no R₂ substituent, and when R₁ is oxygen, the bond between the R₁-bearing carbon and the vicinal amine is a single bond and the bond between the R₁-bearing carbon and oxygen is a double bond (carbonyl bond).

In some embodiments, the composition is used to treat a parasitic disease, including, but not limited to leishmaniasis, African trypanosomiasis, Chagas disease, and malaria. In some aspects, the composition disrupts trypanosomatid tubulin dynamics. In some aspects, the composition disrupts protozoan tubulin dynamics. In some aspects, the composition promotes trypanosomatid tubulin polymerization. In some aspects, the composition promotes protozoan tubulin polymerization. In some aspects, the leishmaniasis is caused by a parasite of the genus Leishmania. In some aspects parasites of the genus Leishmania include Leishmania amazonesis, L. donovani, L. tarentolae, L. tropica, L. major, L. aethiopica, L. Arabica, L. aristidesi, L. forattinii, L. gerbilli, L. infantum, L. killicki, L. Mexicana, L. pifanoi, L. turanica, L. venezuelensis, L. waltoni, L. enriettii, L. macropdoum, L. martiniquensis, L. orientalis, L. adleri, L. agamae, L. ceramodactyli, L. gulikae, L. gymnodactyli, L. helioscopi, L. hemidactyli, L. hoogstraali, L. nicollei, L. platycephala, L. phrynocephali, L. senegalensis, L. sofieffi, L. tarentolae, L. zmeevi, L. zuckermani, L. braziliensis, L. guyanensis, L. lainsoni, L. lindenbergi, L. naiffi, L. panamensis, L. peruviana, L. shawi, L. utingensis, L. colombiensis, L. equatorensis, L. herreri, L. monterogeii, L. schaudinni, L. esmeraldas, L. deanei, L. hertigi, L. australiensis, and L. costaricensis. In some embodiments, the parasitic disease is caused by a parasite of the genus Trypanosoma. In some aspects, parasites of the genus Trypanosoma include Trypanosoma brucei gambiense, Trypanosoma brucei rhodesiense, and Trypanosoma brucei brucei. In some aspects, a parasite of the genus Trypanosoma is Trypanosoma cruzi. In some aspects, the parasitic disease is caused by a parasite of the genus Plasmodium. In some aspects, parasites of the genus Plasmodium include Plasmodium falciparum, P. vivax, P. ovale, P. malariae, and P. knowlesi. In some embodiments, the parasitic disease is caused by a parasite of the genus Cryptosporidium. In some aspects, parasites of the genus Cryptosporidium include C. hominis and C. parvum. In some aspects, the parasitic disease is caused by a parasite of the genus Toxoplasma. In some embodiments, a parasite of the genus Toxoplasma is T. gondii. In some aspects, the subject is a human being. In some aspects, the compound inhibits, suppresses, or reduces the population of intracellular or axenic amastigotes or the human infective form of other parasites by about 50%.

In some embodiments, a method for treating or inhibiting trypanosomatid or other parasitic disease in a subject comprising administering to the subject a composition comprising a compound of formula II or III below:

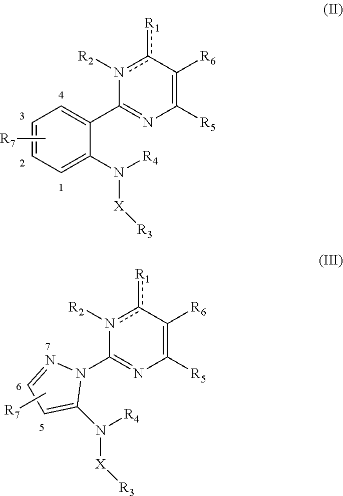

where R₁ is alkyl, oxygen, substituted or unsubstituted amine, alkoxy or substituted or unsubstituted benzoate; R₂ is H, alkyl, or substituted or unsubstituted benzyl; X is H, CO, SO₂, or CONH; when X is not H, R₃ is alkyl, substituted or unsubstituted cycloalkyl, substituted or unsubstituted heterocyclyl, substituted or unsubstituted aryl, or substituted or unsubstituted heteroaryl; R₄ is H, alkyl, substituted or unsubstituted aryl, substituted or unsubstituted heteroaryl, substituted or unsubstituted heterocyclyl, substituted or unsubstituted cycloalkyl, or substituted or unsubstituted benzyl; R₅ and R₆ are each independently selected from H, halide, linear, cyclic or branched alkyl, alkoxy, substituted or unsubstituted aryl, substituted or unsubstituted heteroaryl, substituted or unsubstituted benzyloxy, substituted or unsubstituted benzoate, and substituted or unsubstituted arylsulfonate, or join to form a carbocyclic or heterocyclic ring; R₇ is attached to one or more ring atoms at positions 1, 2, 3, 4, 5, 6, and 7, and each R₇ is independently H, alkyl, or halide; or a pharmaceutically-acceptable salt, or prodrug thereof. In some aspects, when R₁ is alkyl, alkoxy, substituted or unsubstituted amine, or benzoate, the bond between the R₁-bearing carbon and the vicinal amine is a double bond and there is no R₂ substituent, and when R₁ is oxygen, the bond between the R₁-bearing carbon and the vicinal amine is a single bond and the bond between the R₁-bearing carbon and oxygen is a double bond (carbonyl bond).

In some embodiments, a method for treating or inhibiting a parasitic diseases in a subject comprising administering to the subject a composition comprising a compound of formula IV below:

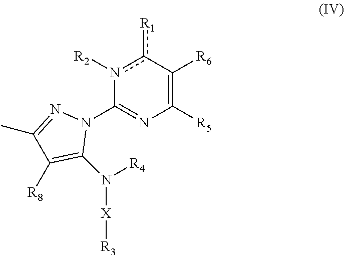

where R₁ is alkyl, alkoxy, substituted or unsubstituted amine, oxygen or substituted or unsubstituted benzoate; R₂ is H, alkyl, or substituted or unsubstituted benzyl; X is H, CO, SO₂, or CONH; when X is not H, R₃ is alkyl, substituted or unsubstituted cycloalkyl, substituted or unsubstituted heterocyclyl, substituted or unsubstituted aryl, or substituted or unsubstituted heteroaryl; R₄ is H, alkyl, substituted or unsubstituted aryl, substituted or unsubstituted heteroaryl, substituted or unsubstituted heterocyclyl, substituted or unsubstituted cycloalkyl, or substituted or unsubstituted benzyl; R₅ and R₆ are each independently selected from H, halide, linear, cyclic or branched alkyl, alkoxy, substituted or unsubstituted aryl, or substituted or unsubstituted heteroaryl, substituted or unsubstituted heteroaryl, substituted or unsubstituted benzyloxy, substituted or unsubstituted benzoate, and substituted or unsubstituted arylsulfonate, or join to form a carbocyclic or heterocyclic ring; and R₈ is H, alkyl, or halide; or a pharmaceutically-acceptable salt, enantiomer, diastereomer, or prodrug thereof. In some aspects, when R₁ is alkyl, alkoxy, substituted or unsubstituted amine, or benzoate, the bond between the R₁-bearing carbon and the vicinal amine is a double bond and there is no R₂ substituent, and when R₁ is oxygen, the bond between the R₁-bearing carbon and the vicinal amine is a single bond and the bond between the R₁-bearing carbon and oxygen is a double bond (carbonyl bond). In some aspects, R₁ is oxygen and R₂ is H. In some aspects, X is CO and R₃ is substituted aromatic. In some aspects, R₅ is methyl and R₆ is ethyl.

In some aspects, a method for treating or inhibiting a parasitic disease in a subject comprising administering to the subject a composition comprising at least one of the following compounds:

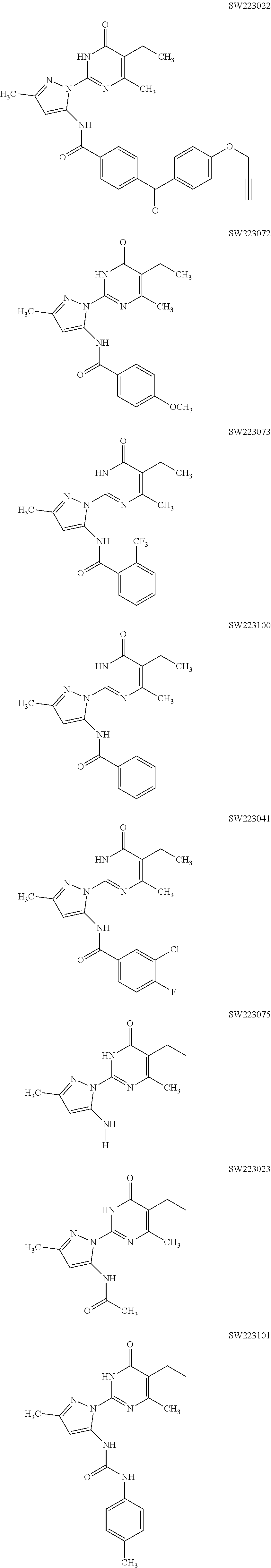

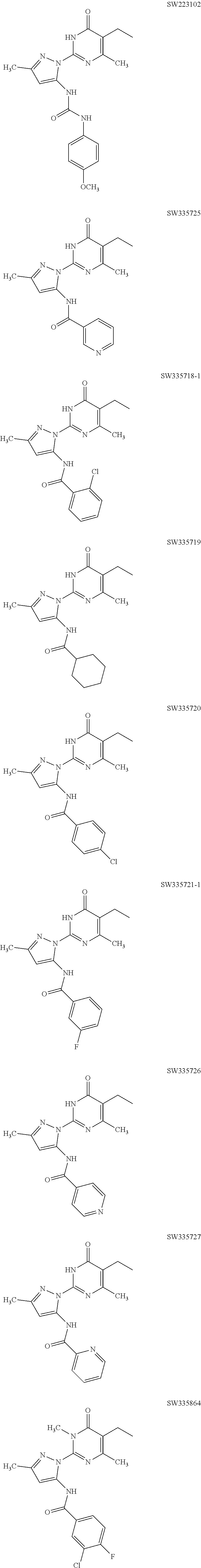

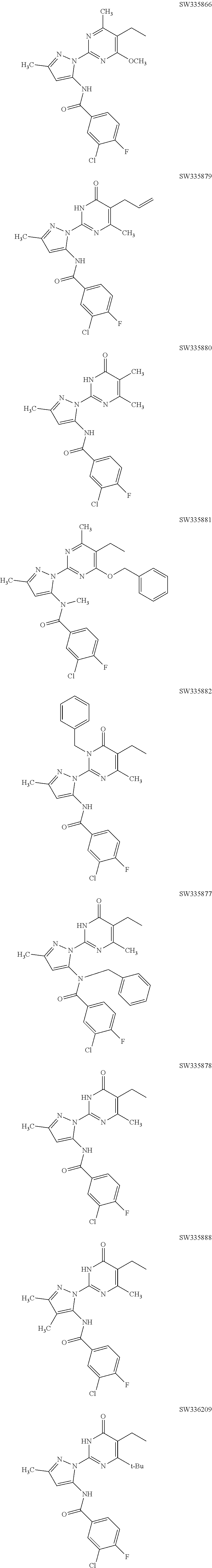

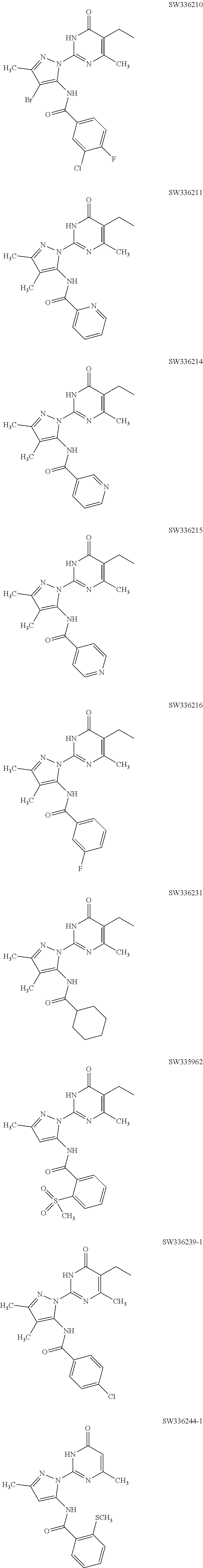

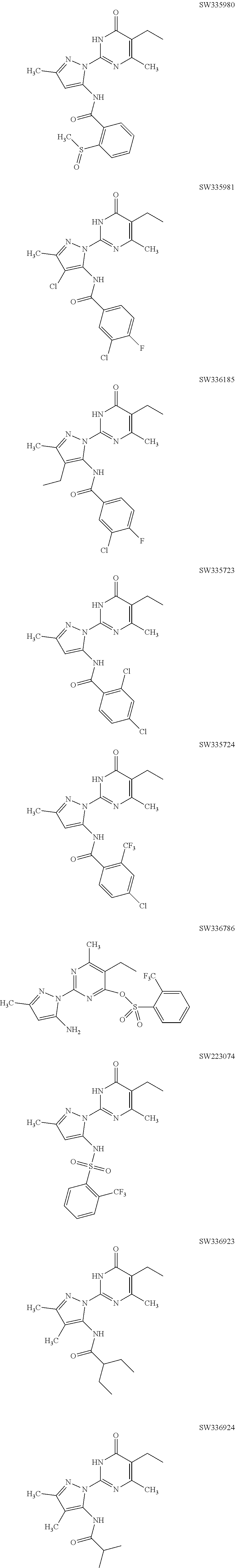

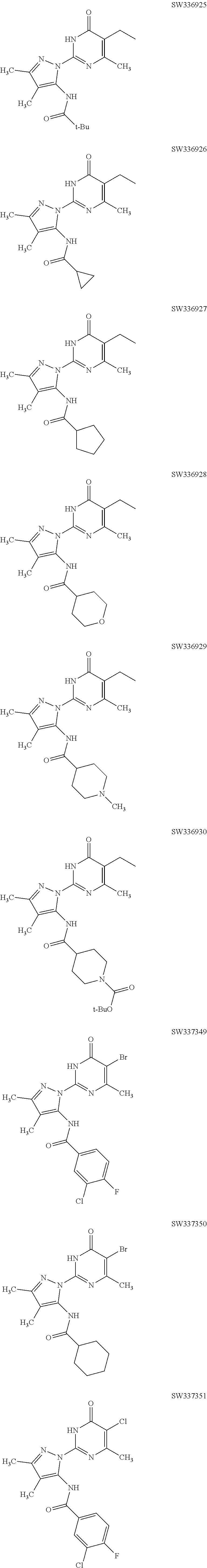

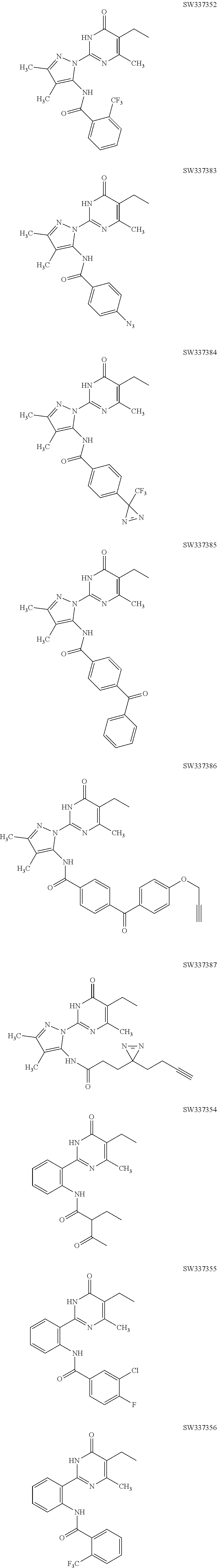

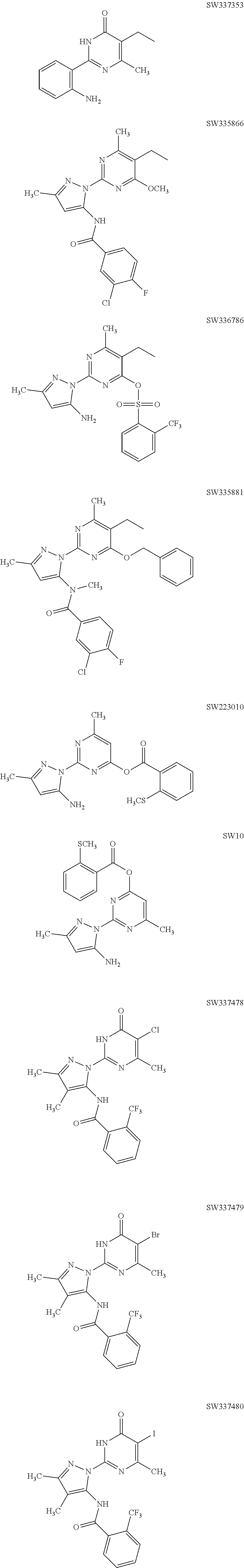

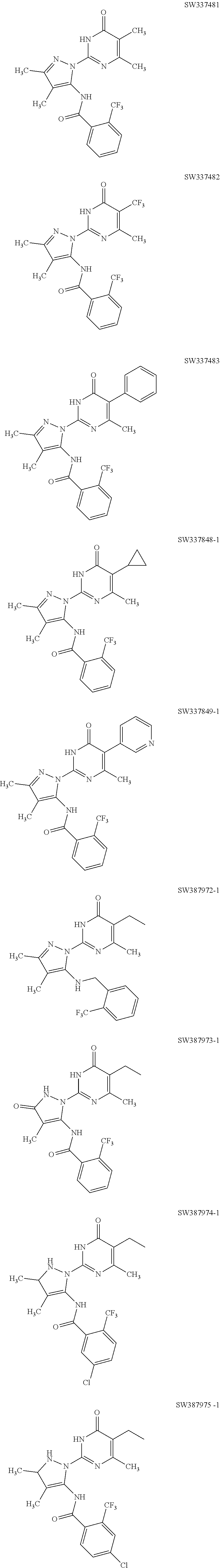

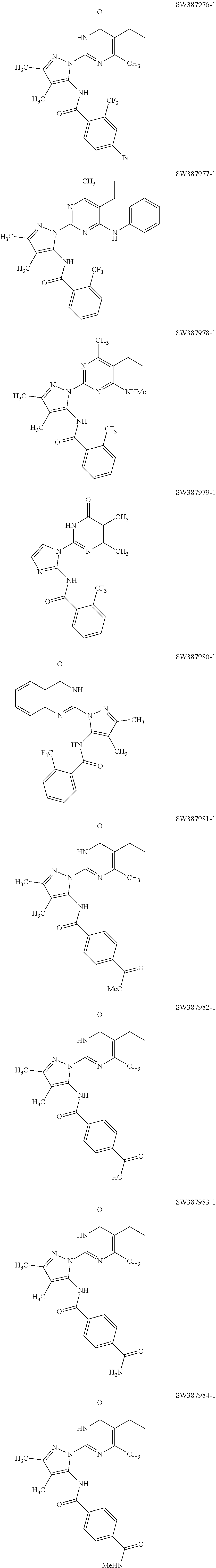

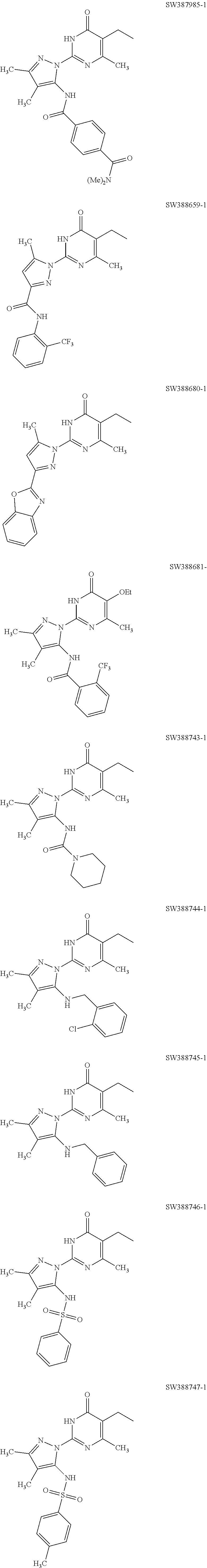

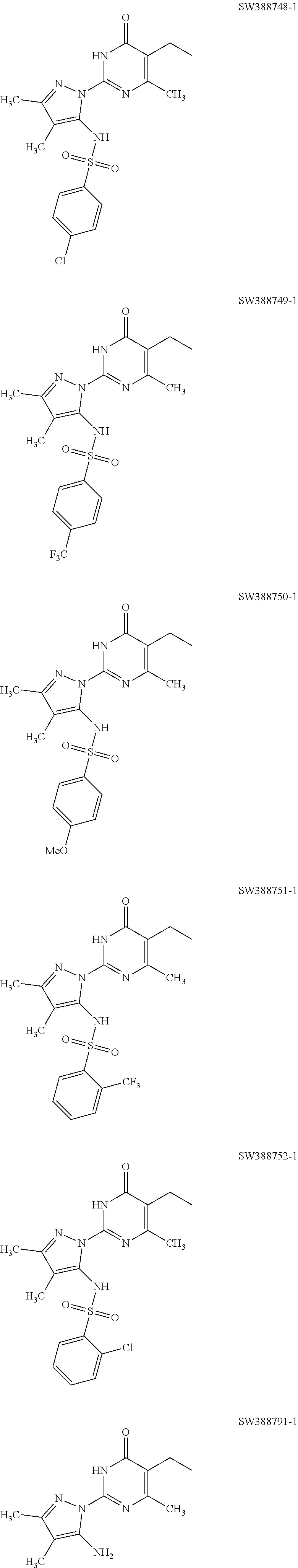

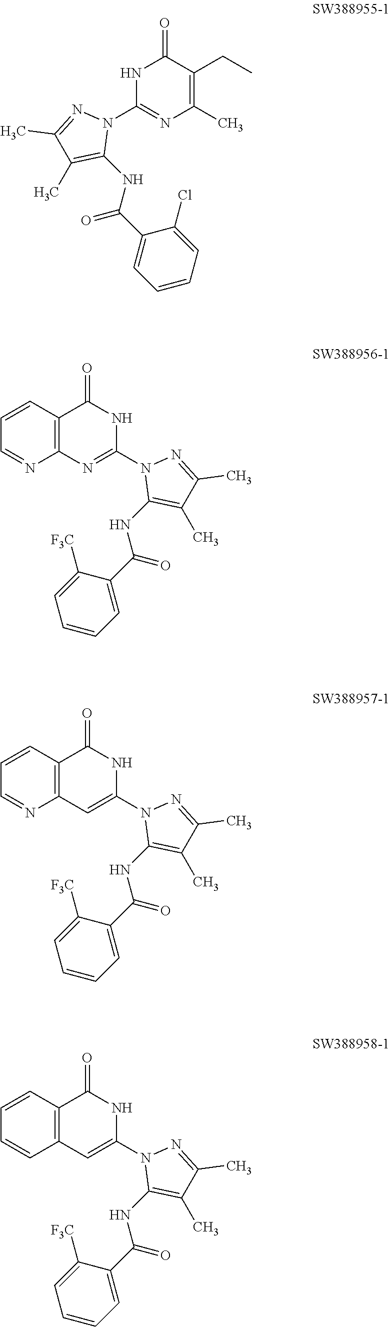 [SMILES: CCc1c(C)nc(-n2nc(C)c(C)c2NC(=O)c2ccccc2Cl)[nH]c1=O ; Cc1nn(-c2nc3ncccc3c(=O)[nH]2)c(NC(=O)c2ccccc2C(F)(F)F)c1C ; Cc1nn(-c2cc3ncccc3c(=O)[nH]2)c(NC(=O)c2ccccc2C(F)(F)F)c1C ; Cc1nn(-c2cc3ccccc3c(=O)[nH]2)c(NC(=O)c2ccccc2C(F)(F)F)c1C]

or a pharmaceutically-acceptable salt, or prodrug thereof. In some aspects, the composition disrupts protozoan, including leishmanial, tubulin dynamics. In some aspects, the composition promotes leishmanial tubulin polymerization.

Some aspects of the disclosure are directed to a composition comprising a compound of formula I below:

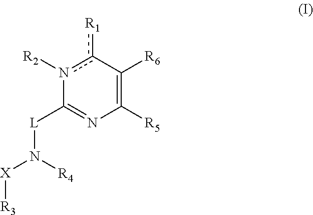

where R₁ is alkyl, oxygen, alkoxy, substituted or unsubstituted amine, or substituted or unsubstituted benzoate; R₂ is H, alkyl, or substituted or unsubstituted benzyl; L is a substituted or unsubstituted aryl or substituted or unsubstituted heteroaryl, including but not limited to phenyl, pyrazole, imidazole, and pyridyl, wherein L optionally includes a carbonyl moiety linking L to the amine group; X is H, CO, SO₂, or CONH; when X is not H, R₃ is alkyl, substituted or unsubstituted cycloalkyl, substituted or unsubstituted heterocyclyl, substituted or unsubstituted aryl, or substituted or unsubstituted heteroaryl; R₄ is H, alkyl, substituted or unsubstituted aryl, substituted or unsubstituted heteroaryl, substituted or unsubstituted heterocyclyl, substituted or unsubstituted cycloalkyl, or substituted or unsubstituted benzyl; R₅ and R₆ are each independently selected from H, halide, linear, cyclic or branched alkyl, alkoxy, substituted or unsubstituted aryl, or substituted or unsubstituted heteroaryl, or join to form a carbocyclic or heterocyclic ring; or a pharmaceutically-acceptable salt, or prodrug thereof. In some aspects, when R₁ is alkyl, alkoxy, substituted or unsubstituted amine, or benzoate, the bond between the R₁-bearing carbon and the vicinal amine is a double bond and there is no R₂ substituent, and when R₁ is oxygen, the bond between the R₁-bearing carbon and the vicinal amine is a single bond and the bond between the R₁-bearing carbon and oxygen is a double bond (carbonyl bond).

Some aspects of the disclosure are directed to a composition comprising a compound of the formula II or III below:

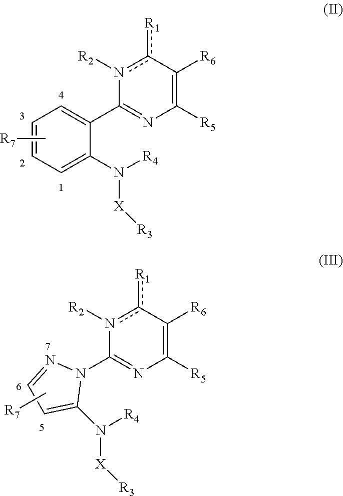

where R₁ is alkyl, oxygen, substituted or unsubstituted amine, or alkoxy or substituted or unsubstituted benzoate; R₂ is H, alkyl, or substituted or unsubstituted benzyl; X is H, CO, SO₂, or CONH; when X is not H, R₃ is alkyl, substituted or unsubstituted cycloalkyl, substituted or unsubstituted aryl, or substituted or unsubstituted heteroaryl; R₄ is H, alkyl, substituted or unsubstituted aryl, substituted or unsubstituted heteroaryl, substituted or unsubstituted heterocyclyl, substituted or unsubstituted cycloalkyl, or substituted or unsubstituted benzyl; R₅ and R₆ are each independently selected from H, halide, linear, cyclic or branched alkyl, alkoxy, substituted or unsubstituted aryl, or substituted or unsubstituted heteroaryl, or join to form a carbocyclic or heterocyclic ring; R₇ is attached to one or more ring atoms at positions 1, 2, 3, 4, 5, 6, and 7, and each R₇ is independently H, alkyl, or halide; or a pharmaceutically-acceptable salt, or prodrug thereof. In some aspects, when R₁ is alkyl, alkoxy, substituted or unsubstituted amine, or benzoate, the bond between the R₁-bearing carbon and the vicinal amine is a double bond, and when R₁ is oxygen, the bond between the R₁-bearing carbon and the vicinal amine is a single bond and the bond between the R₁-bearing carbon and oxygen is a double bond (carbonyl bond).

Some aspects of the disclosure are directed to a composition comprising a compound of the formula IV below:

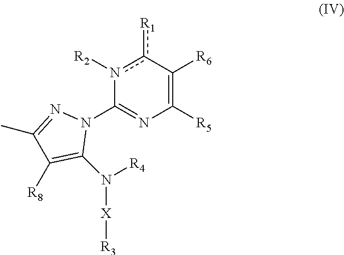

where R₁ is alkyl, oxygen, substituted or unsubstituted amine, or alkoxy; R₂ is H, alkyl, or substituted or unsubstituted benzyl; X is H, CO, SO₂, or CONH; when X is not H, R₃ is alkyl, substituted or unsubstituted cycloalkyl, substituted or unsubstituted heterocyclyl, substituted or unsubstituted aryl, or substituted or unsubstituted heteroaryl; R₄ is H, alkyl, substituted or unsubstituted aryl, substituted or unsubstituted heteroaryl, substituted or unsubstituted heterocyclyl, substituted or unsubstituted cycloalkyl, or substituted or unsubstituted benzyl; R₅ and R₆ are independently selected from H, halide, linear, cyclic or branched alkyl, alkoxy, substituted or unsubstituted aryl, or substituted or unsubstituted heteroaryl, or join to form a carbocyclic or heterocyclic ring; and R₈ is H, alkyl, or halide; or a pharmaceutically-acceptable salt, or prodrug thereof. In some aspects, when R₁ is alkyl, alkoxy, substituted or unsubstituted amine, or benzoate, the bond between the R₁-bearing carbon and the vicinal amine is a double bond and there is no R₂ substituent, and when R₁ is oxygen, the bond between the R₁-bearing carbon and the vicinal amine is a single bond and the bond between the R₁-bearing carbon and oxygen is a double bond (carbonyl bond). In some aspects, R₁ is oxygen and R₂ is H. In some aspects, X is CO and R₃ is substituted aromatic. In some aspects, R₅ is methyl and R₆ is ethyl.

In some embodiments, a compound of the present disclosure is further defined as one of:

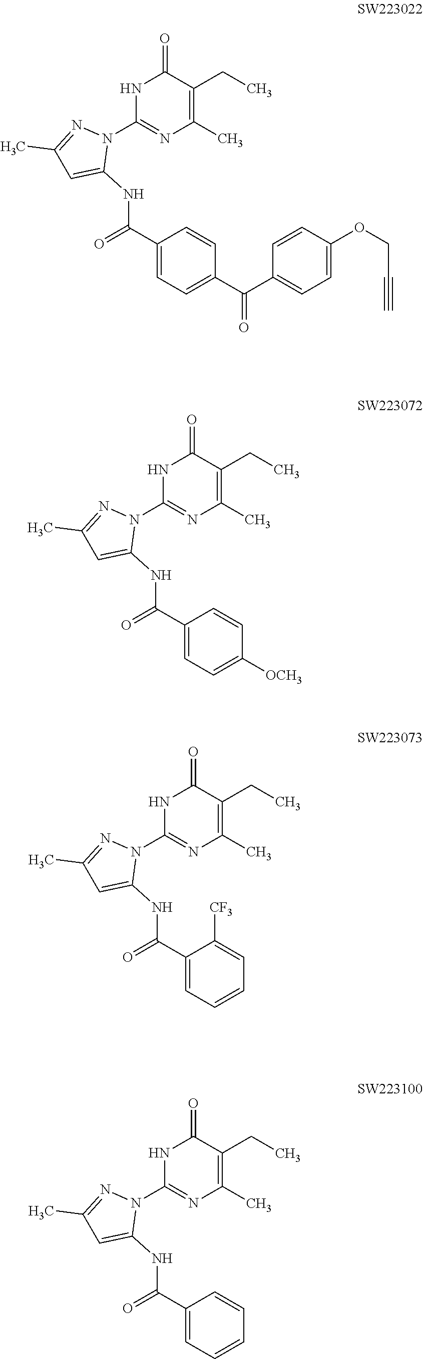

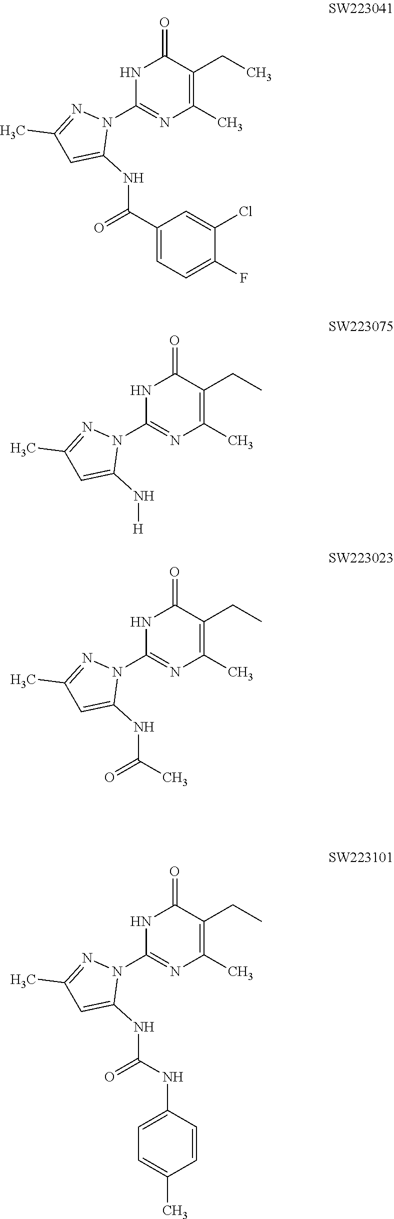

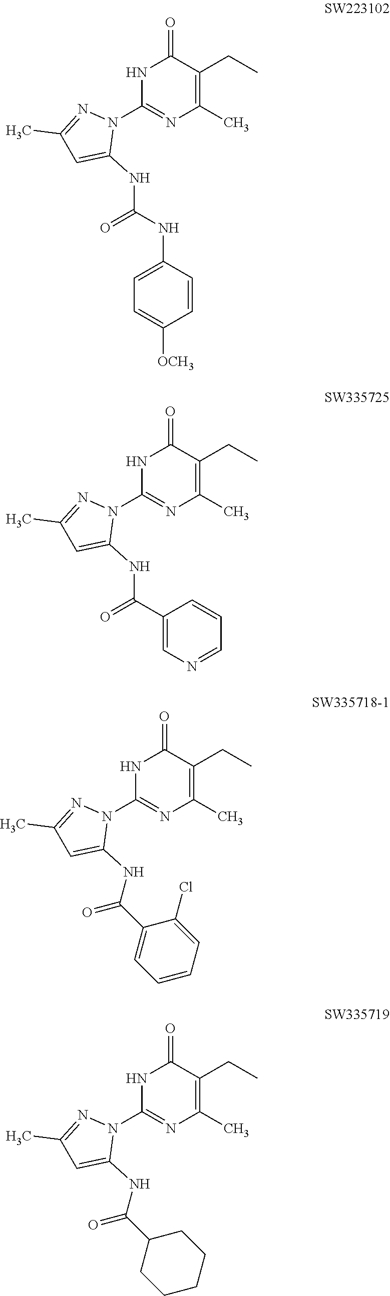

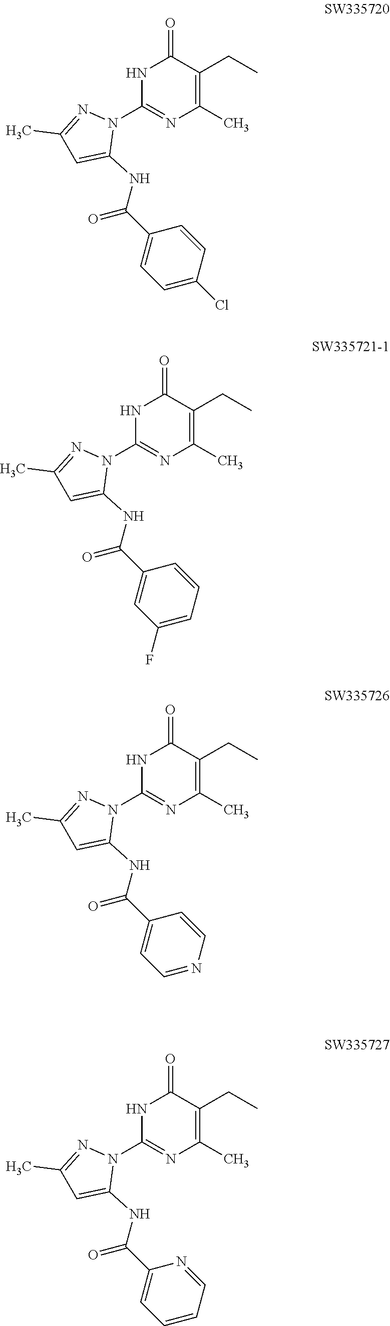

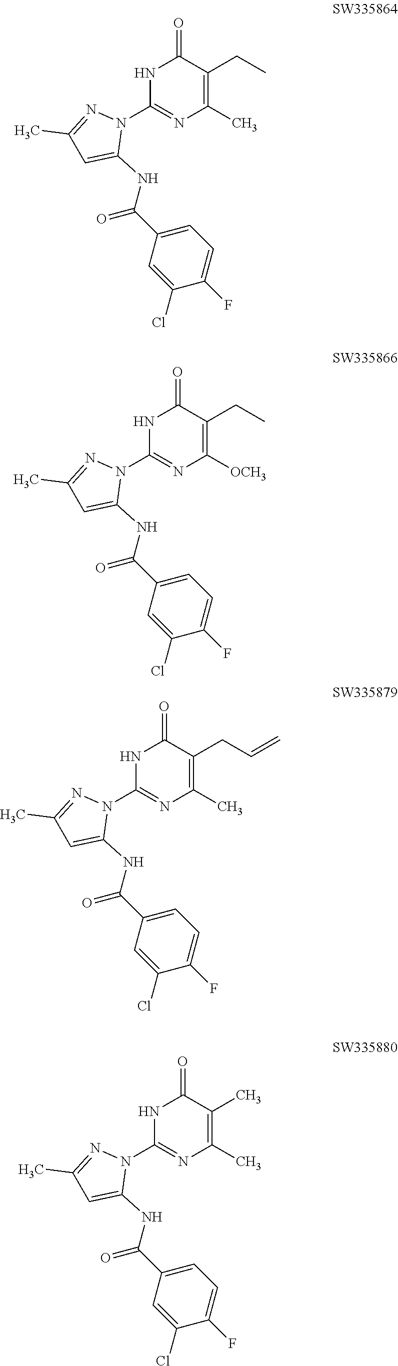

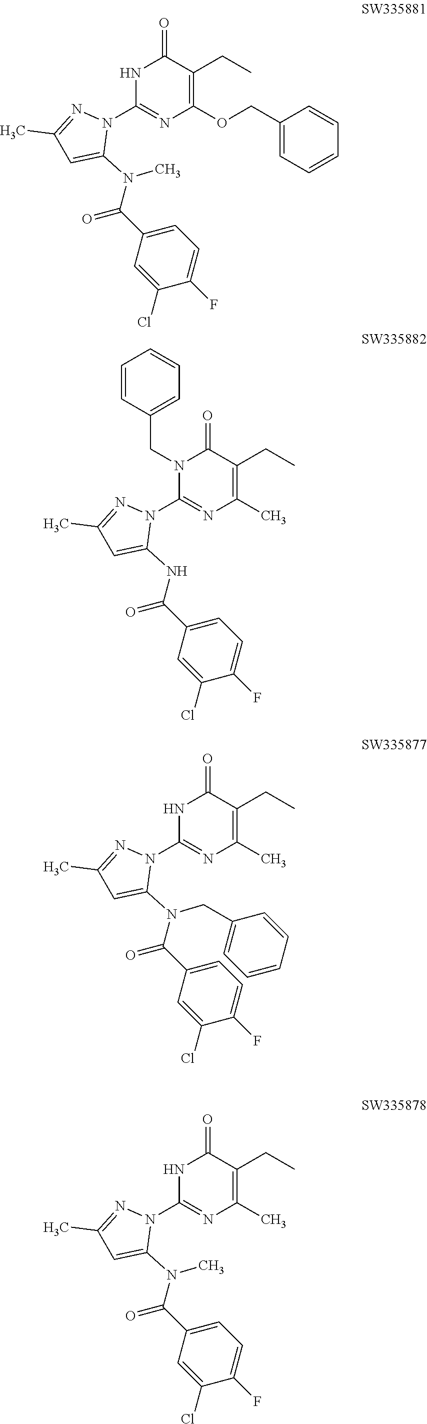

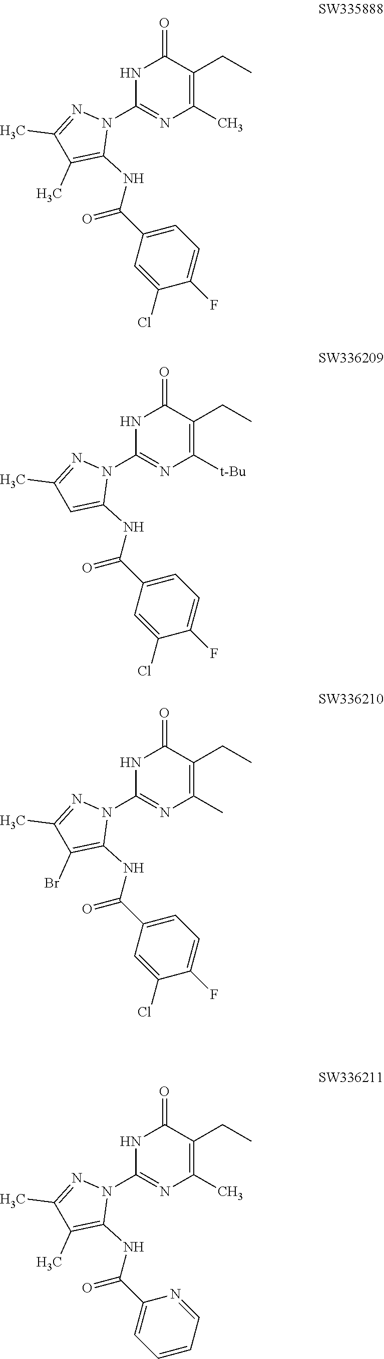

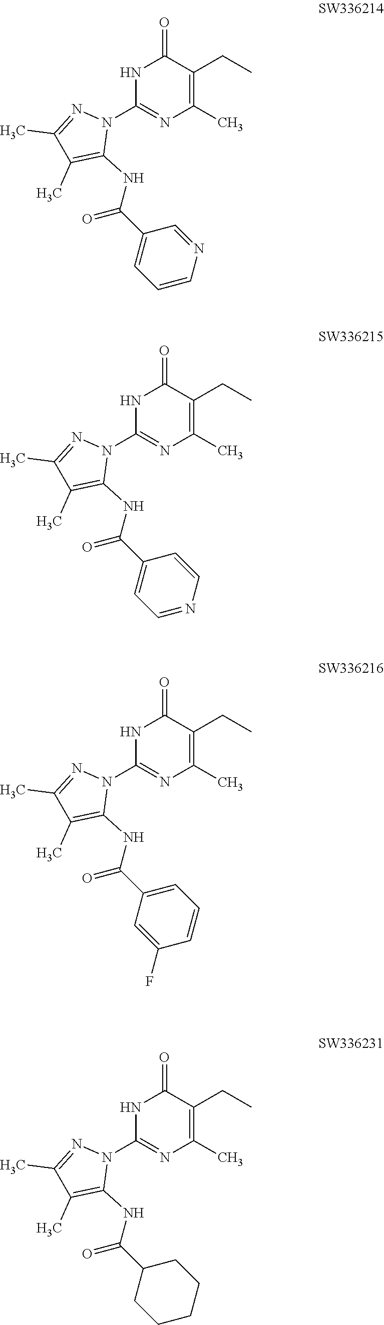

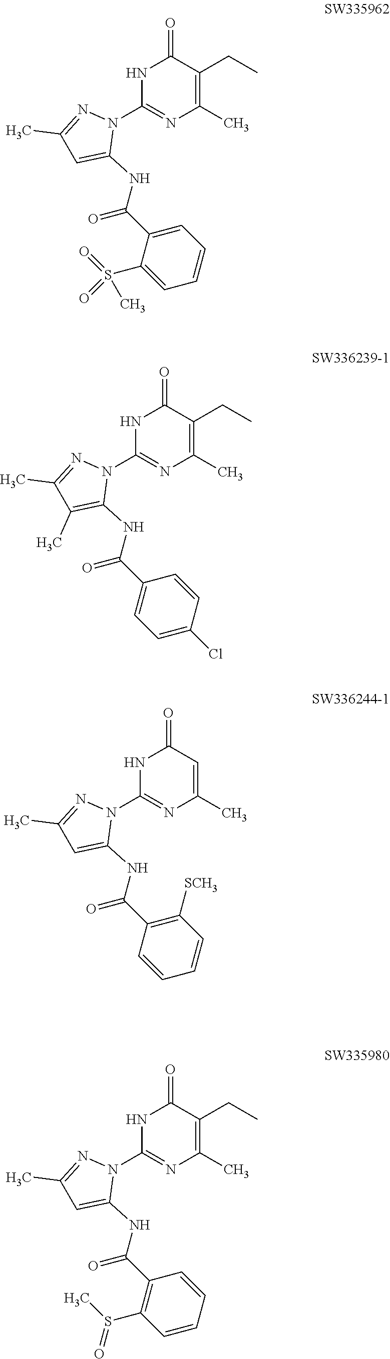

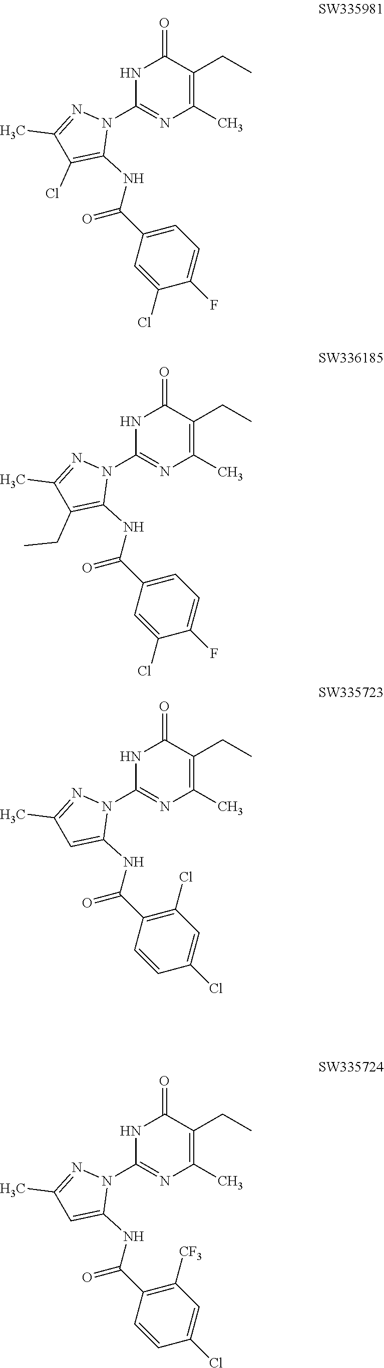

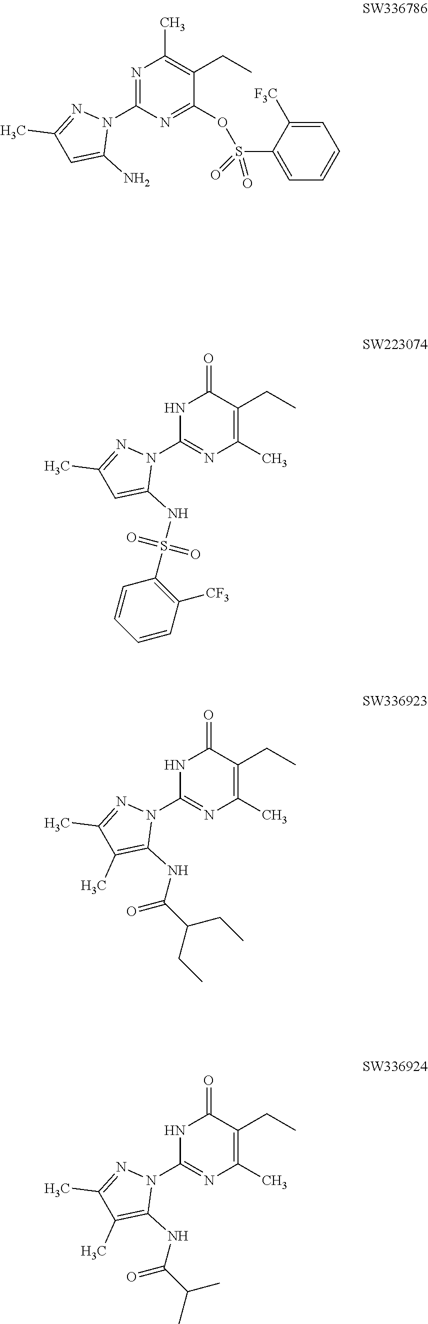

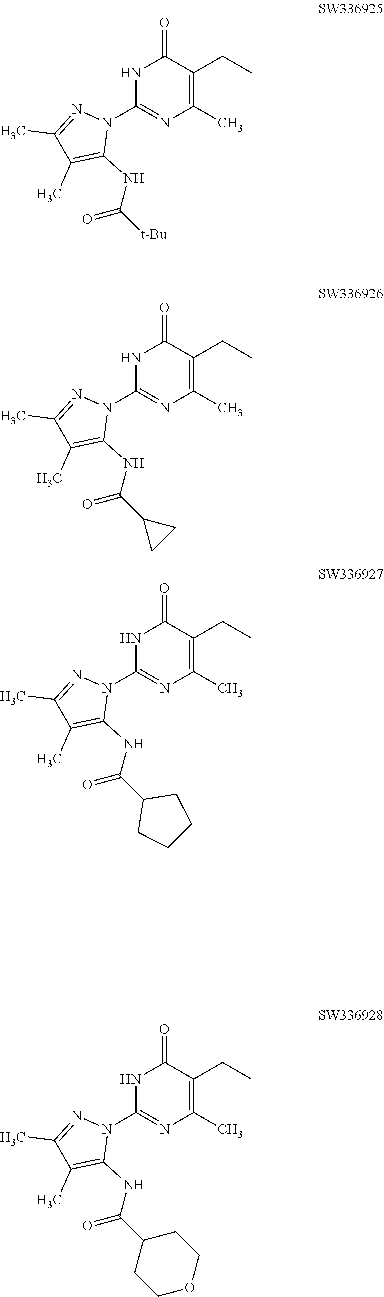

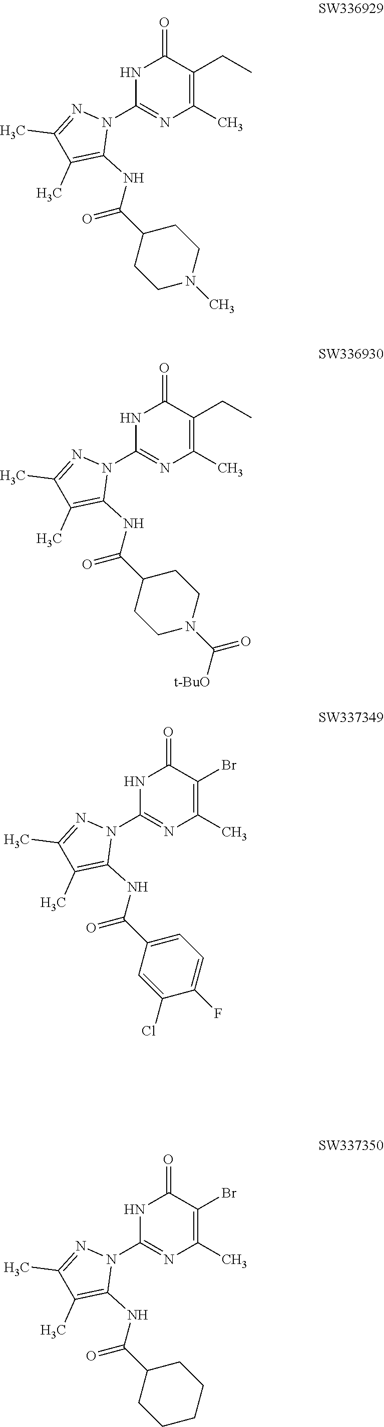

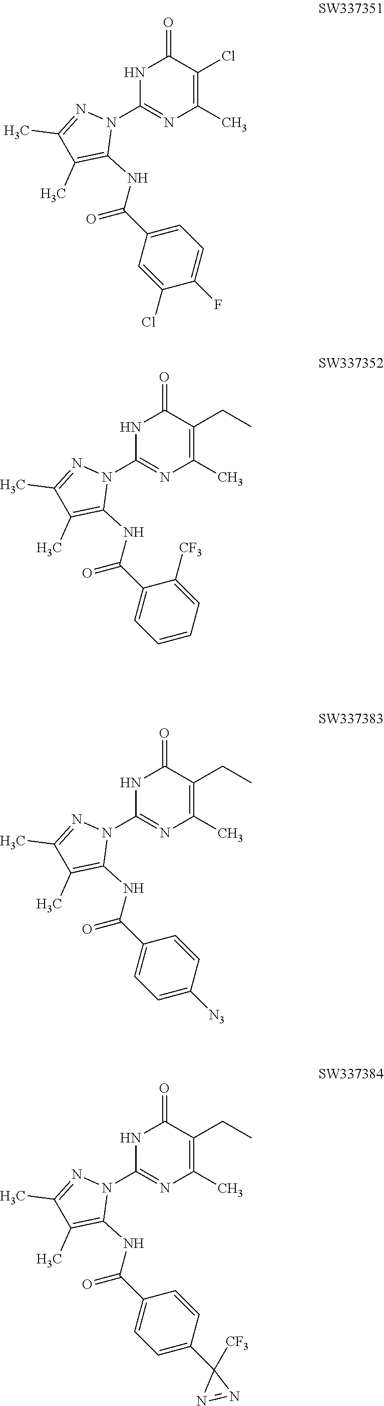

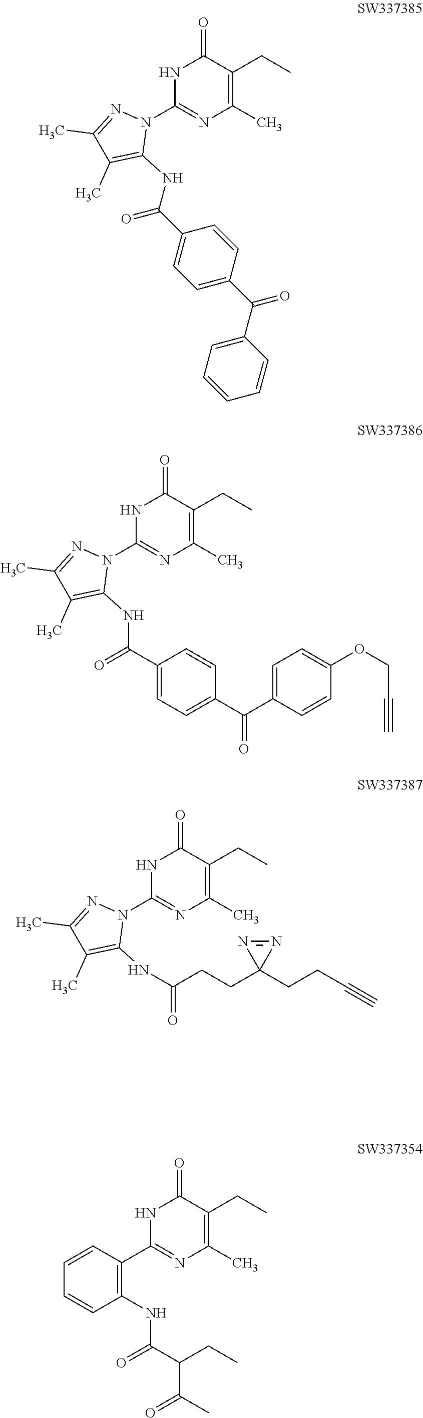

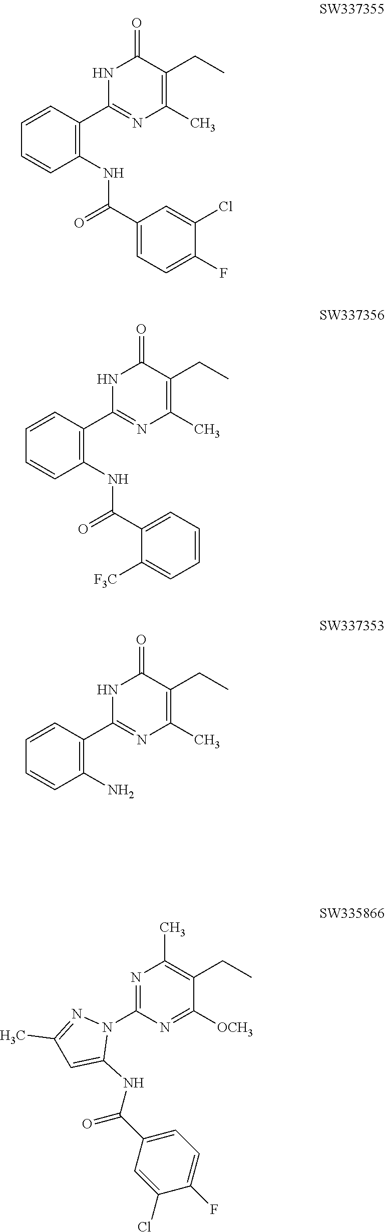

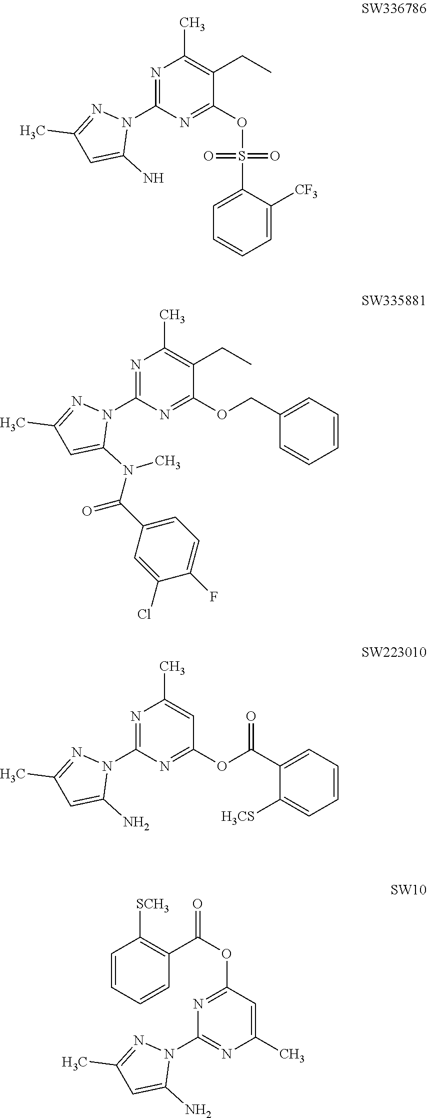

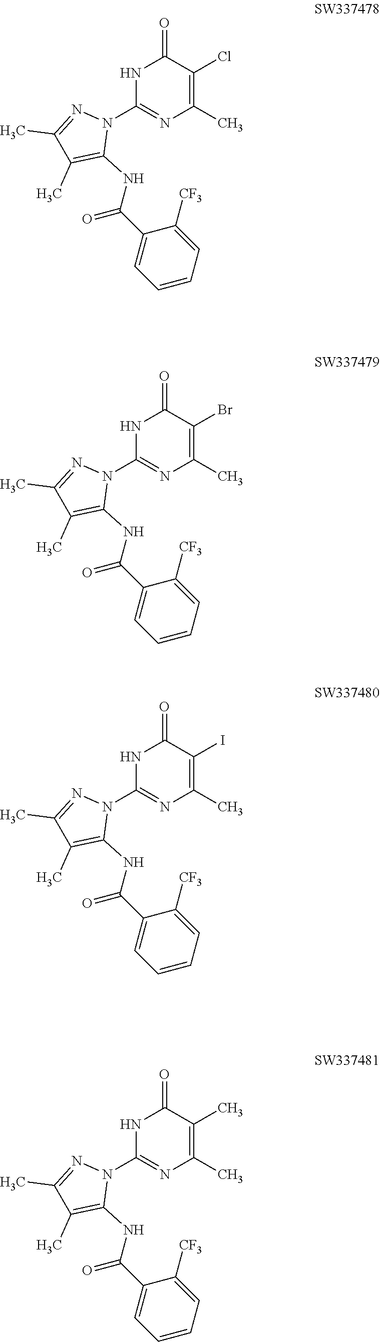

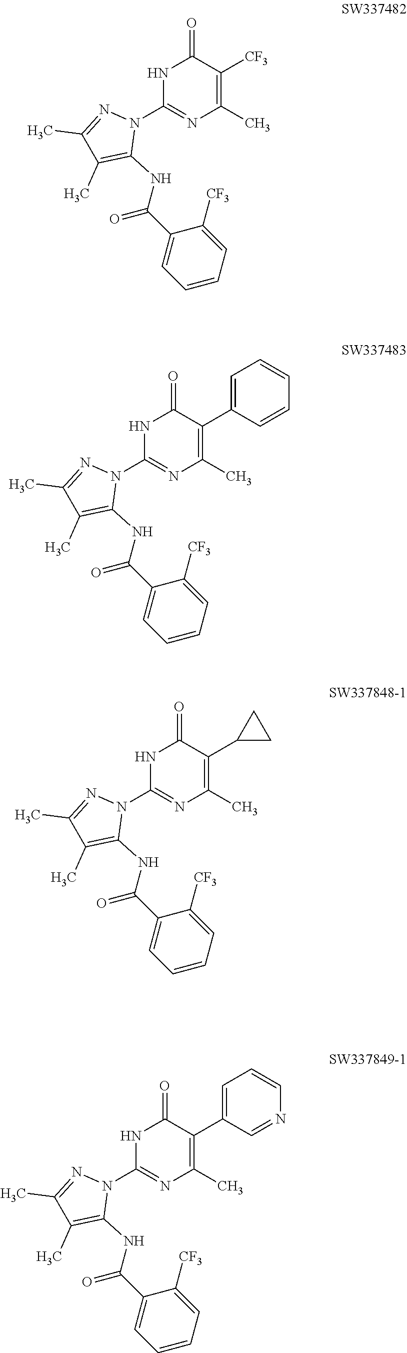 [SMILES: Cc1nn(-c2nc(C)c(C(F)(F)F)c(=O)[nH]2)c(NC(=O)c2ccccc2C(F)(F)F)c1C ; Cc1nn(-c2nc(C)c(-c3ccccc3)c(=O)[nH]2)c(NC(=O)c2ccccc2C(F)(F)F)c1C ; Cc1nn(-c2nc(C)c(C3CC3)c(=O)[nH]2)c(NC(=O)c2ccccc2C(F)(F)F)c1C ; Cc1nn(-c2nc(C)c(-c3cccnc3)c(=O)[nH]2)c(NC(=O)c2ccccc2C(F)(F)F)c1C]

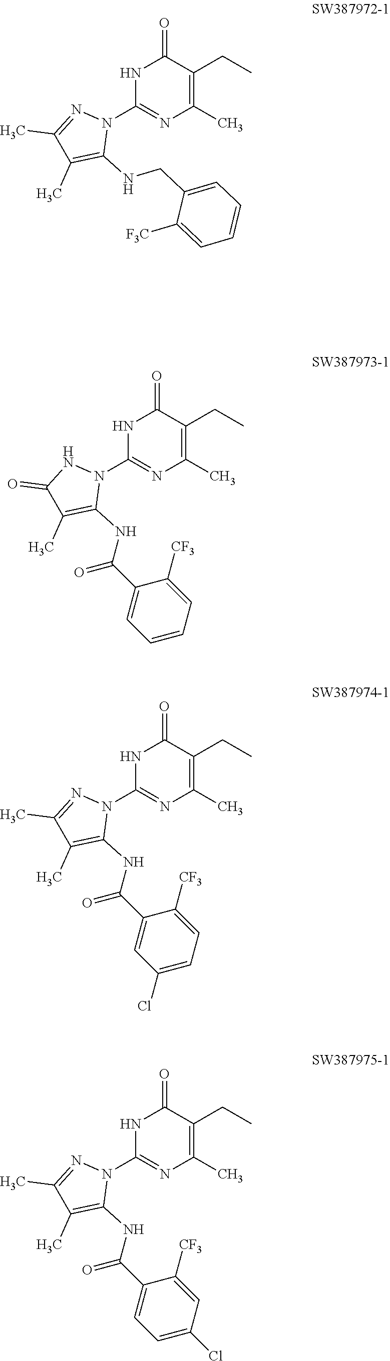

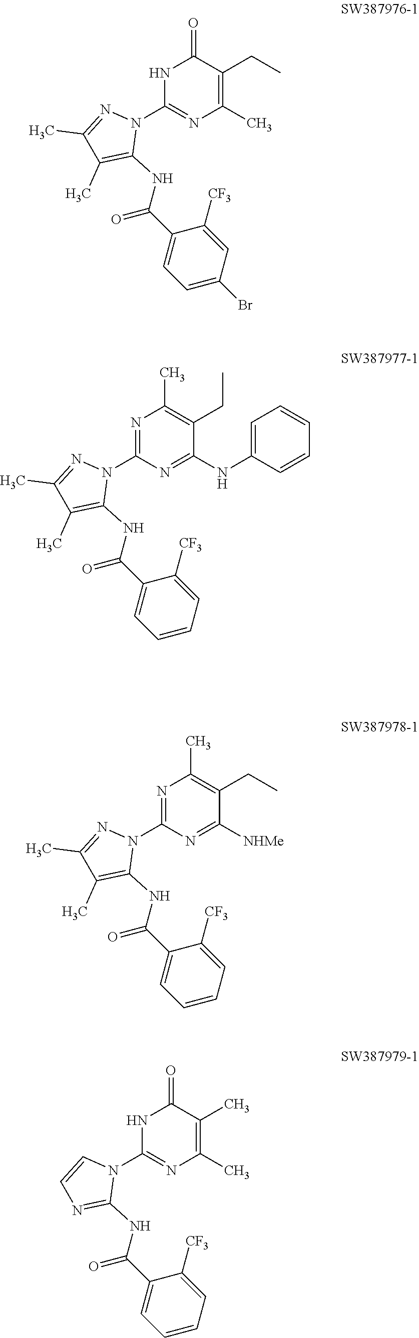

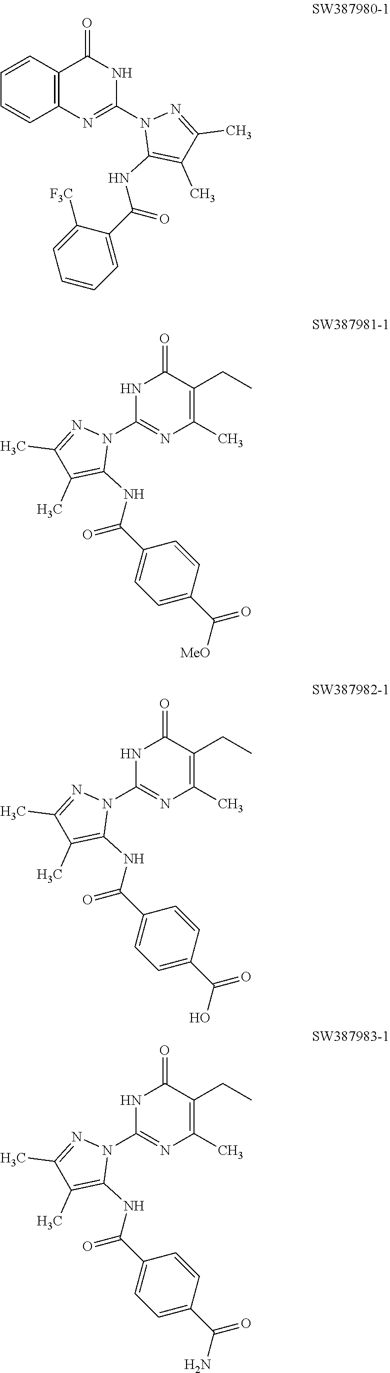

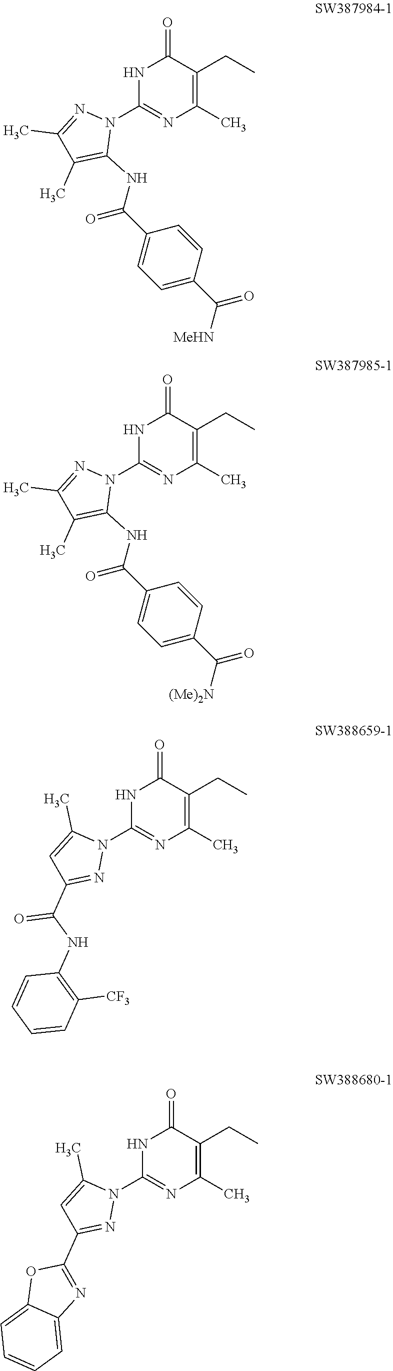

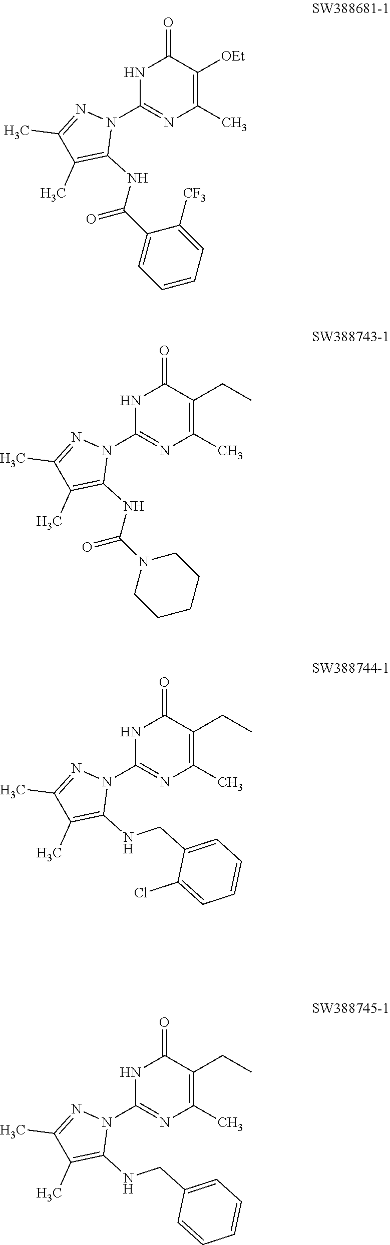

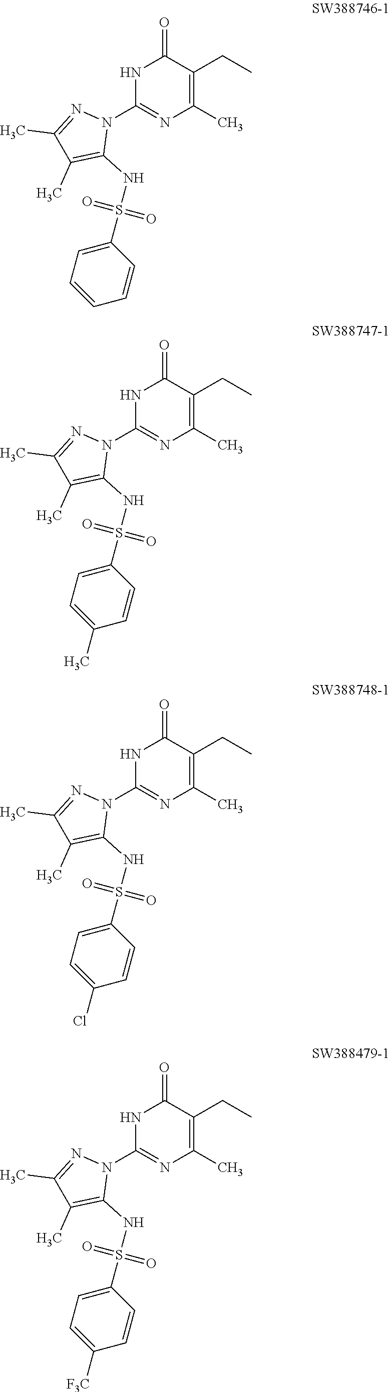

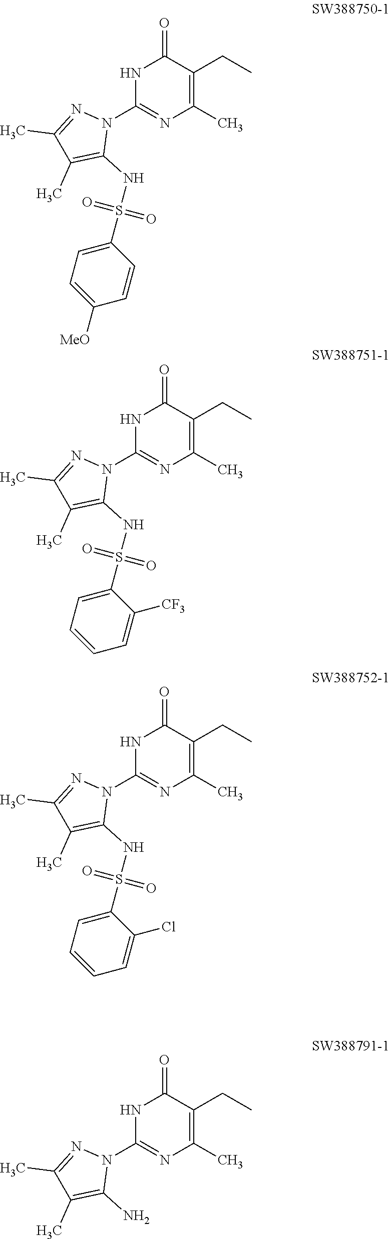

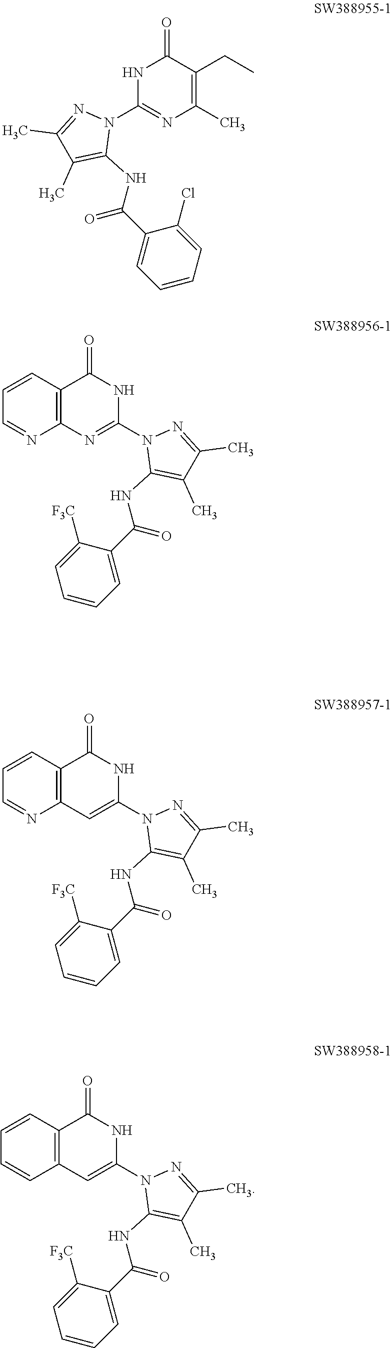 [SMILES: CCc1c(C)nc(-n2nc(C)c(C)c2NC(=O)c2ccccc2Cl)[nH]c1=O ; Cc1nn(-c2nc3ncccc3c(=O)[nH]2)c(NC(=O)c2ccccc2C(F)(F)F)c1C ; Cc1nn(-c2cc3ncccc3c(=O)[nH]2)c(NC(=O)c2ccccc2C(F)(F)F)c1C ; Cc1nn(-c2cc3ccccc3c(=O)[nH]2)c(NC(=O)c2ccccc2C(F)(F)F)c1C]

Other aspects of the invention are discussed throughout this application. Any aspects or embodiment discussed with respect to one aspect applies to other aspects as well and vice versa. Each aspects described herein is understood to be aspects that are applicable to all aspects. It is contemplated that any aspects discussed herein can be implemented with respect to any method or composition, and vice versa. Furthermore, compositions and kits can be used to achieve methods disclosed herein. It is contemplated that any one or more of these embodiments may be excluded in some embodiments.

The terms “effective amount” or “therapeutically effective amount” refer to that amount of a composition of the disclosure that is sufficient to effect treatment, as defined herein, when administered to a mammal in need of such treatment. This amount will vary depending upon the subject and disease condition being treated, the weight and age of the subject, the severity of the disease condition, the particular composition of the disclosure chosen, the dosing regimen to be followed, timing of administration, manner of administration and the like, all of which can readily be determined by one of ordinary skill in the art.

The “numerical values” and “ranges” provided for the various substituents are intended to encompass all integers within the recited range. For example, when defining n as an integer representing a value including from about 1 to 100, where the value typically encompasses the integer specified as n±10% (or for smaller integers from 1 to about 25, +3), it should be understood that n can be an integer from 1 to 100 (e.g., 1, 2, 3, 4, 5, 6, 7, 8, 9, 10, 11, 12, 13, 14, 15, 16, 17, 18, 19, 20, 22, 25, 30, 34, 35, 37, 40, 41, 45, 50, 54, 55, 59, 60, 65, 70, 75, 80, 82, 83, 85, 88, 90, 95, 99, 100, 105 or 110, or any between those listed). The combined terms “about” and “±10%” or “+3” should be understood to disclose and provide specific support for equivalent ranges wherever used.

The term “optional” or “optionally” means that the subsequently described event or circumstance may or may not occur, and that the description includes instances where said event or circumstance occurs and instances in which it does not.

As used herein, “pharmaceutically acceptable carrier” or “pharmaceutically acceptable excipient” includes any and all solvents, dispersion media, coatings, antibacterial and antifungal agents, isotonic and absorption delaying agents and the like. In several embodiments, these media and agents can be used in combination with pharmaceutically active substances. Except insofar as any conventional media or agent is incompatible with the active ingredient, its use in the therapeutic compositions is contemplated. Supplementary active ingredients can also be incorporated into the compositions.

The term “treatment” or “treating” means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms.

The use of the word “a” or “an” when used in conjunction with the term “comprising” in the claims and/or the specification may mean “one,” but it is also consistent with the meaning of “one or more,” “at least one,” and “one or more than one.” Throughout this application, the term “about” is used to indicate that a value includes the standard deviation of error for the device and/or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.” Reduction in the population of intracellular or axenic amastigotes is a reduction in response to administration of one or more anti-leishmanial compounds. The reduction in population is reported as the population of intracellular or axenic amastigotes subsequent to anti-leishmanial compound administration (and after a period of time sufficient to allow the anti-leishmanial compound to elicit an effect) in comparison to the population of intracellular or axenic amastigotes prior to anti-leishmanial compound administration. The population of amastigotes can be evaluated in a number of ways known to those of skill in the art. For example, in the experiments used to create the graph in FIG. 1B, a luciferase assay was used to determine amastigote population. A reduction in the indicated life cycle stage for T. brucei, T. cruzi, and P. falciparum is defined in the same manner.

An amastigote is defined as the human life cycle stage of Leishmania and T. cruzi parasites. A “disease” is defined as a pathological condition of a body part, an organ, or a system resulting from any cause, such as infection, genetic defect, or environmental stress. In particular embodiments, the disease or condition is related to glaucoma.

“Prevention” and “preventing” are used according to their ordinary and plain meaning to mean “acting before” or such an act. In the context of a particular disease or health-related condition, those terms refer to administration or application of an agent, drug, or remedy to a subject or performance of a procedure or modality on a subject for the purpose of blocking the onset.

The terms “inhibit,” “inhibiting,” and “inhibition,” (and grammatical equivalents) are used according to their plain and ordinary meaning in the area of medicine and biology. In the context of a physiological phenomena, e.g., a symptom, in an untreated subject relative to a treated subject, these terms mean to limit, prevent, or block a biological/chemical reaction to achieve a reduction in the quantity and/or magnitude of the physiological phenomena in the treated subject as compared to a differentially treated subject (such as an untreated subject or a subject treated with a different dosage or mode of administration) by any amount that is detectable and/or recognized as clinically relevant by any medically trained personnel. In some embodiments, the quantity and/or magnitude of the physiological phenomena in the treated subject is about, at least about, or at most about 5, 10, 15, 20, 25, 30, 35, 40, 45, 50, 55, 60, 65, 70, 75, 80, 85, 90, 95, or 100% (or any range derivable therein) lower than the quantity and/or magnitude of the physiological phenomena in the differentially treated subject. Alternatively, in other embodiments, the quantity and/or magnitude of the physiological phenomena in the treated subject is about, at least about, or at most about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0 times (or any range derivable therein) lower than the quantity and/or magnitude of the physiological phenomena in the differentially treated subject.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Some aspects of the disclosure are directed towards the use of a composition as disclosed herein in any method disclosed herein. Some embodiments provide for the use of any composition disclosed herein for treating leishmaniasis or other diseases caused by protozoan parasites. It is specifically contemplated that any step or element of an embodiment may be implemented in the context of any other step(s) or element(s) of a different embodiment disclosed herein.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings illustrate by way of example and not limitation. For the sake of brevity and clarity, every feature of a given structure may not be labeled in every figure in which that structure appears. Identical reference numbers do not necessarily indicate an identical structure. Rather, the same reference number may be used to indicate a similar feature or a feature with similar functionality, as may non-identical reference numbers.

FIGS. 1A-1C Concentration-response curves for two MMV Pathogen Box compounds for L. amazonesis axenic and intracellular amastigotes, compared to amphotericin. Log concentration-response curves using alamarBlue (FIG. 1A) and luciferase (FIG. 1B) for MMV676412 (closed squares), MMV676477 (open squares) and amphotericin B (closed triangles in gray) for axenic amastigotes. FIG. 1C Log concentration-response curves for the indicated compounds in L. amazonesis intracellular amastigotes. Data represent the means of three biological replicates, with SD indicated by error bars.

FIGS. 2A-2C Comparison of MMV676477 and its structural analog SW223041's potency against L. amazonesis axenic and intracellular amastigotes and L. donovani axenic amastigotes. Log concentration-response curves for MMV676477 and SW223041 against axenic (FIG. 2A) and intracellular amastigotes of L. amazonesis (FIG. 2B) and axenic amastigotes of L. donovani (FIG. 2C). The arrow shows a shift of the curve towards the left, indicating improved potency of SW223041. Data represent the means of three biological replicates, with SD indicated by error bars.

FIGS. 3A-3C Effect of MMV676477 and analogs on cell division in promastigotes and amastigotes of Leishmania amazonensis. FIG. 3A Exemplar fluorescence microscopy images of promastigotes affected in cell division treated with paclitaxel or MMV676477 for 48 h at the EC₅₀ 72 h concentration. Promastigotes were labeled with anti-α-tubulin antibody (green), anti-gp46 (membrane protein, red), and DAPI (nuclei, blue). Quantification of antimitotic effects by microscopic analysis for miltefosine, paclitaxel, MMV676477 and analog treatment of promastigotes (FIG. 3B) and amastigotes (FIG. 3C). Parasites were labeled as in FIG. 3A except that anti-p8 was used as a membrane protein marker for amastigotes. At least 200 parasites were analyzed per condition per experiment for three independent biological replicates (mean+/−SE). *p<0.05 by ANOVA compared to control conditions.

FIGS. 4A-4I Effect of MMV676477 and analogs on microtubule polymerization in L. amazonesis axenic amastigotes. FIG. 4A L. amazonesis axenic amastigotes were treated with the indicated compounds for 24 h at their EC₅₀ 72 h. Dimeric (unpolymerized, supernatant) and polymeric (polymerized, pellet) tubulin were separated by differential centrifugation and subjected to western blotting using α-tubulin antibody. GAPDH was used as a loading control.

FIG. 4B Densiometric analyses of western blot band intensity from three independent biological replicates+SE. *, #p<0.05 by ANOVA compared to control conditions. FIG. 4C Exemplar confocal immunofluorescence microscopy images for promastigotes treated with DMSO, miltefosine, paclitaxel and MMV676477 at their respective EC₅₀ 72 h concentrations for 24 h. Parasites were labeled with α-tubulin antibody and gp46 for a labeling intensity control. Tubulin intensity in promastigotes FIG. 4D and amastigotes FIG. 4E was quantified using densitometric analysis and normalized to that of the membrane control. Each data point represents mean+SE from three independent biological replicates. Parasites were labeled as in C except that anti-p8 was used as a membrane protein marker for amastigotes. At least 50 parasites per condition per experiment were counted (n=150 parasites). * denotes groups that are significantly brighter than the untreated control, p<0.05, by ANOVA. FIG. 4F Exemplary confocal fluorescence microscopy images demonstrating flagella length in promastigotes treated for 24 h at the EC₅₀^{72 h} concentration with DMSO (control), miltefosine (MLTF), paclitaxel (PTXL) or MMV676477. Promastigotes were labeled with anti-α-tubulin monoclonal antibody YL1/2 (Invitrogen, catalog #MA1-80017) and anti-gp46 (membrane protein). Scale bar=10 m. FIG. 4G Mean flagellar length for compounds. At least 50 parasites were analyzed per condition for three independent biological replicates (mean±SE). *p<0.05 by ANOVA compared to DMSO control. FIG. 4H Exemplary confocal fluorescence microscopy images of pear-shaped or rounded promastigotes treated for 48 h at the EC₅₀^{72 h} concentration with paclitaxel or MMV676477, compared to more typically-shaped promastigotes treated with DMSO (control) and miltefosine. DAPI was also used to label parasite nuclei and kinetoplasts. Scale bar=10 m. FIG. 4I Percentages of pear-shaped or rounded promastigotes, defined as having a cell body with a width of ≥70% of their length. At least 200 parasites were analyzed per condition per experiment for three independent biological replicates (mean±SE). *p<0.05 by ANOVA compared to control conditions.

FIGS. 5A-5B Activity of MMV676477 against purified Leishmania tubulin. FIG. 5A SDS-PAGE gel showing purification of assembly-competent L. tarentolae tubulin by ion exchange chromatography. L. tarentolae lysate (1) was centrifuged (pellet, 2; supernatant, 3), filtered (4), loaded on a DEAE-sepharose column (flow through, 5), and washed (6). Tubulin was eluted with high salt (7, 8). FIG. 5B Representative turbidity curves for MMV676477-treated purified Leishmania tubulin (3 mg/ml tubulin, 1% DMSO). For Leishmania tubulin: MMV676477 EC₅₀=0.5+0.1 μM; paclitaxel EC₅₀=1.3+0.1 μM; A340 10% DMSO=0.4.

FIGS. 6A-6C Comparison between tubulin polymerization and antiparasitic activity for MMV676477 and analogs. FIG. 6A Correlation among MMV676477 analogs between purified L. tarentolae (3 mg/mL) tubulin polymerization activity at 1 μM and 1 μM. FIG. 6B Correlation among MMV676477 analogs between purified L. tarentolae tubulin polymerization activity at 10 μM and antiparasitic EC₅₀72 h data for L. amazonensis axenic amastigotes. FIG. 6C Correlation among MMV676477 analogs between purified L. tarentolae tubulin polymerization activity at 1 μM and antiparasitic EC₅₀72 h data for L. amazonensis axenic amastigotes.

FIGS. 7A-7B Effect of MMV676477 in regulation of mammalian microtubule assembly in vitro. Representative turbidity curves (all concentrations in M) for purified mammalian tubulin (3 mg/ml tubulin, 1% DMSO) treated with MMV676477 (FIG. 7A) and paclitaxel (FIG. 7B). For mammalian tubulin: Paclitaxel EC₅₀=1.5±0.2 μM; MMV676477 EC₅₀=11+3.2 μM. Maximum absorbance (A340, 10% DMSO)=0.36 for paclitaxel and 0.4 for MMV676477.

FIGS. 8A-8B Competition-sensitive fluorescent binding of MMV676477 analogs to tubulin. Following treatment with the SW223022 probe (“P”) in the presence or absence of competitors (“C”), purified L. tarentolae tubulin was subjected to UV crosslinking. Alexa Fluor 532 azide dye was then conjugated to the probe via copper-assisted cycloaddition (CuAAC; “click chemistry”). FIG. 8A Fluorescence imaging of dye-labeled tubulin samples, +competition by MMV676477 and a panel of analogs of varying potency. Competing compounds were added at 100× the probe concentration (1× probe EC₅₀=156 nM; competitors added at 15.6 μM). FIG. 8B Coomassie blue staining of the gel in (A) is used as a loading control.

FIG. 9 The unrelated antileishmanial drug miltefosine has no known activity on tubulin and did not affect tubulin polymerization at 50 μM.

FIG. 10 Tubulin polymerization activity by MMV676477 was not affected by 0.01% Triton-X treatment, suggesting that MMV676477's activity was not merely aggregation-based.

FIG. 11 A table of the synthesized compounds activities half-maximal effective concentrations (EC₅₀) and macrophage toxicities (CC₅₀) against L. amazonensis axenic amastigotes and promastigotes.

FIG. 12 A table of macrophage toxicities (CC₅₀) and/or half-maximal inhibitory concentrations (IC₅₀) against L. amazonensis axenic and intracellular amastigotes, promastigotes, and T. brucei trypomastigotes.

FIG. 13 A table of macrophage toxicities (CC₅₀) and/or half-maximal inhibitory concentrations (IC₅₀) against L. amazonensis axenic and intracellular amastigotes, promastigotes, and T. brucei trypomastigotes.

FIG. 14 A table of half-maximal inhibitory concentrations (IC₅₀) against T. brucei trypomastigotes.

FIG. 15 A table of half-maximal inhibitory concentrations (EC₅₀) against various parasites.

FIG. 16 A table of half-maximal inhibitory concentrations (IC₅₀) against T. brucei and L. amazonensis and macrophage toxicities (CC₅₀).

FIG. 17 A table of half-maximal inhibitory concentrations (IC₅₀) against T. brucei, L. amazonensis, and T. cruzi trypomastigotes and macrophage toxicities (CC₅₀).

FIG. 18 A table of compounds screened against L. amazonensis axenic amastigotes at 5 μM and 1 μM. Relative fluorescence intensity (%) is shown. Lower values represent more potent compounds.

FIG. 19 A table that includes cytotoxicity data (CC₅₀ values) for selected compounds. Values are mean CC₅₀^{72 h} values (nm) calculated from three biological replicates±SE.

### DETAILED DESCRIPTION

Various features and advantageous details are explained more fully with reference to the non-limiting embodiments that are illustrated in the accompanying drawings and detailed in the following description. It should be understood, however, that the detailed description and the specific examples, while indicating embodiments of the invention, are given by way of illustration only, and not by way of limitation. Various substitutions, modifications, additions, and/or rearrangements will become apparent to those of ordinary skill in the art from this disclosure.

In the following description, numerous specific details are provided to provide a thorough understanding of the disclosed embodiments. One of ordinary skill in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

A library of compounds (Pathogen Box) were tested for activity against L. amazonensis axenic amastigotes. This yielded six discovery antileishmanials with EC₅₀ values ranging from 50 to 480 nM. The top hit, MMV676477, also kills intracellular Leishmania spp, T. brucei, and P. falciparum at nanomolar concentrations. An initial structure-activity relationship (SAR) study was performed to optimize the MMV676477 scaffold. Using assays in intact parasites and with purified proteins, molecules derived from the scaffold were found to affect Leishmania cell division and selectively facilitate microtubule polymerization within Leishmania parasites rather than mammalian cells. This work demonstrates that MMV676477 is a potent antiparasitic compound that preferentially promotes Leishmania microtubule polymerization, and that the pharmacophore can serve as a scaffold for additional anti-leishmanial compounds. Since MMV676477 has activity against multiple parasites, this scaffold shows promise for future antiparasitic drug development.

### Chemical Definitions

As used herein, the structure [symbol] indicates that the bond may be a single bond or a double bond. Those of skill in the chemical arts understand that in certain circumstances, a double bond between two particular atoms is chemically feasible and in certain circumstances, a double bond is not chemically feasible. The present invention therefore contemplates that a double bond may be formed only when chemically feasible.

The term “alkyl” includes straight-chain alkyl, branched-chain alkyl, cycloalkyl (alicyclic), cyclic alkyl, heteroatom-unsubstituted alkyl, heteroatom-substituted alkyl, heteroatom-unsubstituted Cn-alkyl, and heteroatom-substituted Cn-alkyl. Specifically included within the definition of “alkyl” are those alkyl groups that are optionally substituted. In certain embodiments, lower alkyls are contemplated. The term “lower alkyl” refers to alkyls of 1-6 carbon atoms (that is, 1, 2, 3, 4, 5 or 6 carbon atoms). The term “heteroatom-unsubstituted Cn-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cn-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CH₂F, —CH₂Cl, —CH₂Br, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The term “heteroalkyl,” by itself or in combination with another term, means, unless otherwise stated, a linear or branched chain having at least one carbon atom and at least one heteroatom selected from the group consisting of O, N, S, P, and Si. In certain embodiments, the heteroatoms are selected from the group consisting of 0 and N. The heteroatom(s) may be placed at any interior position of the heteroalkyl group or at the position at which the alkyl group is attached to the remainder of the molecule. Up to two heteroatoms may be consecutive.

The following groups are all non-limiting examples of heteroalkyl groups: trifluoromethyl, —CH₂F, —CH₂Cl, —CH₂Br, —CH₂OH, —CH₂OCH₃, —CH₂OCH₂, CF₃, —CH₂OC(O)CH₃, —CH₂NH₂, —CH₂NHCH₃, —CH₂N(CH₃)₂, —CH₂CH₂Cl, —CH₂CH₂OH, —CH₂CH₂OC(O)CH₃, —CH₂CH₂NHCO₂C(CH₃)₃, and —CH₂Si(CH₃)₃.

The term “heterocycle” refers to a fully saturated monocyclic, bicyclic, tricyclic or other polycyclic ring system having one or more constituent heteroatom ring atoms independently selected from O, N (it is understood that one or two additional groups may be present to complete the nitrogen valence and/or form a salt), or S. Specifically included within the definition of “heterocycle” are those heterocycle groups that are optionally substituted. The heteroatom or ring carbon can be the point of attachment of the heterocyclyl substituent to another moiety. Any atom can be optionally substituted, e.g., by one or more substituents. Heterocycle groups can include, e.g., tetrahydrofuryl, tetrahydropyranyl, piperidyl (piperidino), piperazinyl, morpholinyl (morpholino), pyrrolinyl, and pyrrolidinyl. By way of example, the phrase “heterocyclic ring containing from 5-6 ring atoms, wherein 1-2 of the ring atoms is independently selected from N, NH, N(C₁-C₆ alkyl), NC(O)(C₁-C₆ alkyl), 0, and S; and wherein said heterocyclic ring is optionally substituted with 1-3 independently selected Ra” would include (but not be limited to) tetrahydrofuryl, tetrahydropyranyl, piperidyl (piperidino), piperazinyl, morpholinyl (morpholino), pyrrolinyl, and pyrrolidinyl.

The terms “cycloalkyl” and “heterocyclyl,” by themselves or in combination with other terms, means cyclic versions of “alkyl” and “heteroalkyl”, respectively. Additionally, for heterocyclyl, a heteroatom can occupy the position at which the heterocycle is attached to the remainder of the molecule. Specifically included within the definition of “cycloalkyl” are those cycloalkyl groups that are optionally substituted. Specifically included within the definition of “heterocyclyl” are those heterocycle groups that are optionally substituted.

The term “aryl” includes heteroatom-unsubstituted aryl, heteroatom-substituted aryl, heteroatom-unsubstituted Cn-aryl, heteroatom-substituted Cn-aryl, heteroaryl, heterocyclic aryl groups, carbocyclic aryl groups, biaryl groups, and single-valent radicals derived from polycyclic fused hydrocarbons (PAHs). Specifically included within the definition of “aryl” are those aryl groups that are optionally substituted. The term “heteroatom-unsubstituted Cn-aryl” refers to a radical, having a single carbon atom as a point of attachment, wherein the carbon atom is part of an aromatic ring structure containing only carbon atoms, further having a total of n carbon atoms, 5 or more hydrogen atoms, and no heteroatoms. For example, a heteroatom-unsubstituted C₆-C₁₀-aryl has 6 to 10 carbon atoms. Non-limiting examples of heteroatom-unsubstituted aryl groups include phenyl (Ph), methylphenyl, (dimethyl)phenyl, —C₆H₄CH₂CH₃, —C₆H₄CH₂CH₂CH₃, —C₆H₄CH(CH₃)₂, —C₆H₄CH(CH₂)₂, —C₆H₃(CH₃)CH₂CH₃, —C₆H₄CH═CH₂, —C₆H₄CH≡CHCH₃, —C₆H₄C≡CH, —C₆H₄C≡CCH₃, naphthyl, and the radical derived from biphenyl. The term “heteroatom-substituted Cn-aryl” refers to a radical, having either a single aromatic carbon atom or a single aromatic heteroatom as the point of attachment, further having a total of n carbon atoms, at least one hydrogen atom, and at least one heteroatom, further wherein each heteroatom is independently selected from the group consisting of N, O, F, Cl, Br, I, Si, P, and S. For example, a heteroatom-unsubstituted C₁-C₁₀-heteroaryl has 1 to 10 carbon atoms. Non-limiting examples of heteroatom-substituted aryl groups include the groups: —C₆H₄F, —C₆H₄C₁, —C₆H₄Br, —C₆H₄I, —C₆H₄₀H, —C₆H₄₀CH₃, —C₆H₄₀CH₂CH₃, —C₆H₄₀C(O)CH₃, —C₆H₄NH₂, —C₆H₄NHCH₃, —C₆H₄N(CH₃)₂, —C₆H₄CH₂OH, —C₆H₄CH₂OC(O)CH₃, —C₆H₄CH₂NH₂, —C₆H₄CF₃, —C₆H₄CN, —C₆H₄CHO, —C₆H₄CHO, —C₆H₄C(O)CH₃, —C₆H₄C(O)C₆H₅, —C₆H₄CO₂H, —C₆H₄CO₂CH₃, —C₆H₄CONH₂, —C₆H₄CONHCH₃, —C₆H₄CON(CH₃)₂, furanyl, thienyl, pyridyl, pyrrolyl, pyrimidyl, pyrazinyl, quinolyl, indolyl, and imidazoyl. In certain embodiments, heteroatom-substituted aryl groups are contemplated. In certain embodiments, heteroatom-unsubstituted aryl groups are contemplated. In certain embodiments, an aryl group may be mono-, di-, tri-, tetra- or penta-substituted with one or more heteroatom-containing substituents.

The term “alkoxy” includes straight-chain alkoxy, branched-chain alkoxy, cycloalkoxy, cyclic alkoxy, heteroatom-unsubstituted alkoxy, heteroatom-substituted alkoxy, heteroatom-unsubstituted Cn-alkoxy, and heteroatom-substituted Cn-alkoxy. Specifically included within the definition of “alkoxy” are those alkoxy groups that are optionally substituted. In certain embodiments, lower alkoxys are contemplated. The term “lower alkoxy” refers to alkoxys of 1-6 carbon atoms (that is, 1, 2, 3, 4, 5 or 6 carbon atoms). The term “heteroatom-unsubstituted Cn-alkoxy” refers to a group, having the structure —OR, in which R is a heteroatom-unsubstituted Cn-alkyl, as that term is defined above. Heteroatom-unsubstituted alkoxy groups include: —OCH₃, —OCH₂CH₃, —OCH₂CH₂CH₃, —OCH(CH₃)₂, and —OCH(CH₂)₂. The term “heteroatom-substituted Cn-alkoxy” refers to a group, having the structure —OR, in which R is a heteroatom-substituted Cn-alkyl, as that term is defined above. For example, —OCH₂CF₃ is a heteroatom-substituted alkoxy group.

Various groups, including alkyl, heteroalky, cycloalkyl, heterocyclyl, aryl, heteroaryl, and alkoxy, are described herein as substituted or unsubstituted (i.e., optionally substituted). Optionally substituted groups may include one or more substituents independently selected from: halogen, nitro, cyano, hydroxy, amino, mercapto, thioalkyl, formyl, acetyl, carboxy, oxo, carbamoyl, substituted or unsubstituted alkyl, substituted or unsubstituted heteroalkyl, alkoxy, alkylthio, alkylamino, (alkyl)₂amino, alkylsulfinyl, alkylsulfonyl, arylsulfonyl, substituted or unsubstituted cycloalkyl, substituted or unsubstituted heterocyclyl, substituted or unsubstituted aryl, substituted or unsubstituted heteroaryl, cyclopropyl, tetrahydropyranyl, piperidinyl, tert-butyl carbamoyl, benzoyl, aziridinyl, diazirinyl, azide, oxobutanamide, and propargyl. In certain aspects the optional substituents may be further substituted with one or more substituents independently selected from: halogen, nitro, cyano, hydroxy, amino, mercapto, formyl, carboxy, carbamoyl, unsubstituted alkyl, unsubstituted heteroalkyl, alkoxy, alkylthio, alkylamino, (alkyl)₂amino, alkylsulfinyl, alkylsulfonyl, arylsulfonyl, unsubstituted cycloalkyl, unsubstituted heterocyclyl, unsubstituted aryl, or unsubstituted heteroaryl. Exemplary optional substituents include, but are not limited to: —OH, oxo (═O), —Cl, —F, Br, C₁₋₄alkyl, phenyl, benzyl, —NH₂, —CF₃, —NH(C₁₋₄alkyl), —N(C₁₋₄alkyl)₂, —NO₂, —S(C₁₋₄alkyl), —SO₂(C₁₋₄alkyl), —CO₂(C₁₋₄alkyl), and —O(C₁₋₄alkyl).

The term “amino” means a group having the structure —NR′R″, where R′ and R″ are independently hydrogen or an optionally substituted alkyl, heteroalkyl, cycloalkyl, or heterocyclyl group. The term “amino” includes primary, secondary, and tertiary amines.

Embodiments are also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present invention are contemplated.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Non-limiting examples of inorganic acids which may be used to prepare pharmaceutically acceptable salts include: hydrochloric acid, phosphoric acid, sulfuric acid, hydrobromic acid, hydroiodic acid, phosphorous acid and the like. Examples of organic acids which may be used to prepare pharmaceutically acceptable salts include: aliphatic mono- and dicarboxylic acids, such as oxalic acid, carbonic acid, citric acid, succinic acid, phenyl-heteroatom-substituted alkanoic acids, aliphatic and aromatic sulfuric acids and the like. Pharmaceutically acceptable salts prepared from inorganic or organic acids thus include hydrochloride, hydrobromide, nitrate, sulfate, pyrosulfate, bisulfate, sulfite, bisulfate, phosphate, monohydrogenphosphate, dihydrogenphosphate, metaphosphate, pyrophosphate, hydroiodide, hydrofluoride, acetate, propionate, formate, oxalate, citrate, lactate, p-toluenesulfonate, methanesulfonate, maleate, and the like.

Suitable pharmaceutically acceptable salts may also be formed by reacting the agents of the invention with an organic base such as methylamine, ethylamine, ethanolamine, lysine, ornithine and the like.

Pharmaceutically acceptable salts include the salts formed between carboxylate or sulfonate groups found on some of the compounds of this invention and inorganic cations, such as sodium, potassium, ammonium, or calcium, or such organic cations as isopropylammonium, trimethylammonium, tetramethylammonium, and imidazolium.

Derivatives of compounds of the present invention are also contemplated. In certain aspects, “derivative” refers to a chemically modified compound that still retains the desired effects of the compound prior to the chemical modification. Such derivatives may have the addition, removal, or substitution of one or more chemical moieties on the parent molecule. Non-limiting examples of the types modifications that can be made to the compounds and structures disclosed herein include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, heteroatoms such as N, S, or O can be substituted into the structure instead of a carbon atom.

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

As noted above, compounds of the present invention may exist in prodrug form. As used herein, “prodrug” is intended to include any covalently bonded carriers which release the active parent drug or compounds that are metabolized in vivo to an active drug or other compounds employed in the methods of the invention in vivo when such prodrug is administered to a subject. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a subject, cleaves to form a free hydroxyl, free amino, or carboxylic acid, respectively. Other examples include, but are not limited to, acetate, formate, and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

Pharmaceutical Formulations and Routes of Administration

Pharmaceutical compositions are provided herein that comprise an effective amount of one or more substances and/or additional agents dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one substance or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The compounds of the invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, intramuscularly, systemically, subcutaneously, subconjunctival, intravesicularly, mucosally, intrapericardially, intraumbilically, intraocularly, orally, locally, via inhalation (e.g., aerosol inhalation), via injection, via infusion, via continuous infusion, via localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990).

The actual dosage amount of a composition administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of a compound described herein. In other embodiments, the compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above. An “effective” dose is defined as an amount sufficient to reduce parasite population in a treated subject as evidenced by either a reduction in the presentation of one or more symptoms or a reduced number of parasites as determined by a suitable assay.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal, or combinations thereof.

The substance may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine, or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. It may be preferable to include isotonic agents, such as, for example, sugars, sodium chloride or combinations thereof.

In other embodiments, one may use eye drops, nasal solutions or sprays, aerosols or inhalants. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in certain embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the substance is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. In certain embodiments, carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof, a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar, or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, certain methods of preparation may include vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein.

In particular embodiments, prolonged absorption of an injectable composition can be brought about by the use in the compositions of agents delaying absorption, such as, for example, aluminum monostearate, gelatin, or combinations thereof.

Anti-Parasitic Compound Identification and Mechanism of Action

The few current frontline anti-trypanosomatid drugs are poorly effective and extremely toxic to humans. To accelerate drug discovery, the Medicine for Malaria Venture (MMV) has coordinated screens of >5 million compounds against parasitic diseases, generating approximately 20,000 starting points for drug discovery and development. Previously, a representative set of 400 compounds, called the Malaria Box, made a significant impact beyond the malaria field and have stimulated medicinal chemistry efforts against many diseases, including leishmaniasis. Due to the success of the Malaria Box, the MMV distributed another collection of 400 drug-like compounds (likely to show acceptable oral absorption), called the Pathogen Box to target an expanded set of pathogens. Its name derives from the fact that each compound in the box has known activity against one or more pathogenic bacterial, fungal, or parasitic organisms. Known antiparasitic drugs and current antiparasitic lead compounds are also included. Similar to the Malaria Box, these compounds reflect a cross-section of the chemical diversity available in MMV's 20,000 hits, providing 374 starting points for oral drug discovery.

To search for new drugs for trypanosomatid diseases, compounds available in the Medicines for Malaria Venture “Pathogen Box” were tested against L. amazonensis axenic amastigotes using a viability assay. Preliminary screening yielded six discovery anti-leishmanials with EC₅₀ values ranging from 50 to 480 nM. Subsequent concentration-response luciferase-based assays demonstrated that the best hit, MMV676477, retains nanomolar-range cytocidal potency against intracellular Leishmania amastigotes, as well as Trypanosoma brucei and Plasmodium falciparum, suggesting broad antiparasitic activity. Structure-activity relationships (SAR) of a small group of MMV676477 analogs was explored against axenic Leishmania amastigotes and a wide potency range (20 to 5000 nM) was observed. Compared to MMV676477, the most potent compound, SW223041, kills L. amazonensis and L. donovani with four to five-fold improved potency. Multiple lines of evidence suggest that MMV676477 selectively disrupts Leishmania tubulin dynamics. Initial morphological studies suggested that this scaffold (MMV676477) affected L. amazonensis cell division. Differential centrifugation and quantitative immunofluorescent confocal microscopy showed that MMV676477 promoted the partitioning of cellular tubulin towards the polymeric form in MMV676477-treated parasites than in controls. Turbidity assays with purified Leishmania and mammalian tubulin demonstrated that MMV676477 specifically promotes leishmanial tubulin polymerization in a concentration-dependent manner. The antiparasitic activity of MMV676477 analogs significantly correlated with their ability to facilitate purified Leishmania tubulin polymerization. Using a chemical crosslinking approach, competition between the most active, moderately active and less active compounds indicated that the MMV676477 scaffold directly bound purified endogenous Leishmania tubulin. These results demonstrate that MMV676477 is a potent antiparasitic compound that preferentially promotes Leishmania microtubule polymerization. Due to its selectivity for and broad-spectrum activity against multiple parasites, this scaffold shows promise for future antiparasitic drug development.

Since they are structurally very different, the compounds in the Pathogen Box would be expected to act against a wide variety of targets. Indeed, multiple targets for antileishmanial drug discovery have been proposed over the years. Tubulin has been considered an attractive antileishmanial drug target because Leishmania requires tubulin polymerization for multiple essential functions during its life cycle. As in mammalian cells, parasite tubulin is necessary for chromosome segregation and flagellar motility, but unlike in mammalian cells, multiple subpellicular microtubule-based shape changes are required for Leishmania to complete its cell cycle. Sequence alignment of human and Leishmania tubulin suggests that differences can be exploited; identity between Leishmania and human a or R tubulin ranges from 68-84%, depending on species and isoform, while similarity ranges from −80% (α-tubulin) to 90% (β-tubulin). At low temperatures, Leishmania tubulin is comparatively more stable than that of many higher eukaryotes, and Leishmania tubulin is also differentially sensitive from mammalian tubulin to many clinically used anti-tubulin/antimitotic therapies.

### EXAMPLES

Compounds

The Pathogen Box was provided by the Medicines for Malaria Venture as 10 mM stocks in DMSO (10 μL each) and stored at −20° C. The antileishmanial reference drugs amphotericin B and miltefosine (Sigma) were prepared in deionized water, and stored at −20° C. The maximum final DMSO concentration was 0.2% v/v in all experiments.

Parasite Cultures

Leishmania amazonensis promastigotes (strain IFLA/BR/67/PH8, provided by Norma W. Andrews, University of Maryland, College Park, MD) and L. tarentolae (Parrot strain, ATCC) were maintained at 26° C. in Schneider's Drosophila medium supplemented with 15% heat-inactivated, endotoxin-free FBS and 10 μg/ml gentamicin. L. amazonensis amastigotes were grown axenically at 32° C. in M199 (Invitrogen) at pH 4.5, supplemented with 20% FBS, 1% penicillin-streptomycin, 0.1% hemin (25 mg/ml in 50% triethanolamine), 10 mM adenine, 5 mM L-glutamine, 0.25% glucose, 0.5% Trypticase, and 40 mM sodium succinate.

A transgenic luciferase-expressing line of L. amazonensis parasites (L. amazonensisluc) was generated. Briefly, the 1.66 kb luciferase-coding region of pGL3-Basic (Promega, Madison, WI) was cloned in the expression vector pLEXSY. hyg2 (Jenabioscience, Jena, Germany). The final construct containing the luciferase gene and hygromycin resistance marker was integrated into the 18S rRNA locus of the nuclear DNA of L. amazonensis using the Human T-Cell Nucleofector kit and the Amaxa Nucleofector electroporator (program U-033). Following transfections, after 24 hours at 26° C., transfectants were selected by 100 μg/ml hygromycin in Schneider's Drosophila medium. Clones were isolated by limiting dilution. L. amazonensisluc parasites were maintained as above but the media was supplemented with 100 μg/ml hygromycin. L. amazonensis virulence was maintained by passage in C57B/6 mice.

For other parasites, L. donovani, strain MHOM/SD/62/1S-C12D, was grown as previously described. Plasmodium falciparum parasites of the 3D7 strain were cultured in RPMI 1640 medium supplemented with 37.5 mM HEPES, 10 mM D-glucose, 2 mM L-glutamine, 100 μM hypoxanthine, 25 μg/mL gentamicin, 4% (v/v) human serum and 0.25% (v/v) Albumax II, at a 2% haematocrit in an atmosphere of 1% 02, 3% CO₂ and 96% N₂. Staging and parasitemia of the in vitro culture were assessed by light microscopy of Giemsa-stained thin blood smears.

The parasites were synchronized using sequential sorbitol lysis treatment, with experiments carried out at least one intra-erythrocytic cycle later. T. brucei single marker (SM) cells were maintained at log phase growth (<1.5×10⁶ cells/mL) in HMI-19 media supplemented with 10% FBS (Gibco) and 2.5 μg/mL G418 (Life Technologies) at 37° C. and 5% CO₂.

Cell Cultures

RAW 264.7 cells (ATCC TIB-71) and HepG2 (ATCC, HB-806) were grown in Dulbecco's modified Eagle's medium (DMEM) supplemented with 10% heat-inactivated fetal bovine serum (FBS; Invitrogen, Grand Island, NY). Bone-marrow-derived (BM) primary macrophages (Mϕs) were isolated from the tibias and femurs of wildtype mice and differentiated into BM primary Mϕs over 7 days in DMEM supplemented with 10% FBS and 20% supernatant from L929 cells.

Concentration-Response Assays in L. amazonensis, T. Brucei, T. Cruzi, and P. falciparum

Leishmania amazonensis axenic amastigotes (100 μL, 2×10⁶ cells/mL) were added to 96-multiwell plates containing 100 μL of amastigote culture medium with an appropriate compound dilution series. 0.1% DMSO (no drug) served as a positive control (100%), and 5 μM amphotericin B served as a negative growth control (0%). Only internal wells were used to minimize edge effects from evaporation. Axenic amastigotes were incubated at 32° C. with a compound/drug for 72 h prior to measurement. Similarly, the cytotoxicity of each compound was determined against RAW 264.7 cells and HepG2 cell lines, following 72 h incubation at 37° C. (promastigotes at 26° C.).

10% alamarBlue® (Thermo Fisher Scientific) was used to measure the growth of both parasite and mammalian cells. Conversion from a blue oxidized state to a pink reduced state was assessed visually at 6 hr. The fluorescence signal was measured with a BioTek Synergy H1 plate reader (530 nm excitation, 570 nm emission). For bioluminescence assays using L. amazonensisluc, relative luminescence units (RLU) were measured using Britelite Plus (PerkinElmer, USA). Luminescence produced by this luciferase reaction is proportional to the amount of luciferase-expressing, viable L. amazonensisluc. Following five-minute incubation at room temperature, the luminescence was measured with a BioTek Synergy H₁ plate reader.

Compounds were tested against the blood-stage Trypanosoma brucei (48 hr endpoint) and intra-erythrocytic asexual stages Plasmodium falciparum (3D7) using CellTiter-Glo® luminescent and Malaria SYBR green I fluorescence (MSF) assays, respectively. Similar methodology will be used for future experiments.

GFP-transfected T. cruzi epimastigotes were grown in media and treated with serial dilutions of the compounds indicated. Data shown was obtained by flow cytometry and/or microscopy after 72 hrs of growth. Experiments for additional compounds will be done similarly. —Intracellular T. cruzi amastigotes that express luciferase will be tested as described for Leishmania.

All experiments were conducted as three technical triplicates on the same plate, with at least three independent biological repeats of each plate performed. For all assays, percent growth was expressed as a proportion of the untreated (positive) control (i.e. 100%) and plotted against drug/compound concentration. The concentration was then log₁₀ transformed and EC₅₀ values were determined using nonlinear regression (sigmoidal dose-response/variable slope equation) in GraphPad Prism v5.0 (GraphPad Software, Inc., San Diego, CA).

For Toxoplasma gondii, GFP-transfected RH tachyzoites will be grown in human foreskin fibroblasts and incubated with the compounds described. The number of T. gondii parasites that have survived treatment will be determined by microscopy at 36 hrs and compared to negative (no drug) and positive (100% kill) controls.

For Cryptosporidium parvum, luciferase-expressing sporozoites will be added to 70% confluent HCT-8 cells at 37° C. and infection will occur for 48-72 hours with or without drug. Cells will be lysed and the assay will be read as described for the luciferase assay for Leishmania intracellular amastigotes below.

Intramacrophage—L. amazonensis Assays

Intracellular EC₅₀s were estimated with L. amazonensisluc parasites. Briefly, RAW 264.7 cells or BM primary Mϕs were starved overnight, then infected with metacyclic promastigotes at a Multiplicity of Infection (MOI) of 15 and incubated for another 24 h at 37° C. The plates containing infected RAW 264.7 cells/BM primary M4s were washed five times with serum-free DMEM. Serially diluted compounds were added, and plates were incubated at 37° C. for 72 hrs. Bioluminescence signal was measured as described above.

Intracellular LD₅₀ₛ were measured. Briefly, RAW 264.7 cells were seeded to 96-multiwell plates at a density of 2×10⁵ cells/mL (200 μL) and starved overnight at 37° C. The cells were infected with metacyclic promastigotes at a Multiplicity of Infection (MOI) of 15 and incubated for another 24 h at 37° C. The plates containing infected RAW 264.7 cells/BM primary M #s were washed five times with serum-free DMEM. Serially diluted compounds were added, and plates were incubated at 37° C. for 72 hrs. Wells were washed five times with DMEM, and the RAW 264.7 cells were lysed with 100 μL of 2 mg/mL saponin in DMEM for 5 min at room temperature, and further lysis was stopped with 100% FBS. After centrifugation, 200 μL acidic promastigotes media was replaced, plates were incubated at 26° C. for 96 h. Fluorescence intensity (alamarBlue®) was measured as described above.

Microscopy

Promastigotes or amastigotes were treated with the indicated compound concentrations and allowed to adhere to poly-L-lysine coated plates. All cells were fixed with 4% paraformaldehyde and permeabilized/blocked with 0.01% Triton X-100 and 2% BSA in PBS. Promastigotes and amastigotes were incubated with mouse anti-gp46 or anti-p8, respectively at 1:50 or 1:1000 and rat anti-alpha-tubulin antibody at 1:1000. Samples were then probed with A568 anti-mouse and A488 anti-mouse secondary antibodies (Molecular Probes). DNA was labeled with Hoescht. Samples were visualized on a Zeiss LSM 880 Inverted confocal Airyscan microscope at 63×; full Z-thicknesses through parasites were obtained. Maximal intensity projections were formulated and quantified with Image J. For tubulin intensity analyses, at least 50 promastigotes or amastigotes were quantified per condition per experiment after 24 hrs incubation at compounds' EC₅₀, and the average intensity plus standard error for three experiments was calculated. For microscopic analysis of mitotic arrest, at least 200 parasites were analyzed per condition per experiment after 48 hrs incubation at compounds' EC₅₀, and the average plus standard error for three experiments was calculated. Representative images were selected from the maximal intensity projections and linearly processed in Adobe Photoshop CS6.

Polymerized Vs. Unpolymerized Tubulin in Parasites

SDS-PAGE was performed using 12% polyacrylamide gels. Protein purity and concentration were assessed using Coomassie Blue staining and the bicinchoninic acid (BCA) (Pierce Biotechnology) assay, respectively, following the manufacturers' protocols. Bovine serum albumin (BSA) was included to generate a standard curve. Western blotting was then performed. Total proteins were resolved by polyacrylamide gel electrophoresis and transferred to PVDF membranes using a Mini Trans-Blot Cell (BioRad). The membranes were blocked in 5% non-fat dry milk in Tris-buffered saline (TBS) (20 mM Tris (pH 7.6), 150 mM NaCl). The membranes were incubated with mouse anti-alpha-tubulin antibody, 1:1000 in 5% BSA and TBS-T overnight at 4° C. The membranes were then incubated with a 1:8000 dilution of goat-anti-mouse H1RP-conjugated secondary antibody in TBS-T (5% non-fat dry milk) for 1 hour. Finally, membranes were incubated in SuperSignal West Pico Chemiluminescent Substrate (Thermo Scientific) for 5 minutes and visualized by the ImageQuant LAS 4000 (GE). GAPDH (Santa Cruz) was used at 1:1000 as a loading control.

Tubulin Purification from Leishmania tarentolae

L. tarentolae (Parrot strain from ATCC) tubulin was purified. Briefly, promastigotes of L. tarentolae were grown to a high density (˜1×10⁸ cells/mL), harvested, and resuspended in PME+P buffer containing 100 mM piperazine N, N′-bis (2-ethanesulfonic acid) (PIPES) buffer (pH 6.9), 1 mM glycol ether diamine tetraacetic acid (EGTA), 1 mM MgCl₂, 1 mM benzamidine, 0.5 mM phenylmethylsulfonyl fluoride, 25 μg/ml leupeptin. The resulting suspension was extensively sonicated on ice with a probe sonicator (Misonix), cooled on ice for 30 minutes and centrifuged at 40,000×g for 1 hour at 4° C. using an ultracentrifuge (Beckman, Fullerton, CA). The resulting supernatant was filtered through a glass wool or 0.45-micron filter before loading into an equilibrated DEAE-Sepharose Fast Flow matrix (Amersham Biosciences). The column was washed with two column volumes of PME+P and subsequently four column volumes PME+P containing 0.1 M KCl and 0.25 M glutamate (pH 6.9). Tubulin was eluted with two column volumes PME+P containing 0.3 M KCl and 0.75 M glutamate (pH 6.9), and tubulin-rich fractions were confirmed by tubulin polymerization assays. The assembly-competent tubulin was pooled together and diluted into 1×PME buffer, 2 mM GTP, 10 mM MgCl₂ and 8% DMSO (v/v). Tubulin was incubated in this buffer for 45 minutes at 30° C. to promote assembly, centrifuged at 50,000×g at 30° C. for 30 min, and re-suspended in 1 mL of ice-cold PME. Tubulin was further solubilized with three 4-5 s bursts at 10 W using the probe sonicator and incubated on ice for 45 minutes. The tubulin-rich solution was then centrifuged at 50,000×g at 4° C. for 45 minutes, and the supernatant containing heterodimeric tubulin was collected and stored at −80° C. until use. Purity was assessed by SDS-PAGE and Coomassie Blue.

Tubulin Polymerization Assays

Tubulin polymerization assays were performed in 96-well half-area microplates (Costar) in a final volume of 100 μL. Briefly, Leishmanial or porcine tubulin (>99% pure, Cat. #T240, Cytoskeleton, Inc.) in 50 μL volume was pre-treated with drugs on ice for 5 minutes before adding 50 μL ice cold buffer, to provide a final concentration of 3 mg/ml tubulin in 80 mM PIPES pH 6.9, 2 mM MgCl₂, 0.5 mM EGTA, 1 mM GTP, and 1% DMSO unless specified otherwise. The absorbance at 340 nm was recorded in a Synergy H1 microplate reader (BioTek) for up to 45 min at 37° C. (porcine tubulin) or 30° C. (L. tarentolae tubulin).

Fluorescent Microtubule Assembly Assays

Fluorescence images of microtubule assembly were performed. Briefly, 1.5 mg/ml purified porcine tubulin (>99% pure, Cat. #T240, Cytoskeleton, Inc.) was treated for 40 minutes in assembly buffer containing 80 mM PIPES (pH 6.9), 1 mM EGTA, 1 mM MgCl₂, 1 mM GTP and 1% DMSO at 37° C. The mixture was cross-linked by diluting it 10-fold using assembly buffer containing 1% glutaraldehyde. After 3 min, the reaction was quenched by diluting 5-fold with assembly buffer containing 20 mM Tris pH 6.8. Pedestals were inserted into centrifuge tubes and a poly-L-lysine coated coverslip was placed on top. 20% glycerol in assembly buffer with no GTP was assembled, and the poly-L-lysine coated coverslips were covered with 3 mL of cushion. 50 μL of the quenched, cross-linked reactions were gently layered on top and spun through the cushion onto the poly-L-lysine coated coverslips using an ultracentrifuge (22,500×g at 20° C. for 45 minutes or 4000×g for 12 hours at 20° C.). Coverslips were washed three times with assembly buffer (no GTP), fixed with ice-cold methanol, and stained for 20 minutes with FITC-DM1α (Sigma-Aldrich) diluted 250× in PBS+BSA. The coverslips were washed three times with assembly buffer and imaged by epifluorescence.

Synthetic Methods. General Procedure for Synthesis of MMV6766477 Analogs

Each tested compounds has a purity of >95% as judged by HPLC analysis (UV detection at 210 nM). Chemical shifts 6 are in ppm, and spectra were referenced using the residual solvent peak. The following abbreviations are used: singlet (s), doublet (d), triplet (t), quartet (q), double doublet (dd), quintet (quin), multiplet (m), broad signal (br s). Mass spectra (m/z) were recorded on an Agilent LC-MS 1100 or 1290 Infinity using ESI ionization. All chemicals were used as received unless otherwise noted.

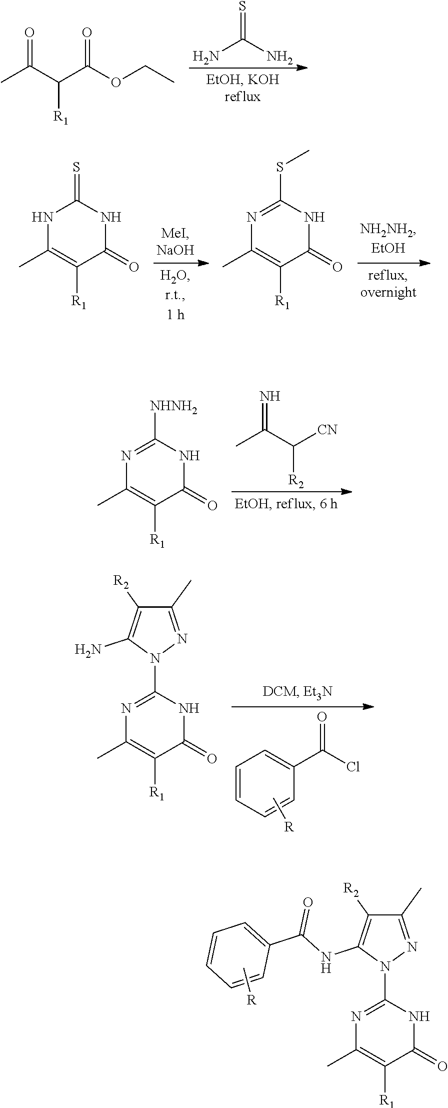

### Step 1

Ethyl 2-ethyl-3-oxobutanoate (1.6 mL, 10 mmol) was added to a stirred suspension of thiourea (766 mg, 10 mmol) and KOH (677 mg, 12 mmol) in EtOH (20 mL). The solution was refluxed for 5 h and completion of the reaction was confirmed by LCMS. The solid formed was collected by filtration and then dissolved in H₂O. The solution was then acidified with 1N HCl to pH 1 to give white precipitate of 5-ethyl-6-methyl-2-thioxo-2,3-dihydropyrimidin-4(1H)-one that was filtered and dried under vacuum (1.39 g, 82% yield). ¹H NMR (400 MHz, DMSO-d₆) δ 12.29 (s, 1H), 12.06 (s, 1H), 2.24 (q, J=8.0 Hz, 2H), 2.11 (s, 3H), 0.92 (t, J=8.0 Hz, 3H). ESI-MS (m/z): 171.1 [M+H]⁺.

### Step 2

NaOH (176 mg, 4.4 mmol) was added to a suspension of 5-ethyl-6-methyl-2-thioxo-2,3-dihydropyrimidin-4(1H)-one (685 mg, 4.0 mmol) in H₂O (12 mL) and the resulting mixture was allowed to stir to clarity. Then, the solution was cooled in an ice bath, and iodomethane (0.28 mL, 4.4 mmol) was added dropwise to the solution. The reaction was warmed to room temperature and continued to stir for 1 h. The solution was then acidified with 1N HCl to pH 7, precipitate formed was filtered, washed with H₂O and dried under vacuum to yield 5-ethyl-6-methyl-2-(methylthio)pyrimidin-4(3H)-one as a white solid (677 mg, 92% yield). ¹H NMR (400 MHz, DMSO-d₆) δ 12.44 (s, 1H), 2.44 (s, 3H), 2.35 (q, J=8.0 Hz, 2H), 2.20 (s, 3H), 0.96 (t, J=8.0 Hz, 3H). ESI-MS (m/z): 185.1 [M+H]⁺.

### Step 3

5-Ethyl-6-methyl-2-(methylthio)pyrimidin-4(3H)-one (619 mg, 3.3 mmol), hydrazine monohydrate (0.24 mL, 5 mmol), and EtOH (4 mL) were combined in a microwave tube and heated at 100° C. for 12 h. Then, the solution was filtered and the solid was washed with EtOH and dried overnight. The filtrate was condensed to give purple oil, and EtOH and EtOAc were added to precipitate out any remaining product. Some solid formed and was filtered and dried overnight. The samples were combined to give 5-ethyl-2-hydrazineyl-6-methylpyrimidin-4(3H)-one as a light purple solid (352 mg, 62% yield). ¹H NMR (400 MHz, DMSO-d₆) δ 2.26 (q, J=8.0 Hz, 2H), 2.05 (s, 3H), 0.91 (t, J=8.0 Hz, 3H). ESI-MS (m/z): 169.1 [M+H]⁺.

### Step 4

3-Iminobutanenitrile (197 mg, 2.4 mmol) was added to a stirred solution of 5-ethyl-2-hydrazineyl-6-methylpyrimidin-4(3H)-one (344 mg, 2.0 mmol) in EtOH (6 mL). After heating at 100° C. for 6 h, the mixture was cooled to room temperature and concentrated to give yellow solid. The solid was recrystallized from EtOAc/Hexanes to provide 2-(5-amino-3-methyl-1H-pyrazol-1-yl)-5-ethyl-6-methylpyrimidin-4(3H)-one (SW223075) as a yellow solid (820 mg, 88% yield). ¹H NMR (400 MHz, DMSO-d₆) δ 11.39 (br s, 1H), 6.84 (br s, 2H), 5.25 (s, 1H), 2.40 (q, J=8.0 Hz, 2H), 2.27 (s, 3H), 2.08 (s, 3H), 0.99 (t, J=8.0 Hz, 3H). ESI-MS (m/z): 234.1 [M+H]⁺.

### Step 5

The acid chlorides are either commercially available or prepared from the corresponding carboxylic acid.

Acylation Procedure A: Acid chloride (0.15 mmol) was added to a stirred solution of 2-(5-amino-3-methyl-1H-pyrazol-1-yl)-5-ethyl-6-methylpyrimidin-4(3H)-one (23 mg, 0.1 mmol) in DCM (4 mL) and triethylamine (0.35 mL) at −40° C. The solution was stirred for 2 h and monitored by LCMS. After the reaction was complete, the reaction was quenched with NaHCO₃ solution, and was allowed to stir for 5 minutes. Then the aqueous phase was extracted with DCM (3×10 mL), and washed with 1N HCl (1×10 mL) and brine (1×10 mL). It was dried with Na₂SO₄ and condensed. The resulting residue was then purified by column on silica gel to give the product.

Acylation Procedure B: Acid chloride (0.6 mmol) was added to a stirred solution of 2-(5-amino-3-methyl-1H-pyrazol-1-yl)-5-ethyl-6-methylpyrimidin-4(3H)-one (23 mg, 0.1 mmol) in DCM (4 mL) and triethylamine (55 μL, 0.4 mmol) at 0° C. The solution was stirred for 30 minutes at room temperature and monitored by LCMS. After the reaction was complete, the reaction mixture was diluted with water and extracted with DCM (3×10 mL). The combined organic layer was dried over Na₂SO₄ and concentrated under reduced pressure. Flash column chromatography of the residue furnished the N,O-diacylated product.

To a solution of the above N,O-diacylated compound in MeOH (4 mL) was added solid K₂CO₃ (41 mg, 0.3 mmol) and the resulting mixture was stirred for 30 minutes at room temperature. After that, the reaction was quenched by addition of water. The aqueous layer was extracted with EtOAc (3×10 mL), and the combined organic layer was dried over anhydrous Na₂SO₄, evaporated under reduced pressure and purified by flash column chromatography to give the product.

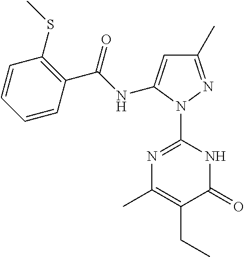 [SMILES: CCc1c(C)nc(-n2nc(C)cc2NC(=O)c2ccccc2SC)[nH]c1=O]

### N-(1-(5-Ethyl-4-methyl-6-oxo-1,6-dihydropyrimidin-2-yl)-3-methyl-1H-pyrazol-5-yl)-2-(methylthio)benzamide (MMV676477)

The target compound (85% yield) was obtained by the general procedure B described above. ¹H NMR (400 MHz, CDCl₃) δ 12.29 (s, 1H), 7.72 (d, J=8.0 Hz, 1H), 7.50 (t, J=8.0 Hz, 1H), 7.37 (d, J=8.0 Hz, 1H), 7.23 (t, J=8.0 Hz, 1H), 6.89 (s, 1H), 2.52 (q, J=8.0 Hz, 2H), 2.48 (s, 3H), 2.28 (s, 3H), 2.26 (s, 3H), 1.10 (t, J=8.0 Hz, 3H); ¹³C NMR (101 MHz, CDCl₃) δ 164.2, 161.3, 157.5, 153.6, 146.2, 141.3, 140.8, 132.0, 127.9, 126.1, 124.3, 122.9, 98.9, 21.2, 19.2, 16.1, 14.3, 12.9. ESI-MS (m/z): 384.1 [M+H]⁺.

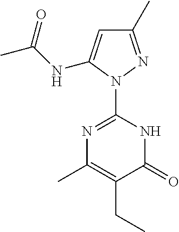 [SMILES: CCc1c(C)nc(-n2nc(C)cc2NC(C)=O)[nH]c1=O]

### N-(1-(5-Ethyl-4-methyl-6-oxo-1,6-dihydropyrimidin-2-yl)-3-methyl-1H-pyrazol-5-yl) acetamide (SW223023)

The target compound (57% yield) was obtained by the general procedure A described above. ¹H NMR (400 MHz, CDCl₃) δ 11.62 (s, 1H), 6.68 (s, 1H), 2.51 (q, J=6.8, 5.9 Hz, 2H), 2.33 (s, 3H), 2.24 (s, 3H), 2.23 (s, 3H), 1.10 (t, J=7.5 Hz, 3H); ¹³C NMR (101 MHz, CDCl₃) δ 166.4, 161.2, 157.3, 153.1, 140.4, 122.6, 98.5, 24.4, 21.2, 18.8, 14.1, 12.8. ESI-MS (m/z): 276 [M+H]⁺.

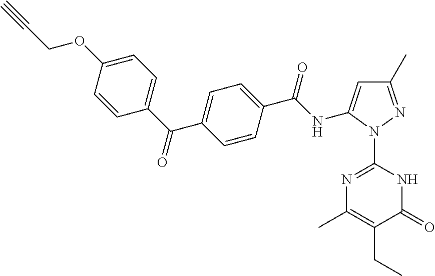 [SMILES: C#CCOc1ccc(C(=O)c2ccc(C(=O)Nc3cc(C)nn3-c3nc(C)c(CC)c(=O)[nH]3)cc2)cc1]

### N-(1-(5-Ethyl-4-methyl-6-oxo-1,6-dihydropyrimidin-2-yl)-3-methyl-1H-pyrazol-5-yl)-4-(4-(prop-2-yn-1-yloxy)benzoyl)benzamide (SW223022)

The target compound (600% yield) was obtained by the general procedure B described above. ¹H NM/R (400 MHz, CDCl₃) δ 12.59 (s, 1H), 10.27 (br s, 1H), 8.11 (d, J=8.0 Hz, 2H), 7.90 (d, J=8.0 Hz, 2H), 7.85 (d, J=12.0 Hz, 2H), 7.07 (d, J=12.0 Hz, 2H), 6.89 (s, 1H), 4.80 (d, J=2.4 Hz, 2H), 2.60-2.54 (m, 3H), 2.41 (s, 3H), 2.32 (s, 3H), 1.13 (t, J=8.0 Hz, 3H); ¹³C NMR (101 MHz, CDCl₃) δ 194.5, 162.7, 161.6, 160.9, 157.0, 153.7, 146.2, 141.9, 140.7, 136.1, 132.7, 130.2, 127.4, 123.3, 114.8, 99.1, 77.6, 76.3, 56.1, 21.4, 19.1, 14.4, 12.9. ESI-MS (m/z): 496.1 [M+H]⁺.

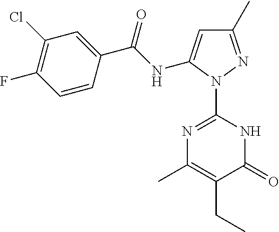 [SMILES: CCc1c(C)nc(-n2nc(C)cc2NC(=O)c2ccc(F)c(Cl)c2)[nH]c1=O]

### 3-Chloro-N-(1-(5-ethyl-4-methyl-6-oxo-1,6-dihydropyrimidin-2-yl)-3-methyl-1H-pyrazol-5-yl)-4-fluorobenzamide (SW223041)

The target compound (88% yield) was obtained by the general procedure B described above. ¹H NMR (400 MHz, CDCl₃) δ 12.54 (s, 1H), 8.05 (dd, J=8.0, 4.0 Hz, 1H), 7.97-7.91 (m, 1H), 7.21 (t, J=8.0 Hz, 1H), 6.82 (s, 1H), 2.56 (q, J=8.0 Hz, 2H), 2.43 (s, 3H), 2.30 (s, 3H), 1.12 (t, J=8.0 Hz, 3H); ¹³C NMR (101 MHz, CDCl₃) δ 162.1, 161.2, 159.6, 157.2, 153.6, 140.4, 130.6 (d), 129.8, 128.1 (d), 123.2, 122.2, 122.0, 117.6, 117.3, 98.9, 21.4, 19.0, 14.3, 12.9. ESI-MS (m/z): 390.1 [M+H]⁺.

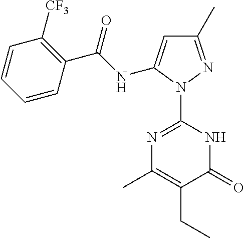 [SMILES: CCc1c(C)nc(-n2nc(C)cc2NC(=O)c2ccccc2C(F)(F)F)[nH]c1=O]

### N-(1-(5-Ethyl-4-methyl-6-oxo-1,6-dihydropyrimidin-2-yl)-3-methyl-1H-pyrazol-5-yl)-2-(trifluoromethyl)benzamide (SW223073)

The target compound (91% yield) was obtained by the general procedure B described above. ¹H NMR (400 MHz, CDCl₃) δ 12.11 (s, 1H), 7.84-7.80 (m, 1H), 7.74-7.64 (m, 3H), 6.89 (s, 1H), 2.50 (q, J=8.0 Hz, 2H), 2.31 (s, 3H), 2.07 (s, 3H), 1.08 (t, J=8.0 Hz, 3H); ¹³C NMR (101 MHz, CDCl₃) δ 163.6, 161.1, 157.3, 153.4, 145.9, 140.0, 134.9, 132.2, 131.0, 128.4, 127.0 (q), 124.8, 122.8, 122.9, 122.0, 99.3, 20.9, 18.9, 14.2, 12.8. ESI-MS (m/z): 406.1 [M+H]⁺.

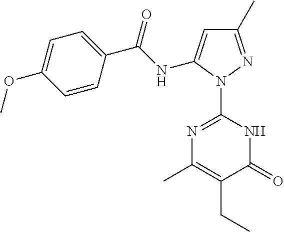 [SMILES: CCc1c(C)nc(-n2nc(C)cc2NC(=O)c2ccc(OC)cc2)[nH]c1=O]

### N-(1-(5-Ethyl-4-methyl-6-oxo-1,6-dihydropyrimidin-2-yl)-3-methyl-1H-pyrazol-5-yl)-4-methoxybenzamide (SW223072)

The target compound (85% yield) was obtained by the general procedure B described above. ¹H NMR (400 MHz, CDCl₃) δ 12.32 (s, 1H), 7.96 (d, J=8.0 Hz, 2H), 7.00 (d, J=8.0 Hz, 2H), 6.83 (s, 1H), 3.90 (s, 3H), 2.56 (q, J=8.0 Hz, 2H), 2.42 (s, 3H), 2.29 (s, 3H), 1.13 (t, J=8.0 Hz, 3H); ¹³C NMR (101 MHz, CDCl₃) δ 163.2, 161.1, 157.2, 153.7, 146.2, 141.2, 129.4, 125.7, 123.0, 114.2, 98.5, 55.7, 21.3, 19.0, 14.3, 12.9. ESI-MS (m/z): 368.1 [M+H]⁺.

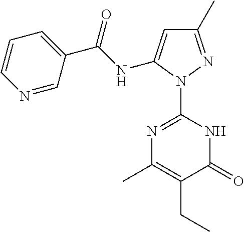 [SMILES: CCc1c(C)nc(-n2nc(C)cc2NC(=O)c2cccnc2)[nH]c1=O]

### N-(1-(5-Ethyl-4-methyl-6-oxo-1,6-dihydropyrimidin-2-yl)-3-methyl-1H-pyrazol-5-yl) nicotinamide (SW223074)

The target compound (80% yield) was obtained by the general procedure B described above. ¹H NMR (400 MHz, CDCl₃) δ 12.61 (s, 1H), 10.27 (br s, 1H), 9.22 (d, J=1.2 Hz, 1H), 8.84 (dd, J=4.8, 1.6 Hz, 1H), 8.33 (td, J=8.0, 1.6 Hz, 1H), 7.51 (dd, J=7.6, 4.8 Hz, 1H), 6.87 (s, 1H), 2.57 (q, J=8.0 Hz, 2H), 2.43 (s, 3H), 2.32 (s, 3H), 1.13 (t, J=8.0 Hz, 3H); ¹³C NMR (101 MHz, CDCl₃+CD₃OD) δ 161.5, 153.3, 153.0, 152.9, 147.9, 140.1 135.9, 129.4, 124.1, 99.0, 21.3, 18.9, 14.0, 12.8. ESI-MS (m/z): 339.2 [M+H]⁺.

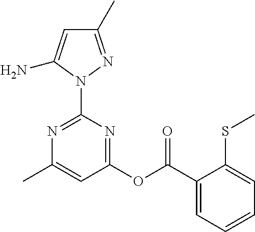 [SMILES: CSc1ccccc1C(=O)Oc1cc(C)nc(-n2nc(C)cc2N)n1]

### 2-(5-Amino-3-methyl-1H-pyrazol-1-yl)-6-methylpyrimidin-4-yl2-(methylthio)benzoate (SW233010)

The target compound (41% yield) was obtained by the similar procedure A described above. ¹H NMR (400 MHz, DMSO-d₆) δ 8.21 (dd, J=7.9, 1.5 Hz, 1H), 7.73-7.69 (m, 1H), 7.49 (d, J=8.2 Hz, 1H), 7.36-7.32 (m, 1H), 7.18 (s, 1H), 6.71 (s, 2H), 5.25 (s, 1H), 2.56 (s, 3H), 2.48 (s, 3H), 2.05 (s, 3H); ¹³C NMR (101 MHz, DMSO-d₆) δ 172.4, 166.0, 162.8, 157.4, 151.8, 151.2, 145.3, 134.9, 132.5, 125.6, 124.4, 124.0, 108.4, 88.7, 24.2, 15.2, 14.4. ESI-MS (m/z): 356 [M+H]⁺.

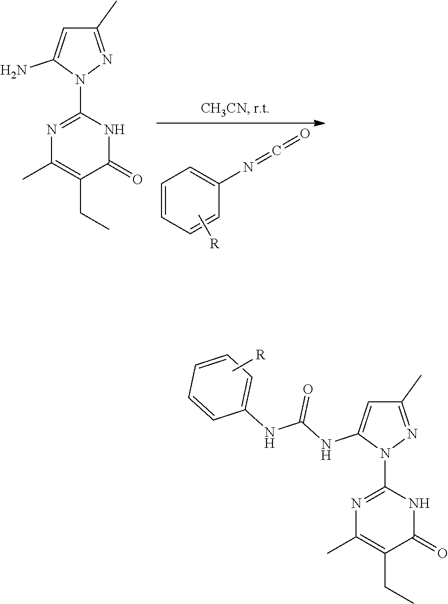

To a suspension of 2-(5-amino-3-methyl-1H-pyrazol-1-yl)-5-ethyl-6-methylpyrimidin-4(3H)-one (23 mg, 0.1 mmol) in CH₃CN (5 mL) was added isocyanate (0.15 mmol). The mixture was stirred at room temperature and monitored by LCMS. After the reaction was complete, the solution was condensed and resulting residue was purified by flash chromatography on silica gel to provide the product.

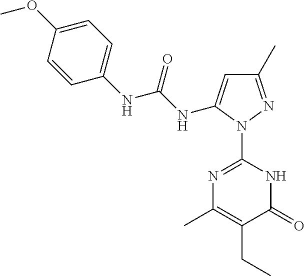 [SMILES: CCc1c(C)nc(-n2nc(C)cc2NC(=O)Nc2ccc(OC)cc2)[nH]c1=O]

### 1-(1-(5-Ethyl-4-methyl-6-oxo-1,6-dihydropyrimidin-2-yl)-3-methyl-1H-pyrazol-5-yl)-3-(4-methoxyphenyl)urea (SW223102)

The target compound (64% yield) was obtained by the general procedure described above. ¹H NMR (400 MHz, DMSO-d₆) δ 10.55 (s, 1H), 10.06 (s, 1H), 7.43 (d, J=8.4 Hz, 2H), 6.88 (d, J=8.5 Hz, 2H), 6.55 (s, 1H), 3.75-3.55 (m, 5H), 2.42 (s, 3H), 2.23 (s, 3H), 1.04 (t, J=7.5 Hz, 3H); ¹³C NMR (101 MHz, DMSO-d₆) δ 155.4, 152.0, 150.7, 149.7, 142.3, 132.6, 121.2, 120.2, 118.9, 114.4, 114.4, 96.8, 55.6, 19.9, 18.5, 14.1, 13.0. ESI-MS (m/z): 383 [M+H]⁺.

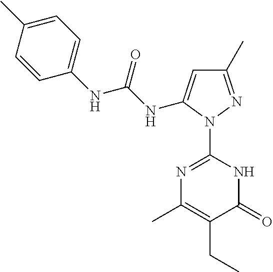 [SMILES: CCc1c(C)nc(-n2nc(C)cc2NC(=O)Nc2ccc(C)cc2)[nH]c1=O]

### 1-(1-(5-Ethyl-4-methyl-6-oxo-1,6-dihydropyrimidin-2-yl)-3-methyl-1H-pyrazol-5-yl)-3-(p-tolyl)urea (SW223101)

The target compound (66% yield) was obtained by the general procedure described above. ¹H NMR (400 MHz, DMSO-d₆) δ 10.57 (s, 1H), 10.23 (s, 1H), 7.43 (d, J=8.2 Hz, 2H), 7.27 (d, J=3.3 Hz, 1H), 7.09 (d, J=8.1 Hz, 2H), 6.58 (s, 1H), 2.51 (q, J=7.3 Hz, 2H), 2.45 (s, 3H), 2.24 (s, 6H), 1.04 (t, J=7.4 Hz, 3H); ¹³C NMR (101 MHz, DMSO-d₆) δ 151.8, 150.6, 149.5, 142.0, 138.6, 137.1, 131.9, 129.9, 129.6, 129.6, 129.0, 119.4, 118.5, 96.8, 21.2, 20.8, 18.5, 14.2, 13.1. ESI-MS (m/z): 367 [M+H]⁺.

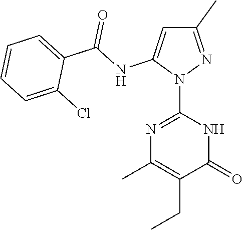 [SMILES: CCc1c(C)nc(-n2nc(C)cc2NC(=O)c2ccccc2Cl)[nH]c1=O]

### 2-Chloro-N-(1-(5-ethyl-4-methyl-6-oxo-1,6-dihydropyrimidin-2-yl)-3-methyl-1H-pyrazol-5-yl)benzamide (SW335718)

The target compound (92% yield) was obtained as a white solid by the general procedure B described above. ¹H NMR (400 MHz, CDCl₃) δ 12.23 (s, 1H), 10.22 (s, 1H), 7.75 (dd, J=7.6, 1.8 Hz, 1H), 7.54-7.37 (m, 3H), 6.89 (s, 1H), 2.52 (q, J=7.5 Hz, 2H), 2.30 (s, 3H), 2.21 (s, 3H), 1.09 (t, J=7.5 Hz, 3H). ¹³C NMR (101 MHz, CDCl₃) δ 162.8, 161.0, 157.6, 153.4, 145.8, 140.2, 134.4, 132.4, 131.3, 130.9, 130.4, 127.4, 123.1, 99.4, 21.0, 18.9, 14.3, 12.8. ESI-MS (m/z): 372.1 [M+H]⁺.

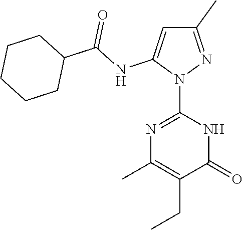 [SMILES: CCc1c(C)nc(-n2nc(C)cc2NC(=O)C2CCCCC2)[nH]c1=O]

### N-(1-(5-Ethyl-4-methyl-6-oxo-1,6-dihydropyrimidin-2-yl)-3-methyl-1H-pyrazol-5-yl)cyclohexane carboxamide (SW335719)

The target compound (91% yield) was obtained as a white solid by the general procedure B described above. ¹H NMR (400 MHz, CDCl₃) δ 11.58 (s, 1H), 10.22 (s, 1H), 6.70 (s, 1H), 2.55 (q, J=7.5 Hz, 2H), 2.36-2.33 (m, 4H), 2.25 (s, 3H), 2.10-2.06 (m, 2H), 1.88-1.85 (m, 2H), 1.77-1.70 (m, 1H), 1.55-1.46 (m, 2H), 1.42-1.24 (m, 3H), 1.12 (t, J=7.5 Hz, 3H). ¹³C NMR (101 MHz, CDCl₃) δ 172.9, 161.0, 157.3, 153.5, 145.9, 140.8, 122.9, 98.6, 46.0, 29.7, 25.9, 25.7, 21.2, 19.0, 14.3, 12.9. ESI-MS (m/z): 344.2 [M+H]⁺.

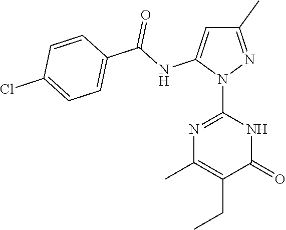 [SMILES: CCc1c(C)nc(-n2nc(C)cc2NC(=O)c2ccc(Cl)cc2)[nH]c1=O]

### 4-Chloro-N-(1-(5-ethyl-4-methyl-6-oxo-1,6-dihydropyrimidin-2-yl)-3-methyl-1H-pyrazol-5-yl)benzamide (SW335720)

The target compound (90% yield) was obtained as a white solid by the general procedure B described above. ¹H NMR (400 MHz, CDCl₃) δ 12.45 (s, 1H), 10.26 (s, 1H), 7.94 (d, J=8.5 Hz, 2H), 7.52 (d, J=8.5 Hz, 2H), 6.84 (s, 1H), 2.57 (q, J=7.5 Hz, 2H), 2.40 (s, 3H), 2.30 (s, 3H), 1.13 (t, J=7.5 Hz, 4H). ¹³C NMR (101 MHz, CDCl₃) δ 162.5, 160.9, 157.0, 153.7, 146.2, 140.7, 139.2, 131.8, 129.4, 128.8, 123.2, 98.9, 21.3, 19.0, 14.3, 12.9. ESI-MS (m/z): 372.2 [M+H]⁺.

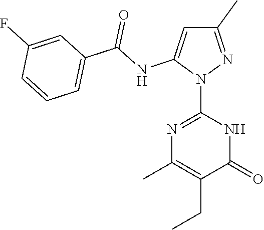 [SMILES: CCc1c(C)nc(-n2nc(C)cc2NC(=O)c2cccc(F)c2)[nH]c1=O]

### N-(1-(5-Ethyl-4-methyl-6-oxo-1,6-dihydropyrimidin-2-yl)-3-methyl-1H-pyrazol-5-yl)-3-fluorobenzamide (SW335721)

The target compound (90% yield) was obtained as a white solid by the general procedure B described above. ¹H NMR (400 MHz, CDCl₃) δ 12.50 (s, 1H), 7.79 (d, J=7.8 Hz, 1H), 7.70 (dt, J=9.3, 2.1 Hz, 1H), 7.52 (td, J=8.0, 5.5 Hz, 1H), 7.32 (td, J=8.2, 2.6 Hz, 1H), 6.85 (s, 1H), 2.57 (q, J=7.5 Hz, 3H), 2.42 (s, 3H), 2.31 (s, 3H), 1.13 (t, J=7.5 Hz, 4H). ¹³C NMR (101 MHz, CDCl₃) δ 164.3, 162.2, 161.8, 160.9, 157.1, 153.6, 146.2, 140.6, 135.6, 135.5, 130.7 (d, J=31.6 Hz), 123.2, 123.1 (d, J=12.5 Hz), 119.9, 119.7, 114.8, 114.6, 98.9, 21.2, 19.0, 14.3, 12.9. ESI-MS (m/z): 356.1 [M+H]⁺.

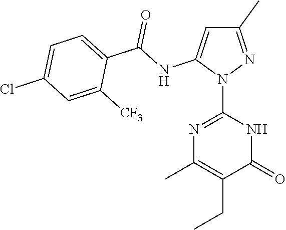 [SMILES: CCc1c(C)nc(-n2nc(C)cc2NC(=O)c2ccc(Cl)cc2C(F)(F)F)[nH]c1=O]

### 4-Chloro-N-(1-(5-ethyl-4-methyl-6-oxo-1,6-dihydropyrimidin-2-yl)-3-methyl-1H-pyrazol-5-yl)-2-(trifluoromethyl)benzamide (SW335724)

The target compound (92% yield) was obtained as a white solid by the general procedure B described above. ¹H NMR (400 MHz, CDCl₃) δ 12.13 (s, 1H), 7.81 (s, 1H), 7.67 (s, 2H), 6.87 (s, 1H), 2.51 (q, J=7.5 Hz, 2H), 2.31 (s, 3H), 2.10 (s, 3H), 1.09 (t, J=7.5 Hz, 3H). ¹³C NMR (101 MHz, CDCl₃) δ 162.5, 161.0, 157.2, 153.4, 145.9, 139.8, 137.4, 133.2, 132.3, 129.9, 127.6-127.4 (m), 124.0, 123.1, 121.2, 99.5, 21.0, 18.9, 14.2, 12.8. ESI-MS (m/z): 440.1 [M+H]⁺.

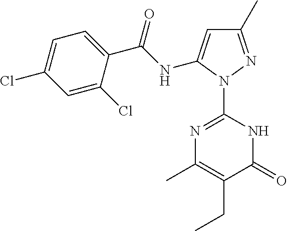 [SMILES: CCc1c(C)nc(-n2nc(C)cc2NC(=O)c2ccc(Cl)cc2Cl)[nH]c1=O]

### 2,4-Dichloro-N-(1-(5-ethyl-4-methyl-6-oxo-1,6-dihydropyrimidin-2-yl)-3-methyl-1H-pyrazol-5-yl)benzamide (SW335723)

The target compound (93% yield) was obtained as a white solid by the general procedure B described above. ¹H NMR (400 MHz, CDCl₃) δ 12.25 (s, 1H), 7.70 (d, J=8.3 Hz, 1H), 7.52 (d, J=2.0 Hz, 1H), 7.39 (dd, J=8.3, 2.0 Hz, 1H), 6.87 (s, 1H), 2.52 (q, J=7.5 Hz, 2H), 2.29 (s, 3H), 2.22 (s, 3H), 1.09 (t, J=7.5 Hz, 3H). ¹³C NMR (101 MHz, CDCl₃) δ 161.8, 161.0, 157.5, 153.4, 145.9, 139.9, 138.1, 132.8, 132.1, 131.5, 130.7, 127.8, 123.1, 99.5, 21.1, 18.9, 14.3, 12.8. ESI-MS (m/z): 406.1 [M+H]⁺.

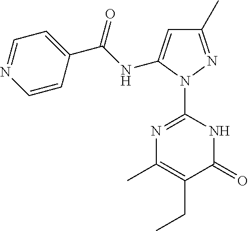 [SMILES: CCc1c(C)nc(-n2nc(C)cc2NC(=O)c2ccncc2)[nH]c1=O]

### N-(1-(5-Ethyl-4-methyl-6-oxo-1,6-dihydropyrimidin-2-yl)-3-methyl-1H-pyrazol-5-yl)isonicotinamide (SW335726)

The target compound (82% yield) was obtained as a light yellow solid by the general procedure B described above. ¹H NMR (400 MHz, CDCl₃) δ 12.62 (s, 1H), 10.27 (s, 1H), 8.88-8.85 (m, 2H), 7.82 (d, J=6.1 Hz, 1H), 6.88 (s, 1H), 2.57 (q, J=7.5 Hz, 2H), 2.41 (s, 3H), 2.32 (s, 3H), 1.14 (t, J=7.5 Hz, 3H). ¹³C NMR (101 MHz, CDCl₃) Difficult to collect because of very poor solubility. ESI-MS (m/z): 339.2 [M+H]⁺.

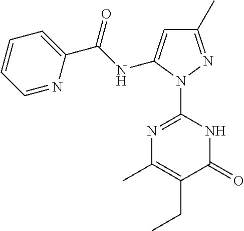 [SMILES: CCc1c(C)nc(-n2nc(C)cc2NC(=O)c2ccccn2)[nH]c1=O]

### N-(1-(5-Ethyl-4-methyl-6-oxo-1,6-dihydropyrimidin-2-yl)-3-methyl-1H-pyrazol-5-yl)picolinamide (SW335727)

The target compound (81% yield) was obtained as a light yellow solid by the general procedure B described above. ¹H NMR (400 MHz, CDCl₃) δ 13.63 (s, 1H), 8.71-8.64 (m, 1H), 8.34-8.25 (m, 1H), 7.94 (td, J=7.7, 1.7 Hz, 1H), 7.58-7.50 (m, 1H), 6.91 (s, 1H), 2.62-2.48 (m, 5H), 2.33 (s, 3H), 1.15 (t, J=7.5 Hz, 3H). ¹³C NMR (101 MHz, CDCl₃) δ 161.9, 159.0, 153.1, 148.9, 140.2, 137.7, 127.1, 122.9, 98.6, 20.9, 18.9, 14.2, 12.7. ESI-MS (m/z): 339.2 [M+H]⁺.

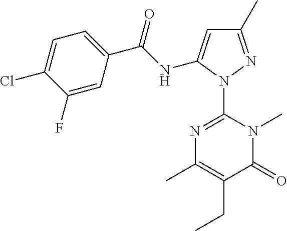 [SMILES: CCc1c(C)nc(-n2nc(C)cc2NC(=O)c2ccc(Cl)c(F)c2)n(C)c1=O]

### 4-Chloro-N-(1-(5-ethyl-1,4-dimethyl-6-oxo-1,6-dihydropyrimidin-2-yl)-3-methyl-1H-pyrazol-5-yl)-3-fluorobenzamide (SW335864)

### Step 1. Synthesis of 2-(5-amino-3-methyl-1H-pyrazol-1-yl)-5-ethyl-3,6-dimethylpyrimidin-4(3H)-one

To a solution of compound SW223075 (50 mg, 0.21 mmol) in acetone (4 mL) were added K₂CO₃ (35 mg, 0.25 mmol) and Mel (20 μL, 0.31 mmol). The resulting mixture was refluxed for 30 min. Then the solvent was evaporated and the reaction mixture was quenched with H₂O, extracted with EtOAc (3×15 mL) and dried over anhydrous Na₂SO₄. The organic layer was then concentrated under reduced pressure and purified by flash chromatography to afford 2-(5-amino-3-methyl-1H-pyrazol-1-yl)-5-ethyl-3,6-dimethylpyrimidin-4(3H)-one (62% yield) as a white solid.

Step 2. 4-Chloro-3-fluorobenzoyl chloride (46 mg, 0.24 mmol) was added to a stirred solution of above amine (25 mg, 0.12 mmol) in DCM (2 mL) and triethylamine (50 μL, 0.36 mmol) at 0° C. The solution was stirred for 30 min. at rt and monitored by LCMS. After the reaction was complete, the reaction mixture was diluted with water and extracted with DCM (3×10 mL). The combined organic layer was dried over Na₂SO₄ and concentrated under reduced pressure. Flash column chromatography of the residue furnished the desired product (91% yield) as a white solid. ¹H NMR (400 MHz, CDCl₃) δ 11.33 (s, 1H), 7.94 (dd, J=6.8, 2.3 Hz, 1H), 7.86-7.81 (m, 1H), 7.33-7.24 (m, 1H), 6.76 (s, 1H), 3.76 (s, 3H), 2.59 (q, J=7.5 Hz, 2H), 2.39 (s, 3H), 2.32 (s, 3H), 1.13 (t, J=7.5 Hz, 3H). ¹³C NMR (101 MHz, CDCl₃) δ 163.1, 162.0, 161.1, 159.5, 154.6, 151.9, 146.9, 140.3, 130.8, 130.7, 129.8, 127.9 (d, J=29.2 Hz), 123.6, 122.1, 117.5, 117.3, 98.9, 35.4, 21.1, 19.9, 14.3, 12.7. ESI-MS (m/z): 404.1 [M+H]⁺.

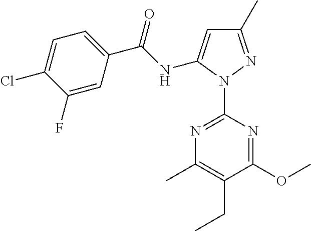 [SMILES: CCc1c(C)nc(-n2nc(C)cc2NC(=O)c2ccc(Cl)c(F)c2)nc1OC]

### 4-Chloro-N-(1-(5-ethyl-4-methoxy-6-methylpyrimidin-2-yl)-3-methyl-1H-pyrazol-5-yl)-3-fluorobenzamide (SW335866)

### Step 1. Synthesis of 1-(5-ethyl-4-methoxy-6-methylpyrimidin-2-yl)-3-methyl-1H-pyrazol-5-amine

To a solution of compound SW223075 (50 mg, 0.21 mmol) in DMF (3 mL) were added K₂CO₃ (35 mg, 0.25 mmol) and Mel (16 μL, 0.25 mmol) at rt. The resulting mixture was stirred for 30 min at rt. Then the reaction mixture was diluted with H₂O, extracted with EtOAc (3×20 mL) and dried over anhydrous Na₂SO₄. The organic layer was then concentrated under reduced pressure and purified by flash chromatography to afford 1-(5-ethyl-4-methoxy-6-methylpyrimidin-2-yl)-3-methyl-1H-pyrazol-5-amine (68% yield) as a white solid.

Step 2. 4-Chloro-3-fluorobenzoyl chloride (46 mg, 0.24 mmol) was added to a stirred solution of above amine (25 mg, 0.12 mmol) in DCM (3 mL) and triethylamine (50 μL, 0.36 mmol) at 0° C. The solution was stirred for 30 min. at rt and monitored by LCMS. After the reaction was complete, the reaction mixture was diluted with H₂O and extracted with DCM (3×10 mL). The combined organic layer was dried over Na₂SO₄ and concentrated under reduced pressure. Flash column chromatography of the residue furnished the desired product (90% yield) as a white solid. ¹H NMR (400 MHz, CDCl₃) δ 13.04 (s, 1H), 8.09-8.02 (m, 1H), 7.99-7.89 (m, 1H), 7.33-7.24 (m, 1H), 6.87 (s, 1H), 4.15 (s, 3H), 2.62 (q, J=7.5 Hz, 2H), 2.55 (s, 3H), 2.38 (s, 3H), 1.12 (t, J=7.5 Hz, 3H). ¹³C NMR (101 MHz, CDCl₃) δ 169.3, 163.0, 161.8, 161.4, 159.3, 154.8, 152.4, 140.5, 131.6 (d, J=9.6 Hz), 129.8, 128.2 (d, J=33.2 Hz), 121.9, 121.8, 118.1, 117.4, 117.2, 98.4, 54.8, 21.3, 18.5, 14.7, 13.1. ESI-MS (m/z): 404.1 [M+H]⁺.

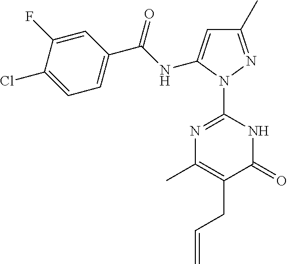 [SMILES: C=CCc1c(C)nc(-n2nc(C)cc2NC(=O)c2ccc(Cl)c(F)c2)[nH]c1=O]

### N-(1-(5-Allyl-4-methyl-6-oxo-1,6-dihydropyrimidin-2-yl)-3-methyl-1H-pyrazol-5-yl)-4-chloro-3-fluorobenzamide (SW335879)

The target compound (84% yield) was obtained as a white solid by the general procedure B described above. ¹H NMR (400 MHz, CDCl₃) δ 12.47 (s, 1H), 8.04 (dd, J=6.7, 2.4 Hz, 1H), 7.95-7.90 (m, 1H), 7.31 (t, J=8.5 Hz, 1H), 6.83 (s, 1H), 5.95-5.80 (m, 1H), 5.13-5.03 (m, 2H), 3.32 (d, J=6.0 Hz, 2H), 2.42 (s, 3H), 2.30 (s, 3H). ¹³C NMR (101 MHz, CDCl₃) δ 162.2, 161.2, 159.6, 158.7, 153.9, 140.6, 134.0, 130.5 (d, J=18.0 Hz), 129.8, 128.2 (d, J=30.4 Hz), 122.3, 122.1, 119.0, 117.6, 117.4, 115.8, 99.1, 29.5, 21.7, 14.4. ESI-MS (m/z): 402.1 [M+H]⁺.

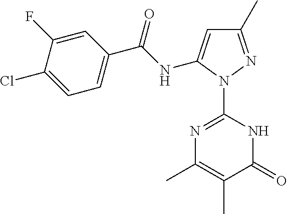 [SMILES: Cc1cc(NC(=O)c2ccc(Cl)c(F)c2)n(-c2nc(C)c(C)c(=O)[nH]2)n1]

### 4-Chloro-N-(1-(4,5-dimethyl-6-oxo-1,6-dihydropyrimidin-2-yl)-3-methyl-1H-pyrazol-5-yl)-3-fluorobenzamide (SW335880)

The target compound (81% yield) was obtained as a white solid by the general procedure B described above. ¹H NMR (400 MHz, CDCl₃) δ 12.47 (s, 1H), 8.05 (dd, J=6.8, 2.3 Hz, 1H), 7.96-7.90 (m, 1H), 7.31 (t, J=8.5 Hz, 1H), 6.83 (s, 1H), 2.43 (s, 3H), 2.31 (s, 3H), 2.10 (s, 3H). ¹³C NMR (101 MHz, CDCl₃) δ 162.1, 161.4, 159.5, 153.4, 140.4, 129.8, 128.1, 122.2, 117.55, 98.8, 14.1, 10.9. ESI-MS (m/z): 376.1 [M+H]⁺.

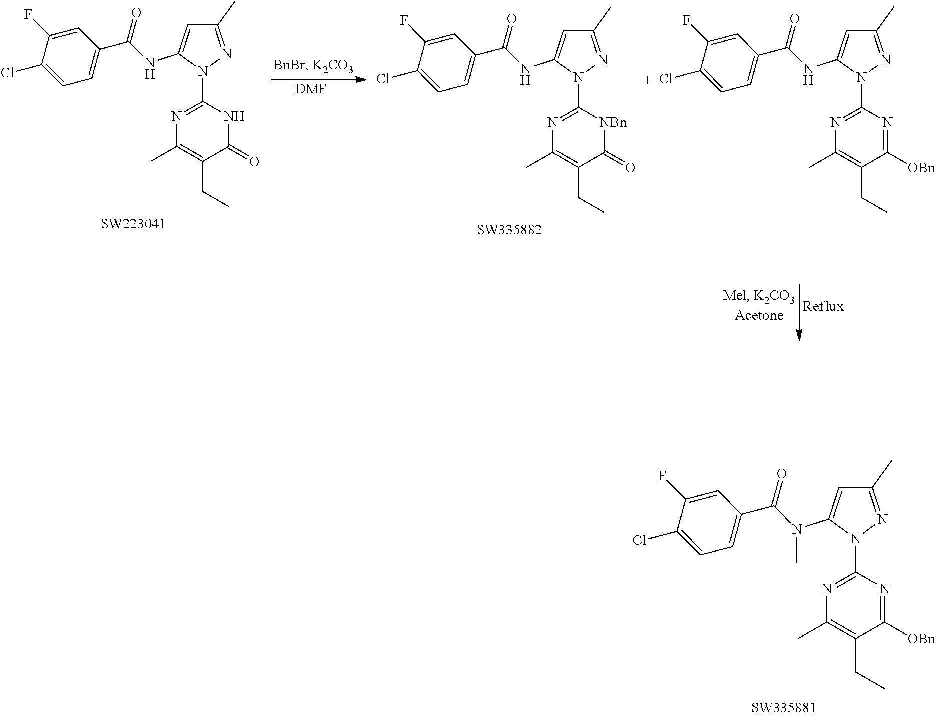

### N-(1-(4-(Benzyloxy)-5-ethyl-6-methylpyrimidin-2-yl)-3-methyl-1H-pyrazol-5-yl)-4-chloro-3-fluoro-N-methylbenzamide (SW335881)

### Step 1. Synthesis of N-(1-(4-(benzyloxy)-5-ethyl-6-methylpyrimidin-2-yl)-3-methyl-1H-pyrazol-5-yl)-2-(4-chloro-3-fluorophenyl)acetamide and N-(1-(1-Benzyl-5-ethyl-4-methyl-6-oxo-1,6-dihydropyrimidin-2-yl)-3-methyl-1H-pyrazol-5-yl)-4-chloro-3-fluorobenzamide (SW335882)

To a solution of compound SW223041 (100 mg, 0.25 mmol) in DMF (5 mL) were added K₂CO₃ (42 mg, 0.30 mmol) and BnBr (32 μL, 0.27 mmol). The resulting mixture was stirred for 30 min at rt. Then the reaction mixture was diluted with H₂O, extracted with EtOAc (3×20 mL) and dried over anhydrous Na₂SO₄. The organic layer was then concentrated under reduced pressure and purified by flash chromatography to afford N-(1-(4-(benzyloxy)-5-ethyl-6-methylpyrimidin-2-yl)-3-methyl-1H-pyrazol-5-yl)-2-(4-chloro-3-fluorophenyl)acetamide (40% yield) and N-(1-(1-Benzyl-5-ethyl-4-methyl-6-oxo-1,6-dihydropyrimidin-2-yl)-3-methyl-1H-pyrazol-5-yl)-4-chloro-3-fluorobenzamide (SW335882) (42% yield) as a white solid. ¹H NMR (400 MHz, CDCl₃) δ 13.06 (s, 1H), 8.07 (d, J=6.8 Hz, 1H), 7.94 (bs, 1H), 7.65-7.14 (m, 6H), 6.89 (s, 1H), 5.60 (s, 2H), 2.66 (q, J=7.6 Hz, 2H), 2.57 (s, 3H), 2.40 (s, 3H), 1.13 (t, J=7.5 Hz, 3H). ¹³C NMR (101 MHz, CDCl₃) δ 168.7, 163.2, 161.8, 161.3, 159.2, 154.7, 152.4, 140.5, 136.0, 131.5 (d, J=12.0 Hz), 129.7, 128.6, 128.4-128.2 (m), 128.1, 122.0, 121.8, 118.2, 117.4, 117.2, 98.3, 69.2, 21.3, 18.6, 14.7, 13.1. ESI-MS (m/z): 480.2 [M+H]⁺.

Step 2. To a solution of N-(1-(4-(benzyloxy)-5-ethyl-6-methylpyrimidin-2-yl)-3-methyl-1H-pyrazol-5-yl)-2-(4-chloro-3-fluorophenyl)acetamide (30 mg, 0.06 mmol) in acetone (2 mL) were added K₂CO₃ (14 mg, 0.10 mmol) and Mel (10 μL, 0.15 mmol). The resulting mixture was refluxed for 48 h. Then acetone was evaporated and the reaction mixture was quenched with H₂O, extracted with ethyl acetate (3×10 mL) and dried over anhydrous Na₂SO₄. The organic layer was then concentrated under reduced pressure and purified by flash chromatography to afford target compound (89% yield) as a white solid. ¹H NMR (400 MHz, CDCl₃) δ 7.83-7.76 (m, 1H), 7.59-7.51 (m, 1H), 7.23-7.14 (m, 3H), 7.03-6.93 (m, 3H), 5.60 (s, 2H), 5.54 (s, 1H), 2.63 (q, J=7.5 Hz, 2H), 2.27 (s, 3H), 2.24 (s, 3H), 2.23 (s, 3H), 1.17 (t, J=7.5 Hz, 3H). ¹³C NMR (101 MHz, CDCl₃) δ 168.3, 162.9, 160.5, 158.0, 156.7, 150.7, 145.0, 143.2, 136.4, 131.3, 128.8, 128.6, 128.5, 128.2, 128.2, 126.2, 116.3, 105.3, 46.1, 20.9, 20.3, 14.2, 12.6. ESI-MS (m/z): 494.2 [M+H]⁺.

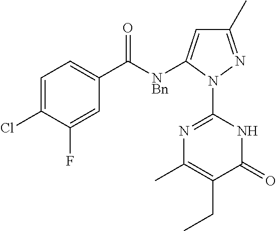 [SMILES: CCc1c(C)nc(-n2nc(C)cc2N(Cc2ccccc2)C(=O)c2ccc(Cl)c(F)c2)[nH]c1=O]

### N-Benzyl-4-chloro-N-(1-(5-ethyl-4-methyl-6-oxo-1,6-dihydropyrimidin-2-yl)-3-methyl-1H-pyrazol-5-yl)-3-fluorobenzamide (SW335877)

### Step 1. Synthesis of 2-(5-(benzylamino)-3-methyl-1H-pyrazol-1-yl)-5-ethyl-6-methylpyrimidin-4(3H)-one

To a solution of SW223075 (100 mg, 0.42 mmol) in DMF (4 mL) were added K₂CO₃ (71 mg, 0.51 mmol) and BnBr (61 μL, 0.51 mmol). The resulting mixture was stirred for 30 min at rt. Then the reaction mixture was diluted with H₂O, extracted with ethyl acetate (3×20 mL) and dried over anhydrous Na₂SO₄. The organic layer was then concentrated under reduced pressure and purified by flash chromatography to afford the target compound (31% yield).

Step 2. The target compound (72% yield) was obtained as a white solid by the general procedure B described above. ¹H NMR (400 MHz, CDCl₃) δ 11.00 (s, 1H), 7.87 (dd, J=6.8, 2.3 Hz, 1H), 7.78-7.73 (m, 1H), 7.30-7.22 (m, 1H), 7.20-7.15 (m, 3H), 7.03-6.98 (m, 2H), 6.71 (s, 1H), 5.97 (s, 2H), 2.60 (q, J=7.5 Hz, 2H), 2.38 (s, 3H), 2.31 (s, 3H), 1.13 (t, J=7.5 Hz, 3H). ¹³C NMR (101 MHz, CDCl₃) δ 162.8, 162.0, 161.0, 159.5, 154.6, 151.9, 146.2, 140.2, 136.7, 130.8, 129.8, 128.6, 127.9-127.6 (m), 124.4, 122.2, 122.06, 117.5, 117.3, 98.6, 48.1, 21.1, 20.1, 14.3, 12.6. ESI-MS (m/z): 480.2 [M+H]⁺.

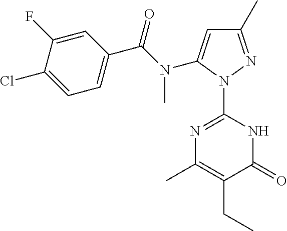 [SMILES: CCc1c(C)nc(-n2nc(C)cc2N(C)C(=O)c2ccc(Cl)c(F)c2)[nH]c1=O]

### 4-Chloro-N-(1-(5-ethyl-4-methyl-6-oxo-1,6-dihydropyrimidin-2-yl)-3-methyl-1H-pyrazol-5-yl)-3-fluoro-N-methylbenzamide (SW335878)

To a solution of compound SW335881 (20 mg, 0.04 mmol) in DCM (2 mL) was added BBr₃ (80 μL, 0.08 mmol, 1M in hexane) dropwise at −78° C. The resulting mixture was stirred for 2 h at the same temperature. Then the reaction mixture was diluted with H₂O, extracted with ethyl acetate (3×15 mL) and dried over anhydrous Na₂SO₄. The organic layer was then concentrated under reduced pressure and purified by flash chromatography to afford the target compound (53% yield) as a white solid. ¹H NMR (400 MHz, CDCl₃) δ 7.41 (s, 1H), 7.14 (s, 1H), 6.95 (s, 1H), 6.15 (s, 1H), 3.34 (s, 3H), 2.54 (q, J=7.5 Hz, 2H), 2.34 (s, 3H), 2.24 (s, 3H), 1.12 (t, J=7.5 Hz, 3H). ESI-MS (m/z): 404.1 [M+H]⁺.

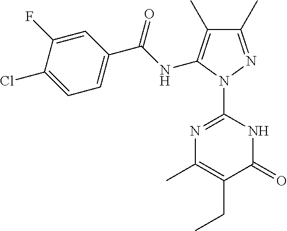 [SMILES: CCc1c(C)nc(-n2nc(C)c(C)c2NC(=O)c2ccc(Cl)c(F)c2)[nH]c1=O]

### 4-Chloro-N-(1-(5-ethyl-4-methyl-6-oxo-1,6-dihydropyrimidin-2-yl)-3,4-dimethyl-1H-pyrazol-5-yl)-3-fluorobenzamide (SW335888)

The target compound (89% yield) was obtained as a white solid by the general procedure B described above. ¹H NMR (400 MHz, CDCl₃) δ 11.5 (s, 1H), 8.08 (dt, J=6.9, 1.9 Hz, 1H), 7.95-7.89 (m, 1H), 7.33-7.22 (m, 1H), 2.50 (q, J=7.7 Hz, 2H), 2.28 (s, 3H), 2.21 (s, 3H), 2.10 (s, 3H), 1.12-1.03 (t, J=7.7 Hz, 3H). ¹³C NMR (101 MHz, CDCl₃) δ 162.1, 161.4, 159.5, 157.3, 153.6, 136.7, 130.8 (d, J=13.6 Hz), 130.4, 128.2 (d, J=29.2 Hz), 122.9, 122.2, 122.0, 117.4, 117.1, 110.4, 21.3, 19.0, 12.8 (d, J=32.8 Hz), 10.1. ESI-MS (m/z): 404.1 [M+H]⁺.

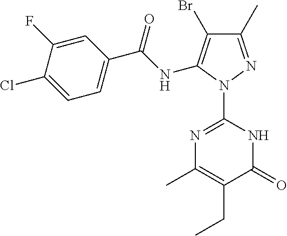 [SMILES: CCc1c(C)nc(-n2nc(C)c(Br)c2NC(=O)c2ccc(Cl)c(F)c2)[nH]c1=O]

### N-(4-Bromo-1-(5-ethyl-4-methyl-6-oxo-1,6-dihydropyrimidin-2-yl)-3-methyl-1H-pyrazol-5-yl)-4-chloro-3-fluorobenzamide (SW336210)

### Step 1. Synthesis of 2-(5-amino-4-bromo-3-methyl-1H-pyrazol-1-yl)-5-ethyl-6-methylpyrimidin-4(3H)-one

NBS (42 mg, 0.23 mmol) was added to a solution of SW223075 (25 mg, 0.21 mmol) in dry DCM (3 mL) and stirred 3 h at rt. After completion of reaction, solvent was evaporated and purified by flash chromatography to furnish the desired product (82% yield) as a light yellow solid.

Step 2. The target compound (92% yield) was obtained as a white solid by the general procedure B described above. ¹H NMR (400 MHz, CDCl₃) δ 11.32 (s, 1H), 8.10 (dd, J=6.8, 2.3 Hz, 1H), 7.97-7.92 (m, 1H), 7.32 (t, J=8.5 Hz, 1H), 2.53 (q, J=7.5 Hz, 2H), 1.09 (t, J=7.5 Hz, 3H). ¹³C NMR (101 MHz, CDCl₃) δ 162.3, 160.5, 159.7, 157.3, 152.6, 138.1, 130.6, 130.4, 128.4 (d, J=32.4 Hz), 123.8, 122.4, 122.2, 117.5, 117.3, 92.4, 21.3, 19.1, 13.3, 12.8. ESI-MS (m/z): 468.0 [M+H]⁺.

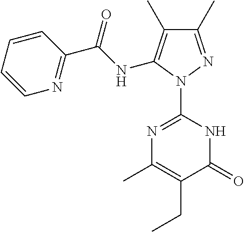 [SMILES: CCc1c(C)nc(-n2nc(C)c(C)c2NC(=O)c2ccccn2)[nH]c1=O]

### N-(1-(5-Ethyl-4-methyl-6-oxo-1,6-dihydropyrimidin-2-yl)-3,4-dimethyl-1H-pyrazol-5-yl)picolinamide (SW336211)

The target compound (67% yield) was obtained as a light yellow solid by the general procedure B described above. ¹H NMR (400 MHz, CDCl₃) δ 12.50 (s, 1H), 8.69-8.66 (m, 1H), 8.27 (dt, J=7.8, 1.1 Hz, 1H), 7.92 (td, J=7.7, 1.7 Hz, 1H), 7.52 (ddd, J=7.6, 4.7, 1.2 Hz, 1H), 2.53 (q, J=7.5 Hz, 2H), 2.42 (s, 3H), 2.23 (s, 3H), 2.15 (s, 3H), 1.10 (t, J=7.5 Hz, 3H). ¹³C NMR (101 MHz, CDCl₃) δ 161.5, 153.1, 149.5, 148.3, 145.0, 137.7, 136.3, 126.9, 123.0, 122.6, 110.8, 21.0, 18.9, 12.8, 10.1. ESI-MS (m/z): 353.2 [M+H]⁺.

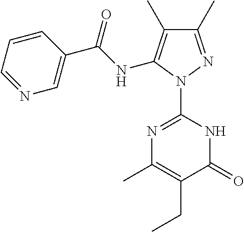 [SMILES: CCc1c(C)nc(-n2nc(C)c(C)c2NC(=O)c2cccnc2)[nH]c1=O]

### N-(1-(5-Ethyl-4-methyl-6-oxo-1,6-dihydropyrimidin-2-yl)-3,4-dimethyl-1H-pyrazol-5-yl)nicotinamide (SW336214)

The target compound (73% yield) was obtained as a light yellow solid by the general procedure B described above. ¹H NMR (400 MHz, CDCl₃) δ 11.57 (s, 1H), 10.52 (s, 1H), 9.25 (dd, J=2.3, 0.9 Hz, 1H), 8.83 (ddt, J=4.9, 1.7, 0.8 Hz, 1H), 8.33 (d, J=7.8 Hz, 1H), 7.52-7.44 (m, 1H), 2.49 (q, J=7.5 Hz, 2H), 2.25 (s, 3H), 2.21 (s, 3H), 2.11 (s, 3H), 1.06 (t, J=7.5 Hz, 3H). ¹³C NMR (101 MHz, CDCl₃) δ 161.8, 161.3, 157.3, 153.4, 153.2, 148.5, 145.8, 136.4, 135.8, 129.4, 123.8, 122.9, 110.6, 21.2, 18.9, 12.8, 10.0. ESI-MS (m/z): 353.2 [M+H]⁺.

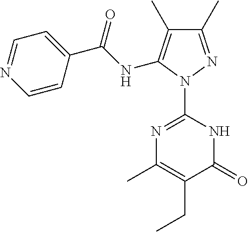 [SMILES: CCc1c(C)nc(-n2nc(C)c(C)c2NC(=O)c2ccncc2)[nH]c1=O]

### N-(1-(5-Ethyl-4-methyl-6-oxo-1,6-dihydropyrimidin-2-yl)-3,4-dimethyl-1H-pyrazol-5-yl)isonicotinamide (SW336215)

The target compound (72% yield) was obtained as a white solid by the general procedure B described above. ¹H NMR (400 MHz, CDCl₃) δ 11.66 (s, 1H), 8.91-8.85 (m, 2H), 7.93-7.87 (m, 2H), 2.52 (q, J=7.5 Hz, 2H), 2.27 (s, 3H), 2.25 (s, 3H), 2.14 (s, 3H), 1.09 (t, J=7.5 Hz, 3H). ¹³C NMR (101 MHz, CDCl₃) δ 161.5, 153.6, 150.3, 145.8, 141.4, 136.2, 123.2, 121.6, 110.9, 21.2, 19.0, 12.8, 10.1. ESI-MS (m/z): 353.2 [M+H]⁺.

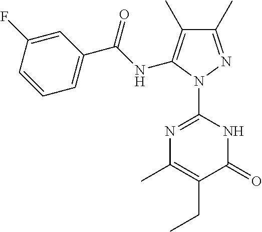 [SMILES: CCc1c(C)nc(-n2nc(C)c(C)c2NC(=O)c2cccc(F)c2)[nH]c1=O]

### N-(1-(5-Ethyl-4-methyl-6-oxo-1,6-dihydropyrimidin-2-yl)-3,4-dimethyl-1H-pyrazol-5-yl)-3-fluorobenzamide (SW336216)

The target compound (92% yield) was obtained as a white solid by the general procedure B described above. ¹H NMR (400 MHz, CDCl₃) δ 11.48 (s, 1H), 10.32 (s, 1H), 7.81-7.77 (m, 1H), 7.73-7.70 (m, 1H), 7.55-7.45 (m, 1H), 7.42-7.27 (m, 1H), 2.50 (q, =7.5 Hz, 2H), 2.26 (s, 3H), 2.22 (s, 3H), 2.12 (s, 3H), 1.08 (t, J=7.5, 3H). ¹³C NMR (101 MHz, CDCl₃) δ 164.2, 162.3 (d, J=10.8 Hz), 161.7, 161.1, 157.3, 153.5, 145.7, 136.7, 135.9, 135.8, 130.6, 130.6, 123.3 (d, J=12.4 Hz), 122.9, 119.8, 119.6, 115.1, 114.8, 110.4, 21.2, 18.9, 12.8, 10.1. ESI-MS (m/z): 370.1 [M+H]⁺.

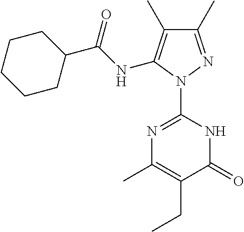 [SMILES: CCc1c(C)nc(-n2nc(C)c(C)c2NC(=O)C2CCCCC2)[nH]c1=O]

### N-(1-(5-Ethyl-4-methyl-6-oxo-1,6-dihydropyrimidin-2-yl)-3,4-dimethyl-1H-pyrazol-5-yl)cyclohexanecarboxamide (SW336231)

The target compound (91% yield) was obtained as a white solid by the general procedure B described above. ¹H NMR (400 MHz, CDCl₃) δ 10.29 (s, 1H), 2.51 (q, J=7.4 Hz, 2H), 2.36-2.27 (m, 4H), 2.16 (s, 3H), 2.10-2.00 (m, 2H), 1.98 (s, 3H), 1.88-1.80 (m, 2H), 1.77-1.67 (m, 1H), 1.58-1.46 (m, 2H), 1.42-1.17 (m, 3H), 1.09 (t, J=7.5 Hz, 3H). ¹³C NMR (101 MHz, CDCl₃) δ 173.2, 157.7, 153.1, 136.6, 122.7, 110.5, 46.1, 29.7, 25.7, 21.2, 18.9, 12.7, 9.8. ESI-MS (m/z): 358.2 [M+H]⁺.

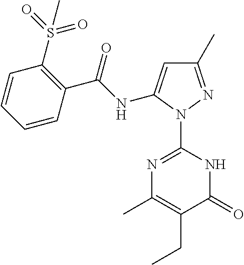 [SMILES: CCc1c(C)nc(-n2nc(C)cc2NC(=O)c2ccccc2S(C)(=O)=O)[nH]c1=O]

### N-(1-(5-Ethyl-4-methyl-6-oxo-1,6-dihydropyrimidin-2-yl)-3-methyl-1H-pyrazol-5-yl)-2-(methylsulfonyl)benzamide (SW335962)

The target compound (71% yield) was obtained as a white solid by the general procedure B described above. ¹H NMR (400 MHz, CDCl₃) δ 12.67 (s, 1H), 8.42 (d, J=7.9 Hz, 1H), 7.92-7.83 (m, 2H), 7.65 (t, J=7.5 Hz, 1H), 6.81 (s, 1H), 2.55 (q, J=7.6 Hz, 2H), 2.36 (s, 3H), 2.29 (s, 3H), 1.12 (t, J=7.4 Hz, 3H). ¹³C NMR (101 MHz, CDCl₃) δ 162.3, 156.9, 153.6, 150.5, 140.2, 133.8, 130.4, 126.3, 125.3, 123.3, 99.1, 45.0, 21.2, 19.0, 14.3, 12.9. ESI-MS (m/z): 416.1 [M+H]⁺.

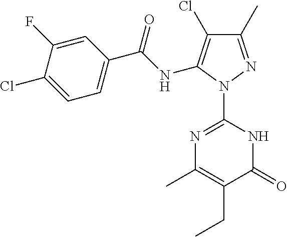 [SMILES: CCc1c(C)nc(-n2nc(C)c(Cl)c2NC(=O)c2ccc(Cl)c(F)c2)[nH]c1=O]

### 4-Chloro-N-(4-chloro-1-(5-ethyl-4-methyl-6-oxo-1,6-dihydropyrimidin-2-yl)-3-methyl-1H-pyrazol-5-yl)-3-fluorobenzamide (SW335981)

NCS (15 mg, 0.11 mmol) was added to a solution of SW223041 (40 mg, 0.10 mmol) in dry CH₃CN (2 mL) and stirred overnight at rt. After completion of reaction, solvent was evaporated and purified by flash chromatography to furnish the desired product (89% yield) as a white solid. ¹H NMR (400 MHz, CDCl₃) δ 11.35 (s, 1H), 8.10 (dd, J=6.8, 2.3 Hz, 1H), 7.97-7.92 (m, 1H), 7.32 (t, J=8.5 Hz, 1H), 2.53 (q, J=7.5 Hz, 2H), 2.31 (s, 3H), 2.29 (s, 3H), 1.10 (t, J=7.5 Hz, 3H). ¹³C NMR (101 MHz, CDCl₃) δ 162.3, 160.7, 159.7, 151.4, 145.3, 136.1, 130.6, 130.2 (d, J=12.8 Hz), 128.4, 123.9, 122.4, 122.2, 117.5, 117.3, 106.2, 21.2, 19.1, 12.8, 12.3. ESI-MS (m/z): 424.1 [M+H]⁺.

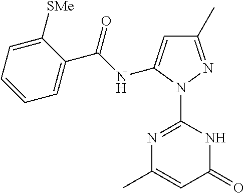 [SMILES: CSc1ccccc1C(=O)Nc1cc(C)nn1-c1nc(C)cc(=O)[nH]1]

### N-(3-Methyl-1-(4-methyl-6-oxo-1,6-dihydropyrimidin-2-yl)-1H-pyrazol-5-yl)-2-(methylthio) benzamide (SW336244)

The target compound (62% yield) was obtained as a white solid by the general procedure B described above. ¹H NMR (400 MHz, CDCl₃) δ 12.28 (s, 1H), 7.71 (d, J=7.8 Hz, 1H), 7.55-7.46 (m, 1H), 7.38 (d, J=8.1 Hz, 1H), 7.23 (d, J=7.7 Hz, 1H), 6.93 (s, 1H), 6.07 (s, 1H), 2.48 (s, 3H), 2.32 (s, 3H), 2.26 (s, 3H). ¹³C NMR (101 MHz, CDCl₃) δ 164.2, 154.2, 148.8, 141.4, 141.1, 132.1, 127.8, 126.2, 124.3, 109.1, 99.2, 23.7, 16.1, 14.3. ESI-MS (m/z): 356.0 [M+H]⁺.

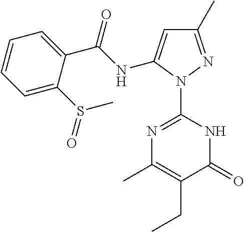 [SMILES: CCc1c(C)nc(-n2nc(C)cc2NC(=O)c2ccccc2S(C)=O)[nH]c1=O]

### N-(1-(5-Ethyl-4-methyl-6-oxo-1,6-dihydropyrimidin-2-yl)-3-methyl-1H-pyrazol-5-yl)-2-(methylsulfinyl)benzamide (SW335980)

To a solution of MMV676477 (50 mg, 0.13 mmol) in MeOH/H₂O (4 mL, 3:1) was added NaIO₄ (30 mg, 0.14 mmol) and stirred for 1 h at 50° C. After that water and EtOAc were added and the aqueous layer was extracted with EtOAc (3×15 mL). The combined organic layer dried over anhydrous Na₂SO₄, concentrated in vacuo and subjected to flash chromatography to give target compound (73% yield) as a white solid. ¹H NMR (400 MHz, CDCl₃) δ 12.67 (s, 1H), 8.47-8.40 (m, 1H), 7.91-7.85 (m, 2H), 7.65 (t, J=7.5 Hz, 1H), 6.83 (s, 1H), 2.96 (s, 3H), 2.56 (q, J=7.6 Hz, 2H), 2.37 (s, 3H), 2.30 (s, 3H), 1.13 (t, J=7.4 Hz, 3H). ¹³C NMR (101 MHz, CDCl₃) δ 162.1, 161.9, 160.8, 156.9, 153.6, 150.6, 146.1, 140.2, 133.8, 130.68-130.32 (m), 127.2, 126.4, 125.3, 123.4, 99.1, 45.1, 21.3, 19.1, 14.3, 12.8. ESI-MS (m/z): 400.2 [M+H]⁺.

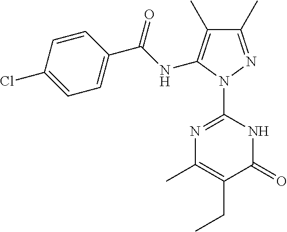 [SMILES: CCc1c(C)nc(-n2nc(C)c(C)c2NC(=O)c2ccc(Cl)cc2)[nH]c1=O]

### 4-Chloro-N-(1-(5-ethyl-4-methyl-6-oxo-1,6-dihydropyrimidin-2-yl)-3,4-dimethyl-1H-pyrazol-5-yl)benzamide (SW336239)

The target compound (91% yield) was obtained as a white solid by the general procedure B described above. ¹H NMR (400 MHz, CDCl₃) δ 11.46 (s, 1H), 7.96 (d, J=8.5 Hz, 2H), 7.50 (d, J=8.4 Hz, 2H), 2.52 (q, J=7.4 Hz, 2H), 2.25 (s, 3H), 2.24 (s, 3H), 2.12 (s, 3H), 1.09 (t, J=7.4 Hz, 3H). ¹³C NMR (101 MHz, CDCl₃) δ 162.7, 153.5, 145.8, 139.1, 136.9, 132.0, 129.2, 122.9, 110.4, 21.2, 19.02, 12.8, 10.1. ESI-MS (m/z): 386.1 [M+H]⁺.

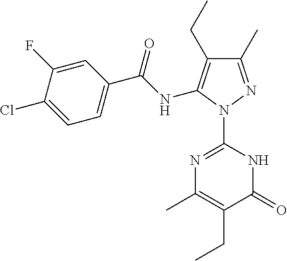 [SMILES: CCc1c(C)nn(-c2nc(C)c(CC)c(=O)[nH]2)c1NC(=O)c1ccc(Cl)c(F)c1]

### 4-Chloro-N-(4-ethyl-1-(5-ethyl-4-methyl-6-oxo-1,6-dihydropyrimidin-2-yl)-3-methyl-1H-pyrazol-5-yl)-3-fluorobenzamide (SW336185)

The target compound (82% yield) was obtained as a white solid by the general procedure B described above. ¹H NMR (400 MHz, CDCl₃) δ 11.64 (s, 1H), 8.10 (dd, J=6.8, 2.2 Hz, 1H), 7.97-7.92 (m, 1H), 7.29 (t, J=8.2 Hz, 1H), 2.65 (q, J=7.5 Hz, 2H), 2.52 (q, J=7.3 Hz, 2H), 2.29 (s, 3H), 2.26 (s, 3H), 1.16-1.04 (m, 6H). ¹³C NMR (101 MHz, CDCl₃) δ 162.1, 161.4, 159.5, 157.3, 153.2, 145.9, 136.3, 130.9 (d, J=12.4 Hz), 130.4, 128.3 (d, J=31.6 Hz), 122.9, 122.0, 117.4, 117.2, 116.3, 21.3, 19.0, 17.9, 13.8, 12.9 (d, J=30.8 Hz). ESI-MS (m/z): 418.1 [M+H]⁺.

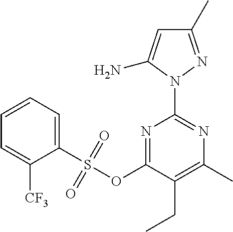 [SMILES: CCc1c(C)nc(-n2nc(C)cc2N)nc1OS(=O)(=O)c1ccccc1C(F)(F)F]

### 2-(5-Amino-3-methyl-1H-pyrazol-1-yl)-5-ethyl-6-methylpyrimidin-4-yl 2-(trifluoromethyl) benzenesulfonate (SW336786)

2-(Trifluoromethyl)benzenesulfonyl chloride (20 μL, 0.13 mmol) was added to a stirred solution of 2-(5-amino3-methyl-1H-pyrazol-1-yl)-5-ethyl-6-methylpyrimidin-4(3H)-one (25 mg, 0.10 mmol) in DCM (1 mL) and triethylamine (22 μL, 0.16 mmol) at 0° C. The solution was stirred for 30 minutes at rt and monitored by LCMS. After the reaction was complete, the reaction mixture was diluted with water and extracted with DCM (3×5 mL). The combined organic layer was dried over Na₂SO₄ and concentrated under reduced pressure. Flash column chromatography of the residue furnished the desired product (72% yield) as a white solid. ¹H NMR (400 MHz, CDCl₃) δ 8.57-8.50 (m, 1H), 7.99-7.92 (m, 1H), 7.88-7.77 (m, 2H), 5.32 (s, 1H), 2.65-2.55 (m, 5H), 2.23 (s, 3H), 1.10 (t, J=7.6 Hz, 3H). ¹³C NMR (101 MHz, CDCl₃) δ 171.7, 162.0, 153.3, 152.9, 149.7, 134.8, 133.7, 132.8, 128.7 (q, J=25.6 Hz), 126.4, 123.7, 120.9, 118.7, 90.3, 22.3, 18.5, 14.4, 13.1. ESI-MS (m/z): 442.0 [M+H]⁺.

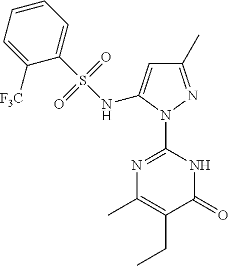 [SMILES: CCc1c(C)nc(-n2nc(C)cc2NS(=O)(=O)c2ccccc2C(F)(F)F)[nH]c1=O]

### N-(1-(5-Ethyl-4-methyl-6-oxo-1,6-dihydropyrimidin-2-yl)-3-methyl-1H-pyrazol-5-yl)-2-(trifluoromethyl)benzenesulfonamide (SW223074-2)

### Step 1. Synthesis of 1-(4-(benzyloxy)-5-ethyl-6-methylpyrimidin-2-yl)-3-methyl-1H-pyrazol-5-amine

To a solution of compound SW223075 (200 mg, 0.85 mmol) in DMF (3 mL) were added K₂CO₃ (142 mg, 1.03 mmol) and benzyl bromide (0.12 mL, 1.03 mmol). The resulting mixture was stirred for 1 h at rt. After that diluted with water (10 mL) and then extracted with EtOAc (3×10 mL). The combined organic layer was dried over anhydrous Na₂SO₄, and concentrated under reduced pressure. The crude product was purified by flash chromatography to obtain 1-(4-(benzyloxy)-5-ethyl-6-methylpyrimidin-2-yl)-3-methyl-1H-pyrazol-5-amine (40% yield).

### Step 2. Synthesis of N-(1-(4-(benzyloxy)-5-ethyl-6-methylpyrimidin-2-yl)-3-methyl-1H-pyrazol-5-yl)-2-(trifluoromethyl)benzenesulfonamide

2-(trifluoromethyl)benzenesulfonyl chloride (38 mg, 0.15 mmol) was added to a stirred solution of above amine (50 mg, 0.15 mmol) in DCM (2 mL), DIPEA (32 μL, 0.18 mmol) and DMAP (6 mg, 0.05 mmol) at 0° C. The solution was stirred for 48 h at rt. After the completion of reaction, the reaction mixture was diluted with water and extracted with DCM (3×10 mL). The combined organic layer was dried over Na₂SO₄ and concentrated under reduced pressure. The crude was used for next reaction without any further purification.

Step 3. To a solution of the above O-benzylated derivative in methanol (5 mL) was added a spatula tip of 10 wt % Pd on activated carbon and the resulting mixture was stirred under hydrogen atmosphere for 1 h at rt. Then the catalyst was removed by filtration over Celite and concentrated under reduced pressure. Flash column chromatography of the residue furnished the target compound (87% yield) as a white solid. H NMR (400 MHz, CDCl₃) δ 11.69 (s, 1H), 8.29 (dd, J=5.7, 3.6 Hz, 1H), 7.90 (dd, J=5.6, 3.5 Hz, 1H), 7.79-7.70 (m, 2H), 6.13 (s, 1H), 2.53 (q, J=7.4 Hz, 2H), 2.34 (s, 3H), 2.19 (s, 3H), 1.10 (t, J=7.5 Hz, 3H). ¹³C NMR (101 MHz, CDCl₃) δ 161.0, 157.9, 152.8, 145.5, 139.7, 137.1, 133.8, 132.5, 132.1, 129.0 (q, J=23.7 Hz), 127.8 (q, J=131.0 Hz), 124.3, 123.3, 121.6, 95.9, 20.7, 19.0, 14.2, 12.7. ESI-MS (m/z): 442.1 [M+H]⁺.

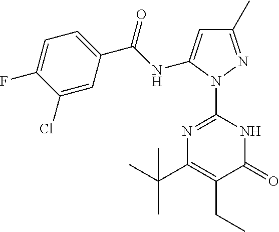 [SMILES: CCc1c(C(C)(C)C)nc(-n2nc(C)cc2NC(=O)c2ccc(F)c(Cl)c2)[nH]c1=O]

### N-(1-(4-(tert-butyl)-5-ethyl-6-oxo-1,6-dihydropyrimidin-2-yl)-3-methyl-1H-pyrazol-5-yl)-3-chloro-4-fluorobenzamide (SW336209)

The target compound (79% yield) was obtained as a white solid by the general procedure B described above. ¹H NMR (400 MHz, MeOD) δ 8.06 (s, 1H), 7.88 (s, 1H), 7.44 (t, J=8.8 Hz, 1H), 6.82 (s, 1H), 2.77 (q, J=7.3 Hz, 2H), 2.34 (s, 3H), 1.31 (s, 9H), 1.18 (t, J=7.3 Hz, 3H). ESI-MS (m/z): 432.2 [M+H]⁺.

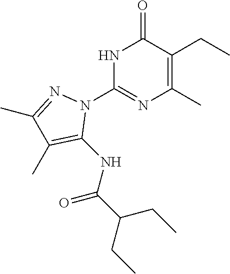 [SMILES: CCc1c(C)nc(-n2nc(C)c(C)c2NC(=O)C(CC)CC)[nH]c1=O]

### 2-Ethyl-N-(1-(5-ethyl-4-methyl-6-oxo-1,6-dihydropyrimidin-2-yl)-3,4-dimethyl-1H-pyrazol-5-yl)butanamide (SW336923)

The target compound (85% yield) was obtained by the general procedure B described above. 1H NMR (400 MHz, CDCl₃) δ 10.25 (s, 1H), 2.51 (q, J=7.5 Hz, 2H), 2.30 (s, 3H), 2.17 (s, 3H), 2.00 (s, 3H), 1.80-1.68 (m, 2H), 1.66-1.54 (m, Hz, 2H), 1.10 (t, J=6.8 Hz, 3H), 0.96 (t, J=7.4 Hz, 6H). 13C NMR (101 MHz, CDCl₃) δ 173.14, 161.27, 157.68, 153.15, 145.52, 136.45, 122.71, 110.71, 52.31, 25.88, 21.32, 18.99, 12.87, 12.72, 12.22, 9.97. ESI-MS (m/z): 346.2 [M+H]⁺.

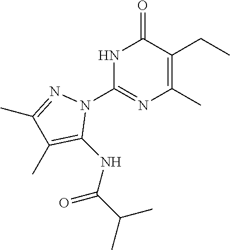 [SMILES: CCc1c(C)nc(-n2nc(C)c(C)c2NC(=O)C(C)C)[nH]c1=O]

### N-(1-(5-ethyl-4-methyl-6-oxo-1,6-dihydropyrimidin-2-yl)-3,4-dimethyl-1H-pyrazol-5-yl)isobutyramide (SW336924)

The target compound (72% yield) was obtained by the general procedure B described above. ¹H NMR (400 MHz, CDCl₃) δ 10.25 (s, 1H), 2.61 (p, J=7.0 Hz, 1H), 2.51 (q, J=7.5 Hz, 2H), 2.30 (s, 3H), 2.16 (s, 3H), 1.98 (s, 3H), 1.29 (d, J=6.9 Hz, 6H), 1.09 (t, J=7.5 Hz, 3H). 13C NMR (101 MHz, CDCl₃) δ 173.99, 161.16, 157.58, 153.02, 145.41, 136.39, 122.60, 110.55, 36.38, 21.19, 19.40, 18.85, 12.77, 12.61, 9.71. ESI-MS (m/z): 318.2 [M+H]⁺.

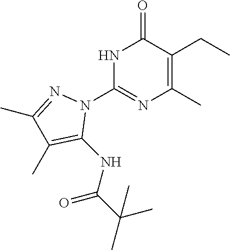 [SMILES: CCc1c(C)nc(-n2nc(C)c(C)c2NC(=O)C(C)(C)C)[nH]c1=O]

### N-(1-(5-ethyl-4-methyl-6-oxo-1,6-dihydropyrimidin-2-yl)-3,4-dimethyl-1H-pyrazol-5-yl)pivalamide (SW336925)

The target compound (85% yield) was obtained by the general procedure B described above ¹H NMR (400 MHz, CDCl₃) δ 10.32 (s, 1H), 2.51 (q, J=7.5 Hz, 2H), 2.29 (s, 3H), 2.16 (s, 3H), 1.95 (s, 3H), 1.33 (s, 9H), 1.08 (t, J=7.5 Hz, 3H). ¹³C NMR (100 MHz, CDCl₃) δ 175.74, 161.16, 157.63, 153.02, 145.45, 136.59, 122.61, 110.37, 39.63, 27.52, 21.07, 18.84, 12.79, 12.70, 9.84. ESI-MS (m/z): 332.2 [M+H]⁺.

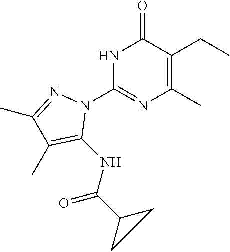 [SMILES: CCc1c(C)nc(-n2nc(C)c(C)c2NC(=O)C2CC2)[nH]c1=O]

### N-(1-(5-ethyl-4-methyl-6-oxo-1,6-dihydropyrimidin-2-yl)-3,4-dimethyl-1H-pyrazol-5-yl)cyclopropanecarboxamide (SW336926)

The target compound (78% yield) was obtained by the general procedure B described above ¹H NMR (400 MHz, CDCl₃) δ 10.55 (s, 1H), 2.52 (q, J=7.5 Hz, 2H), 2.32 (s, 3H), 2.16 (s, 3H), 1.98 (s, 3H), 1.17-1.05 (m, 6H), 0.93 (m, 2H). 13C NMR (101 MHz, CDCl₃) δ 170.98, 161.21, 158.01 153.07, 145.46, 136.52, 122.53, 110.48, 21.25, 18.86, 15.54, 12.77, 12.62, 9.65, 8.49. ESI-MS (m/z): 316.1 [M+H]⁺.

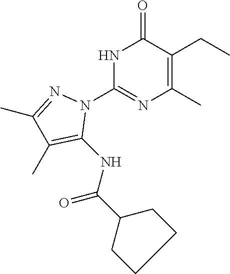 [SMILES: CCc1c(C)nc(-n2nc(C)c(C)c2NC(=O)C2CCCC2)[nH]c1=O]

### N-(1-(5-ethyl-4-methyl-6-oxo-1,6-dihydropyrimidin-2-yl)-3,4-dimethyl-1H-pyrazol-5-yl)cyclopentanecarboxamide (SW336927)

The target compound (80% yield) was obtained by the general procedure B described above ¹H NMR (400 MHz, CDCl₃) δ 10.23 (s, 1H), 2.82 (p, J=8.1 Hz, 1H), 2.52 (q, J=7.5 Hz, 2H), 2.31 (s, 3H), 2.17 (s, 3H), 1.99 (s, 3H), 1.97-1.84 (m, 4H), 1.82-1.72 (m, 2H), 1.70-1.57 (m, 2H), 1.10 (t, J=7.5 Hz, 3H). ¹³C NMR (101 MHz, CDCl₃) δ 173.47, 153.16, 145.56, 136.63, 122.68, 110.66, 46.55, 30.33, 26.04, 21.26, 12.90, 12.74, 9.85. ESI-MS (m/z): 344.2 [M+H]⁺.

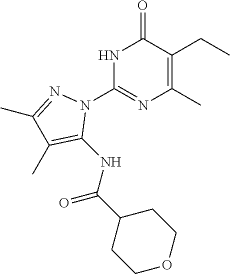 [SMILES: CCc1c(C)nc(-n2nc(C)c(C)c2NC(=O)C2CCOCC2)[nH]c1=O]

### N-(1-(5-ethyl-4-methyl-6-oxo-1,6-dihydropyrimidin-2-yl)-3,4-dimethyl-1H-pyrazol-5-yl)tetrahydro-2H-pyran-4-carboxamide (SW336928)

The target compound (62% yield) was obtained by the general procedure B described above ¹H NMR (400 MHz, CDCl₃) δ 10.36 (s, 1H), 4.12-4.03 (m, 2H), 3.49 (td, J=11.3, 2.8 Hz, 2H), 2.68-258 (m 1H) 2.53 (q, J=7.5 Hz, 2H), 2.31 (s, 3H), 2.19 (s, 3H), 2.00 (s, 3H), 1.98-1.92 (m, 2H), 1.95-1.80 (m, 2H), 1.10 (t, J=7.5 Hz, 3H). ESI-MS (m/z): 360.2 [M+H]⁺.

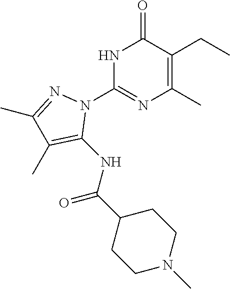 [SMILES: CCc1c(C)nc(-n2nc(C)c(C)c2NC(=O)C2CCN(C)CC2)[nH]c1=O]

### N-(1-(5-ethyl-4-methyl-6-oxo-1,6-dihydropyrimidin-2-yl)-3,4-dimethyl-1H-pyrazol-5-yl)-1-methylpiperidine-4-carboxamide (SW336929)

The target compound (74% yield) was obtained by the general procedure B described above ¹H NMR (400 MHz, CDCl₃) δ 10.36 (s, 1H), 3.03-2.94 (m, 2H), 2.52 (q, J=7.5 Hz, 2H), 2.34 (s, 3H), 2.30 (s, 3H), 2.18 (s, 3H), 2.15 (s, 2H), 2.12-2.04 (m, 2H), 1.99 (s, 3H), 1.96-1.84 (m, 2H), 1.10 (t, J=7.5 Hz, 3H). 13C NMR (101 MHz, CDCl₃) δ 171.91, 153.13, 145.81, 136.32, 122.72, 110.76, 54.85, 46.17, 28.53, 19.01, 12.92, 12.75, 9.90. ESI-MS (m/z): 373.2 [M+H]⁺.

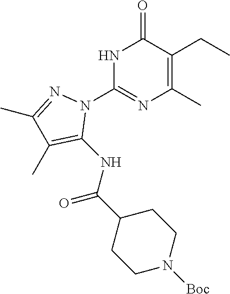 [SMILES: CCc1c(C)nc(-n2nc(C)c(C)c2NC(=O)C2CCN(C(=O)OC(C)(C)C)CC2)[nH]c1=O]

### Tert-butyl4-((1-(5-ethyl-4-methyl-6-oxo-1,6-dihydropyrimidin-2-yl)-3,4-dimethyl-1H-pyrazol-5-yl)carbamoyl)piperidine-1-carboxylate (SW336930)

The target compound (80% yield) was obtained by the general procedure B described above ¹H NMR (400 MHz, CDCl₃) δ 10.36 (s, 1H), 4.19-4.14 (m, 2H), 2.90-2.79 (m, 2H), 2.52 (q, J=7.4 Hz, 3H), 2.30 (s, 3H), 2.20-2.16 (m, 3H), 2.01 (d, J=3.9 Hz, 1H), 1.99 (s, 5H), 1.80-1.65 (m, 2H), 1.46 (s, 9H), 1.10 (t, J=7.5 Hz, 3H). ¹³C NMR (101 MHz, CDCl₃) δ 154.69, 145.61, 136.27, 110.89, 79.91, 44.01, 28.66, 28.53, 18.98, 12.88, 12.73, 9.85. ESI-MS (m/z): 459.2 [M+H]⁺.

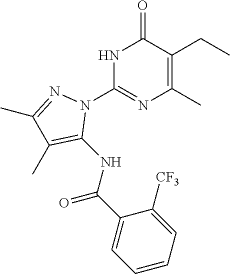 [SMILES: CCc1c(C)nc(-n2nc(C)c(C)c2NC(=O)c2ccccc2C(F)(F)F)[nH]c1=O]

### N-(1-(5-ethyl-4-methyl-6-oxo-1,6-dihydropyrimidin-2-yl)-3,4-dimethyl-1H-pyrazol-5-yl)-2-(trifluoromethyl)benzamide) (SW337352)

The target compound (86% yield) was obtained by the general procedure B described above ¹H NMR (400 MHz, CDCl₃) δ 10.73 (s, 1H), 7.80 (dd, J=7.0, 2.0 Hz, 1H), 7.77-7.72 (m, 1H), 7.71-7.59 (m, 2H), 2.48 (q, J=7.5 Hz, 2H), 2.23 (s, 3H), 2.13 (s, 3H), 2.04 (s, 3H), 1.08 (t, J=7.5 Hz, 3H). ¹³C NMR (101 MHz, CDCl₃) δ 164.05, 161.17, 157.50, 153.34, 145.50, 135.74, 135.25, 132.11, 130.82, 128.39, 127.03 (q, J=5.0 Hz), 124.97, 122.90, 122.25, 111.50, 20.94, 18.98, 12.83, 12.75, 9.61. ESI-MS (m/z): 420.1[M+H]⁺.

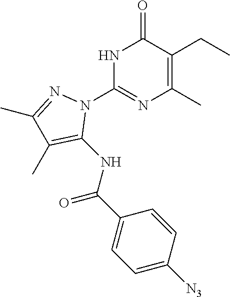 [SMILES: CCc1c(C)nc(-n2nc(C)c(C)c2NC(=O)c2ccc(N=[N+]=[N-])cc2)[nH]c1=O]

### 4-azido-N-(1-(5-ethyl-4-methyl-6-oxo-1,6-dihydropyrimidin-2-yl)-3,4-dimethyl-1H-pyrazol-5-yl)benzamide (SW337383)

The target compound (70% yield) was obtained by the general procedure B described above ¹H NMR (400 MHz, CDCl₃) δ 11.42 (s, 1H), 8.01 (d, J=8.5 Hz, 2H), 7.14 (d, J=8.6 Hz, 2H), 2.50 (q, J=7.5, 1.5 Hz, 2H), 2.25 (s, 3H), 2.22 (s, 3H), 2.10 (s, 3H), 1.08 (d, J=7.5 Hz, 3H). ¹³C NMR (101 MHz, CDCl₃) δ 162.72, 153.51, 145.78, 144.61, 137.01, 130.01, 129.59, 122.81, 119.33, 110.29, 21.27, 18.98, 12.89, 12.81, 10.11. ESI-MS (m/z): 393.1 [M+H]⁺.

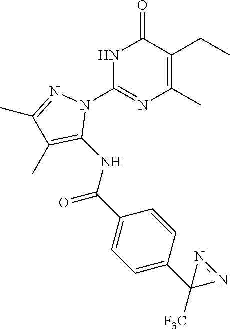 [SMILES: CCc1c(C)nc(-n2nc(C)c(C)c2NC(=O)c2ccc(C3(C(F)(F)F)N=N3)cc2)[nH]c1=O]

### N-(1-(5-ethyl-4-methyl-6-oxo-1,6-dihydropyrimidin-2-yl)-3,4-dimethyl-1H-pyrazol-5-yl)-4-(3-(trifluoromethyl)-3H-diazirin-3-yl)benzamide (SW337384)

The target compound (82% yield) was obtained by the general procedure B described above ¹H NMR (400 MHz, CDCl₃) δ 11.49 (s, 1H), 8.05 (d, J=8.2 Hz, 2H), 7.33 (d, J=8.2 Hz, 2H), 2.52 (q, J=7.4 Hz, 2H), 2.24 (d, J=6.0 Hz, 6H), 2.11 (s, 3H), 1.09 (t, J=7.5 Hz, 3H). ¹³C NMR (101 MHz, CDCl₃) δ 162.45, 161.15, 157.29, 153.56, 145.77, 136.69, 134.70, 133.64, 128.19, 126.89, 122.98, 110.58, 21.34, 19.01, 12.90, 12.83, 10.14. ESI-MS (m/z): 460.0 [M+H]

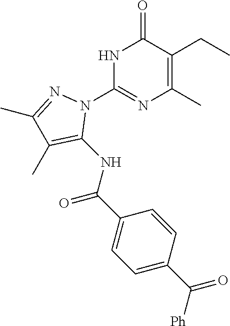 [SMILES: CCc1c(C)nc(-n2nc(C)c(C)c2NC(=O)c2ccc(C(=O)c3ccccc3)cc2)[nH]c1=O]

### 4-benzoyl-N-(1-(5-ethyl-4-methyl-6-oxo-1,6-dihydropyrimidin-2-yl)-3,4-dimethyl-1H-pyrazol-5-yl)benzamide (SW337385)

The target compound (80% yield) was obtained by the general procedure B described above ¹H NMR (500 MHz, CDCl₃) δ 11.59 (s, 1H), 8.14 (d, J=8.1 Hz, 2H), 7.94 (d, J=8.5 Hz, 2H), 7.82 (dd, J=8.4, 1.3 Hz, 2H), 7.67-7.61 (m, 1H), 7.52 (t, J=8.1, 7.3 Hz, 2H), 2.52 (q, J=7.5 Hz, 2H), 2.27 (s, 3H), 2.25 (s, 3H), 2.16 (s, 3H), 1.09 (t, J=7.5 Hz, 3H). ¹³C NMR (101 MHz, CDCl₃) δ 195.88, 162.79, 161.12, 158.03, 153.57, 145.79, 141.13, 137.04, 136.77, 133.18, 130.38, 130.22, 128.66, 127.75, 123.01, 21.31, 19.00, 12.88, 12.81, 10.16. ESI-MS (m/z): 456.1 [M+H]⁺.

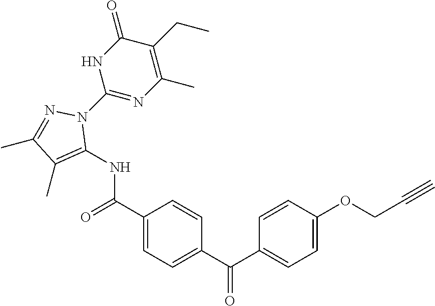 [SMILES: C#CCOc1ccc(C(=O)c2ccc(C(=O)Nc3c(C)c(C)nn3-c3nc(C)c(CC)c(=O)[nH]3)cc2)cc1]

### N-(1-(5-ethyl-4-methyl-6-oxo-1,6-dihydropyrimidin-2-yl)-3,4-dimethyl-1H-pyrazol-5-yl)-4-(4-(prop-2-yn-1-yloxy)benzoyl)benzamide (SW337386)

The target compound (42% yield) was obtained by the general procedure B described above ¹H NMR (400 MHz, CDCl₃) δ 11.59 (s, 1H), 8.14 (d, J=8.0 Hz, 2H), 7.90 (d, J=8.0 Hz, 2H), 7.85 (d, J=8.5 Hz, 2H), 7.08 (d, J=8.5 Hz, 2H), 4.80 (d, J=2.4 Hz, 2H), 2.62-2.56 (m, 1H), 2.56-2.47 (m, 2H), 2.28 (s, 3H), 2.23 (s, 3H), 2.15 (s, 3H), 1.09 (t, J=7.2 Hz, 3H). ¹³C NMR (101 MHz, CDCl₃) δ 194.60, 162.86, 161.57, 136.40, 132.63, 130.36, 130.09, 127.72, 114.79, 77.74, 76.47, 56.04, 21.31, 19.02, 12.89, 10.20. ESI-MS (m/z): 510.1 [M+H]⁺.

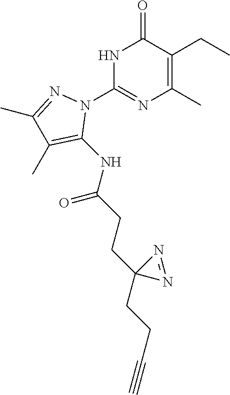 [SMILES: C#CCCC1(CCC(=O)Nc2c(C)c(C)nn2-c2nc(C)c(CC)c(=O)[nH]2)N=N1]

### 3-(3-(but-3-yn-1-yl)-3H-diazirin-3-yl)-N-(1-(5-ethyl-4-methyl-6-oxo-1,6-dihydropyrimidin-2-yl)-3,4-dimethyl-1H-pyrazol-5-yl)propenamide (SW337387)

To a solution of 3-(3-(but-3-yn-1-yl)-3H-diazirin-3-yl)propanoic acid (12 mg, 0.072 mmol) in DCM (2 mL) were added 2-(5-amino-3,4-dimethyl-1H-pyrazol-1-yl)-5-ethyl-6-methylpyrimidin-4(3H)-one (21 mg, 0.086 mmol), T₃P (0.091 mL, 0.288 mmol) and TEA (0.040 mL, 0.288 mmol). The mixture was stirred at rt overnight and monitored by LCMS, after complete Aq. NaHCO₃ was added to quench the reaction. The aqueous mixture was extracted with dichloromethane, and the combined organic phases were washed with brine, dried over anhydrous Na₂SO₄ and concentrated to deliver the crude product, which was then purified by flash chromatography on silica gel to obtain amide compound (12 mg, 38% yield). ¹H NMR (400 MHz, CDCl₃) δ 10.26 (s, 1H), 2.54 (q, J=8.3, 4.1 Hz, 2H), 2.33 (s, 3H), 2.19 (s, 5H), 2.08-1.91 (m, 8H), 1.69 (t, J=7.4, 1.8 Hz, 2H), 1.11 (t, J=7.5, 1.7 Hz, 3H). ¹³C NMR (101 MHz, CDCl₃) δ 168.36, 156.82, 80.46, 63.62, 33.27, 31.26, 29.84, 28.27, 27.85, 21.25, 16.84, 13.47, 12.41, 9.94. ESI-MS (m/z): 396.1 [M+H]⁺.

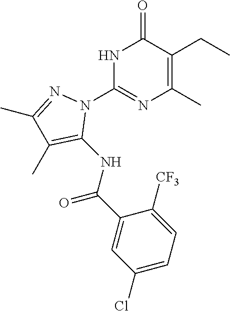 [SMILES: CCc1c(C)nc(-n2nc(C)c(C)c2NC(=O)c2cc(Cl)ccc2C(F)(F)F)[nH]c1=O]

### 5-chloro-N-(1-(5-ethyl-4-methyl-6-oxo-1,6-dihydropyrimidin-2-yl)-3,4-dimethyl-1H-pyrazol-5-yl)-2-(trifluoromethyl)benzamide (SW387974)

The target compound (75% yield) was obtained by the general procedure B described above ¹H NMR (400 MHz, CDCl₃) δ 10.77 (s, 1H), 7.95 (s, 1H), 7.82 (d, J=8.2 Hz, 1H), 7.65 (d, J=8.2 Hz, 1H), 2.50 (q, J=7.5 Hz, 2H), 2.24 (s, 3H), 2.11 (s, 3H), 2.08 (s, 3H), 1.09 (t, J=7.5 Hz, 3H). ¹³C NMR (151 MHz, MeOD) δ 165.79, 153.78, 138.06, 135.07, 133.89, 131.77, 130.39, 130.09, 129.88, 129.80, 125.19, 123.72, 120.96, 112.15, 21.19, 18.93, 12.72, 12.59, 9.13. ESI-MS (m/z): 454.0 [M+H]⁺.

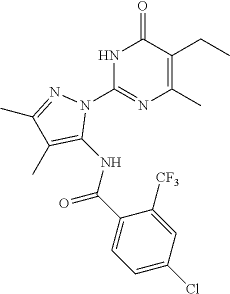 [SMILES: CCc1c(C)nc(-n2nc(C)c(C)c2NC(=O)c2ccc(Cl)cc2C(F)(F)F)[nH]c1=O]

### 4-chloro-N-(1-(5-ethyl-4-methyl-6-oxo-1,6-dihydropyrimidin-2-yl)-3,4-dimethyl-1H-pyrazol-5-yl)-2-(trifluoromethyl)benzamide (SW387975)

The target compound (78% yield) was obtained by the general procedure B described above ¹H NMR (400 MHz, CDCl₃) δ 10.73 (s, 1H), 7.78-7.72 (m, 2H), 7.61 (d, J=8.5 Hz, 1H), 2.52 (q, J=7.4 Hz, 2H), 2.25 (s, 3H), 2.16 (s, 3H), 2.11 (s, 3H), 1.10 (t, J=7.5 Hz, 3H). ¹³C NMR (101 MHz, CDCl₃) δ 162.46, 153.40, 145.16, 138.63, 136.25, 135.35, 132.55, 131.42, 130.93, 128.72 (q, J=5.0 Hz), 127.08, 126.69, 125.26, 124.62, 123.38, 122.20, 109.36, 22.63, 21.02, 19.02, 12.83, 12.74, 9.94, 9.54. ESI-MS (m/z): 454.0 [M+H]⁺.

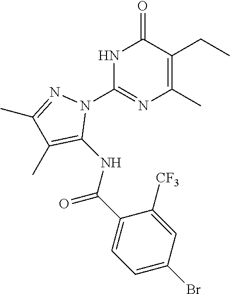 [SMILES: CCc1c(C)nc(-n2nc(C)c(C)c2NC(=O)c2ccc(Br)cc2C(F)(F)F)[nH]c1=O]

### 4-bromo-N-(1-(5-ethyl-4-methyl-6-oxo-1,6-dihydropyrimidin-2-yl)-3,4-dimethyl-1H-pyrazol-5-yl)-2-(trifluoromethyl)benzamide (SW387976)

The target compound (80% yield) was obtained by the general procedure B described above ¹H NMR (400 MHz, CDCl₃) δ 10.78 (s, 1H), 7.80 (d, J=2.0 Hz, 1H), 7.73 (d, J=8.2 Hz, 1H), 7.65 (dd, J=8.3, 2.0 Hz, 1H), 2.50 (q, J=7.5 Hz, 2H), 2.24 (s, 3H), 2.11 (s, 3H), 2.08 (s, 3H), 1.09 (t, J=7.5 Hz, 3H). ¹³C NMR (151 MHz, MeOD) δ 162.31, 153.40, 137.12, 134.38, 133.25, 131.69, 129.88, 129.63, 127.35 (q, J=5.1 Hz), 123.62, 18.76, 13.49, 12.48, 9.09. ESI-MS (m/z): 499.9 [M+H]⁺.

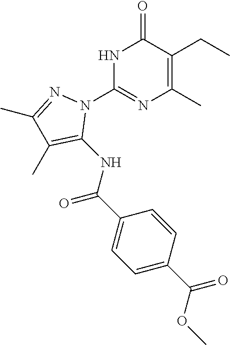 [SMILES: CCc1c(C)nc(-n2nc(C)c(C)c2NC(=O)c2ccc(C(=O)OC)cc2)[nH]c1=O]

### Methyl 4-((1-(5-ethyl-4-methyl-6-oxo-1,6-dihydropyrimidin-2-yl)-3,4-dimethyl-1H-pyrazol-5-yl)carbamoyl)benzoate (SW387981)

The target compound (84% yield) was obtained by the general procedure B described above ¹H NMR (400 MHz, CDCl₃) δ 11.57 (s, 1H), 8.20 (d, J=8.4 Hz, 2H), 8.09 (d, J=8.4 Hz, 2H), 3.98 (s, 3H), 2.53 (q, J=7.5 Hz, 2H), 2.27 (s, 6H), 2.14 (s, 3H), 1.09 (t, J=7.5 Hz, 3H). ¹³C NMR (101 MHz, CDCl₃) δ 167.31, 162.79, 154.81, 146.93, 138.26, 136.78, 133.79, 130.14, 127.80, 122.99, 112.37, 51.79, 21.92, 19.00, 13.35, 12.80, 9.20. ESI-MS (m/z): 410.1 [M+H]⁺.

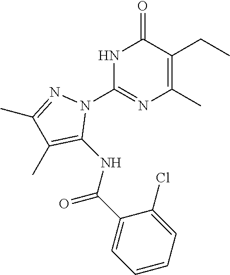 [SMILES: CCc1c(C)nc(-n2nc(C)c(C)c2NC(=O)c2ccccc2Cl)[nH]c1=O]

### 2-chloro-N-(1-(5-ethyl-4-methyl-6-oxo-1,6-dihydropyrimidin-2-yl)-3,4-dimethyl-1H-pyrazol-5-yl)benzamide (SW388955)

The target compound (82% yield) was obtained by the general procedure B described above H NMR (400 MHz, CDCl₃) δ 11.01 (s, 1H), 7.78 (dd, J=7.6, 1.8 Hz, 1H), 7.51 (dd, J=8.0, 1.4 Hz, 1H), 7.46 (td, J=7.6, 1.8 Hz, 1H), 7.39 (td, J=7.4, 1.5 Hz, 1H), 2.50 (q, J=7.5 Hz, 2H), 2.25 (s, 3H), 2.15 (s, 3H), 2.12 (s, 3H), 1.08 (t, J=7.5 Hz, 3H). ¹³C NMR (101 MHz, CDCl₃) δ 163.01, 161.12, 153.92, 145.42, 137.81, 134.57, 133.18, 131.65, 130.88, 128.85, 126.34, 121.91, 113.44, 20.97, 18.96, 12.84, 12.79, 8.98. ESI-MS (m/z): 386.1 [M+H]⁺.

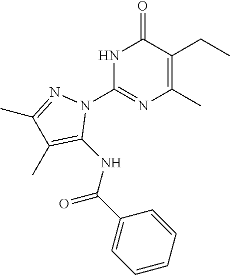 [SMILES: CCc1c(C)nc(-n2nc(C)c(C)c2NC(=O)c2ccccc2)[nH]c1=O]

### N-(1-(5-ethyl-4-methyl-6-oxo-1,6-dihydropyrimidin-2-yl)-3,4-dimethyl-1H-pyrazol-5-yl)benzamide (SW389121)

The target compound (88% yield) was obtained by the general procedure B described above ¹H NMR (400 MHz, CDCl₃) δ 11.43 (s, 1H), 8.01 (d, J=7.1 Hz, 2H), 7.68-7.57 (m, 1H), 7.52 (d, J=8.3 Hz, 2H), 2.50 (q, J=7.5 Hz, 2H), 2.25 (s, 3H), 2.22 (s, 3H), 2.12 (s, 3H), 1.08 (t, J=7.5 Hz, 3H). ¹³C NMR (101 MHz, CDCl₃) δ 165.21, 160.69, 157.04, 151.44, 144.03, 137.54, 134.61, 132.69, 129.45, 127.74, 123.92, 111.46, 21.20, 19.45, 13.73, 12.81, 10.92. ESI-MS (m/z): 352.1 [M+H]⁺.

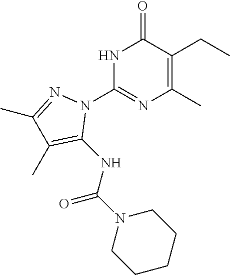 [SMILES: CCc1c(C)nc(-n2nc(C)c(C)c2NC(=O)N2CCCCC2)[nH]c1=O]

### N-(1-(5-ethyl-4-methyl-6-oxo-1,6-dihydropyrimidin-2-yl)-3,4-dimethyl-1H-pyrazol-5-yl)piperidine-1-carboxamide (SW388743)

The target compound (74% yield) was obtained by the general procedure B described above ¹H NMR (400 MHz, CDCl₃) δ 10.05 (s, 1H), 3.61-3.45 (m, 4H), 2.52 (q, J=7.5 Hz, 2H), 2.28 (s, 3H), 2.18 (s, 3H), 1.99 (s, 3H), 1.71-1.59 (m, 6H), 1.10 (t, J=7.5 Hz, 3H). ¹³C NMR (101 MHz, CDCl₃) δ 154.41, 152.86, 146.07, 137.48, 48.77, 26.01, 23.88, 19.38, 12.95, 11.94, 9.29. ESI-MS (m/z): 359.1 [M+H]⁺.

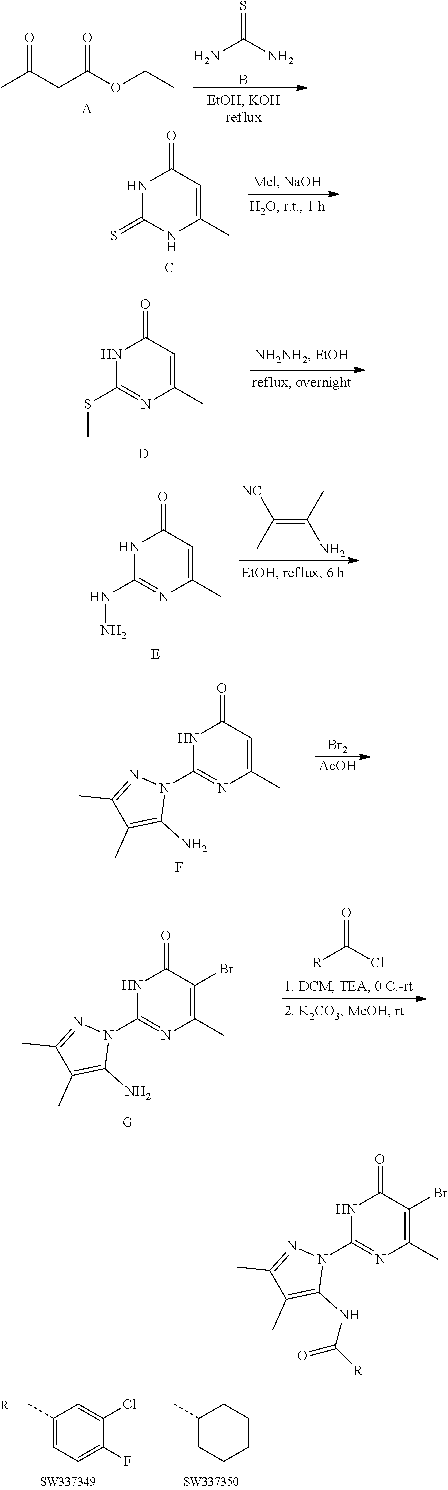

### Synthesis of 6-methyl-2-thioxo-2,3-dihydropyrimidin-4(1H)-one (C)

Ethyl 3-oxobutanoate (1.3 mL, 10 mmol) was added to a stirred suspension of thiourea (766 mg, 10 mmol) and KOH (672 mg, 12 mmol) in EtOH (20 mL). The solution was refluxed for 5 h and completion of the reaction was confirmed by LCMS. The solid formed was collected by filtration and then dissolved in H₂O. The solution was then acidified with 1N HCl to pH 1 to give white precipitate of 5-ethyl-6-methyl-2-thioxo-2,3-dihydropyrimidin-4(1H)-one that was filtered and dried under vacuum (1.19 g, 84% yield). ESI-MS (m/z): 143.0 [M+H]⁺.

### Synthesis of 6-methyl-2-(methylthio)pyrimidin-4(3H)-one (D)

NaOH (176 mg, 4.4 mmol) was added to a suspension of 6-methyl-2-thioxo-2,3-dihydropyrimidin-4(1H)-one (C) (568 mg, 4.0 mmol) in H₂O (12 mL) and the resulting mixture was allowed to stir to clarity. Then, the solution was cooled in an ice bath, and iodomethane (0.28 mL, 4.4 mmol) was added dropwise to the solution. The reaction was warmed to room temperature and continued to stir for 1 h, precipitate formed was filtered, washed with H₂O and dried under vacuum to yield 6-methyl-2-(methylthio)pyrimidin-4(3H)-one as a white solid (536 mg, 86% yield). ¹H NMR (400 MHz, Chloroform-d) δ 6.06 (s, 1H), 2.57 (s, 3H), 2.27 (s, 3H). ESI-MS (m/z): 157.1 [M+H]⁺.

### Synthesis of 2-hydrazineyl-6-methylpyrimidin-4(3H)-one (E)

6-methyl-2-(methylthio)pyrimidin-4(3H)-one (D)(514 mg, 2.2 mmol), hydrazine monohydrate (0.193 mL, 5 mmol), and EtOH (4 mL) were combined in a microwave tube and heated at 100° C. for 12 h. Then, the solution was filtered and the solid was washed with EtOH and dried overnight. The filtrate was condensed to give purple oil, and EtOH and EtOAc were added to precipitate out any remaining product. Some solid formed and was filtered and dried overnight. The samples were combined to give 2-hydrazineyl-6-methylpyrimidin-4(3H)-one (E) as a light purple solid. ESI-MS (m/z): 141.0 [M+H]⁺.

### Synthesis of 2-(5-amino-3,4-dimethyl-1H-pyrazol-1-yl)-6-methylpyrimidin-4(3H)-one (F)

3-Iminobutanenitrile (230 mg, 2.4 mmol) was added to a stirred solution of 2-hydrazineyl-6-methylpyrimidin-4(3H)-one (E) (280 mg, 2.0 mmol) in EtOH (6 mL). After heating at 100° C. for 6 h, the mixture was cooled to room temperature and concentrated to give yellow solid. The solid was recrystallized from EtOAc/Hexanes to provide 2-(5-amino-3,4-dimethyl-1H-pyrazol-1-yl)-6-methylpyrimidin-4(3H)-one (F) as a yellow solid (309 mg, 86% yield)¹H NMR (500 MHz, CDCl₃) δ 5.99 (s, 1H), 5.59 (s, 2H), 2.27 (s, 3H), 2.12 (s, 4H), 1.82 (s, 3H). ESI-MS (m/z): 220.1[M+H]⁺.

### 2-(5-amino-3,4-dimethyl-1H-pyrazol-1-yl)-5-bromo-6-methylpyrimidin-4(3H)-one (G)

To a solution of the above 2-(5-amino-3,4-dimethyl-1H-pyrazol-1-yl)-6-methylpyrimidin-4(3H)-one (F) (250 mg, 1.14 mmol) in glacial acetic acid (6 mL) was added bromine (199 mg, 1.25 mmol) and the resulting mixture was stirred for 30 minutes at room temperature and monitored by LCMS, after the reaction was complete, the reaction mixture was diluted with water and extracted with DCM (3×10 mL). and the combined organic layer was dried over anhydrous Na₂SO₄, evaporated under reduced pressure and purified by flash column chromatography to give the product 2-(5-amino-3,4-dimethyl-1H-pyrazol-1-yl)-5-bromo-6-methylpyrimidin-4(3H)-one (G)(230 mg 68% yield)¹H NMR (400 MHz, DMSO-d₆) δ 6.80 (s, 2H), 2.42 (s, 3H), 2.07 (s, 3H), 1.80 (s, 3H). ESI-MS (m/z): 298.9 [M+H]⁺.

Acylation of Compound G was Carried about According to General Procedure B.

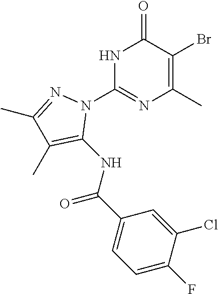 [SMILES: Cc1nn(-c2nc(C)c(Br)c(=O)[nH]2)c(NC(=O)c2ccc(F)c(Cl)c2)c1C]

### N-(1-(5-bromo-4-methyl-6-oxo-1,6-dihydropyrimidin-2-yl)-3,4-dimethyl-1H-pyrazol-5-yl)-3-chloro-4-fluorobenzamide (SW337349)

The target compound (85% yield) was obtained by the general procedure B described above ¹H NMR (400 MHz, DMSO-d₆) δ 10.56 (s, 1H), 8.16 (d, J=6.9 Hz, 1H), 8.00-7.92 (m, 1H), 7.60 (t, J=8.9 Hz, 1H), 2.22 (s, 3H), 2.17 (s, 3H), 1.90 (s, 3H). ¹³C NMR (101 MHz, DMSO-d₆) δ 163.46, 160.79, 158.28, 151.17, 134.85, 131.23, 130.42, 129.32, 129.24, 120.14, 119.96, 117.58, 117.36, 114.31, 12.46, 7.84. ESI-MS (m/z): 455.8 [M+H]⁺.

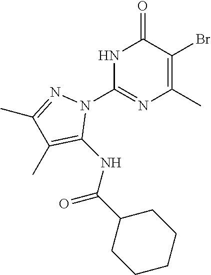

### N-(1-(5-bromo-4-methyl-6-oxo-1,6-dihydropyrimidin-2-yl)-3,4-dimethyl-1H-pyrazol-5-yl)cyclohexanecarboxamide (SW337350)

The target compound (85% yield) was obtained by the general procedure B described above ¹H NMR (500 MHz, CDCl₃) δ 9.88 (s, 1H), 2.53 (s, 3H), 2.41-2.28 (m, 1H), 2.23 (s, 3H), 2.10-2.06 (m, 2H), 2.02 (s, 3H), 1.93-1.83 (m, 2H), 1.80-1.73 (m, 1H), 1.55-1.50 (m, 1H), 1.41-1.28 (m, 4H). ¹³C NMR (101 MHz, CDCl₃) δ 173.18, 161.52, 157.20, 154.34, 146.27, 136.58, 132.84, 128.14, 111.79, 107.30, 46.13, 29.80, 29.71, 25.83, 25.70, 24.99, 22.11, 13.03, 12.85, 9.88. ESI-MS (m/z): 410.0 [M+H]⁺.

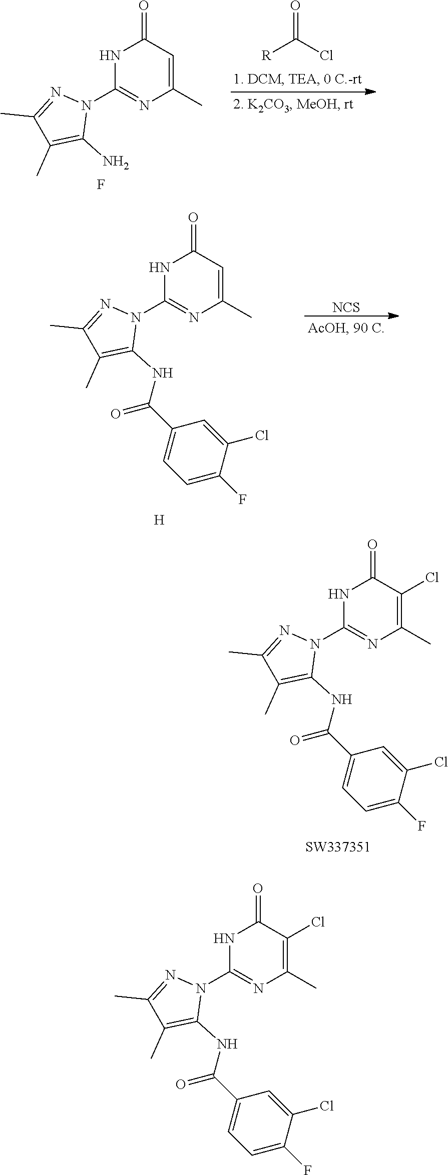

### 3-Chloro-N-(1-(5-chloro-4-methyl-6-oxo-1,6-dihydropyrimidin-2-yl)-3,4-dimethyl-1H-pyrazol-5-yl)-4-fluorobenzamide (SW337351)

To a solution of the above 2-(5-amino-3,4-dimethyl-1H-pyrazol-1-yl)-6-methylpyrimidin-4(3H)-one (F) (250 mg, 1.14 mmol) in glacial acetic acid (6 mL) was added NCS (166 mg, 1.25 mmol) and the resulting mixture was stirred for 30 minutes at room temperature and monitored by LCMS, after the reaction was complete, the reaction mixture was diluted with water and extracted with DCM (3×10 mL) and the combined organic layer was dried over anhydrous Na₂SO₄, evaporated under reduced pressure and purified by flash column chromatography to give the 2-(5-amino-3,4-dimethyl-1H-pyrazol-1-yl)-5-chloro-6-methylpyrimidin-4(3H)-one (H) (179 mg 62% yield). ¹H NMR (400 MHz, CDCl₃) δ 11.03 (s, 1H), 8.07 (dd, J=6.8, 2.3 Hz, 1H), 7.92 (ddd, J=8.6, 4.5, 2.3 Hz, 1H), 7.31 (t, J=8.5 Hz, 1H), 2.44 (s, 3H), 2.25 (s, 3H), 2.11 (s, 3H). ¹³C NMR (101 MHz, CDCl₃) δ 162.22, 161.42, 159.67, 158.89, 156.79, 154.69, 145.67, 136.60, 130.37, 128.23, 128.14, 117.54, 117.32, 116.77, 111.52, 29.85, 22.51, 12.92, 10.11. ESI-MS (m/z): 411.9 [M+H]⁺.

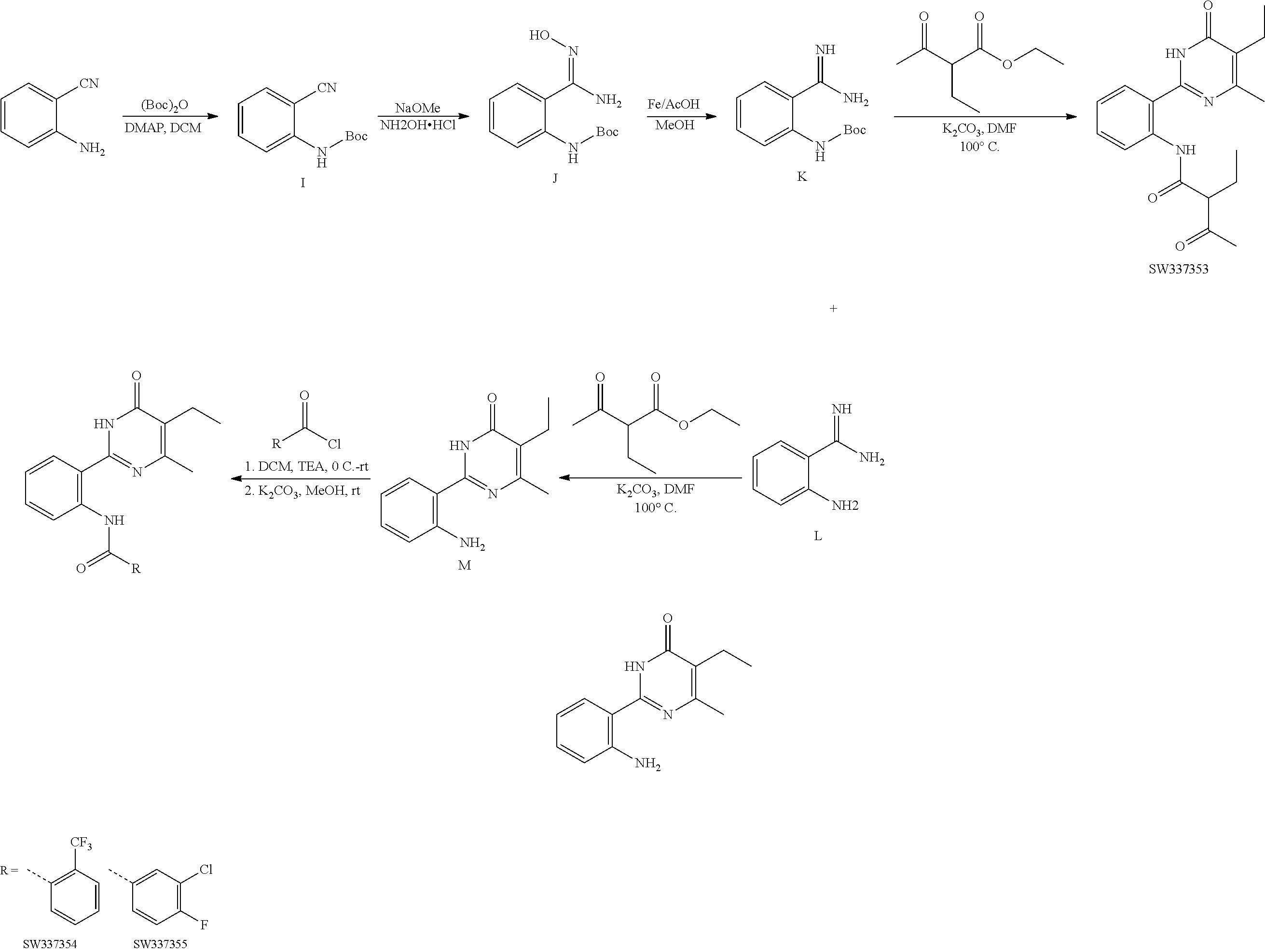

### 2-(2-aminophenyl)-5-ethyl-6-methylpyrimidin-4(3H)-one (SW337353)

### Step 1: Tert-butyl (2-cyanophenyl)carbamate (I)

To a solution of 2-aminobenzonitrile (2 g, 16.94 mmol) in DCM (20 mL) were added trimethylamine (3.353 ml, 25.423 mmol), di-tert-butyl dicarbonate (4.43 g, 20.338 mmol) and DMAP (206 mg, 1.69 mmol). The mixture was stirred at room temperature for 4 h, after that the reaction mixture was diluted with water and extracted with DCM (3×50 mL), and the combined organic layer was dried over anhydrous Na₂SO₄, evaporated under reduced pressure and purified by flash column chromatography to give tert-butyl (2-cyanophenyl)carbamate (I) (3.17 g, 85% yield). ¹H NMR (500 MHz, Chloroform-d) δ 8.23 (d, J=8.5 Hz, 1H), 7.63-7.47 (m, 2H), 7.08 (td, J=7.6, 1.1 Hz, 1H), 7.02 (s, 1H), 1.53 (s, 9H). ESI-MS (m/z): 219.0 [M+H]⁺.

### Step 2: Tert-butyl (E)-(2-(N′-hydroxycarbamimidoyl)phenyl)carbamate(J)

To a solution of tert-butyl (2-cyanophenyl) carbamate (I) (1 g, 4.58 mmol) in MeOH (10 mL) were added NaOMe (247 mg, 4.58 mmol) and NH₂OH·HCl (190 mg, 2.75 mmol). The solution was refluxed for 18 h and completion of the reaction was confirmed by LCMS, The crude reaction mixture was then purified by flash chromatography on silica gel to obtain the desired product as tert-butyl (E)-(2-(N′-hydroxycarbamimidoyl)phenyl)carbamate (J) (750 mg, 65% yield)¹H NMR (500 MHz, Chloroform-d) δ 9.46 (s, 1H), 8.26 (d, J=8.5 Hz, 1H), 7.43 (dd, J=7.8, 1.6 Hz, 1H), 7.36 (ddd, J=8.6, 7.4, 1.6 Hz, 1H), 7.03 (td, J=7.6, 1.2 Hz, 1H), 4.96 (s, 2H), 1.51 (s, 9H). ESI-MS (m/z): 252.1 [M+H]⁺.

### Step 3: Tert-butyl (2-carbamimidoylphenyl)carbamate (K)

To a solution of tert-butyl (E)-(2-(N′-hydroxycarbamimidoyl)phenyl)carbamate (J) (300 mg, 1.27 mmol) in MeOH (4 mL) and H₂O were added Fe powder (69 mg, 1.271 mmol) and acetic acid (4 ml). The reaction mixture was stirred at 50° C. and completion of the reaction was confirmed by LCMS. After completion of the reaction, solvent was removed and the mixture was acidified by 1N HCl to get solid, which was purified by flash chromatography on silica gel to obtain one desired product tert-butyl (2-carbamimidoylphenyl)carbamate (k) (145 mg, 52% yield) ESI-MS (m/z): 236.1 [M+H]⁺. Additionally, 2-aminobenzimidamide (L) (72 mg, 42% yield) was isolated. ESI-MS (m/z): 136.1 [M+H].

Step 4: 2-aminobenzimidamide (300 mg, 2.2 mmol), Ethyl 2-ethyl-3-oxobutanoate (687 mg, 4.41 mmol), and EtOH (10 mL) were combined in a microwave tube and heated at 100° C. for 12 h. Then, the solution was removed and the crude solid was purified by flash chromatography on silica gel to obtain the desired product as (SW337353). ¹H NMR (500 MHz, CDCl₃) δ 7.64 (d, J=8.1 Hz, 1H), 7.31-7.06 (m, 1H), 6.75 (s, 2H), 2.59 (q, J=7.5 Hz, 2H), 2.38 (s, 3H), 1.15 (t, J=7.5 Hz, 3H). ESI-MS (m/z): 230.1 [M+H]⁺.

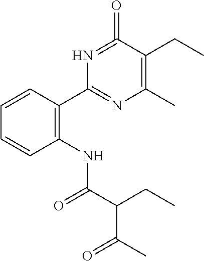

### 2-Ethyl-N-(2-(5-ethyl-4-methyl-6-oxo-1,6-dihydropyrimidin-2-yl)phenyl)-3-oxobutanamide

(SW337354)

Tert-butyl (2-carbamimidoylphenyl) carbamate (k) (200 mg, 0.793 mmol), Ethyl 2-ethyl-3-oxobutanoate (247 mg, 1.587 mmol), and EtOH (6 mL) were combined in a microwave tube and heated at 100° C. for 12 h. Then, the solution was removed and the crude solid was purified by flash chromatography on silica gel to obtain the desired product as SW337354 (211 mg, 78% yield). ¹H NMR (400 MHz, CDCl₃) δ 12.51 (s, 1H), 8.66 (dd, J=8.5, 1.2 Hz, 1H), 8.08 (dd, J=8.1, 1.5 Hz, 1H), 7.51 (t, J=8.7, 7.3, 1.4 Hz, 1H), 7.21 (t, J=7.7, 1.3 Hz, 1H), 3.37 (t, J=7.5 Hz, 1H), 2.62 (q, J=7.5 Hz, 2H), 2.53 (s, 3H), 2.26 (s, 3H), 2.05-1.92 (m, 2H), 1.17 (t, J=7.4 Hz, 3H), 0.99 (t, J=7.4 Hz, 3H). 13C NMR (101 MHz, CDCl₃) δ 205.16, 167.41, 164.63, 159.15, 153.47, 138.89, 132.66, 128.42, 125.12, 123.69, 121.65, 118.11, 66.42, 28.59, 22.72, 21.24, 19.16, 12.60, 12.12. ESI-MS (m/z): 342.2 [M+H]⁺.

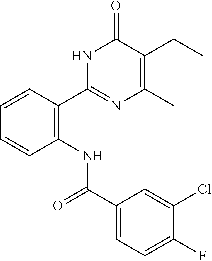 [SMILES: CCc1c(C)nc(-c2ccccc2NC(=O)c2ccc(F)c(Cl)c2)[nH]c1=O]

### 3-Chloro-N-(2-(5-ethyl-4-methyl-6-oxo-1,6-dihydropyrimidin-2-yl)phenyl)-4-fluorobenzamide (SW337355)

Acylation was performed according to General procedure B in 75% yield. ¹H NMR (400 MHz, DMSO-d₆) δ 8.53 (d, J=8.3 Hz, 1H), 8.13 (dd, J=7.1, 2.2 Hz, 1H), 8.09-8.00 (m, 2H), 7.64 (t, J=8.9 Hz, 1H), 7.55 (t, J=7.1 Hz, 1H), 7.26 (t, J=7.8 Hz 1H), 2.50 (q, J=1.9 Hz, 10H), 2.32 (s, 3H), 1.05 (t, J=7.3 Hz, 3H). ³C NMR (101 MHz, DMSO) δ 163.33, 161.01, 158.50, 149.65, 138.68, 131.87, 130.26, 129.94, 129.27, 129.19, 123.92, 121.50, 117.99, 117.77, 117.05, 115.43, 21.05, 18.87, 13.05. ESI-MS (m/z): 386.0 [M+H]⁺.

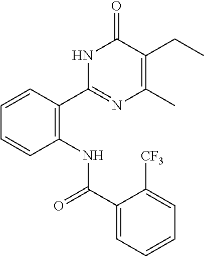 [SMILES: CCc1c(C)nc(-c2ccccc2NC(=O)c2ccccc2C(F)(F)F)[nH]c1=O]

### N-(2-(5-ethyl-4-methyl-6-oxo-1,6-dihydropyrimidin-2-yl)phenyl)-2-(trifluoromethyl)benzamide (SW337356)

Acylation was performed according to General procedure B in 82% yield. ¹H NMR (400 MHz, CDCl₃) δ 13.20 (s, 1H), 8.86 (d, 1H), 8.17 (d, 1H), 7.78 (d, J=7.7 Hz, 1H), 7.72-7.54 (m, 4H), 7.28-7.16 (m, 1H), 2.54 (q, J=7.5 Hz, 2H), 1.92 (s, 3H), 1.12 (t, J=7.5 Hz, 3H). ¹³C NMR (101 MHz, CDCl₃) δ 166.00, 164.58, 158.19, 153.41, 139.59, 136.90, 136.88, 136.86, 136.83, 133.05, 132.03, 130.15, 128.35, 128.10, 128.03, 127.71, 126.82 (q, J=4.9 Hz), 125.17, 125.08, 123.75, 122.36, 121.60, 117.17, 20.62, 19.08, 12.51. ESI-MS (m/z): 402.0 [M+H]⁺.

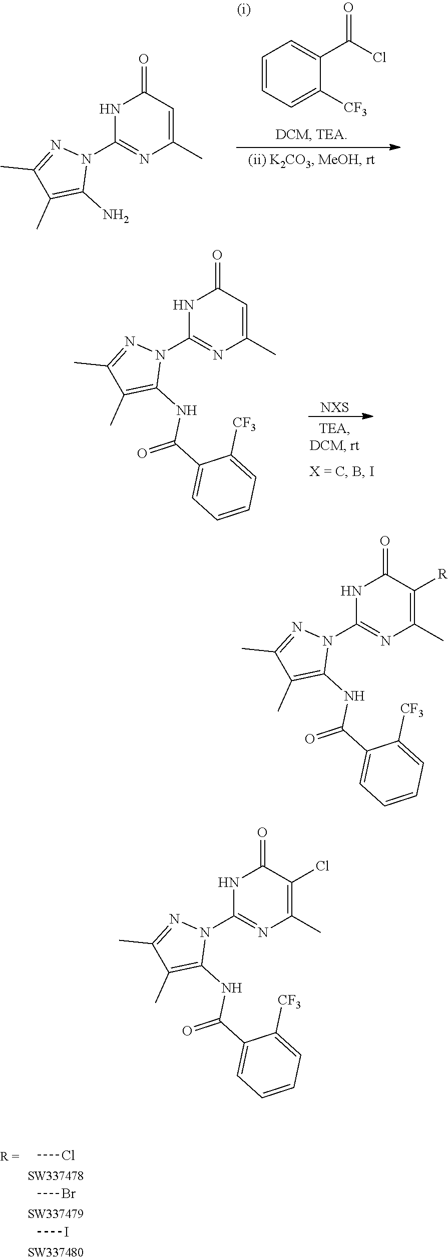

### N-(1-(5-chloro-4-methyl-6-oxo-1,6-dihydropyrimidin-2-yl)-3,4-dimethyl-1H-pyrazol-5-yl)-2-(trifluoromethyl)benzamide (SW337478)

### Step 1: Synthesis of N-(3,4-dimethyl-1-(4-methyl-6-oxo-1,6-dihydropyrimidin-2-yl)-1H-pyrazol-5-yl)-2-(trifluoromethyl)benzamide(N)

Acylation was performed according to General procedure B ¹H NMR (400 MHz, DMSO-d₆) δ 10.64 (s, 1H), 7.95 (s, 1H), 7.92-7.80 (m, 2H), 7.73 (t, J=7.7 Hz, 1H), 6.20 (s, 1H), 2.27 (s, 3H), 2.22 (s, 3H), 1.91 (s, 3H). ESI-MS (m/z): 392.1 [M−H]⁺.

### Step 2: N-(1-(5-chloro-4-methyl-6-oxo-1,6-dihydropyrimidin-2-yl)-3,4-dimethyl-1H-pyrazol-5-yl)-2-(trifluoromethyl)benzamide (SW337478)

To a solution of N-(3,4-dimethyl-1-(4-methyl-6-oxo-1,6-dihydropyrimidin-2-yl)-1H-pyrazol-5-yl)-2-(trifluoromethyl)benzamide (N) (100 mg, 0.255 mmol) in dichloromethane (6 mL) was added NCS (50 mg, 0.382 mmol) and the resulting mixture was stirred for 30 minutes at room temperature and monitored by LCMS, after the reaction was complete, the reaction mixture was diluted with water and extracted with DCM (3×10 mL) and the combined organic layer was dried over anhydrous Na₂SO₄, evaporated under reduced pressure and purified by flash column chromatography to give the 2-(5-amino-3,4-dimethyl-1H-pyrazol-1-yl)-5-chloro-6-methylpyrimidin-4(3H)-one(H) (82 mg, 76% yield). ¹H NMR (400 MHz, CDCl₃) δ 11.54 (s, 1H), 7.85-7.75 (m, 2H), 7.70-7.61 (m, 2H), 2.07 (s, 6H), 1.87 (s, 3H). ¹³C NMR (101 MHz, CDCl₃) δ 167.00, 163.09, 160.19, 152.89, 151.88, 139.41, 134.88, 132.61, 132.09, 130.20, 129.85, 129.29, 128.56, 128.08, 127.03 (q, J=4.9 Hz), 126.98, 124.29, 122.65, 121.26, 115.22, 112.07, 29.85, 21.13, 11.57, 9.39. ESI-MS (m/z): 424.0 [M−H]⁺.

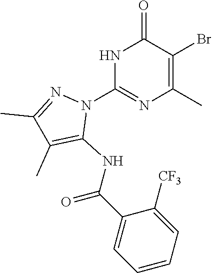

### N-(1-(5-bromo-4-methyl-6-oxo-1,6-dihydropyrimidin-2-yl)-3,4-dimethyl-1H-pyrazol-5-yl)-2-(trifluoromethyl)benzamide (SW337479)

To a solution of N-(3,4-dimethyl-1-(4-methyl-6-oxo-1,6-dihydropyrimidin-2-yl)-1H-pyrazol-5-yl)-2-(trifluoromethyl)benzamide (N) (100 mg, 0.255 mmol) in dichloromethane (6 mL) was added NBS (67 mg, 0.382 mmol) and the resulting mixture was stirred for 30 minutes at room temperature and monitored by LCMS. After the reaction was complete, the reaction mixture was diluted with water and extracted with DCM (3×10 mL) and the combined organic layer was dried over anhydrous Na₂SO₄, evaporated under reduced pressure and purified by flash column chromatography to give the 2-(5-amino-3,4-dimethyl-1H-pyrazol-1-yl)-5-chloro-6-methylpyrimidin-4(3H)-one(H) (92 mg, 70% yield). ¹H NMR (500 MHz, DMSO-d₆) δ 10.66 (s, 1H), 7.95-7.81 (m, 2H), 7.75 (t, J=7.9 Hz, 2H), 2.45 (s, 3H), 2.25 (s, 3H), 1.93 (s, 3H). ¹³C NMR (101 MHz, DMSO) δ 165.03, 150.47, 135.07, 134.12, 132.54, 130.56, 128.70, 126.46, 126.02 (q, J=4.7 Hz), 125.76, 125.02, 113.40, 23.36, 12.26, 7.42. ESI-MS (m/z): 471.9 [M+H]⁺.

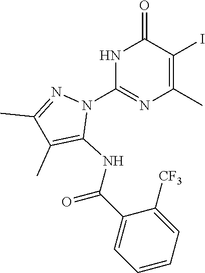

### N-(1-(5-iodo-4-methyl-6-oxo-1,6-dihydropyrimidin-2-yl)-3,4-dimethyl-1H-pyrazol-5-yl)-2-(trifluoromethyl)benzamide (SW337480)

To a solution of the N-(3,4-dimethyl-1-(4-methyl-6-oxo-1,6-dihydropyrimidin-2-yl)-1H-pyrazol-5-yl)-2-(trifluoromethyl)benzamide(N) (100 mg, 0.255 mmol) in dichloromethane (6 mL) was added NIS (85 mg, 0.382 mmol) and the resulting mixture was stirred for 30 minutes at room temperature and monitored by LCMS. After the reaction was complete, the reaction mixture was diluted with water and extracted with DCM (3×10 mL) and the combined organic layer was dried over anhydrous Na₂SO₄, evaporated under reduced pressure and purified by flash column chromatography to give the 2-(5-amino-3,4-dimethyl-1H-pyrazol-1-yl)-5-chloro-6-methylpyrimidin-4(3H)-one(H) (95 mg, 80% yield). ¹H NMR (400 MHz, DMSO-d₆) δ 10.63 (s, 1H), 7.96-7.79 (m, 3H), 7.72 (t, J=7.7 Hz, 1H), 2.49 (s, 3H), 2.22 (s, 3H), 1.91 (s, 3H). ¹³C NMR (101 MHz, DMSO) δ 165.76, 152.67, 135.02, 133.69, 132.53, 130.59, 128.70, 127.98, 126.36 (q, J=5.1 Hz), 126.05, 125.02, 122.30, 113.44, 12.28, 7.35. ESI-MS (m/z): 518.0 [M+H]⁺.

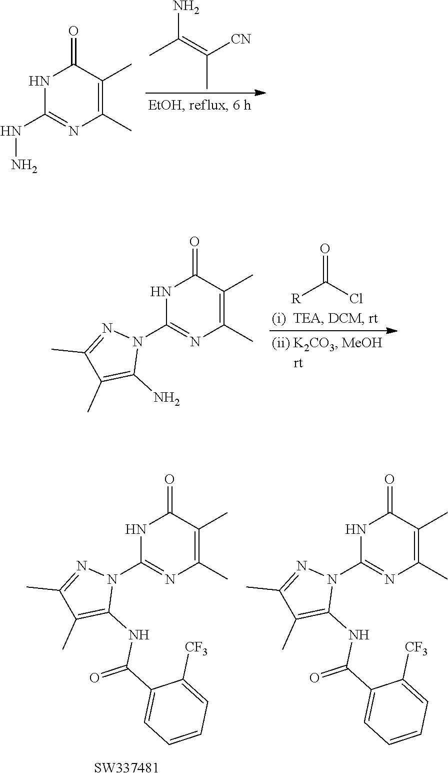

### N-(1-(4,5-dimethyl-6-oxo-1,6-dihydropyrimidin-2-yl)-3,4-dimethyl-1H-pyrazol-5-yl)-2-(trifluoromethyl)benzamide (SW337481)

Acylation was performed according to General procedure B (50 mg, 58% yield). ¹H NMR (400 MHz, CDCl₃) δ 10.72 (s, 1H), 7.81 (d, J=6.9 Hz, 1H), 7.75 (d, J=7.1 Hz, 1H), 7.71-7.61 (m, 2H), 2.25 (s, 3H), 2.13 (s, 3H), 2.04 (s, 3H), 2.02 (s, 3H). ¹³C NMR (101 MHz, CDCl₃) δ 164.13, 160.81, 157.21, 154.74, 147.88, 135.81, 135.20, 132.74, 130.85, 128.72, 127.12 (q, J=4.8 Hz), 124.43, 122.72, 116.94, 111.67, 21.47, 12.74, 11.37, 9.61. ESI-MS (m/z): 406.1 [M+H]⁺.

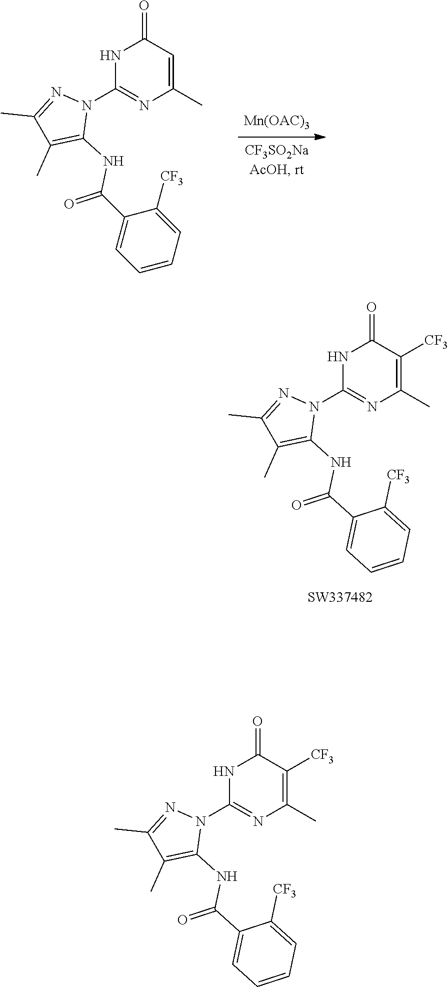

### N-(3,4-dimethyl-1-(4-methyl-6-oxo-5-(trifluoromethyl)-1,6-dihydropyrimidin-2-yl)-1H-pyrazol-5-yl)-2-(trifluoromethyl)benzamide (SW337482)

To a solution of N-(3,4-dimethyl-1-(4-methyl-6-oxo-1,6-dihydropyrimidin-2-yl)-1H-pyrazol-5-yl)-2-(trifluoromethyl)benzamide (50 mg, 0.127 mmol) in acetic acid (3 mL) were added Mn (OAc)3 (102 mg, 0.382 mmol) and CF3SO2Na (60 mg, 0.382 mmol). The resulting mixture was stirred for 12 h at rt. After that the mixture was diluted with water and then extracted with EtOAc. The combined organic layer was dried over anhydrous Na2SO4 and concentrated under reduced pressure. The crude product was purified by flash chromatography to obtain SW337482 (39 mg, 68% yield). ¹H NMR (600 MHz, CDCl₃) δ 10.33 (s, 1H), 7.82 (d, J=7.2 Hz, 1H), 7.75-7.71 (m, 1H), 7.71-7.65 (m, 2H), 2.27 (s, 3H), 2.24 (q, J=2.7 Hz, 3H), 2.13 (s, 3H). ¹³C NMR (151 MHz, CDCl₃) δ 166.62, 164.02, 156.15, 145.39, 135.98, 134.81, 132.27, 130.56, 128.24, 128.03, 127.11 (q, J=4.9 Hz), 124.27, 122.60, 121.49, 113.19, 110.56, 25.24, 16.14, 8.61. ESI-MS (m/z): 460.0 [M+H]⁺.

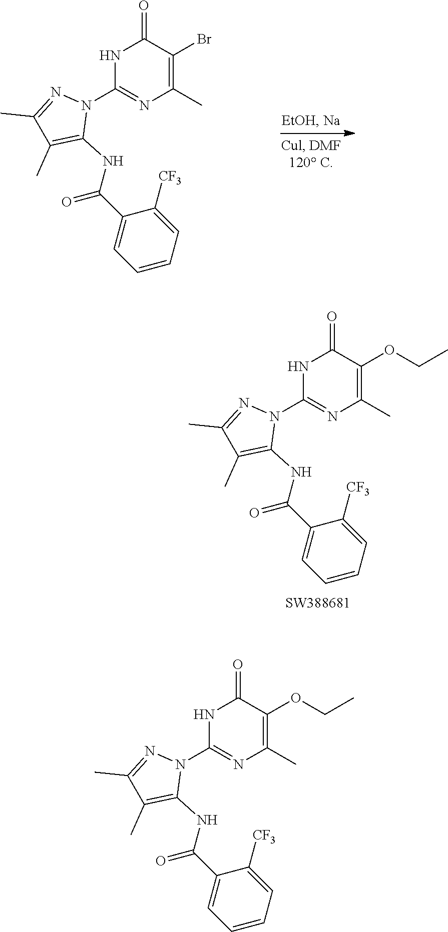

### N-(1-(5-ethoxy-4-methyl-6-oxo-1,6-dihydropyrimidin-2-yl)-3,4-dimethyl-1H-pyrazol-5-yl)-2-(trifluoromethyl)benzamide (SW388681)

To a solution of compound SW337479 (100 mg, 0.212 mmol) in mixture of EtOH (4 mL) and DMF (1 mL) were added Na metal (9 mg, 0.425 mmol) and CuI (20 mg, 0.106 mmol). The resulting mixture was degassed with nitrogen gas and heated to 140 C for 6 hr., and monitored by LCMS. After that the mixture was diluted with water and then extracted with EtOAc. The combined organic layer was dried over anhydrous Na₂SO₄ and concentrated under reduced pressure. The crude product was purified by flash chromatography to obtain SW337479 (38 mg, 41% yield). ¹H NMR (400 MHz, CDCl₃) δ 10.48 (s, 1H), 7.81 (d, J=6.9 Hz, 1H), 7.75 (d, J=7.1 Hz, 1H), 7.70-7.62 (m, 2H), 4.26-4.00 (m, 2H), 2.27-2.18 (m, 6H), 2.13 (s, 3H), 2.08-1.98 (m, 3H), 1.37-1.30 (m, 3H). ¹³C NMR (101 MHz, CDCl₃) δ 164.23, 157.71, 154.63, 150.89, 144.88, 139.65, 136.42, 132.89, 130.86, 128.92, 127.11 (q, J=5.0 Hz), 126.16, 124.99, 121.13, 112.07, 30.22, 19.31, 15.68, 13.74, 9.54. ESI-MS (m/z): 436.0 [M+H]⁺.

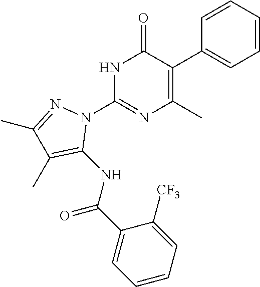 [SMILES: Cc1nn(-c2nc(C)c(-c3ccccc3)c(=O)[nH]2)c(NC(=O)c2ccccc2C(F)(F)F)c1C]

### N-(3,4-dimethyl-1-(4-methyl-6-oxo-5-phenyl-1,6-dihydropyrimidin-2-yl)-1H-pyrazol-5-yl)-2-(trifluoromethyl)benzamide (SW337483)

To a mixture of compound SW337479 (50 mg, 0.0968 mmol) in DMF (3 mL) and H₂O (0.5 mL) were added Na₂CO₃ (30 mg, 0.290 mmol) and phenylboronic acid (14 mg, 0.1161 mmol).

Then the resulting mixture was degassed with nitrogen gas, PdCl₂(PPh₃)₂ was added (6 mg, 0.093 mmol). The mixture was heated to 100° C. for 10 hr and monitored by LCMS. It was diluted with water and then extracted with EtOAc. The combined organic layer was dried over anhydrous Na₂SO₄ and concentrated under reduced pressure. The crude product was purified by flash chromatography to give the SW33748 (20 mg, 46% yield). ¹H NMR (400 MHz, CDCl₃) δ 10.71 (s, 1H), 7.85-7.74 (m, 2H), 7.71-7.59 (m, 2H), 7.41 (t, J=7.4 Hz, 2H), 7.34 (t, J=7.3 Hz, 1H), 7.26 (s, 2H), 2.25 (s, 3H), 2.15 (s, 3H), 1.96 (s, 3H). ¹³C NMR (101 MHz, CDCl₃) δ 164.54, 162.25, 159.31, 153.53, 146.93, 136.76, 135.20, 133.20, 132.12, 128.56, 128.40, 128.23, 128.09, 127.06 (d, J=4.9 Hz), 124.99, 123.30, 122.26, 112.84, 22.13, 12.81, 8.65. ESI-MS (m/z): 468.0 [M+H]⁺.

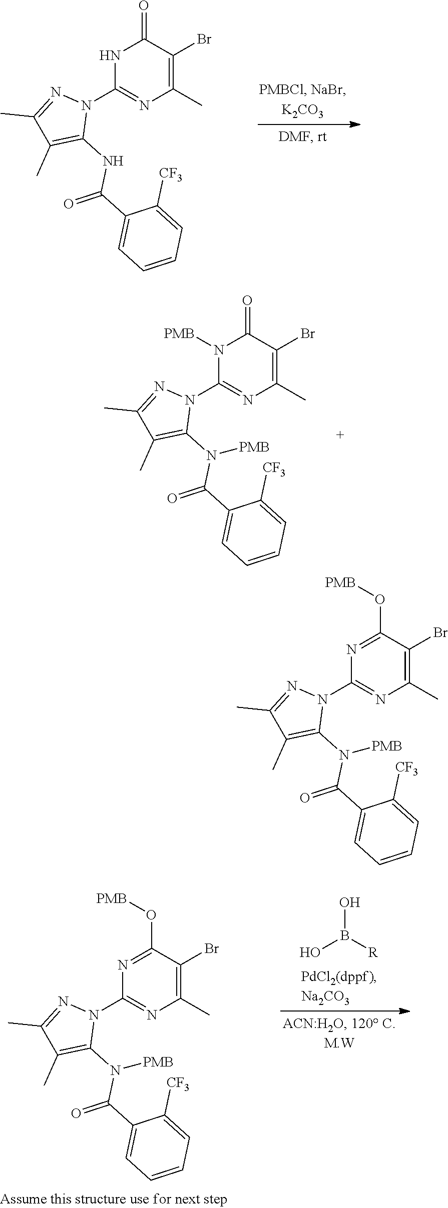

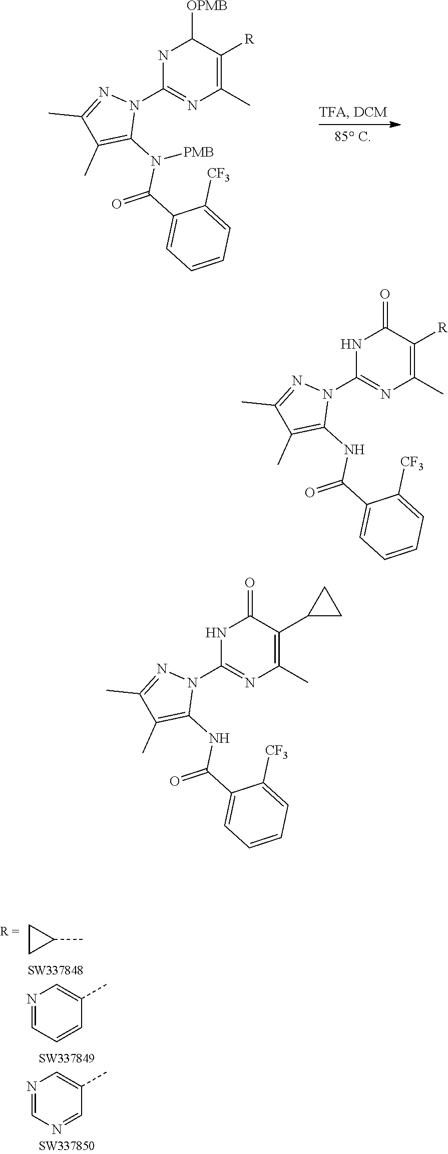

### N-(1-(5-cyclopropyl-4-methyl-6-oxo-1,6-dihydropyrimidin-2-yl)-3,4-dimethyl-1H-pyrazol-5-yl)-2-(trifluoromethyl)benzamide (SW337848)

### Step 1. PMB Protection of Compound SW337479

To a solution of compound SW337479 (1 g, 2.132 mmol) in DMF (5 mL) were added K₂CO₃ (588 mg, 4.264 mmol), PMBCl 0.346 ml, 2.558 mmol) and NaBr (217 mg, 2.132). The resulting mixture was stirred for 6 h at rt. Then the reaction mixture was diluted with H₂O, extracted with EtOAc (3×20 mL) and dried over anhydrous Na₂SO₄. The organic layer was then concentrated under reduced pressure and purified by flash chromatography to afford two isomers P (N-alkylated):Q (O-alkylated)=1:2. (423 mg 28% yield: 815 mg, 54% yield). N-alkylated isomer P: (N-(1-(5-bromo-1-(4-methoxybenzyl)-4-methyl-6-oxo-1,6-dihydropyrimidin-2-yl)-3,4-dimethyl-1H-pyrazol-5-yl)-N-(4-methoxybenzyl)-2-(trifluoromethyl)benzamide): ¹H NMR (400 MHz, Chloroform-d) δ 7.88 (d, J=7.8 Hz, 1H), 7.62 (d, J=7.9 Hz, 1H), 7.38 (t, J=7.7 Hz, 1H), 7.28-7.20 (m, 1H), 7.08 (d, J=8.6 Hz, 2H), 6.78-6.64 (m, 7H), 5.67-5.45 (m, 2H), 4.91 (d, J=14.0 Hz, 1H), 3.76 (s, 3H), 3.62 (s, 3H), 2.42 (s, 3H), 2.02 (s, 3H), 1.19 (s, 3H). O-alkylated isomer

Q: N-(1-(5-bromo-4-((4-methoxybenzyl)oxy)-6-methylpyrimidin-2-yl)-3,4-dimethyl-1H-pyrazol-5-yl)-N-(4-methoxybenzyl)-2-(trifluoromethyl)benzamide: ¹H NMR (400 MHz, Chloroform-d) δ 8.03 (s, 1H), 7.85 (d, J=7.9 Hz, 1H), 7.79-7.66 (m, 1H), 7.62 (d, J=7.9 Hz, 1H), 7.39 (t, J=7.7 Hz, 1H), 7.32-7.25 (m, 1H), 7.15 (t, J=7.6 Hz, 3H), 6.80 (d, J=8.1 Hz, 3H), 5.72 (d, J=14.1 Hz, 1H), 4.14 (d, J=14.2 Hz, 1H), 3.80 (s, 3H), 2.97 (s, 3H), 2.89 (s, 3H), 2.58 (s, 3H), 1.95 (s, 3H), 1.13 (s, 3H). ESI-MS (m/z): 711.1[M+H]⁺.

### Step 2. General Procedure C: For Suzuki Cross-Coupling Reactions and Deprotection of PMB

(i) To a solution of compound Q (0.126 mmol), boronic acid (0.758 mmol) Na₂CO₃ (106 mg, 1.011 mmol) in acetonitrile (4 mL) and water (0.4 mL) under a nitrogen atmosphere was added PdCl₂(PPh₃)₂ (18 mg, 0.093 mmol) (10 mg, 0.0447 mmol). The mixture was heated to 120° C. in microwave for 45 min and monitored by LCMS. Water (10 mL) was added and the mixture was extracted with EtOAc (2×15 mL). The combined organics were washed with brine (10 mL), dried over Na₂SO₄ and concentrated in vacuo. Purification by column chromatography afforded the desired compound.

(ii) To a solution of the above cross-coupling compound (0.1 mmol) in dichloromethane (6 mL) was added TFA (0.035 mL, 0.382 mmol) and the resulting mixture was heated at 85° C. for 2 hr and monitored by LCMS. After the reaction was complete, the solvent mixture was evaporated and quenched with aq. NaHCO₃ and extracted with DCM (3×10 mL) and the combined organic layer was dried over anhydrous Na₂SO₄, evaporated under reduced pressure and purified by flash column chromatography to give the target product. ¹H NMR (400 MHz, CDCl₃) δ 10.72 (s, 1H), 7.83-7.77 (m, 1H), 7.75 (d, J=6.4 Hz, 1H), 7.67 (q, J=7.5, 5.7 Hz, 2H), 2.23 (s, 3H), 2.15 (s, 3H), 2.12 (s, 3H), 1.57-1.40 (m, 1H), 0.92 (qd, J=8.5, 3.2 Hz, 2H), 0.78-0.73 (m, 2H). ¹³C NMR (101 MHz, CDCl₃) δ 165.31, 161.85, 160.16, 152.55, 143.68, 135.80, 135.19, 132.13, 131.21, 128.71, 128.49, 128.39, 128.07, 127.75, 127.69, 127.04 (d, J=4.7 Hz), 126.55, 124.44, 122.25, 120.93, 113.98, 21.79, 12.75, 9.99, 8.08, 6.83. ESI-MS (m/z): 432.0 [M+H]⁺.

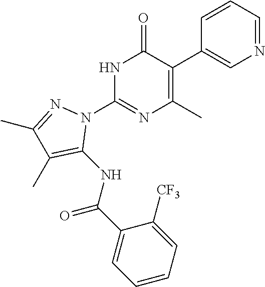 [SMILES: Cc1nn(-c2nc(C)c(-c3cccnc3)c(=O)[nH]2)c(NC(=O)c2ccccc2C(F)(F)F)c1C]

### N-(3,4-dimethyl-1-(4-methyl-6-oxo-5-(pyridin-3-yl)-1,6-dihydropyrimidin-2-yl)-1H-pyrazol-5-yl)-2-(trifluoromethyl)benzamide (SW337849)

The target compound (36% yield) was obtained analogously to SW337848. ¹H NMR (400 MHz, CDCl₃) δ 10.64 (s, 1H), 8.58 (dd, J=4.9, 1.7 Hz, 1H), 8.51 (d, J=2.2 Hz, 1H), 7.83-7.74 (m, 2H), 7.70-7.62 (m, 3H), 7.35 (dd, J=7.9, 4.8 Hz, 1H), 2.25 (s, 3H), 2.15 (s, 3H), 1.99 (s, 3H). ¹³C NMR (101 MHz, CDCl₃) δ 169.91, 164.62, 158.09, 154.50, 149.21, 140.90, 134.89, 130.21, 128.13, 127.73, 125.03, 122.99, 120.21, 118.50, 116.99, 113.70, 111.72, 22.33, 19.03, 13.75, 8.91. ESI-MS (m/z): 469.0 [M+H]⁺.

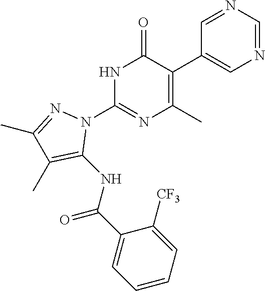 [SMILES: Cc1nn(-c2nc(C)c(-c3cncnc3)c(=O)[nH]2)c(NC(=O)c2ccccc2C(F)(F)F)c1C]

### N-(3,4-dimethyl-1-(4-methyl-6-oxo-1,6-dihydro-[5,5′-bipyrimidin]-2-yl)-1H-pyrazol-5-yl)-2-(trifluoromethyl)benzamide (SW337850)

The target compound (36% yield) was obtained analogously to SW337848. ¹H NMR (400 MHz, CDCl₃) δ 10.47 (s, 1H), 9.19 (s, 1H), 8.72 (s, 2H), 7.87-7.78 (m, 1H), 7.76 (d, J=6.4 Hz, 1H), 7.72-7.63 (m, 2H), 2.28 (s, 3H), 2.15 (s, 3H), 2.03 (s, 3H). ESI-MS (m/z): 470.0 [M+H]⁺.

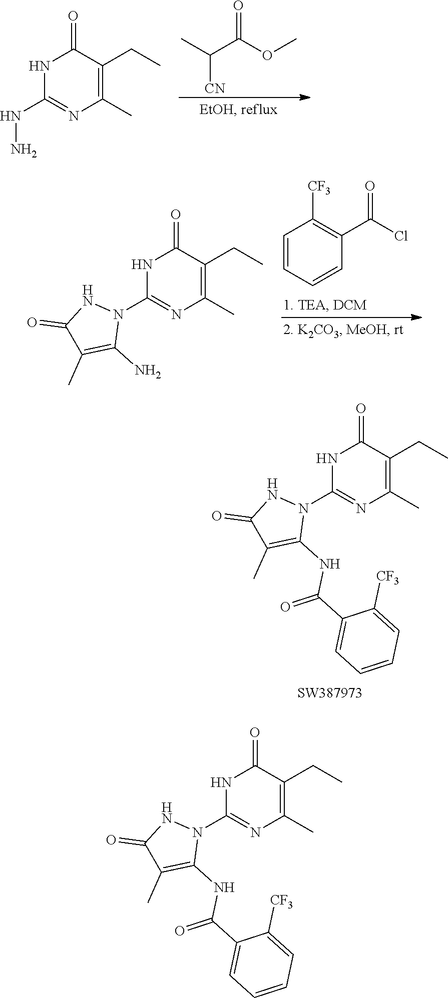

### N-(2-(5-ethyl-4-methyl-6-oxo-1,6-dihydropyrimidin-2-yl)-4-methyl-5-oxo-2,5-dihydro-1H-pyrazol-3-yl)-2-(trifluoromethyl)benzamide (SW387973)

Step 1. Ethyl 2-cyanopropanoate (180 mg, 1.42 mmol) was added to a stirred solution of 5-ethyl-2-hydrazineyl-6-methylpyrimidin-4(3H)-one (200 mg, 1.19 mmol) in EtOH (6 mL).

After heating at 100° C. for 6 h, the mixture was cooled to room temperature and concentrated to give solid. The crude solid was purified by flash chromatography on silica gel to obtain the desired compound 2-(5-amino-4-methyl-3-oxo-2,3-dihydro-1H-pyrazol-1-yl)-5-ethyl-6-methylpyrimidin-4(3H)-one (214 mg, 74% yield)¹H NMR (400 MHz, DMSO-d₆) δ 10.48 (s, 1H), 6.79 (s, 2H), 3.17 (s, 1H), 2.39 (q, J=7.3 Hz, 2H), 2.25 (s, 3H), 1.68 (s, 3H), 0.99 (t, J=7.4 Hz, 3H). ESI-MS (m/z): 250.1[M+H]⁺.

The target compound (78% yield) was obtained by acylation according to General procedure B above. Acylation was performed according to General procedure B. ¹H NMR (600 MHz, DMSO-d₆) δ 10.65 (s, 1H), 7.86-7.77 (m, 3H), 7.68 (t, J=7.5 Hz, 1H), 2.36 (q, J=7.5 Hz, 2H), 2.17 (s, 3H), 1.77 (s, 3H), 0.94 (t, J=7.4 Hz, 3H). ¹³C NMR (151 MHz, DMSO) δ 164.74, 161.64, 135.03, 134.12, 132.55, 130.77, 128.63, 126.69 (q, J=4.7 Hz), 126.49, 126.28, 126.07, 124.60, 122.79, 20.89, 18.83, 13.47, 6.59. ESI-MS (m/z): 422.0 [M+H]⁺.

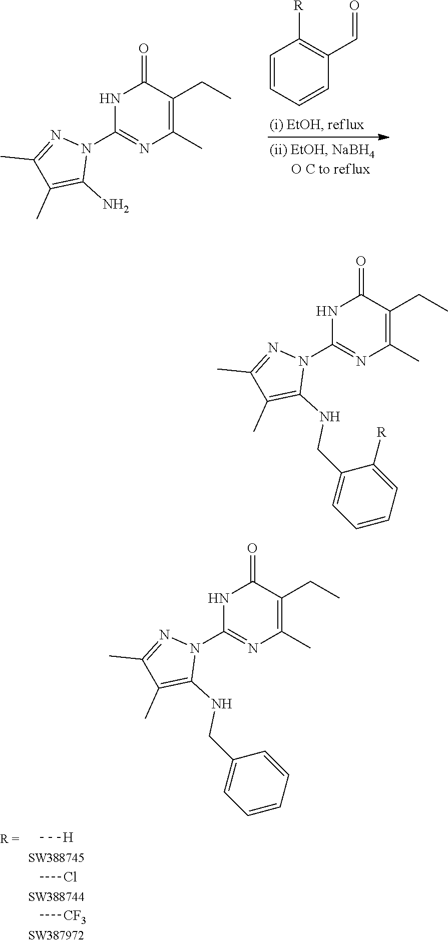

### 2-(5-(benzylamino)-3,4-dimethyl-1H-pyrazol-1-yl)-5-ethyl-6-methylpyrimidin-4(3H)-one (SW388745)

A solution of compound SW388791 (0.202 mmol) and benzaldehyde (0.506 mmol) in EtOH (5 mL) were stirred at reflux and monitored by LCMS. Upon formation of the imine, the reaction mixture was cooled to 0° C., NaBH₄ (15 mg 0.404 mmol) was added, and the resulting mixture was heated at 85° C. for 2 h and monitored by LCMS. After the reaction was complete, the solvent was evaporated and the reaction was quenched with Aq. NH₄C₁ and extracted with DCM (3×10 mL). The combined organic layer was dried over anhydrous Na₂SO₄, evaporated under reduced pressure and purified by flash column chromatography to give the target product. ¹H NMR (400 MHz, CDCl₃) δ 7.97 (s, 1H), 7.30-7.24 (m, 4H), 7.23-7.16 (m, 1H), 4.50 (s, 2H), 2.42 (q, J=7.5 Hz, 2H), 2.09 (s, 3H), 1.99 (s, 3H), 1.88 (s, 3H), 1.01 (t, J=7.5 Hz, 3H). ¹³C NMR (101 MHz, CDCl₃) δ 162.85, 158.18, 154.54, 148.55, 141.72, 129.54, 127.50, 126.92, 120.22, 98.98, 50.66, 22.14, 17.77, 12.96, 12.62, 7.51. ESI-MS (m/z): 338.1 [M+H]⁺.

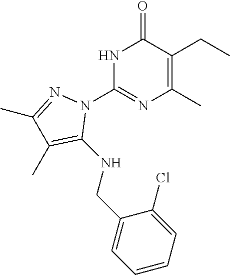 [SMILES: CCc1c(C)nc(-n2nc(C)c(C)c2NCc2ccccc2Cl)[nH]c1=O]

### 2-(5-((2-chlorobenzyl)amino)-3,4-dimethyl-1H-pyrazol-1-yl)-5-ethyl-6-methylpyrimidin-4(3H)-one (SW388744)

The target compound was synthesized analogously to SW388745. ¹H NMR (400 MHz, CDCl₃) δ 7.44-7.36 (m, 2H), 7.32-7.13 (m, 2H), 4.65 (s, 2H), 2.51 (q, J=7.5 Hz, 2H), 2.23 (s, 3H), 2.10 (s, 3H), 1.91 (s, 3H), 1.09 (t, J=7.5 Hz, 3H). ¹³C NMR (101 MHz, CDCl₃) δ 152.79, 147.94, 139.95, 133.53, 130.26, 128.82, 128.55, 127.25, 118.83, 91.69, 47.33, 18.91, 13.00, 12.55, 9.14. ESI-MS (m/z): 372.1 [M+H]⁺.

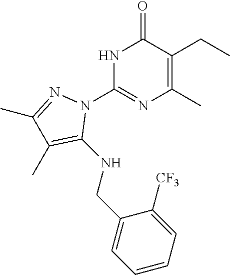 [SMILES: CCc1c(C)nc(-n2nc(C)c(C)c2NCc2ccccc2C(F)(F)F)[nH]c1=O]

### 2-(3,4-dimethyl-5-((2-(trifluoromethyl)benzyl)amino)-1H-pyrazol-1-yl)-5-ethyl-6-methylpyrimidin-4(3H)-one (SW387972)

The target compound was synthesized analogously to SW388745. ¹H NMR (400 MHz, CDCl₃) δ 8.11 (s, 1H), 7.68 (d, J=7.9 Hz, 1H), 7.62 (d, J=7.9 Hz, 1H), 7.54 (t, J=7.6 Hz, 1H), 7.39 (t, J=7.6 Hz, 1H), 4.77 (s, 2H), 2.51 (q, J=7.5 Hz, 2H), 2.20 (s, 3H), 2.09 (s, 3H), 1.85 (s, 3H), 1.09 (t, J=7.5 Hz, 3H). ¹³C NMR (101 MHz, CDCl₃) δ 161.58, 157.62, 151.16, 147.21, 146.34, 140.04, 132.97, 129.88, 127.45, 126.22 (q, J=4.9 Hz), 125.93, 123.20, 93.85, 45.46, 22.86, 19.88, 14.51, 6.76. ESI-MS (m/z): 406.1 [M+H]⁺.

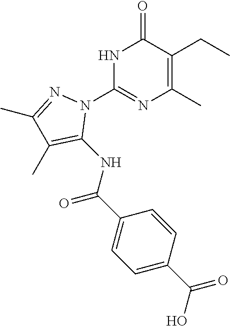 [SMILES: CCc1c(C)nc(-n2nc(C)c(C)c2NC(=O)c2ccc(C(=O)O)cc2)[nH]c1=O]

### 4-((1-(5-ethyl-4-methyl-6-oxo-1,6-dihydropyrimidin-2-yl)-3,4-dimethyl-1H-pyrazol-5-yl)carbamoyl)benzoic acid (SW387982)

To a suspension of SW387981 (50 mg, 0.121 mmol) in THE (2 mL) and H₂O (1 mL) was added LiOH (8 mg, 0.3657 mmol) and the reaction mixture was stirred at room temperature for 4 h. After the reaction was complete, the residue was treated with 1 N HCl until pH 3. The solid formed was collected by filtration and washed with ethyl acetate and small portions of water and dried under vacuum to afford the desired product SW387982 (39 mg 82% yield). ¹H NMR (400 MHz, DMSO-d₆) δ 12.08 (s, 1H), 10.70 (s, 1H), 8.09 (s, 4H), 2.40 (q, J=7.5 Hz, 2H), 2.23 (s, 3H), 2.06 (s, 3H), 1.94 (s, 3H), 0.98 (t, J=7.4 Hz, 3H). ¹³C NMR (151 MHz, DMSO) δ 166.14, 163.02, 137.23, 134.88, 134.59, 133.96, 131.75, 129.51, 128.04, 112.80, 18.26, 12.73, 12.38, 8.08. ESI-MS (m/z): 394.1 [M−H]⁺.

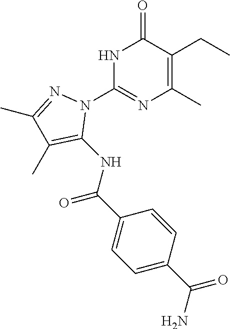 [SMILES: CCc1c(C)nc(-n2nc(C)c(C)c2NC(=O)c2ccc(C(N)=O)cc2)[nH]c1=O]

### N-(1-(5-ethyl-4-methyl-6-oxo-1,6-dihydropyrimidin-2-yl)-3,4-dimethyl-1H-pyrazol-5-yl)terephthalamide (SW387983)

To a suspension of SW387981 (50 mg, 0.121 mmol) in THE (2 mL) and H₂O (1 mL) was added LiOH (8 mg, 0.3657 mmol) and the reaction mixture was stirred at room temperature for 4 h. After the reaction was complete, the residue was treated with 1 N HCl until pH 3. The solid formed was collected by filtration and washed with ethyl acetate and small portions of water and dried under vacuum to afford the desired product SW387982 (39 mg 82% yield). ¹H NMR (600 MHz, DMSO-d₆) δ 10.81 (s, 1H), 8.16 (s, 1H), 8.10-7.99 (m, 4H), 7.57 (s, 1H), 2.39 (q, J=7.4 Hz, 2H), 2.22 (s, 3H), 2.08 (d, J=4.8 Hz, 3H), 1.93 (s, 3H), 0.97 (t, J=7.4 Hz, 3H). ¹³C NMR (151 MHz, DMSO) δ 167.17, 164.33, 152.84, 137.30, 136.89, 135.84, 135.05, 130.14, 127.93, 127.75, 112.55, 20.35, 18.28, 12.75, 11.65, 8.16. ESI-MS (m/z): 395.1 [M+H]⁺.

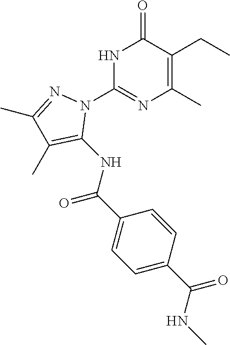 [SMILES: CCc1c(C)nc(-n2nc(C)c(C)c2NC(=O)c2ccc(C(=O)NC)cc2)[nH]c1=O]

### N1-(1-(5-ethyl-4-methyl-6-oxo-1,6-dihydropyrimidin-2-yl)-3,4-dimethyl-1H-pyrazol-5-yl)-N4-methylterephthalamide (SW387984)

The target compound was synthesized analogously to SW387983 (70 mg, 70% yield). ¹H NMR (600 MHz, DMSO-d₆) δ 10.72 (s, 1H), 8.64 (q, J=4.5 Hz, 1H), 8.05 (d, J=8.0 Hz, 2H), 7.98 (d, J=8.0 Hz, 2H), 2.81 (d, J=4.4 Hz, 3H), 2.39 (q, J=7.4 Hz, 2H), 2.22 (s, 3H), 2.08 (s, 3H), 1.93 (s, 3H), 0.97 (t, J=7.4 Hz, 3H). ¹³C NMR (151 MHz, DMSO) δ 165.87, 164.36, 137.49, 135.65, 134.53, 127.81, 127.33, 112.68, 25.96, 18.26, 12.73, 12.38, 8.15. ESI-MS (m/z): 409.1 [M+H]⁺.

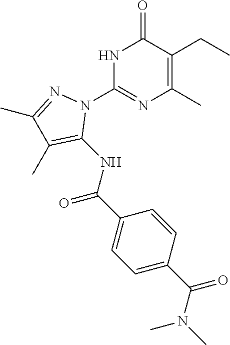 [SMILES: CCc1c(C)nc(-n2nc(C)c(C)c2NC(=O)c2ccc(C(=O)N(C)C)cc2)[nH]c1=O]

### N1-(1-(5-ethyl-4-methyl-6-oxo-1,6-dihydropyrimidin-2-yl)-3,4-dimethyl-1H-pyrazol-5-yl)-N4,N4-dimethylterephthalamide (SW387985)

To a solution of compound SW387982 (0.252 mmol) in DMF (4 mL) were added amine (0.303 mmol), HATU (143 mg, 0.378 mmol) and DIPEA (0.089 mL, 0.505 mmol). The mixture was stirred at room temperature for 4 h and water was added to quench the reaction. The aqueous mixture was extracted with ethyl acetate three times, and the combined organic phases were washed with brine, dried over anhydrous Na₂SO₄ and concentrated to deliver the crude product, which was then purified by flash chromatography on silica gel to obtain a target compound. ¹H NMR (400 MHz, CDCl₃) δ 11.46 (s, 1H), 8.05 (d, J=8.3 Hz, 2H), 7.56 (d, J=8.3 Hz, 2H), 3.13 (s, 3H), 2.97 (s, 3H), 2.50 (q, J=7.5 Hz, 2H), 2.24 (s, 3H), 2.21 (s, 3H), 2.11 (s, 3H), 1.07 (t, J=7.5 Hz, 3H). ¹³C NMR (101 MHz, CDCl₃) δ 170.42, 162.93, 151.80, 145.78, 139.98, 136.83, 133.87, 127.89, 127.62, 122.88, 109.72, 40.46, 35.46, 20.39, 19.70, 13.42, 12.84, 9.38. ESI-MS (m/z): 423.1 [M+H]⁺.

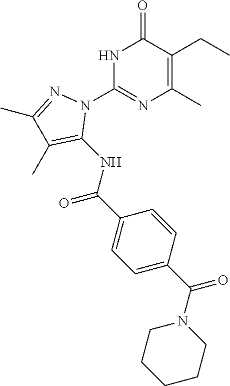 [SMILES: CCc1c(C)nc(-n2nc(C)c(C)c2NC(=O)c2ccc(C(=O)N3CCCCC3)cc2)[nH]c1=O]

### N-(1-(5-ethyl-4-methyl-6-oxo-1,6-dihydropyrimidin-2-yl)-3,4-dimethyl-1H-pyrazol-5-yl)-4-(piperidine-1-carbonyl)benzamide (SW388676)

The target compound was synthesized analogously to SW387985. ¹H NMR (600 MHz, CDCl₃) δ 12.91 (s, 1H), 8.09 (d, J=7.8 Hz, 2H), 7.55 (d, J=7.8 Hz, 2H), 4.08-3.57 (m, 2H), 3.43-3.20 (m, 2H), 2.08 (s, 6H), 1.84 (s, 3H), 1.71 (s, 4H), 1.53 (s, 2H), 0.85 (t, J=7.8 Hz, 3H). ¹³C NMR (151 MHz, CDCl₃) δ 172.06, 169.26, 164.17, 158.16, 152.91, 150.06, 141.20, 137.04, 134.87, 129.16, 127.27, 122.39, 113.08, 48.29, 41.33, 29.81, 26.63, 25.70, 24.61, 21.36, 18.15, 12.86, 8.48. ESI-MS (m/z): 463.1 [M+H]⁺.

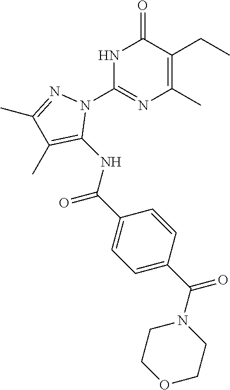 [SMILES: CCc1c(C)nc(-n2nc(C)c(C)c2NC(=O)c2ccc(C(=O)N3CCOCC3)cc2)[nH]c1=O]

### N-(1-(5-ethyl-4-methyl-6-oxo-1,6-dihydropyrimidin-2-yl)-3,4-dimethyl-1H-pyrazol-5-yl)-4-(morpholine-4-carbonyl)benzamide (SW388677)

The target compound was synthesized analogously to SW387985. ¹H NMR (400 MHz, CDCl₃) δ 11.43 (s, 1H), 8.03 (d, J=8.3 Hz, 2H), 7.53 (d, J=8.3 Hz, 2H), 3.88-3.57 (m, 6H), 3.40 (s, 2H), 2.47 (q, J=7.5 Hz, 2H), 2.21 (s, 3H), 2.17 (s, 3H), 2.07 (s, 3H), 1.04 (t, J=7.5 Hz, 3H). ¹³C NMR (101 MHz, CDCl₃) δ 169.13, 162.63, 161.14, 157.18, 153.28, 145.70, 139.28, 136.62, 134.39, 127.98, 127.57, 122.80, 110.40, 65.95, 46.48, 42.61, 21.18, 18.86, 12.75, 12.66, 9.98. ESI-MS (m/z): 465.1 [M+H]⁺.

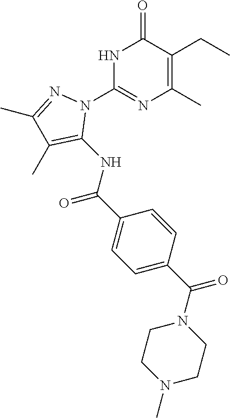 [SMILES: CCc1c(C)nc(-n2nc(C)c(C)c2NC(=O)c2ccc(C(=O)N3CCN(C)CC3)cc2)[nH]c1=O]

### N-(1-(5-ethyl-4-methyl-6-oxo-1,6-dihydropyrimidin-2-yl)-3,4-dimethyl-1H-pyrazol-5-yl)-4-(4-methylpiperazine-1-carbonyl)benzamide (SW388678)

The target compound was synthesized analogously to SW387985. ¹H NMR (600 MHz, CDCl₃) δ 11.49 (s, 1H), 8.06 (d, J=8.3 Hz, 2H), 7.56 (d, J=8.3 Hz, 2H), 3.84 (s, 2H), 3.43 (s, 2H), 2.51 (q, J=8.0, 7.5 Hz, 4H), 2.38 (s, 2H), 2.34 (s, 3H), 2.25 (s, 3H), 2.24 (s, 3H), 2.13 (s, 3H), 1.09 (t, J=7.5 Hz, 3H). ¹³C NMR (151 MHz, CDCl₃) δ 169.15, 162.86, 161.12, 157.33, 153.54, 145.74, 139.87, 136.79, 134.71, 128.05, 127.65, 122.97, 110.49, 55.27, 54.71, 47.62, 46.08, 42.15, 29.84, 21.35, 19.01, 12.90, 12.84, 10.17. ESI-MS (m/z): 478.1 [M+H]⁺.

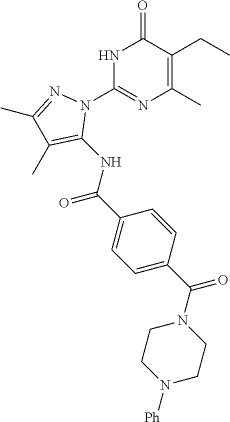 [SMILES: CCc1c(C)nc(-n2nc(C)c(C)c2NC(=O)c2ccc(C(=O)N3CCN(c4ccccc4)CC3)cc2)[nH]c1=O]

### N-(1-(5-ethyl-4-methyl-6-oxo-1,6-dihydropyrimidin-2-yl)-3,4-dimethyl-1H-pyrazol-5-yl)-4-(4-phenylpiperazine-1-carbonyl)benzamide (SW388679)

The target compound was synthesized analogously to SW387985. ¹H NMR (400 MHz, CDCl₃) δ 11.51 (s, 1H), 8.09 (d, J=8.0 Hz, 2H), 7.60 (d, J=8.2 Hz, 2H), 7.32-7.27 (m, 2H), 7.00-6.85 (m, 3H), 3.97 (s, 2H), 3.58 (s, 2H), 3.33-3.08 (m, 4H), 2.57-2.46 (m, 2H), 2.26 (s, 3H), 2.23 (s, 3H), 2.14 (s, 3H), 1.09 (t, J=7.5 Hz, 3H). ¹³C NMR (101 MHz, CDCl₃) δ 169.25, 162.85, 157.31, 154.11, 150.96, 139.74, 134.89, 129.45, 128.13, 127.73, 122.99, 121.02, 116.99, 116.32, 110.52, 50.43, 49.78, 47.77, 42.36, 29.83, 21.34, 19.03, 12.89, 12.76, 10.17. ESI-MS (m/z): 540.1 [M+H]⁺.

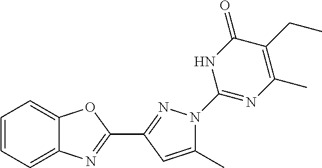 [SMILES: CCc1c(C)nc(-n2nc(-c3nc4ccccc4o3)cc2C)[nH]c1=O]

### 2-(3-(benzo[d]oxazol-2-yl)-5-methyl-1H-pyrazol-1-yl)-5-ethyl-6-methylpyrimidin-4(3H)-one (SW388680)

### Step 1: Ethyl-1-(5-ethyl-4-methyl-6-oxo-1,6-dihydropyrimidin-2-yl)-5-methyl-1H-pyrazole-3-carboxylate (X)

Ethyl 2,4-dioxopentanoate (98 mg, 0.625 mmol) was added to a stirred solution of 5-ethyl-2-hydrazineyl-6-methylpyrimidin-4(3H)-one (100 mg, 0.5952 mmol) in AcOH (6 mL). After heating at 100° C. for 6 h, the mixture was cooled to room temperature and concentrated, The crude solid was purified by flash chromatography on silica gel to obtain the inseparable isomers (W: ethyl 1-(5-ethyl-4-methyl-6-oxo-1,6-dihydropyrimidin-2-yl)-3-methyl-1H-pyrazole-5-carboxylate; X: ethyl 1-(5-ethyl-4-methyl-6-oxo-1,6-dihydropyrimidin-2-yl)-5-methyl-1H-pyrazole-3-carboxylate):(1:9) (117 mg, 68% yield), these are used for next step. ¹H NMR (400 MHz, Chloroform-d) δ 6.58 (s, 1H), 4.30 (q, J=7.1 Hz, 2H), 2.65 (s, 3H), 2.50 (q, J=7.5 Hz, 2H), 2.27 (s, 3H), 1.31 (t, J=7.2 Hz, 3H), 1.05 (t, J=7.5 Hz, 3H). ESI-MS (m/z): 291.0 [M+H]⁺.

### Step 2: 1-(5-ethyl-4-methyl-6-oxo-1,6-dihydropyrimidin-2-yl)-5-methyl-1H-pyrazole-3-carboxylic acid (Z)

To a suspension of above inseparable isomers (W:X) (100 mg, 0.343 mmol) in THE (4 mL) and H₂O (1 mL) was added LiOH (8 mg, 1.030 mmol) and the reaction mixture was stirred at room temperature for 4 h. After the reaction was complete, the residue was treated with 1 N HCl until pH 3. The solid formed was collected by filtration and washed with ethyl acetate and small portions of water and dried under vacuum to afford the desired product as a mixture of inseparable isomers (1:9) (77 mg, 88% yield), which were used for next step. Major: ¹H NMR (400 MHz, DMSO-d₆) δ 12.79 (s, 1H), 6.70 (s, 1H), 2.58 (s, 3H), 2.56-2.51 (m, 2H), 2.39 (s, 3H), 1.07 (t, J=7.3 Hz, 3H). ESI-MS (m/z): 263.0[M+H]⁺.

### Step 3: 2-(3-(benzo[d]oxazol-2-yl)-5-methyl-1H-pyrazol-1-yl)-5-ethyl-6-methylpyrimidin-4(3H)-one (SW388680)

2-Aminophenol (12 mg, 0.114 mmol) was added to a stirred solution of 1-(5-ethyl-4-methyl-6-oxo-1,6-dihydropyrimidin-2-yl)-5-methyl-1H-pyrazole-3-carboxylic acid, the major isomer from the previous step (25 mg, 0.095 mmol) in PPA (2 mL). After heating at 120° C. for 6 h, the mixture was cooled to room temperature and Aq. NaHCO₃ was added to quench the reaction. The aqueous mixture was extracted with ethyl acetate three times, and the combined organic phases were washed with brine, dried over anhydrous Na2SO4 and concentrated to deliver the crude product, which was then purified by flash chromatography on silica gel to obtain a target compound as a single isomer (17 mg, 55% yield). ¹H NMR (400 MHz, CDCl₃) δ 7.83-7.76 (m, 1H), 7.66-7.57 (m, 1H), 7.47-7.35 (m, 2H), 6.90 (s, 1H), 2.75 (d, J=1.0 Hz, 3H), 2.58 (q, J=7.5 Hz, 2H), 2.35 (s, 3H), 1.14 (t, J=7.5 Hz, 3H). ¹³C NMR (101 MHz, CDCl₃) δ 161.96, 159.01, 156.92, 150.68, 144.63, 142.08, 141.64, 126.16, 125.13, 124.45, 120.60, 111.06, 110.81, 29.83, 21.36, 19.09, 15.34, 12.78. ESI-MS (m/z): 336.1 [M+H]⁺.

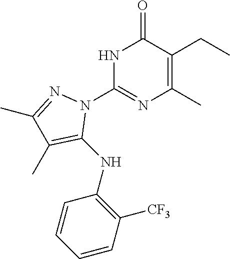 [SMILES: CCc1c(C)nc(-n2nc(C)c(C)c2Nc2ccccc2C(F)(F)F)[nH]c1=O]

### 2-(3,4-dimethyl-5-((2-(trifluoromethyl)phenyl)amino)-1H-pyrazol-1-yl)-5-ethyl-6-methylpyrimidin-4(3H)-one (SW389119)

To a mixture of compound SW388791 (50 mg, 0.201 mmol) and 1,4-dioxane (4 mL) were added Cs₂CO₃ (93 mg, 0.286 mmol) and Xantphos (3 mg, 0.004 mmol). The resulting mixture was degassed with nitrogen gas, then Pd₂(dba)₃ (3.5 mg, 0.004 mmol) was added, and the reaction mixture was refluxed for overnight and monitored by LCMS. The reaction mixture was diluted with water and then extracted with EtOAc. The combined organic layer was dried over anhydrous Na₂SO₄ and concentrated under reduced pressure, the crude product was purified by flash chromatography to give the SW389119 (41 mg, 52% yield). ¹H NMR (400 MHz, CDCl₃) δ 9.50 (s, 1H), 7.62 (d, J=7.8 Hz, 1H), 7.46 (t, J=7.8 Hz, 1H), 7.05 (t, J=7.6 Hz, 1H), 6.92 (d, J=8.2 Hz, 1H), 2.52 (q, J=7.5 Hz, 2H), 2.26 (s, 3H), 2.21 (s, 3H), 1.66 (s, 3H), 1.09 (t, J=7.4 Hz, 3H). ¹³C NMR (101 MHz, CDCl₃) δ 162.46, 159.66, 152.98, 145.73, 141.03, 138.70, 132.68, 126.91 (q, J=5.4 Hz), 125.86, 123.15, 122.50, 121.50, 119.59, 104.05, 29.85, 20.81, 18.97, 12.93, 12.72, 9.56. ESI-MS (m/z): 392.1 [M+H]⁺.

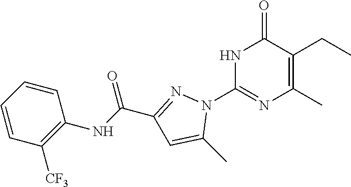 [SMILES: CCc1c(C)nc(-n2nc(C(=O)Nc3ccccc3C(F)(F)F)cc2C)[nH]c1=O]

### 1-(5-ethyl-4-methyl-6-oxo-1,6-dihydropyrimidin-2-yl)-5-methyl-N-(2-(trifluoromethyl)phenyl)-1H-pyrazole-3-carboxamide (SW388659)

To a solution of inseparable isomers from the synthesis of SW388680 (Y:Z 1:9; major=1-(5-ethyl-4-methyl-6-oxo-1,6-dihydropyrimidin-2-yl)-5-methyl-1H-pyrazole-3-carboxylic acid) (20 mg, 0.076 mmol) in ACN (2 mL) were added 2-(trifluoromethyl)aniline (13 mg, 0.083 mmol), T₃P (0.120 mL, 0.190 mmol) and pyridine (0.029 mL, 0.364 mmol). The mixture was heated to 100° C. overnight and aq. NaHCO₃ was added to quench the reaction. The aqueous mixture was extracted with ethyl acetate, and the combined organic phases were washed with brine, dried over anhydrous Na₂SO₄ and concentrated to deliver the crude product, which was then purified by flash chromatography on silica gel to obtain a target compound (20 mg, 68% yield). ¹H NMR (400 MHz, CDCl₃) δ 9.05 (s, 1H), 8.30 (d, J=8.2 Hz, 1H), 7.72-7.56 (m, 2H), 7.34-7.25 (m, 1H), 6.80 (s, 1H), 2.75 (s, 3H), 2.48 (q, J=7.5 Hz, 2H), 2.28 (s, 3H), 1.06 (t, J=7.5 Hz, 3H). ¹³C NMR (101 MHz, CDCl₃) δ 162.61, 159.28, 158.95, 147.61, 145.29, 144.54, 133.03, 126.42 (q, J=5.1 Hz), 125.48, 124.93, 124.65, 124.52, 122.77, 121.19, 120.89, 21.24, 18.94, 15.33, 12.61. ESI-MS (m/z): 406.0 [M+H]⁺.

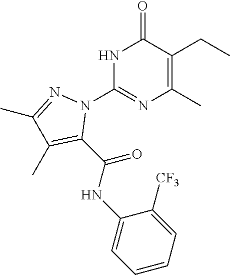 [SMILES: CCc1c(C)nc(-n2nc(C)c(C)c2C(=O)Nc2ccccc2C(F)(F)F)[nH]c1=O]

### 1-(5-ethyl-4-methyl-6-oxo-1,6-dihydropyrimidin-2-yl)-3,4-dimethyl-N-(2-(trifluoromethyl)phenyl)-1H-pyrazole-5-carboxamide (SW389120)

### Step 1: 2-(5-bromo-3,4-dimethyl-1H-pyrazol-1-yl)-5-ethyl-4-((4-methoxybenzyl)oxy)-6-methylpyrimidine (AA)

To a solution of 1-(5-ethyl-4-((4-methoxybenzyl)oxy)-6-methylpyrimidin-2-yl)-3,4-dimethyl-1H-pyrazol-5-amine (80 mg, 0.217 mmol) in ACN (4 mL) was added CuBr2 (48 mg, 0.217 mmol) and isopentyl nitrite (0.075 mL, 0.564 mmol). The mixture was heated to 60° C. for 4 h and monitored by LCMS. After the reaction had gone to completion, 1N HCl was added to quench the reaction. The aqueous mixture was extracted with dichloromethane, and the combined organic phases were washed with 5% Aq. EDTA solutions and brine solution, dried over anhydrous Na2SO4 and concentrated to deliver the crude product, which was then purified by flash chromatography on silica gel to obtain bromide AA (72 mg, 78% yield). 1H NMR (400 MHz, Chloroform-d) δ 2.55 (q, J=7.5 Hz, 2H), 2.36 (s, 3H), 2.25 (s, 3H), 2.00 (s, 3H), 1.12 (t, J=7.5 Hz, 3H). ESI-MS (m/z): 432.1[M+H]⁺.

### Step 2: 1-(5-ethyl-4-((4-methoxybenzyl)oxy)-6-methylpyrimidin-2-yl)-3,4-dimethyl-1H-pyrazole-5-carboxylic acid (BB)

To a solution of bromide AA (50 mg, 0.116 mmol) in anhydrous THE (4 mL) at −78 C, was slowly added n-BuLi (0.087 mL, 0.139 mmol) drop wise to the reaction mixture, The mixture was stirred at −78° C. for 15 min and then CO₂ gas passed to the reaction mixture and monitored by LCMS, after complete 1NHCl was added to quench the reaction. The aqueous mixture was extracted with ethyl acetate, and the combined organic phases were washed with brine, dried over anhydrous Na₂SO₄ and concentrated to deliver the crude product, which was then purified by flash chromatography on silica gel to obtain acid compound BB (22 mg, 48% yield). ESI-MS (m/z): 397.1 [M+H]⁺.

### Step 3: 1-(5-ethyl-4-((4-methoxybenzyl)oxy)-6-methylpyrimidin-2-yl)-3,4-dimethyl-N-(2-(trifluoromethyl)phenyl)-1H-pyrazole-5-carboxamide (CC)

To a solution of above acid compound BB (20 mg, 0.050 mmol) in ACN (2 mL) were added 2-(trifluoromethyl) aniline (10 mg, 0.060 mmol), T₃P (0.080 mL, 0.126 mmol) and pyridine (0.019 mL, 0.242 mmol). The mixture was heated to 100° C. overnight and monitored by LCMS. Aq. NaHCO₃ was added to quench the reaction. The aqueous mixture was extracted with ethyl acetate, and the combined organic phases were washed with brine, dried over anhydrous Na₂SO₄ and concentrated to deliver the crude product, which was then purified by flash chromatography on silica gel to obtain amide compound CC (17 mg, 65% yield)¹H NMR (400 MHz, Chloroform-d) δ 8.66 (s, 1H), 7.88 (s, 1H), 7.60 (d, J=7.9 Hz, 1H), 7.56-7.49 (m, 1H), 7.21 (t, J=7.7 Hz, 1H), 7.01 (d, J=8.2 Hz, 2H), 6.79 (d, J=8.6 Hz, 2H), 5.05 (s, 2H), 3.78 (s, 3H), 2.56 (q, J=7.5 Hz, 2H), 2.49 (s, 3H), 2.35 (s, 3H), 1.05 (t, J=7.5 Hz, 3H). ESI-MS (m/z): 540.1 [M+H]⁺.

Step 4: To a solution of the above amide compound CC (35 mg, 0.064 mmol) in dichloromethane (3 mL) was added TFA (0.2 mL) and the resulting mixture was stirred at rt for 2 hr and monitored by LCMS. After the reaction was complete, the solvent was evaporated and the reaction was quenched with Aq. NaHCO₃ and extracted with DCM (3×10 mL) and the combined organic layer was dried over anhydrous Na₂SO₄, evaporated under reduced pressure and purified by flash column chromatography to give the target product SW389120 (22 mg, 82% yield). ¹H NMR (400 MHz, CDCl₃) δ 9.45 (s, 1H), 8.19 (d, J=8.2 Hz, 1H), 7.77-7.50 (m, 2H), 7.34 (t, J=7.7 Hz, 1H), 2.50 (q, J=7.4 Hz, 2H), 2.26 (s, 3H), 2.17 (s, 3H), 2.12 (s, 3H), 1.07 (t, J=7.5 Hz, 3H). ¹³C NMR (101 MHz, CDCl₃) δ 161.72, 159.45, 158.48, 152.53, 143.86, 135.43, 134.65, 133.00, 128.02, 126.50 (q, J=5.3 Hz), 125.86, 125.30, 123.74, 123.38, 122.59, 122.33, 29.82, 20.99, 19.01, 12.74, 12.10, 8.77. ESI-MS (m/z): 420.1 [M+H]

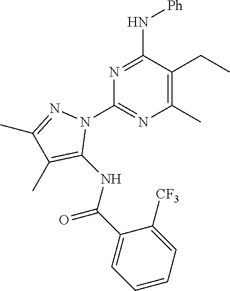 [SMILES: CCc1c(C)nc(-n2nc(C)c(C)c2NC(=O)c2ccccc2C(F)(F)F)nc1Nc1ccccc1]

### N-(1-(5-ethyl-4-methyl-6-(phenylamino)pyrimidin-2-yl)-3,4-dimethyl-1H-pyrazol-5-yl)-2-(trifluoromethyl)benzamide (SW387977)

¹H NMR (400 MHz, CDCl₃) δ 10.15 (s, 1H), 7.60 (d, J=7.9 Hz, 1H), 7.45 (t, J=7.7 Hz, 1H), 7.36 (t, J=7.6 Hz, 1H), 7.25 (d, J=7.5 Hz, 2H), 7.19 (d, J=7.6 Hz, 1H), 7.04 (t, J=7.8 Hz, 2H), 6.78 (t, J=7.4 Hz, 1H), 6.60 (d, J=8.1 Hz, 1H), 2.58 (q, J=7.6 Hz, 2H), 2.44 (s, 3H), 2.30 (s, 3H), 2.06 (s, 3H), 1.19 (d, J=7.7 Hz, 3H). ¹³C NMR (101 MHz, CDCl₃) δ 164.57, 164.07, 158.24, 153.72, 151.11, 137.93, 135.43, 134.39, 131.93, 129.72, 129.09, 127.60, 127.37, 126.62 (q, J=4.8 Hz), 124.87, 124.49, 122.15, 121.68, 119.43, 21.93, 19.20, 12.86, 12.25, 9.47. ESI-MS (m/z): 495.1 [M+H]⁺.

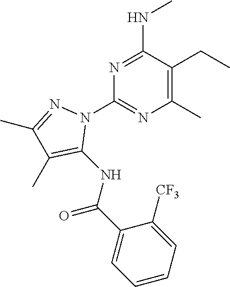 [SMILES: CCc1c(C)nc(-n2nc(C)c(C)c2NC(=O)c2ccccc2C(F)(F)F)nc1NC]

### N-(1-(5-ethyl-4-methyl-6-(methylamino)pyrimidin-2-yl)-3,4-dimethyl-1H-pyrazol-5-yl)-2-(trifluoromethyl)benzamide (SW387978)

¹H NMR (400 MHz, CDCl₃) δ 11.31 (s, 1H), 7.75 (d, J=7.3 Hz, 1H), 7.69 (d, J=6.5 Hz, 1H), 7.65-7.55 (m, 2H), 4.80 (s, 1H), 2.53 (d, J=4.8 Hz, 3H), 2.39 (q, J=7.6 Hz, 2H), 2.30 (d, J=2.4 Hz, 6H), 2.12 (s, 3H), 1.06 (t, J=7.6 Hz, 3H). ¹³C NMR (101 MHz, CDCl₃) δ 163.75, 161.71, 161.15, 154.53, 151.18, 136.17, 135.39, 132.07, 130.36, 128.85, 127.90, 126.66 (q, J=4.9 Hz), 125.02, 122.30, 113.09, 109.83, 27.94, 21.40, 18.94, 12.93, 11.98, 9.67. ESI-MS (m/z): 433.1 [M+H]⁺.

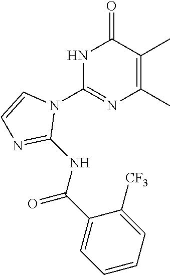 [SMILES: Cc1nc(-n2ccnc2NC(=O)c2ccccc2C(F)(F)F)[nH]c(=O)c1C]

### N-(1-(4,5-dimethyl-6-oxo-1,6-dihydropyrimidin-2-yl)-1H-imidazol-2-yl)-2-(trifluoromethyl)benzamide (SW387979)

¹H NMR (400 MHz, CDCl₃) δ 13.62 (s, 1H), 7.80 (d, J=7.6 Hz, 1H), 7.73 (d, J=7.8 Hz, 1H), 7.66 (d, J=2.7 Hz, 1H), 7.64-7.58 (m, 1H), 7.52 (t, J=7.6 Hz, 1H), 6.77 (d, J=2.8 Hz, 1H), 2.27 (s, 3H), 2.03 (s, 3H). ¹³C NMR (101 MHz, CDCl₃) δ 176.59, 161.36, 150.26, 138.35, 131.97, 129.92, 129.49, 128.13, 127.93, 127.61, 126.71 (q, J=5.4 Hz), 125.40, 122.68, 118.55, 112.49, 110.84, 11.21. ESI-MS (m/z): 378.1 [M+H]⁺.

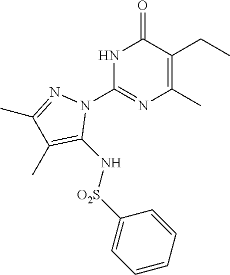 [SMILES: CCc1c(C)nc(-n2nc(C)c(C)c2NS(=O)(=O)c2ccccc2)[nH]c1=O]

### N-(1-(5-ethyl-4-methyl-6-oxo-1,6-dihydropyrimidin-2-yl)-3,4-dimethyl-1H-pyrazol-5-yl)benzenesulfonamide (SW388746)

General Procedure (D): Preparation of Sulfonamides from amine and deprotection of PMB (i) To a mixture of 1-(5-ethyl-4-((4-methoxybenzyl)oxy)-6-methylpyrimidin-2-yl)-3,4-dimethyl-1H-pyrazol-5-amine (0.163 mmol), and substituted benzene sulfonyl chloride (0.179 mmol) in dichloromethane (4 mL) was added pyridine (0.4 mL) and the resulting mixture was stirred at rt overnight. After the reaction was complete, the solvent mixture was evaporated, water was added and extracted with DCM. The combined organic layer was dried over anhydrous Na₂SO₄, evaporated under reduced pressure and purified by flash column chromatography to give the ulfonamide compound

(ii) To a solution of the above sulfonamide (0.115 mmol) in dichloromethane (4 mL) was added TFA (0.1 mL) and the resulting mixture was stirred at rt for 2 hr and monitored by LCMS. After the reaction was complete, the solvent mixture was evaporated and quenched with Aq. NaHCO₃ and extracted with DCM and the combined organic layer was dried over anhydrous Na₂SO₄, evaporated under reduced pressure and purified by flash column chromatography to give the target product.

SW388746: ¹H NMR (400 MHz, CDCl₃) δ 7.54 (d, J=8.2 Hz, 2H), 7.48-7.39 (m, 1H), 7.35-7.24 (m, 2H), 2.43 (q, J=7.5 Hz, 2H), 2.21 (s, 3H), 2.19 (s, 3H), 2.12 (s, 3H), 1.04 (t, J=7.5 Hz, 3H). ¹³C NMR (101 MHz, CDCl₃) δ 161.27, 157.77, 152.87, 144.63, 138.51, 134.55, 133.55, 128.81, 126.98, 122.75, 115.60, 21.12, 18.80, 12.80, 12.79, 8.46. ESI-MS (m/z): 388.1 [M+H]⁺.

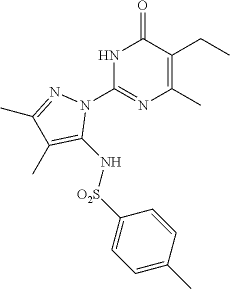 [SMILES: CCc1c(C)nc(-n2nc(C)c(C)c2NS(=O)(=O)c2ccc(C)cc2)[nH]c1=O]

### N-(1-(5-ethyl-4-methyl-6-oxo-1,6-dihydropyrimidin-2-yl)-3,4-dimethyl-1H-pyrazol-5-yl)-4-methylbenzenesulfonamide (SW388747)

The target compound was synthesized analogously to SW388746. ¹H NMR (600 MHz, CDCl₃) δ 9.32 (s, 1H), 7.41 (d, J=8.1 Hz, 2H), 7.09 (d, J=8.0 Hz, 2H), 2.45 (q, J=7.5 Hz, 2H), 2.30 (s, 3H), 2.22 (s, 3H), 2.20 (s, 3H), 2.13 (s, 3H), 1.07 (t, J=7.5 Hz, 3H). ¹³C NMR (151 MHz, CDCl₃) δ 160.90, 157.71, 152.89, 144.72, 144.46, 135.55, 134.75, 129.40, 127.06, 122.90, 115.61, 29.83, 21.64, 21.20, 18.88, 12.91, 12.85, 8.50. ESI-MS (m/z): 402.1[M+H]⁺.

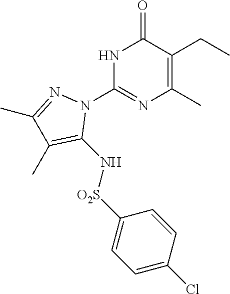 [SMILES: CCc1c(C)nc(-n2nc(C)c(C)c2NS(=O)(=O)c2ccc(Cl)cc2)[nH]c1=O]

### 4-chloro-N-(1-(5-ethyl-4-methyl-6-oxo-1,6-dihydropyrimidin-2-yl)-3,4-dimethyl-1H-pyrazol-5-yl)benzenesulfonamide (SW388748)

The target compound was synthesized analogously to SW388746. ¹H NMR (400 MHz, CDCl₃) δ 9.49 (s, 1H), 7.48 (d, J=8.2 Hz, 2H), 7.29-7.20 (m, 2H), 2.41 (q, J=7.6 Hz, 2H), 2.18 (s, 6H), 2.06 (s, 3H), 1.03 (t, J=7.5 Hz, 3H). ¹³C NMR (101 MHz, CDCl₃) δ 158.49, 153.61, 140.27, 137.27, 129.17, 128.51, 123.02, 114.05, 32.03, 29.81, 21.17, 18.92, 12.84, 8.53. ESI-MS (m/z): 422.0 [M+H]⁺.

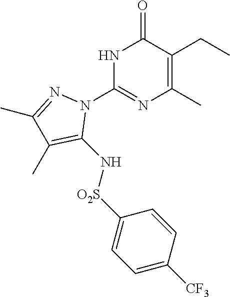 [SMILES: CCc1c(C)nc(-n2nc(C)c(C)c2NS(=O)(=O)c2ccc(C(F)(F)F)cc2)[nH]c1=O]

### N-(1-(5-ethyl-4-methyl-6-oxo-1,6-dihydropyrimidin-2-yl)-3,4-dimethyl-1H-pyrazol-5-yl)-4-(trifluoromethyl)benzenesulfonamide (SW388749)

The target compound was synthesized analogously to SW388746. ¹H NMR (400 MHz, CDCl₃) δ 9.63 (s, 1H), 7.74 (d, J=8.2 Hz, 2H), 7.60 (d, J=8.2 Hz, 2H), 2.44 (q, J=7.5 Hz, 2H), 2.21 (s, 6H), 2.12 (s, 3H), 1.04 (t, J=7.5 Hz, 3H). ¹³C NMR (101 MHz, CDCl₃) δ 160.95, 157.59, 153.03, 144.96, 142.38, 135.43, 135.10, 134.06, 127.73, 125.98 (q, J=3.7 Hz), 124.36, 123.10, 121.64, 115.57, 21.17, 18.85, 12.84, 12.64, 8.55. ESI-MS (m/z): 456.0 [M+H]⁺.

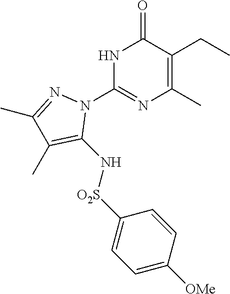 [SMILES: CCc1c(C)nc(-n2nc(C)c(C)c2NS(=O)(=O)c2ccc(OC)cc2)[nH]c1=O]

### N-(1-(5-ethyl-4-methyl-6-oxo-1,6-dihydropyrimidin-2-yl)-3,4-dimethyl-1H-pyrazol-5-yl)-4-methoxybenzenesulfonamide (SW388750)

The target compound was synthesized analogously to SW388746. ¹H NMR (400 MHz, CDCl₃) δ 9.32 (s, 1H), 7.47 (d, J=8.7 Hz, 2H), 6.74 (d, J=8.2 Hz, 2H), 3.76 (s, 3H), 2.45 (q, J=7.5 Hz, 2H), 2.21 (s, 6H), 2.12 (s, 3H), 1.06 (t, J=7.4 Hz, 3H). ¹³C NMR (101 MHz, CDCl₃) δ 163.62, 157.74, 152.82, 145.16, 135.01, 130.09, 129.23, 122.91, 115.48, 114.39, 113.96, 55.76, 29.81, 21.21, 18.89, 12.80, 8.47. ESI-MS (m/z): 418.1[M+H]⁺.

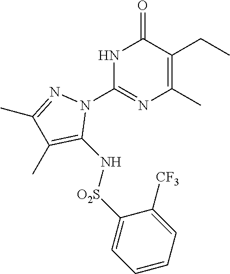 [SMILES: CCc1c(C)nc(-n2nc(C)c(C)c2NS(=O)(=O)c2ccccc2C(F)(F)F)[nH]c1=O]

### N-(1-(5-ethyl-4-methyl-6-oxo-1,6-dihydropyrimidin-2-yl)-3,4-dimethyl-1H-pyrazol-5-yl)-2-(trifluoromethyl)benzenesulfonamide (SW388751)

The target compound was synthesized analogously to SW388746. ¹H NMR (400 MHz, CDCl₃) δ 7.94 (d, J=7.9 Hz, 1H), 7.82 (d, J=7.8 Hz, 1H), 7.70-7.55 (m, 2H), 2.46 (q, J=7.4 Hz, 2H), 2.20 (s, 6H), 1.98 (s, 3H), 1.06 (t, J=7.5 Hz, 3H). ¹³C NMR (101 MHz, CDCl₃) δ 161.46, 158.41, 152.93, 144.90, 138.55, 134.28, 133.52, 132.52, 131.01, 128.38 (q, J=6.3 Hz), 124.02, 123.00, 121.29, 114.33, 20.84, 18.88, 12.79, 12.69, 8.86. ESI-MS (m/z): 456.1 [M+H]⁺.

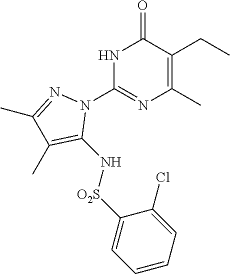 [SMILES: CCc1c(C)nc(-n2nc(C)c(C)c2NS(=O)(=O)c2ccccc2Cl)[nH]c1=O]

### 2-chloro-N-(1-(5-ethyl-4-methyl-6-oxo-1,6-dihydropyrimidin-2-yl)-3,4-dimethyl-1H-pyrazol-5-yl)benzenesulfonamide (SW388752)

The target compound was synthesized analogously to SW388746. ¹H NMR (400 MHz, CDCl₃) δ 10.23 (s, 1H), 7.98 (dd, J=7.9, 1.6 Hz, 1H), 7.48-7.40 (m, 2H), 7.38-7.30 (m, 1H), 2.47 (q, J=7.5 Hz, 2H), 2.21 (s, 3H), 2.15 (s, 3H), 1.96 (s, 3H), 1.07 (t, J=7.5 Hz, 3H)¹³C NMR (101 MHz, CDCl₃) δ 161.12, 157.92, 152.89, 144.96, 137.49, 134.59, 134.47, 132.31, 132.05, 131.10, 127.20, 122.99, 112.92, 21.13, 18.92, 12.79, 12.77, 8.75. ESI-MS (m/z): 422.0 [M+H]

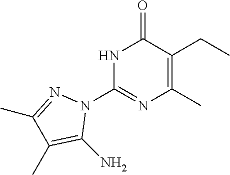 [SMILES: CCc1c(C)nc(-n2nc(C)c(C)c2N)[nH]c1=O]

### 2-(5-amino-3,4-dimethyl-1H-pyrazol-1-yl)-5-ethyl-6-methylpyrimidin-4(3H)-one (SW388791)

The target compound (70% yield) was obtained analogously to General Procedure for Synthesis of MMV6766477 analogs, step 4. ¹H NMR (600 MHz, DMSO-d₆) δ 11.30 (s, 1H), 6.60 (s, 2H), 2.39 (q, J=7.4 Hz, 2H), 2.26 (s, 3H), 2.05 (s, 3H), 1.79 (s, 3H), 0.99 (t, J=7.4 Hz, 3H). ¹³C NMR (151 MHz, DMSO) δ 151.36, 146.82, 119.56, 94.95, 18.23, 12.84, 12.22, 6.58. ESI-MS (m/z): 248.0 [M+H]⁺.

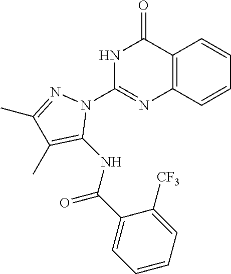 [SMILES: Cc1nn(-c2nc3ccccc3c(=O)[nH]2)c(NC(=O)c2ccccc2C(F)(F)F)c1C]

### N-(3,4-dimethyl-1-(4-oxo-3,4-dihydroquinazolin-2-yl)-1H-pyrazol-5-yl)-2-(trifluoromethyl)benzamide (SW387980)

### Step 1: 2-chloroquinazolin-4(3H)-one (LL)

To a suspension of 2,4-dichloroquinazoline (1 g, 5.07 mmol) in THE (10 mL) was added IN NaOH (10 mL) and the reaction mixture was stirred at room temperature for 4 h. After the reaction was complete, the residue was treated with 1 N HCl until pH 3. The solid formed was collected by filtration and washed with ethyl acetate and small portions of water and dried under vacuum to afford the 2-chloroquinazolin-4(3H)-one (LL) (776 mg 85% yield). ESI-MS (m/z): 181.0 [M+H]⁺.

### Step 2: 2-(5-amino-3,4-dimethyl-1H-pyrazol-1-yl)quinazolin-4(3H)-one (MM)

(i) 2-chloroquinazolin-4(3H)-one (LL) (396 mg, 2.2 mmol), hydrazine monohydrate (0.193 mL, 5 mmol), and EtOH (4 mL) were combined in a microwave tube and heated at 100° C. for 12 h. Then, the solution was filtered and the solid was washed with EtOH and dried overnight. The filtrate was condensed to give purple oil, and EtOH and EtOAc were added to precipitate out any remaining product. Some solid formed and was filtered and dried overnight. The samples were combined to give 2-hydrazineylquinazolin-4(3H)-one (302 mg, 78% yield). ESI-MS (m/z): 177.1 [M+H]⁺.

(ii) (Z)-3-amino-2-methylbut-2-enenitrile (130 mg, 1.363 mmol) was added to a stirred solution of 2-hydrazineylquinazolin-4(3H)-one (200 mg, 1.136 mmol) in EtOH (6 mL). After heating at 100° C. for 6 h, the mixture was cooled to room temperature and evaporated solvent and The crude solid was purified by flash chromatography on silica gel to obtain the 2-(5-amino-3,4-dimethyl-1H-pyrazol-1-yl)quinazolin-4(3H)-one (MM) (197 mg, 68% yield) 1H NMR (400 MHz, Chloroform-d) δ 8.24 (d, J=8.0 Hz, 1H), 7.77-7.64 (m, 1H), 7.57-7.53 (m, 1H), 7.41-7.34 (m, 1H), 5.81 (s, 2H), 2.15 (s, 3H), 1.85 (s, 3H). ESI-MS (m/z): 256.1 [M+H]⁺.

Step 3: Amine MM was acylated according to general procedure B to give SW387980. ¹H NMR (600 MHz, DMSO-d₆) δ 12.56 (s, 1H), 11.04 (s, 1H), 8.37 (d, J=7.9 Hz, 1H), 8.19 (d, J=7.6 Hz, 1H), 8.16-8.05 (m, 3H), 7.99 (t, J=7.8 Hz, 1H), 7.85 (d, J=8.2 Hz, 1H), 7.73 (t, J=7.6 Hz, 1H), 2.50 (s, 3H), 2.19 (s, 3H). ¹³C NMR (151 MHz, DMSO) δ 165.25, 161.40, 150.42, 147.83, 143.90, 135.08, 134.12, 132.62, 130.73, 128.70, 126.67, 126.44 (q, J=4.7 Hz), 124.60, 122.79, 120.62, 113.59, 12.37, 7.40. ESI-MS (m/z): 428.0 [M+H]⁺.

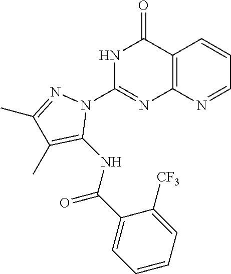 [SMILES: Cc1nn(-c2nc3ncccc3c(=O)[nH]2)c(NC(=O)c2ccccc2C(F)(F)F)c1C]

### N-(3,4-dimethyl-1-(4-oxo-3,4-dihydropyrido[2,3-d]pyrimidin-2-yl)-1H-pyrazol-5-yl)-2-(trifluoromethyl)benzamide (SW388956)

The target compound (78% yield) was obtained by Acylation was performed according to General procedure B above. ¹H NMR (600 MHz, DMSO-d₆) δ 12.60 (s, 1H), 10.77 (s, 1H), 9.02-8.94 (m, 1H), 8.67 (d, J=7.6 Hz, 1H), 8.50 (dd, J=7.8, 2.1 Hz, 1H), 7.92-7.80 (m, 2H), 7.73 (t, J=7.7 Hz, 1H), 7.52 (dd, J=7.8, 4.6 Hz, 1H), 2.28 (s, 3H), 1.95 (s, 3H). ¹³C NMR (151 MHz, DMSO) δ 165.58, 156.29, 151.23, 135.95, 135.27, 134.46, 132.58, 130.42, 129.53, 126.20 (q, J=5.7 Hz), 125.97, 124.72, 122.90, 121.91, 114.97, 12.43, 7.21. ESI-MS (m/z): 429.0 [M+H]⁺.

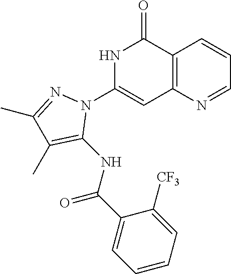 [SMILES: Cc1nn(-c2cc3ncccc3c(=O)[nH]2)c(NC(=O)c2ccccc2C(F)(F)F)c1C]

### N-(3,4-dimethyl-1-(5-oxo-5,6-dihydro-1,6-naphthyridin-7-yl)-1H-pyrazol-5-yl)-2-(trifluoromethyl)benzamide (SW388957)

The target compound (76% yield) was obtained by acylation was performed according to General procedure B above. ¹H NMR (600 MHz, DMSO-d₆) δ 12.03 (s, 1H), 10.68 (s, 1H), 8.92 (dd, J=4.6, 1.8 Hz, 1H), 8.49 (d, J=8.1 Hz, 1H), 7.84 (d, J=7.8 Hz, 1H), 7.78 (t, J=7.5 Hz, 1H), 7.71 (t, J=7.6 Hz, 1H), 7.63 (d, J=7.5 Hz, 1H), 7.50 (dd, J=8.1, 4.5 Hz, 1H), 6.67 (s, 1H), 2.23 (s, 3H), 1.95 (s, 3H). ¹³C NMR (151 MHz, DMSO) δ 166.42, 161.93, 155.23, 153.96, 149.48, 135.27, 134.77, 133.33, 132.71, 130.75, 128.29, 126.67 (q, J=4.9 Hz), 126.29, 126.08, 125.87, 124.48, 122.67, 121.76, 112.68, 98.61, 12.33, 7.27. ESI-MS (m/z): 428.0 [M+H]⁺.

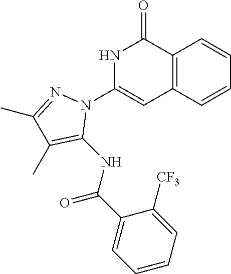 [SMILES: Cc1nn(-c2cc3ccccc3c(=O)[nH]2)c(NC(=O)c2ccccc2C(F)(F)F)c1C]

### N-(3,4-dimethyl-1-(1-oxo-1,2-dihydroisoquinolin-3-yl)-1H-pyrazol-5-yl)-2-(trifluoromethyl)benzamide (SW388958)

The target compound (76% yield) was obtained by Acylation was performed according to General procedure B above. ¹H NMR (600 MHz, DMSO-d₆) δ 11.68 (s, 1H), 10.57 (s, 1H), 8.19 (d, J=8.0 Hz, 1H), 7.84 (d, J=7.8 Hz, 1H), 7.77 (t, J=7.6 Hz, 1H), 7.75-7.70 (m, 2H), 7.68 (t, J=7.7 Hz, 1H), 7.63 (d, J=7.5 Hz, 1H), 7.50 (d, J=8.2 Hz, 1H), 6.57 (s, 1H), 2.22 (s, 3H), 1.94 (s, 3H). ¹³C NMR (151 MHz, DMSO) δ 166.33, 161.89, 148.88, 137.25, 134.88, 134.56, 133.33, 132.99, 132.70, 130.67, 128.49, 126.91, 126.63, 126.61, 126.44, 126.34, 126.23, 126.02, 125.81, 124.86, 124.53, 122.72, 111.81, 97.87, 12.29, 7.35. ESI-MS (m/z): 427.0 [M+H]⁺.

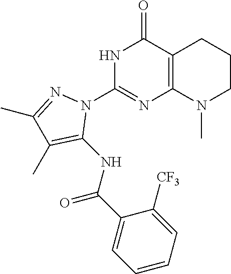 [SMILES: Cc1nn(-c2nc3c(c(=O)[nH]2)CCCN3C)c(NC(=O)c2ccccc2C(F)(F)F)c1C]

### N-(3,4-dimethyl-1-(8-methyl-4-oxo-3,4,5,6,7,8-hexahydropyrido[2,3-d]pyrimidin-2-yl)-1H-pyrazol-5-yl)-2-(trifluoromethyl)benzamide (SW389122)

### Step 1: 2,4-dichloro-5,6,7,8-tetrahydropyrido[2,3-d]pyrimidine (NN)

To a solution of 2,4-dichloropyrido[2,3-d]pyrimidine (1 g, 5 mmol) in EtOH:EtOAc (4:1, 20 mL) was added platinum (IV) oxide (113 mg, 0.5 mmol). After stirring under a hydrogen atmosphere at ambient temperature for 14 hours, the mixture was filtered through a Celite pad and washed with tetrahydrofuran-water (5:1). The filtrate was evaporated under reduced pressure to give 2,4-dichloro-5,6,7,8-tetrahydropyrido[2,3-d]pyrimidine (NN) (527 mg, 52% yield). ¹H NMR (400 MHz, Chloroform-d) δ 6.18 (s, 1H), 3.46 (t, J=5.7 Hz, 2H), 2.72 (t, J=6.4 Hz, 2H), 2.07-1.77 (m, 2H). ESI-MS (m/z): 205.0 [M+H]⁺.

### Step 2: 2-chloro-4-((4-methoxybenzyl)oxy)-8-methyl-5,6,7,8-tetrahydropyrido[2,3-d]pyrimidine (PP)

To a solution of 2,4-dichloro-5,6,7,8-tetrahydropyrido[2,3-d]pyrimidine (NN) (500 mg, 2.463 mmol) in DMF (150 mL) were added iodomethane (0.141 mL, 2.955 mmol) and sodium hydride (60% dispersion in paraffin liquid, 256 mg, 3.694 mmol) at 0° C. After stirring at ambient temperature for 1 hour, the reaction mixture was quenched with saturated ammonium chloride, extracted with ethyl acetate and washed with brine. The organic layer was dried over magnesium sulfate, filtered and evaporated under reduced pressure and purified by flash column chromatography to give 2,4-dichloro-8-methyl-5,6,7,8-tetrahydropyrido[2,3-d]pyrimidine (OO) (507 mg, 95% yield). ¹H NMR (400 MHz, Chloroform-d) δ 3.46-3.33 (m, 2H), 3.15 (s, 3H), 2.70 (t, J=6.5 Hz, 2H), 2.01-1.89 (m, 2H). ESI-MS (m/z): 200.1 [M+H]⁺. A solution of PMBOH (0.207 mL, 1.6512 mmol) in DMF cooled to −20° C., and NaH (110 mg, 1.6512 mmol) was added. The reaction mixture stirred at −20° C. for 30 min, and 2,4-dichloro-8-methyl-5,6,7,8-tetrahydropyrido[2,3-d]pyrimidine (00) (300 mg, 1.376 mmol) in THE (5 mL) was added. The resulting mixture was stirred at rt for 2 h and monitored by LCMS. Water (10 mL) was added and the mixture extracted with EtOAc (2×15 mL). The combined organics were washed with brine (10 mL), dried over Na₂SO₄ and concentrated in vacuo. Purification by column chromatography afforded 2-chloro-4-((4-methoxybenzyl)oxy)-8-methyl-5,6,7,8-tetrahydropyrido[2,3-d]pyrimidine (MM) (289 mg, 66% yield)¹H NMR (400 MHz, Chloroform-d) δ 7.36 (d, J=8.6 Hz, 2H), 6.90 (d, J=8.7 Hz, 2H), 5.28 (s, 2H), 3.81 (s, 3H), 3.34-3.27 (m, 2H), 3.12 (s, 3H), 2.53 (t, J=6.4 Hz, 2H), 1.89-1.80 (m, 2H). ESI-MS (m/z): 320.1[M+H]⁺.

### Step 3: 1-(4-((4-methoxybenzyl)oxy)-8-methyl-5,6,7,8-tetrahydropyrido[2,3-d]pyrimidin-2-yl)-3,4-dimethyl-1H-pyrazol-5-amine (QQ)

The compound pyrazolo amine (QQ) (68% yield) was synthesized according to procedure above step-2 of compound SW387980 through condensation with hydrazine and the appropriate iminonitrile. ¹H NMR (400 MHz, Chloroform-d) δ 7.35 (d, J=8.4 Hz, 2H), 6.87 (d, J=8.7 Hz, 2H), 5.27 (s, 2H), 4.61 (s, 2H), 3.80 (s, 3H), 3.43-3.33 (m, 2H), 3.18 (s, 3H), 2.93 (t, J=6.4 Hz, 2H), 2.13 (s, 3H), 1.87-1.82 (m, 2H), 1.82 (s, 3H). ESI-MS (m/z): 395.1[M+H]⁺.

Step 4: The target compound (86% yield) was obtained by acylation and deprotection of PMB performed according to procedure above step-3 of compound (SW387979). ¹H NMR (400 MHz, CDCl₃) δ 9.38 (s, 1H), 7.63 (d, J=8.2 Hz, 1H), 7.50 (d, J=7.6 Hz, 1H), 7.45 (t, J=7.6 Hz, 1H), 7.42-7.36 (m, 1H), 3.35 (d, J=6.7 Hz, 2H), 3.09 (s, 3H), 2.61 (t, J=6.3 Hz, 2H), 2.24 (s, 3H), 2.01 (s, 3H), 1.92-1.70 (m, 2H). ¹³C NMR (101 MHz, CDCl₃) δ 167.41, 158.23, 150.89, 134.53, 133.14, 132.19, 130.28, 128.36, 128.18, 128.04, 127.72, 127.40, 126.56 (q, J=5.7 Hz) 124.95, 122.23, 114.23, 50.36, 37.48, 21.96, 20.69, 12.73, 7.64. ESI-MS (m/z): 447.1 [M+H]⁺.

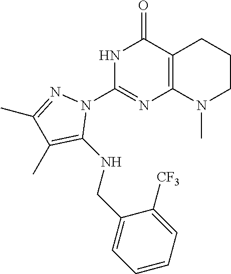 [SMILES: Cc1nn(-c2nc3c(c(=O)[nH]2)CCCN3C)c(NCc2ccccc2C(F)(F)F)c1C]

### 2-(3,4-dimethyl-5-((2-(trifluoromethyl)benzyl)amino)-1H-pyrazol-1-yl)-8-methyl-5,6,7,8-tetrahydropyrido[2,3-d]pyrimidin-4(3H)-one (SW389123)

The target compound was prepared following reductive amination analogously to the synthesis of SW388745. ¹H NMR (600 MHz, Methanol-d₄) δ 7.60 (d, J=7.8 Hz, 1H), 7.50-7.45 (m, 1H), 7.41 (d, J=7.7 Hz, 1H), 7.35 (t, J=7.6 Hz, 1H), 4.31 (s, 2H), 3.41 (d, J=6.6 Hz, 2H), 3.16 (s, 3H), 2.38 (t, J=6.3 Hz, 2H), 2.09 (s, 3H), 1.82-1.77 (m, 3H), 1.75 (s, 3H). ¹³C NMR (151 MHz, MeOD) δ 158.04, 151.70, 145.86, 137.95, 132.58, 130.08, 128.00, 127.91, 127.79, 126.38 (q, J=5.7 Hz), 125.76, 123.95, 103.00, 50.59, 47.83, 37.54, 21.96, 20.94, 12.42, 7.44. ESI-MS (m/z): 433.1 [M+H]⁺.

Chemical Cross-Linking Experiments

A probe compound (SW223022) with benzophenone and alkyne modifications was synthesized. Briefly, purified mammalian or parasite tubulin at 10 μM (starting concentration) was plated in 96-well plates, treated with the probe in the presence or absence of highly active and less active competitors, and tubulin was polymerized for 1 h at 37° C. (mammalian tubulin) and 30° C. The samples were UV cross-linked by placing the 96 well plates on ice approximately 3-4 inches below the bulbs in a stratalinker and then exposed to 15 minutes of UVB radiation. The samples were immediately solubilized in 1% SDS, with benzonase (Sigma) diluted 1:20,000 in buffer containing 50 mM HEPES 7.4, 10 mM KCl and 2 mM MgCl₂. The samples were normalized for protein concentrations using the BCA assay (Life Technologies). Equal amounts of sample were subjected to a click reaction with 100 μM TBTA (dissolved in 4:1 DMSO:t-butanol), 1 mM TCEP, 2 mM CuSO₄ and 25 μM Alexafluor-532 azide for 1 h at 25° C. with agitation. SDS sample buffer was then added to the samples to quench the reaction, and proteins were resolved by SDS-PAGE. A typhoon scanner with a 532 nm excitation laser and a 555 nm emission filter was used to scan the gels for fluorescently labeled proteins.

Screening the Pathogen Box Identifies Multiple Hit Antileishmanial Compounds

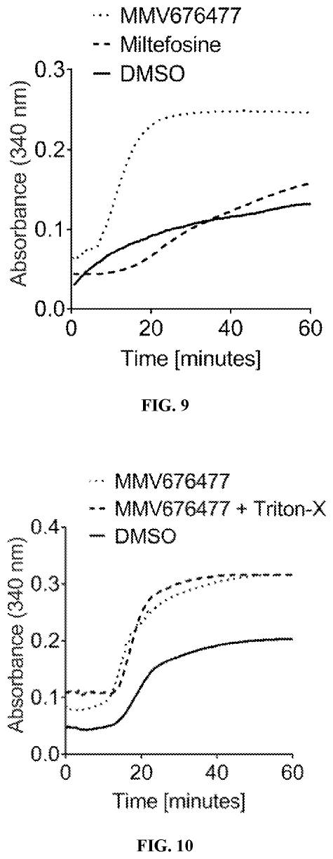

To identify antileishmanial compounds, a microplate-based alamarBlue® assay was used to quickly triage the 400 drug-like compounds available in the MMV Pathogen Box collection. The clinically-used antileishmanials amphotericin B and miltefosine were included as additional controls. A three-step process was used to screen the Pathogen Box: 1) identify hits against axenic amastigotes, 2) confirm the inhibitory concentrations of these hits against axenic amastigotes, 3) evaluate hits for potency against intracellular parasites using fluorescence and bioluminescence-based intracellular assays. The MMV Pathogen Box resource was tested at two concentrations (5 μM and 1 μM, 72 h endpoint) on axenic amastigotes. Full results are shown in (FIG. 9).

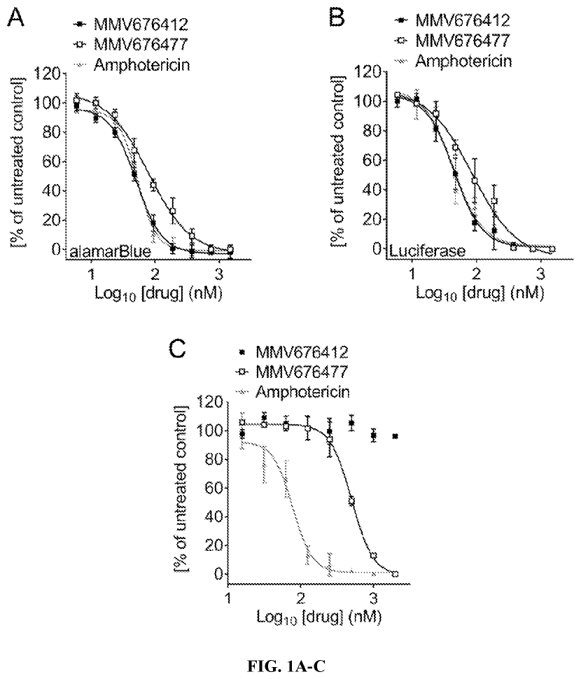

Hits were defined as compounds that, at a concentration of 1 μM, decreased the relative fluorescence intensity signal to ≤70% of that produced by parasites incubated in vehicle (0.06% DMSO) only (FIG. 9). A total of 10 hits were identified, including four reference compounds: buparvaquone, delamanid, auranofin and nitazoxanide. All 10 hits were analyzed to determine their EC₅₀ at 72 h (EC₅₀^{72 h}). The top hit (MMV676477) displayed similar potency to that of buparvaquone, delamanid and amphotericin B. Exemplar log-concentration-response curves for MMV676412, with comparison to amphotericin B, are shown in FIG. 1A. MMV676477 [EC₅₀^{72 h} (95% CI)=79 nM (62 to 100)] is nearly as potent as amphotericin B [EC₅₀^{72 h} (95% CI)=53 nM (46 to 60)] in vitro (FIG. 1A). Since EC₅₀^{72 h} data for MMV676477 was acquired using the alamarBlue® assay, the reliability of these data and alamarBlue® itself was independently confirmed by a bioluminescence assay, using transgenic luciferase-expressing parasites (L. amazonesisluc) (FIG. 1B). The EC₅₀^{72 h} value determined using either assay format were almost identical.

Compounds supplied in the MMV Pathogen Box have been tested for cytotoxicity against human cell lines, and this information is available online (https://www.pathogenbox.org/about-pathogen-box/supporting-information). Prior to testing the activity of active molecules against intracellular parasites, their cytotoxicity against the macrophage (Mϕ) cell line, RAW 264.7 was examined. The CC₅₀ values at 72 h are in accordance with those reported in the ChEMBL database (FIG. 10).

MMV676477 is a Potent and Selective Antiparasitic Hit Compound

In order to be selected as a hit, at minimum, a compound should be cytocidal, with an intracellular EC₅₀ of less than 10 μM. The most potent compound, MMV676477, was analyzed against intracellular L. amazonensis amastigotes, along with amphotericin as a positive control. RAW 264.7 cells were used to perform a bioluminescence assay with transgenic luciferase-expressing L. amazonensis (FIG. 1C). Both amphotericin [EC₅₀^{72 h} (95% CI)=77 nM (8 to 109)] and MMV676477 [EC₅₀^{72 h} (95% CI)=510 nM (470 to 550)] affected intracellular parasites.

All routine in vitro quantification of antileishmanial drug potency is quantification of “cytostatic” potency. Molecular studies of drug resistance have identified the need to rapidly define a hit's cytocidal activity early in the drug development process and have emphasized prioritizing compounds that kill parasites rather than merely inhibiting their growth. Therefore, the cytocidal action of MMV676477 was quantified by obtaining an LD₅₀ at 72 h (a Lethal Dose that kills 50% of the bulk population of the parasites relative to untreated control) and compared it to the known cytocidal drug amphotericin. An intracellular washout assay was employed. MMV67477's LD₅₀72 h was 620 nM and amphotericin's LD₅₀72 h was 82 nM. The ratio of LD₅₀:EC₅₀ was calculated for MMV676477 and amphotericin. Lower ratios indicate that the LD₅₀ is similar to the EC₅₀ value, meaning that the estimated EC₅₀ values represent killing of the parasites rather than simple growth inhibition. Amphotericin B, a known cytocidal drug, has an estimated LD₅₀ and EC₅₀ ratio of 1. MMV676477 had an LD₅₀/EC₅₀ ratio of 1.2, indicating cytocidal activity.

MMV676477 was tested against several other parasites: the axenic amastigotes of Leishmania donovani and Leishmania tarentolae, blood-stage Plasmodium falciparum, and Trypanosoma brucei. The compound's activity was measured using alamarBlue®, Malaria SYBR green I fluorescence (MSF) and CellTiter-Glo® luminescent assays. MMV676477 demonstrated broad activity against all three parasites. In addition, MMV676477 has also been recently reported as having potent activity against Neospora caninum, Cryptosporidium parvum, Toxoplasma gondii, and Entamoeba histolytica.

Structure-Activity-Relationship (SAR) of MMV676477: Initial Characterization

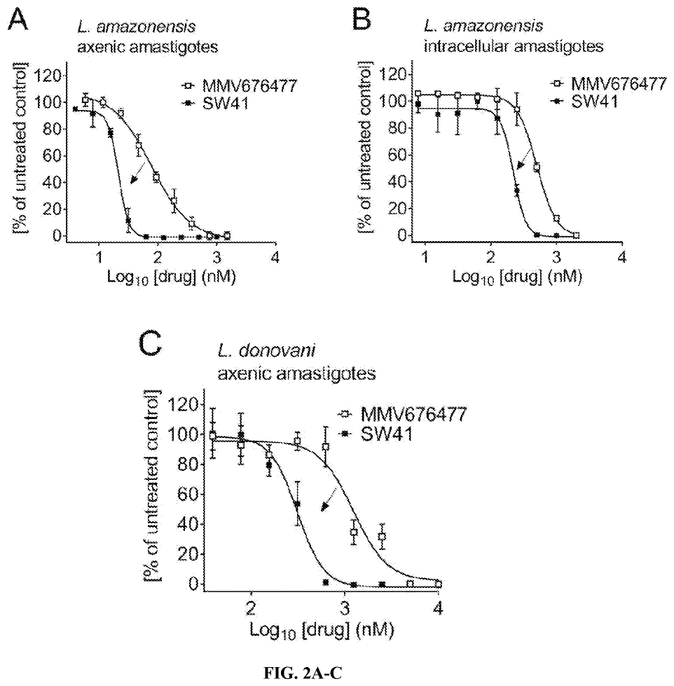

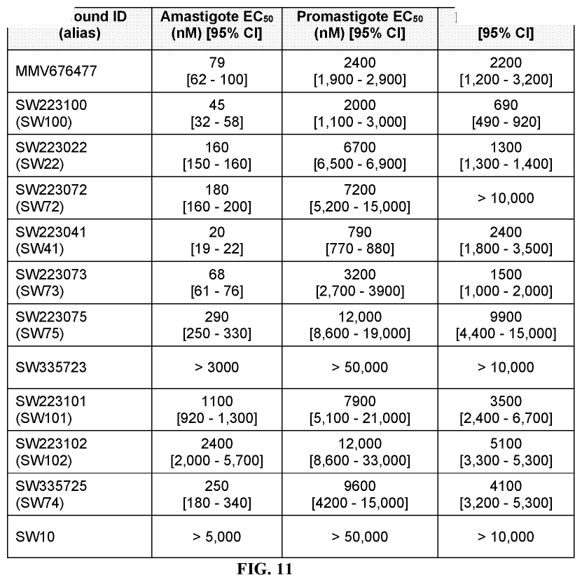

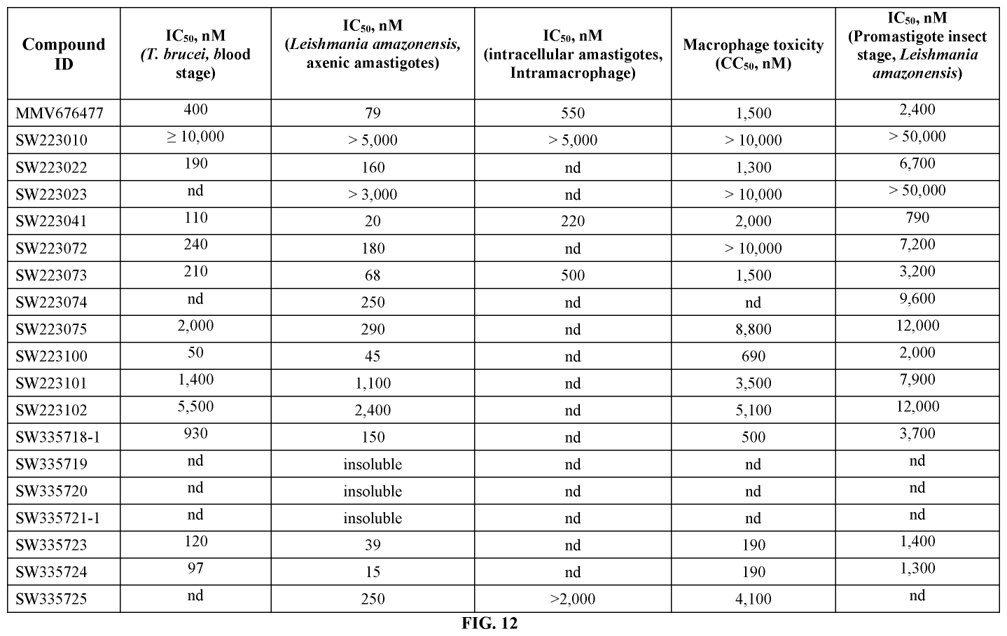

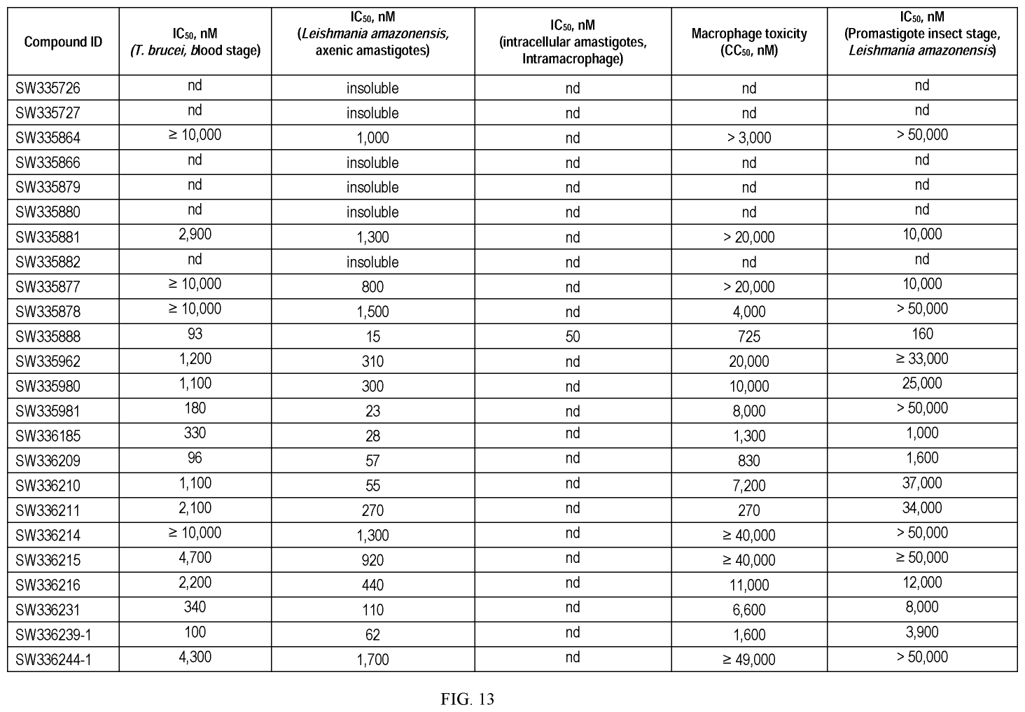

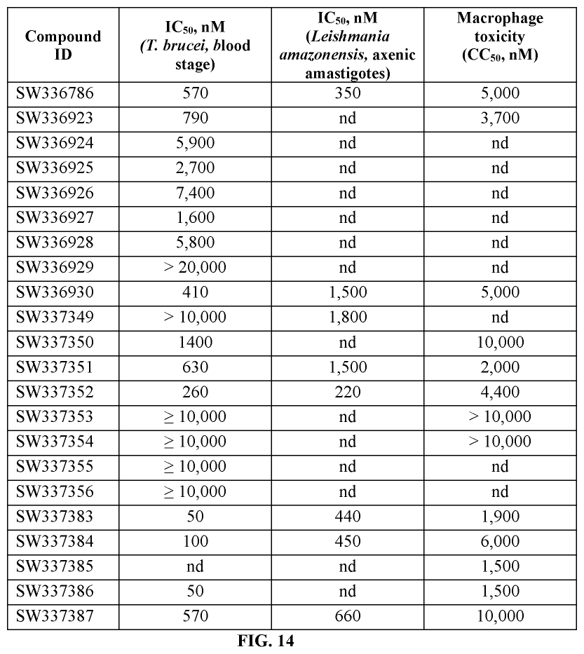

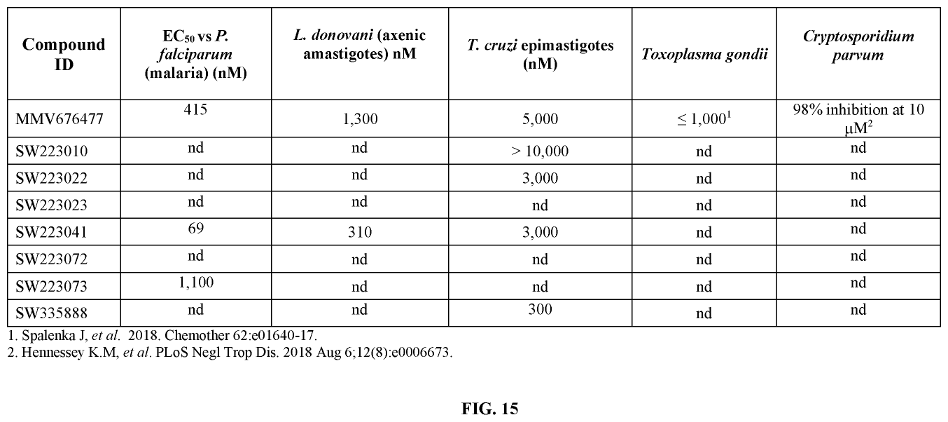

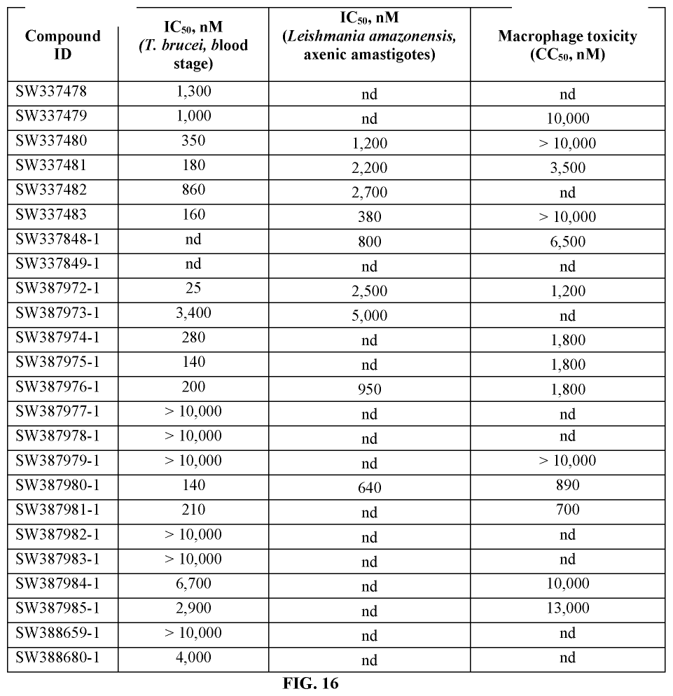

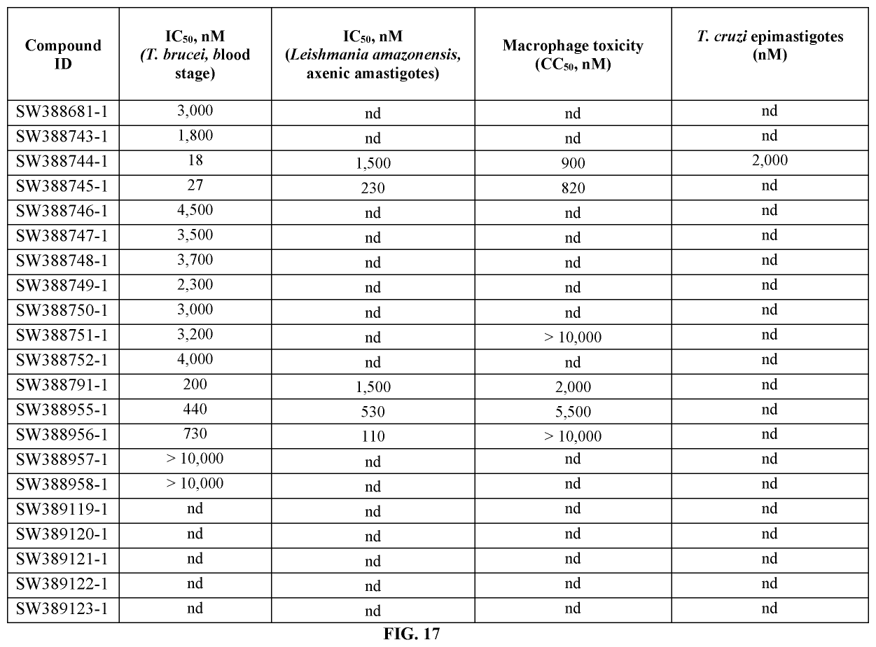

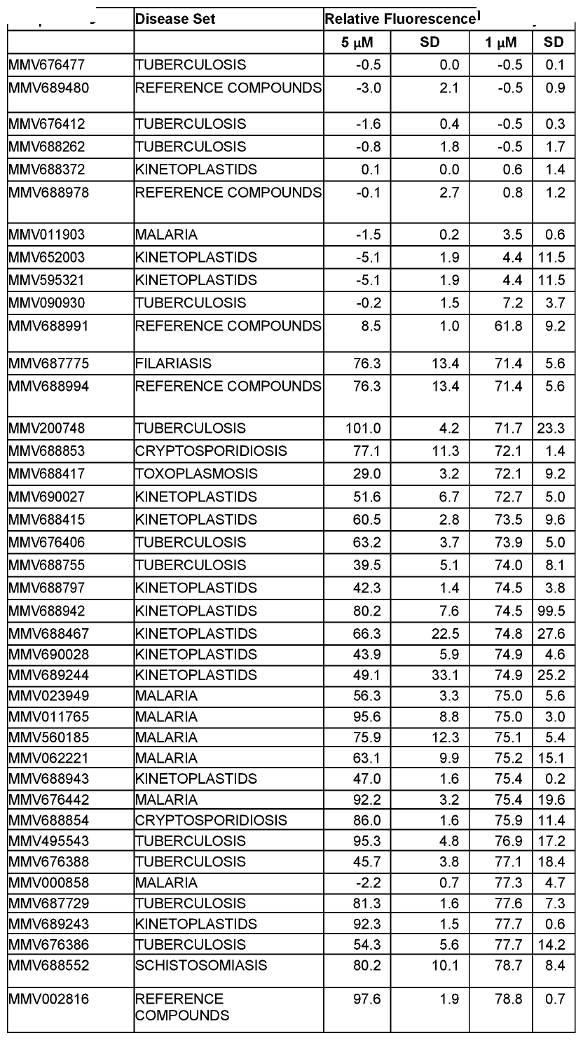

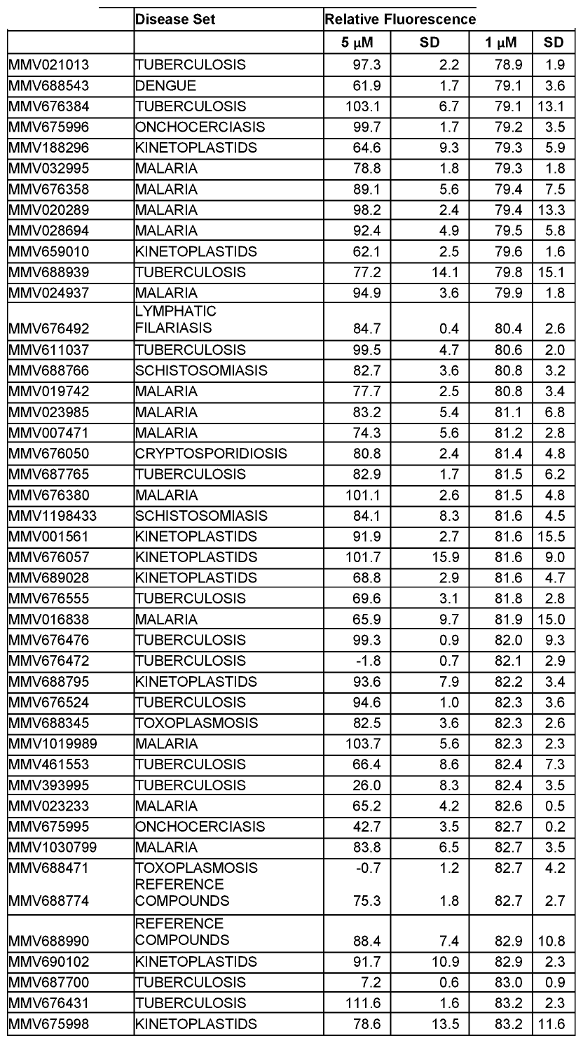

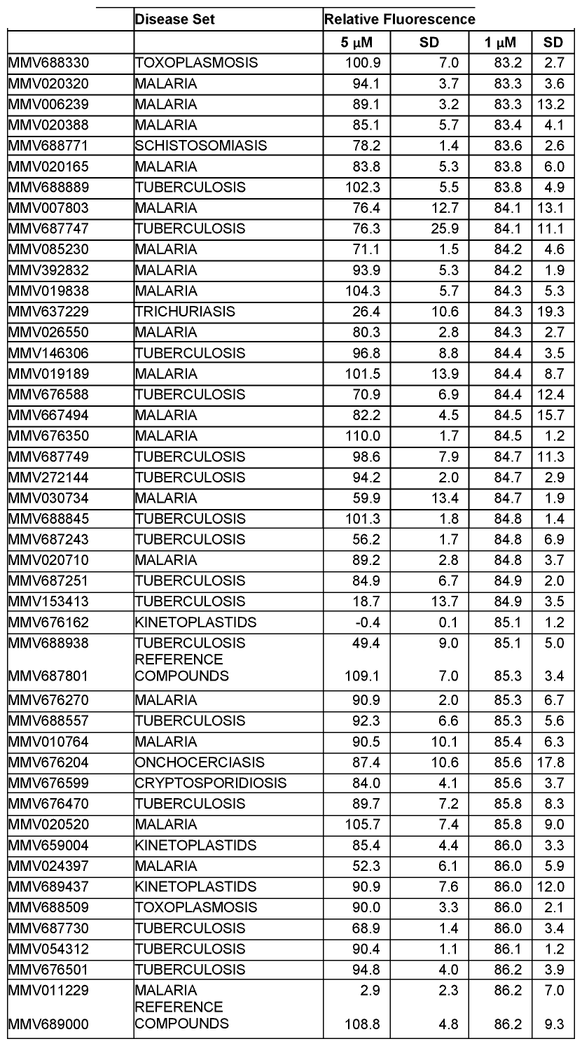

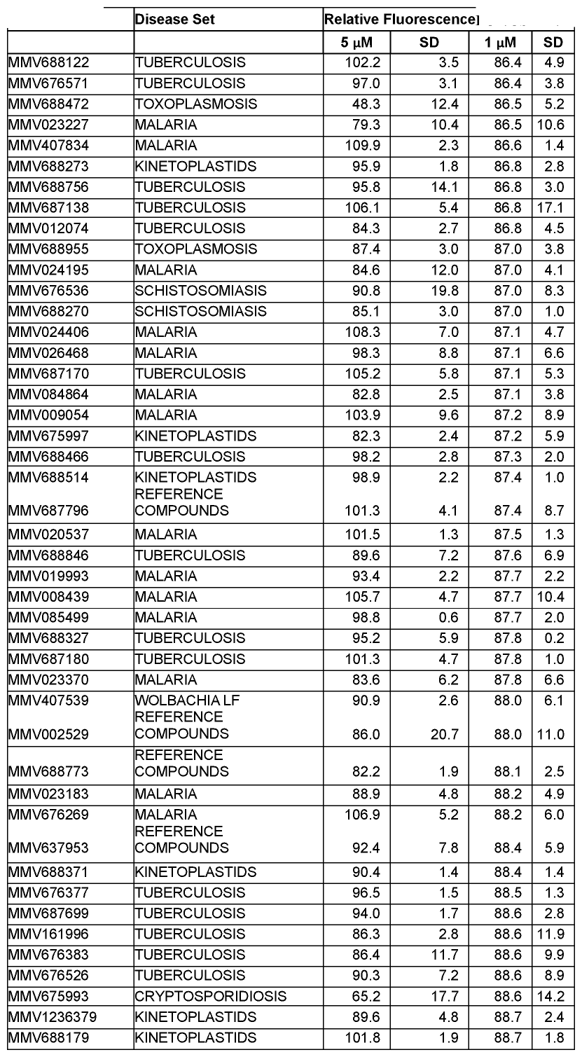

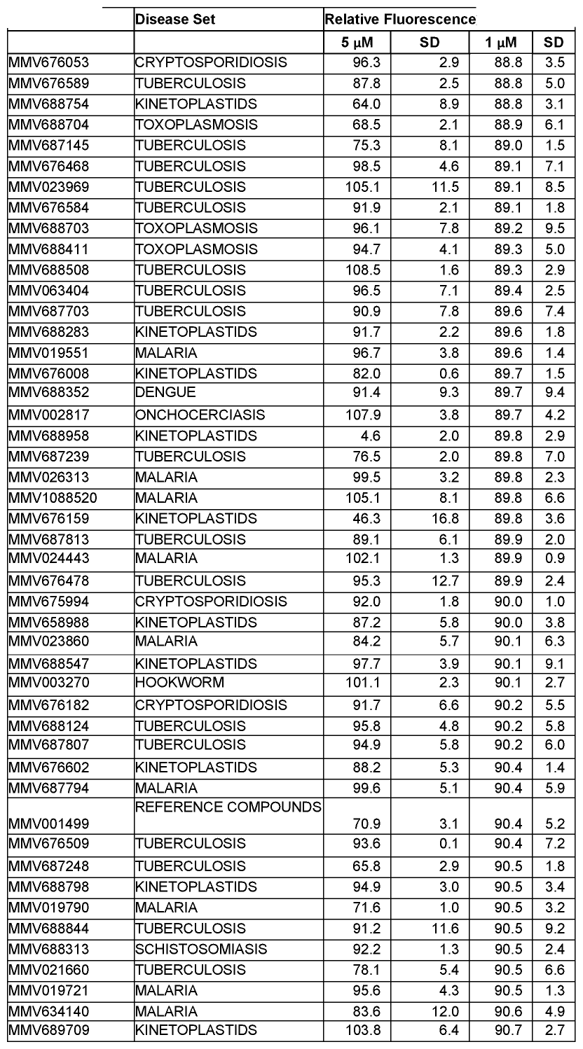

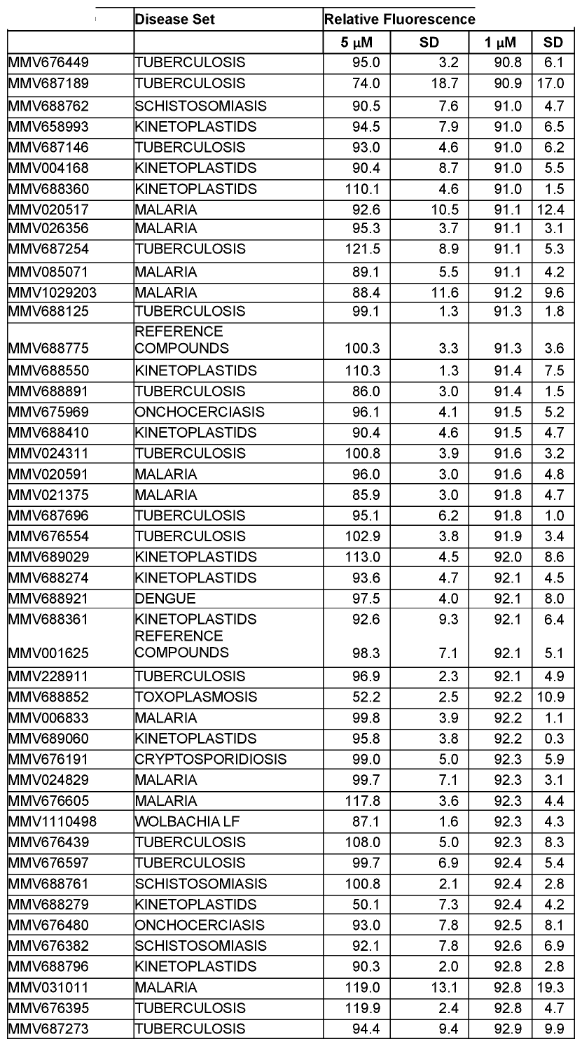

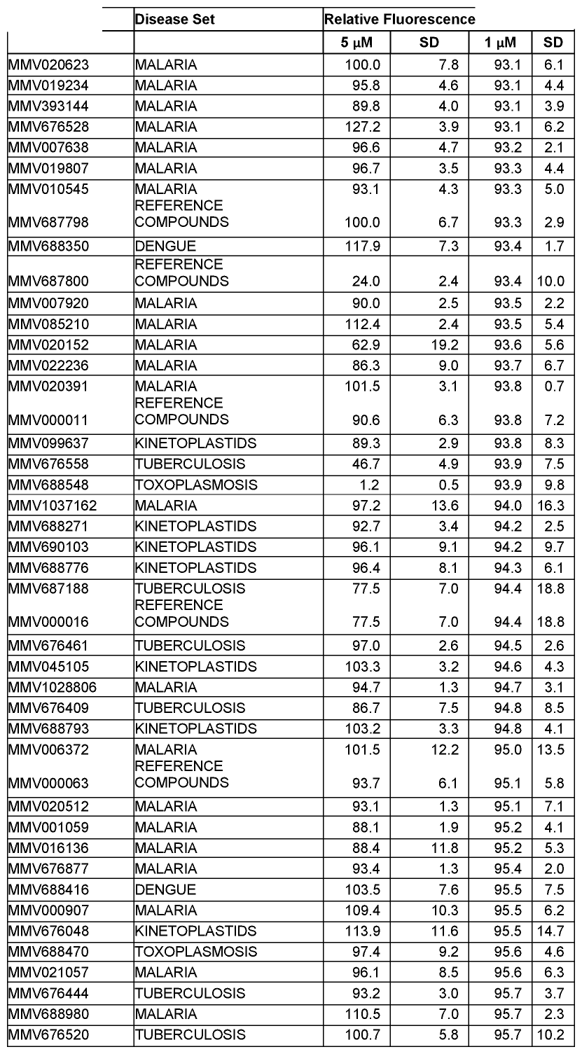

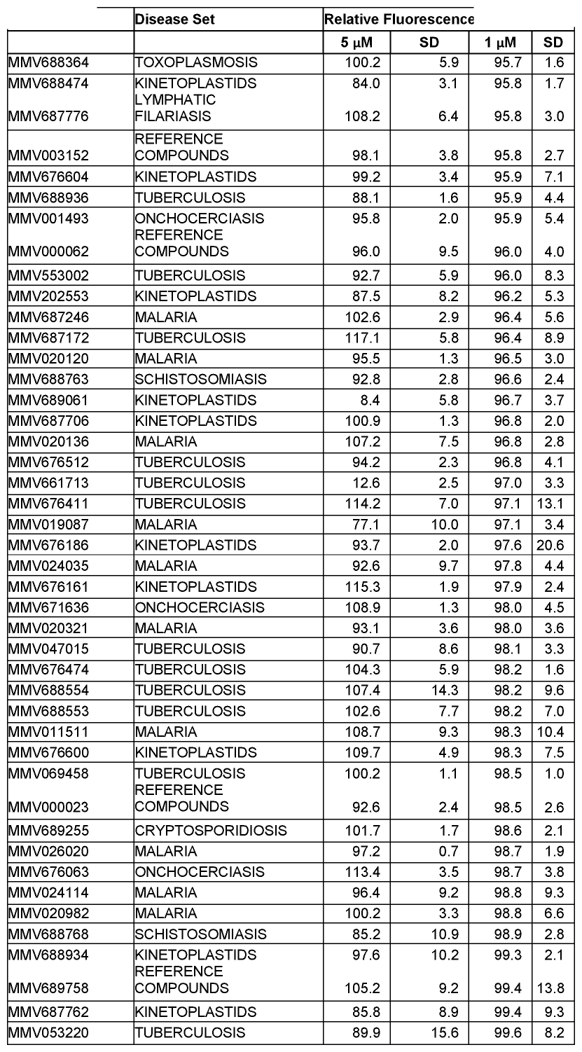

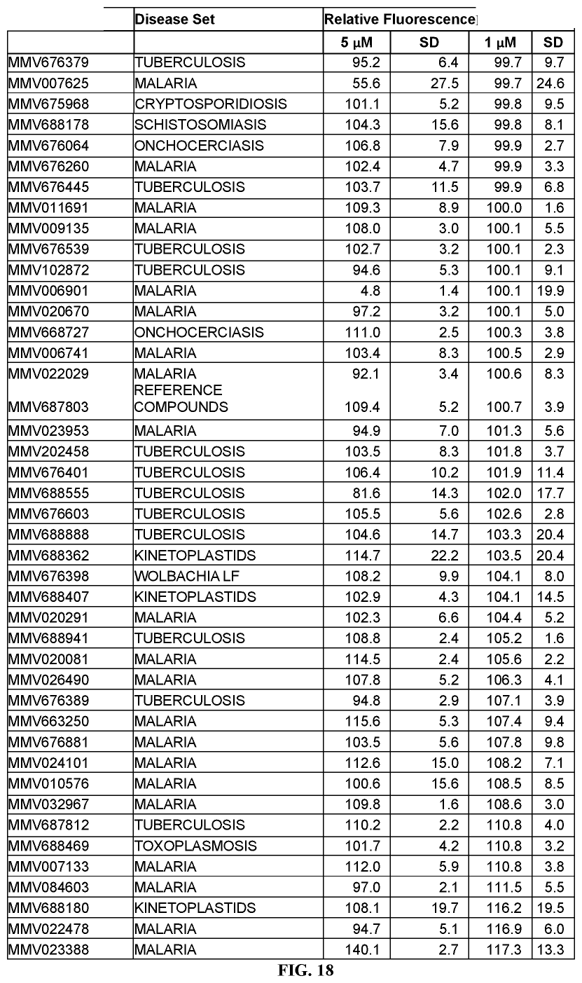

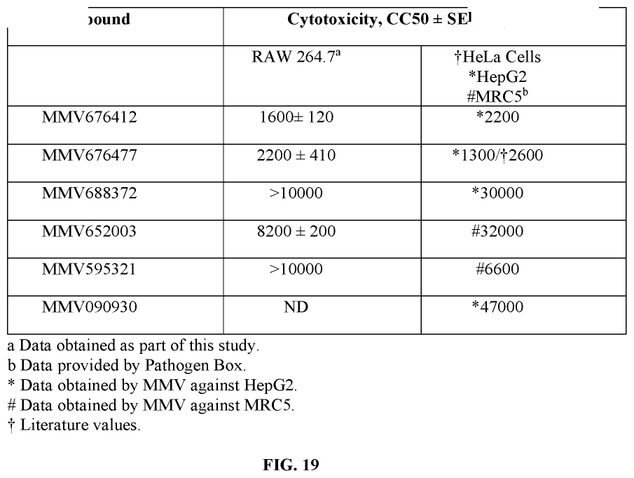

Re-synthesis of MMV676477 confirmed its activity to be the same as the activity identified during the initial screen. To improve the potency and selectivity of MMV676477, as well as to identify regions of the compound that could be functionalized for mode-of-action studies, 11 analogs were initially synthesized. MMV676477 is subsequently used as a control in this application to demonstrate antiparasitic activity. The structure of MMV676477 is characterized by a central pyrazole ring linked through N1 to a pyrimidinone moiety. An N-acylated amino group at the pyrazole C5 position provides an additional opportunity for diversification. Preliminary SAR survey modified the pyrimidinone and N-acyl group. The synthesized compounds exhibited range of activities from 20 nM to 5000 nM against L. amazonensis axenic amastigotes (FIG. 11). SW223041, the most potent compound, shows about four-fold improved activity against L. amazonensis [EC₅₀^{72 h} (95% CI)=20 nM (19 to 22)], compared to MMV676477, with similar cytotoxicity to that of MMV676477 [CC₅₀^{72 h} (95% CI)=2400 nM (1800 to 3500)]. SW223041 was also tested against L. donovani axenic amastigotes, blood-stage P. falciparum (3D7 strain) and L. amazonensis intracellular amastigotes (FIG. 2). SW223041 is 3-4-fold more potent than MMV676477 against all three parasites (FIG. 2). Moreover, its therapeutic index is increased relative to the initial hit as a consequence of improved potency vs. parasite without a corresponding increase in potency towards macrophages. For the 12 compounds initially tested, the antileishmanial activity ranks as follows: SW223041>MMV676477>SW223073>SW223022>SW335725>SW223075>SW223100/SW223101>SW223102>SW223023/SW10.

MMV676477 Promotes Cellular Microtubule Polymerization

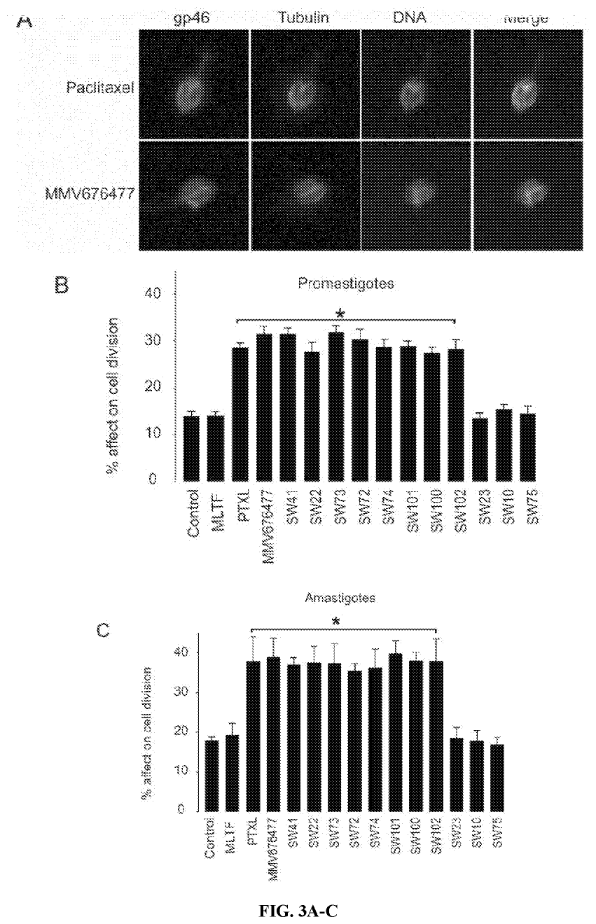

The molecular target of MMV676477 was investigated. MMV676477-treated L. amazonensis microscopically resembled parasites treated with the anti-cancer drug paclitaxel (“Taxol”, EC₅₀^{72 h} (95% CI) for La=760 nM (750 to 770). Paclitaxel stabilizes tubulin and prevents mitosis in cancer cells. Paclitaxel was used as a positive benchmark control for tubulin polymerization activity. An unrelated antileishmanial drug, miltefosine, was used as a negative control. To test the hypothesis that MMV676477 affected Leishmania cell division, both promastigote and amastigote forms of L. amazonensis were treated with MMV676477 and analogs at their 72 h EC₅₀ concentration (EC₅₀⁷² h), but only for 48 hrs to ensure that parasites were assessed prior to their death, and examined them by confocal immunofluorescence microscopy (FIG. 3A). Similar to paclitaxel, a significant two-fold increase was identified in the percentage of L. amazonensis that were undergoing cell division in MMV676477 and analog-treated promastigotes and amastigotes (FIGS. 3B-3C). Inactive analogs and miltefosine did not affect L. amazonensis cell division. The ability of analogs to affect L. amazonensis cell division was completely consistent with their antiparasitic activity, with the exception of SW223075, which has antiparasitic activity [EC₅₀^{72 h} (95% CI)=290 nM (250 to 330)] but no effect on cell division.

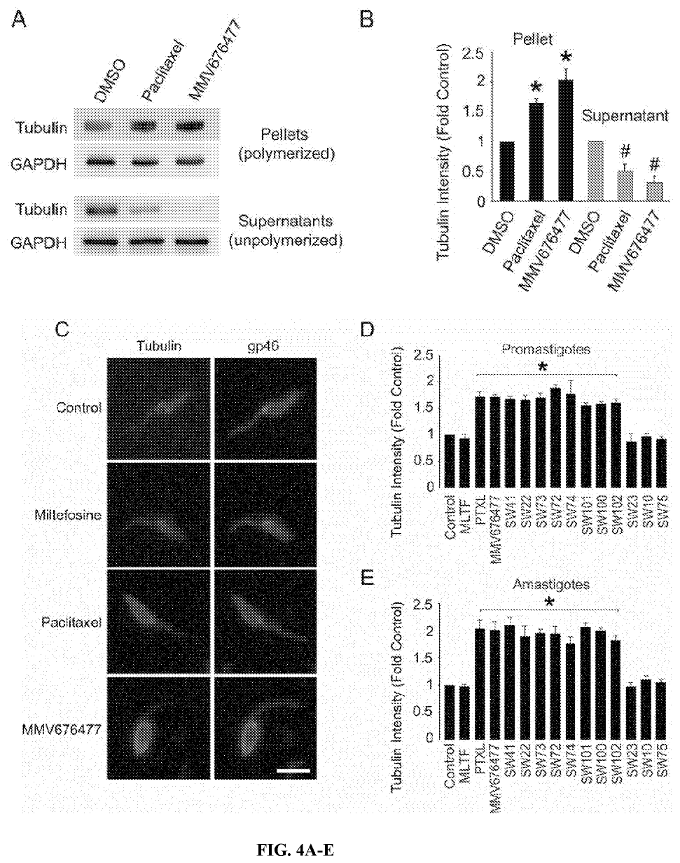

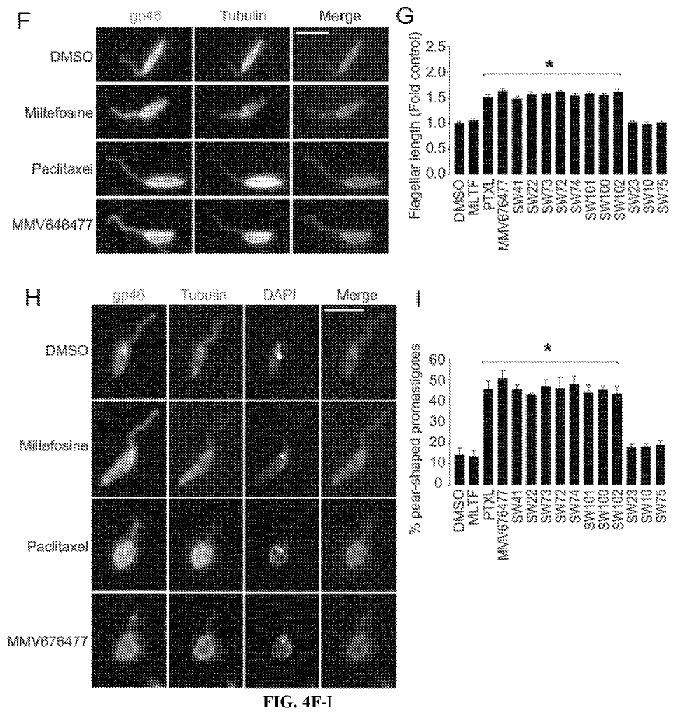

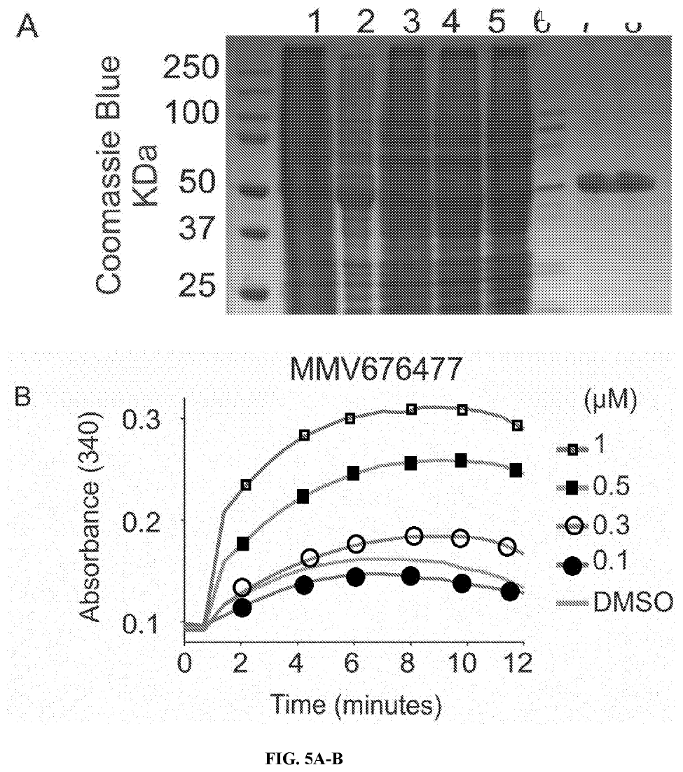

One reason why MMV676477 might affect parasite cell division is that, like paclitaxel, it might affect microtubule polymerization, either directly or indirectly. The degree to which MMV676477 affected microtubules polymerization in cells was examined. First, cell extracts were prepared from compound-treated and untreated L. amazonensis after treatment for 24 h at the EC₅₀^{72 h}. Centrifugation of these extracts allowed separation of insoluble polymeric (pellet) and soluble dimeric (supernatant) tubulin. Western blot analysis revealed that both paclitaxel and MMV676477 promoted the partitioning of cellular tubulin toward the polymeric form (FIG. 4A). Densitometric analysis of these blots showed that both MMV676477 and paclitaxel significantly increased the proportion of cellular polymeric tubulin (FIG. 4B). Due to both sectioning and extraction effects, polymerized tubulin is brighter by confocal microscopy when labelled with a tubulin antibody, and this method has been used as a proxy for quantifying the degree of microtubule polymerization. To determine whether the MMV676477 scaffold increased tubulin intensity by confocal microscopy, both promastigotes and amastigotes of L. amazonesis were treated with MMV676477 and its analogs, along with paclitaxel and miltefosine, for 24 hrs at their respective EC₅₀^{72 h} concentrations and performed confocal immunofluorescence microscopy (FIG. 4C). The intensity of α-tubulin staining was examined by normalizing it to the intensity of a membrane protein (gp46 for promastigotes in FIG. 4D; p8 for amastigotes in FIG. 4E). Paclitaxel, MMV676477 and all the active analogs had a significant two-fold increase in tubulin intensity over control (DMSO-treated) in L. amazonensis promastigotes and amastigotes, while the two inactive analogs, SW223075, and miltefosine did not increase tubulin intensity in either L. amazonensis promastigotes or amastigotes.

MMV676477 Promotes Purified Tubulin Polymerization

Because experiments with intact parasites suggested that the MMV676477 scaffold promotes microtubule polymerization in cells, the effects on tubulin activity was examined. To determine whether MMV676477 directly regulates microtubule assembly, assembly-competent tubulin from Leishmania tarentolae was purified and analyzed (FIG. 5A). L. tarentolae tubulin is highly conserved with other Leishmania species, including L. amazonesis (>98% identity). High concentrations of both tubulin and compound are needed in turbidity assays because the measurement is stoichiometric. Tubulin at or below 5 mg/mL does not polymerize in turbidity assays under experimental conditions unless a strong tubulin enhancer is added to the reaction (e.g. paclitaxel). 3 mg/mL purified L. tarentolae tubulin (3 mg/mL) was treated with serial dilutions of MMV676477 while the absorbance was measured at 340 nm over time. Representative turbidity curves are shown in FIG. 5B. MMV676477 promoted L. tarentolae tubulin polymerization in a concentration-dependent manner (MMV676477 EC₅₀=0.5+0.1 μM; paclitaxel EC₅₀=1.3+0.1 μM).

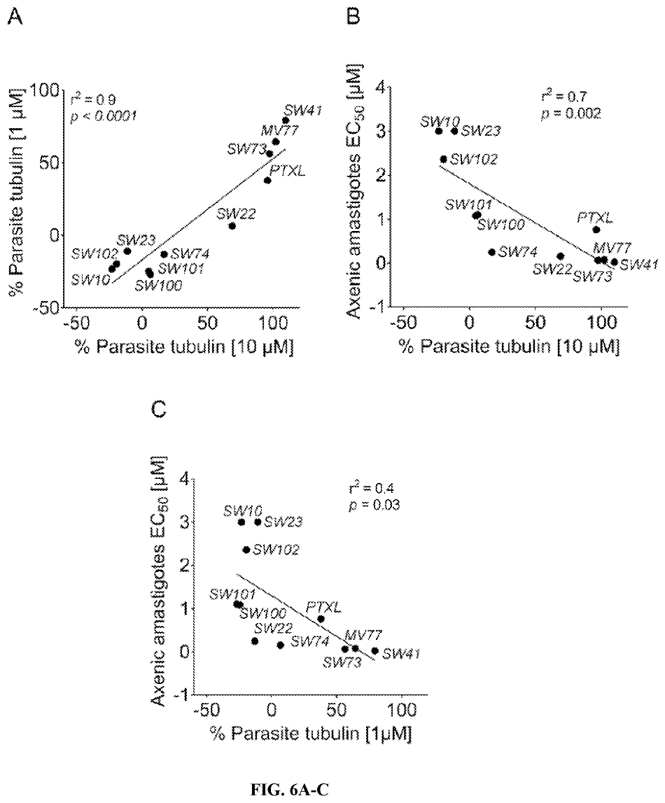

The effects of the initial dozen MMV676477 analogs were tested on L. tarentolae tubulin polymerization at 10 and 1 μM. Linear regression analysis was performed by plotting the relative tubulin polymerization activity at 10 and 1 μM on the X and Y axes (FIG. 6A). This plot showed a strong and significant correlation (p<0.0001, r²=0.9) and allowed ranking of the compounds from most to least polymerization activity: SW223041>MMV676477>SW223073>SW223022>SW335725>SW223100/SW223101>SW223102/SW223023/SW10. This ranking was exactly the same as the ranking of compounds' antiparasitic activity. Linear regression analysis enabled comparison of in vitro polymerization activity (1 and 10 μM) and growth inhibition of axenic amastigotes (FIG. 6B-6C). The 10 μM polymerization data showed a strong and significant correlation with antiparasitic EC₅₀^{72 h} data (p=0.002, r²=0.7) (FIG. 6B). Using the more stringent cut-off criteria of polymerization activity at 1 μM, the correlation with antiparasitic EC₅₀72 h data was weaker, but significant (p=0.02, r²=0.4) (FIG. 6C).

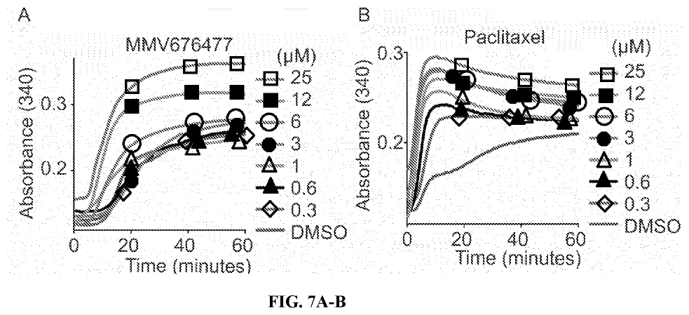

To determine the selectivity of MMV676477, purified porcine tubulin was exposed to a serial dilution of the drug (0 to 25 μM) and absorbance was measured at 340 nm over time. Representative turbidity curves of MMV676477 at different concentrations (0 to 25 μM) are shown in FIG. 7A. MMV676477 stimulated tubulin polymerization in a concentration-dependent manner (EC₅₀ value: MMV676477, 11+13.3 μM). Notably, the 20-fold selectivity of MMV676477 for purified Leishmania tubulin over mammalian tubulin (0.5 vs 11 μM EC₅₀) is consistent with the in vitro selectivity of MMV676477 for L. amazonensis over mammalian cells (78 vs 2200 nM). For comparison, paclitaxel was included in these experiments, which stimulated tubulin polymerization in a concentration-dependent manner (EC₅₀ value: paclitaxel, 1.5±0.2 μM (FIG. 7B). The unrelated antileishmanial drug miltefosine has no known activity on tubulin and did not affect tubulin polymerization at 50 μM (FIG. 9). At micromolar concentrations, some drugs self-associate into colloidal aggregates, resulting in non-specific effects on the target protein. Tubulin polymerization activity by MMV676477 was not affected by 0.01% Triton-X treatment, suggesting that MMV676477's activity was not merely aggregation-based (FIG. 10). To ensure that the absorbance seen at 340 nm reflected microtubule formation and not precipitation, a fluorescent microtubule assembly assay was employed. The fluorescence micrographs in FIG. 7C confirmed that many more microtubules were formed by paclitaxel and MMV676477-treated mammalian tubulin than the control.

MMV676477 Directly Binds to Purified Leishmania Tubulin

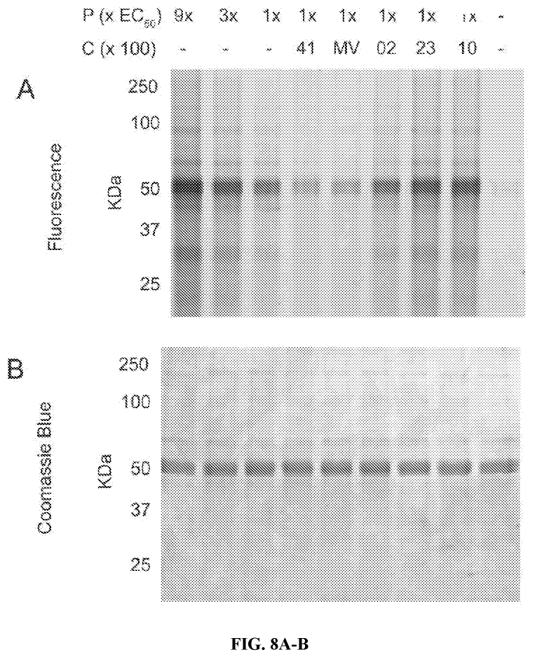

The above studies using purified tubulin implied that MMV676477 directly binds to tubulin in order to facilitate polymerization. However, no tubulin preparation is completely free of microtubule-associated proteins. To further investigate whether the MMV676477 scaffold directly bound tubulin, analog SW223022 was used as a probe for binding experiments (FIG. 11). SW223022 was modified to include (1) a benzophenone that facilitates crosslinking to binding partners under UV irradiation and (2) an alkyne group that facilitates conjugation to azide-containing dyes by copper-assisted cycloaddition (CuAAC; “click chemistry”). Purified tubulin was first treated with SW223022 (probe, EC₅₀=160 nM). The tubulin was then UV crosslinked to the probe, and Alexa Fluor 532-azide dye was conjugated to the probe via CuAAC. SDS-PAGE and fluorescent imaging allowed visualization of probe-bound tubulin (FIG. 8A). The intensity of a 50 kDa fluorescent band (consistent with tubulin) correlates positively with probe concentration (1× to 9× antiparasitic EC₅₀72 h). Some samples were simultaneously treated with both probe compound (P) and a 100-fold excess of competitors (MMV676477 and analogs). Dimming of the fluorescent tubulin band was representative of competition between the probe and active analogs. Consistent with their antiparasitic activity, the band was almost completely absent in samples treated with SW223041 (EC₅₀72 h=20 nM) and MMV676477 (EC₅₀72 h=78 nM), whereas SW223102, which has weak antiparasitic activity (EC₅₀72 h=2400 nM), caused less-pronounced dimming (FIGS. 8A-8B). No apparent competition by the inactive compounds SW223023 and SW10 was observed. Thus, competition with the SW223022 probe correlates with in vitro potency. These results support the conclusion that SW223022, MMV676477, and its analogs directly bind tubulin.

Due to tubulin's conservation across the protozoa and our scaffold's activity against multiple parasites, there is significant potential for this scaffold to allow development of broad-spectrum antiparasitic agents that will treat a multitude of devastating protozoal infections.

The claims are not to be interpreted as including means-plus- or step-plus-function limitations, unless such a limitation is explicitly recited in a given claim using the phrase(s) “means for” or “step for,” respectively.

1. A method for treating or inhibiting a parasitic disease in a subject comprising administering to the subject a composition comprising a compound of the formula below: 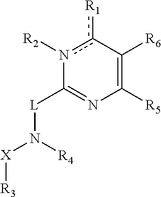
where:
R₁ is alkyl, oxygen, alkoxy, substituted or unsubstituted amine, or substituted or unsubstituted benzoate;
R₂ is H, alkyl, or substituted or unsubstituted benzyl;
L is a substituted or unsubstituted aryl or substituted or unsubstituted heteroaryl, wherein L optionally includes a carbonyl moiety linking L to the amine group;
X is H, CO, SO₂, or CONH;
when X is not H, R₃ is alkyl, substituted or unsubstituted cycloalkyl, substituted or unsubstituted heterocyclyl, substituted or unsubstituted aryl, or substituted or unsubstituted heteroaryl;
R₄ is H, alkyl, substituted or unsubstituted aryl, substituted or unsubstituted heteroaryl, substituted or unsubstituted heterocyclyl, substituted or unsubstituted cycloalkyl, or substituted or unsubstituted benzyl; and
R₅ and R₆ are independently selected from H, halide, linear, cyclic or branched alkyl, alkoxy, substituted or unsubstituted aryl, or substituted or unsubstituted heteroaryl, substituted or unsubstituted benzyloxy, substituted or unsubstituted benzoate, and substituted or unsubstituted arylsulfonate, or join to form a carbocyclic or heterocyclic ring;
wherein when R₁ is alkyl, alkoxy, substituted or unsubstituted amine, or benzoate, the bond between the R₁-bearing carbon and the vicinal amine is a double bond and there is no R₂ substituent, and when R₁ is oxygen, the bond between the R₁-bearing carbon and the vicinal amine is a single bond and the bond between the R₁-bearing carbon and oxygen is a double bond (carbonyl bond);
or a pharmaceutically-acceptable salt, or prodrug thereof.

2. The method of claim 1, wherein the composition disrupts trypanosomatid tubulin dynamics.

3. The method of claim 2, wherein the composition promotes trypanosomatid tubulin polymerization.

4. The method of claim 1, wherein the composition disrupts protozoan tubulin dynamics.

5. The method of claim 4, wherein the composition promotes protozoan tubulin polymerization.

6. The method of claim 1, wherein the subject is a human being.

7. The method of claim 1, wherein an effective dose of the compound inhibits, suppresses, or reduces the population of intracellular or axenic amastigotes or the human infective form of other parasites by about 50%.

8. The method of claim 1, wherein the parasitic disease is leishmaniasis, African trypanosomiasis, Chagas disease, malaria, cryptosporidiosis, or toxoplasmosis.

9. The method of claim 1, wherein the composition comprises a pharmaceutically acceptable carrier, excipient, diluent or solvent.

10. The method of claim 1, wherein the composition is administered orally, intraadiposally, intraarterially, intraarticularly, intracranially, intradermally, intralesionally, intramuscularly, intraperitoneally, intrapleurally, intranasally, intraocularly, intrapericardially, intraprostatically, intrarectally, intrathecally, intratumorally, intraumbilically, intravaginally, intravenously, intravesicularly, intravitreally, liposomally, locally, mucosally, orally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, via localized perfusion, bathing target cells directly, or any combination thereof.

11. The method of claim 10, wherein the orally-administered composition is provided in the form of capsule, tablet, syrup, concentrate, powder, granule, aerosol, or beads.

12. The method of claim 8, wherein the leishmaniasis is caused by a parasite of the genus Leishmania.

13. The method of claim 12, wherein the parasite of the genus Leishmania is Leishmania amazonesis, L. donovani, L. tarentolae, L. tropica, L. major, or L. aethiopica.

14. The method of claim 8, wherein the trypanosomiasis is caused by a parasite of the genus Trypanosoma.

15. The method of claim 14, wherein the parasite of the genus Trypanosoma parasite is Trypanosoma brucei gambiense, Trypanosoma brucei rhodesiense, or Trypanosoma brucei brucei.

16. The method of claim 14, wherein the parasite of the genus Trypanosoma parasite is Trypanosoma cruzi.

17. The method of claim 8, wherein the parasitic disease is caused by a parasite of the genus Plasmodium.

18. The method of claim 17, wherein the parasite of the genus Plasmodium is Plasmodium falciparum, P. vivax, P. ovale, P. malariae, or P. knowlesi.

19. The method of claim 8, wherein the parasitic disease is caused by a parasite of the genus Cryptosporidium.

20. The method of claim 19, wherein the parasite of the genus Cryptosporidium is C. parvum or C. hominis.

21. The method of claim 8, wherein the parasitic disease is caused by a parasite of the genus Toxoplasma.

22. The method of claim 21, wherein the parasite of the genus Toxoplasma is T. gondii.

23. The method of claim 1, wherein the compound of Formula I is further defined as at least one of:

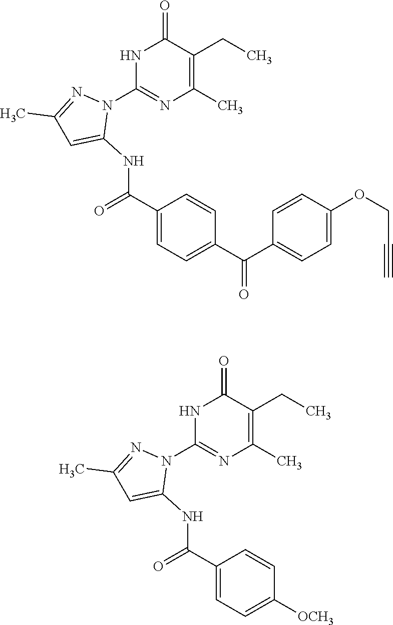

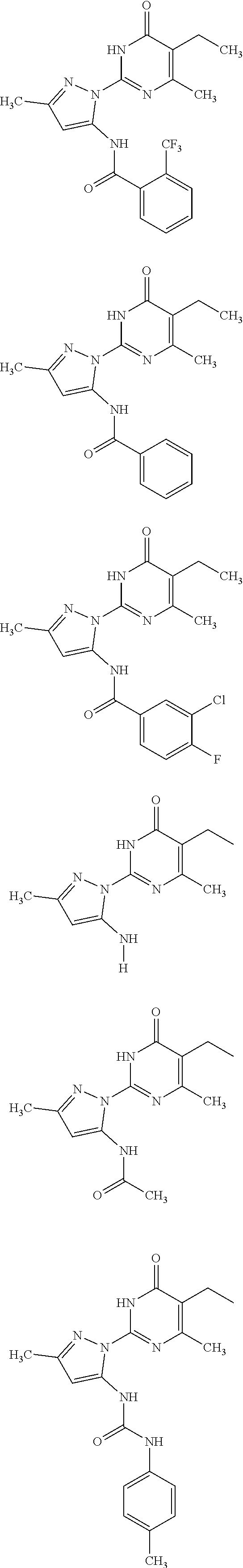

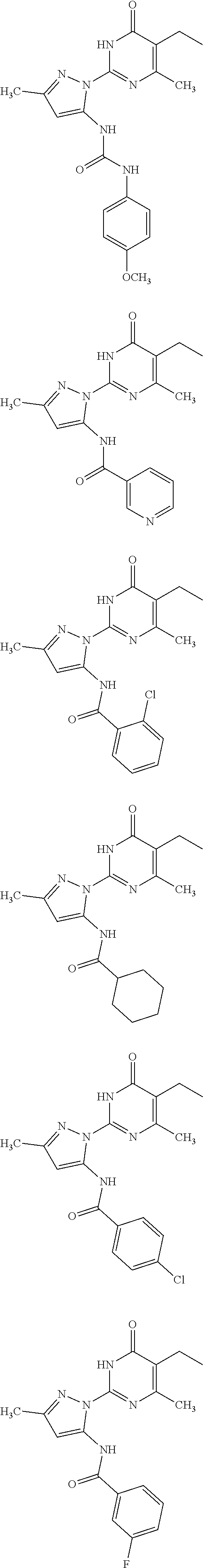

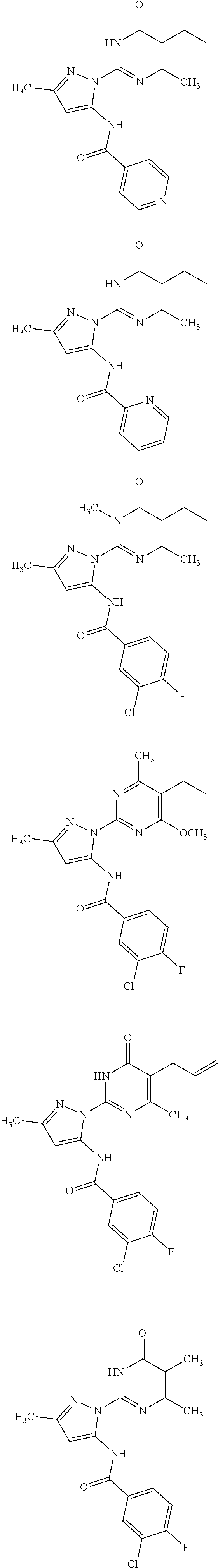

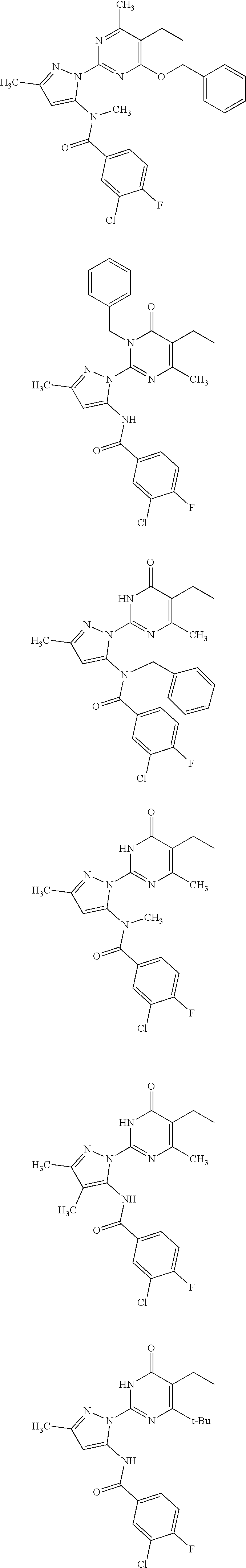

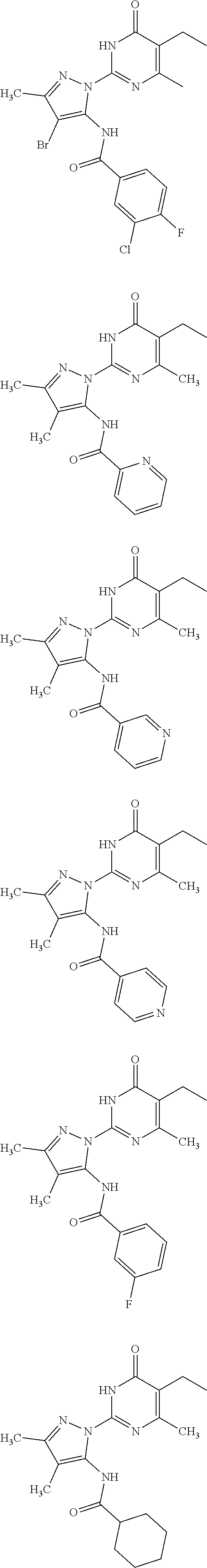

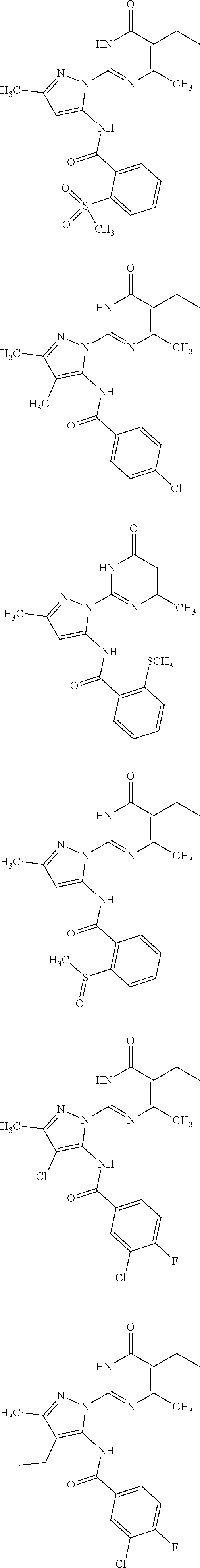

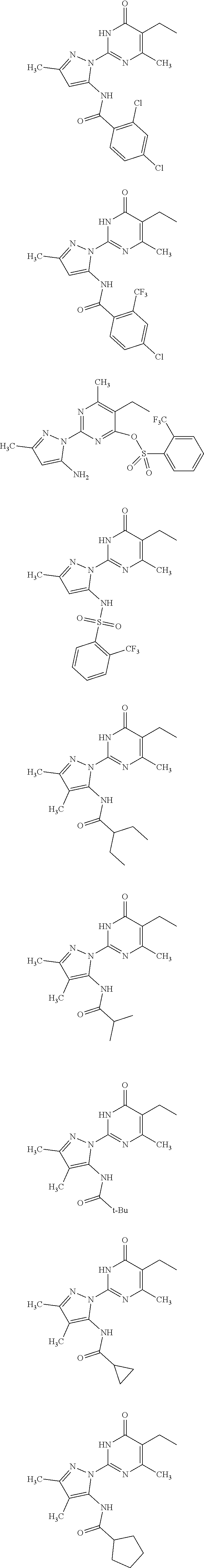

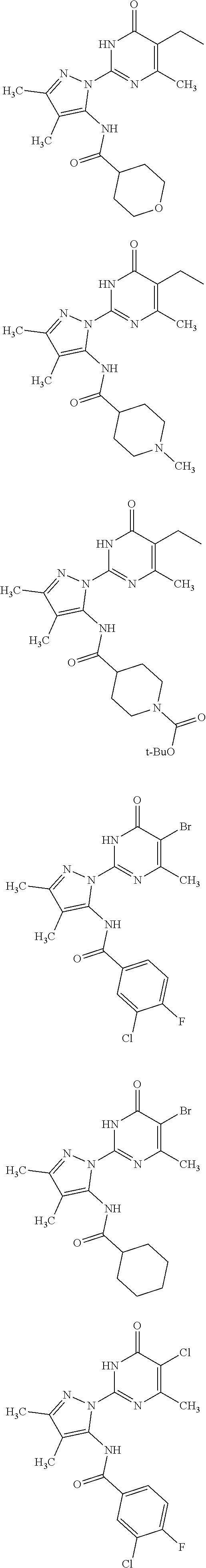

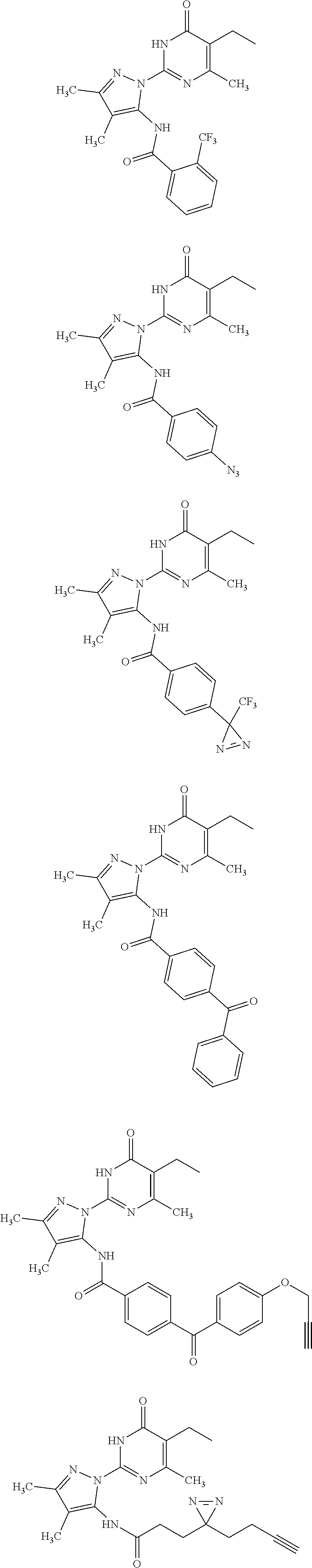

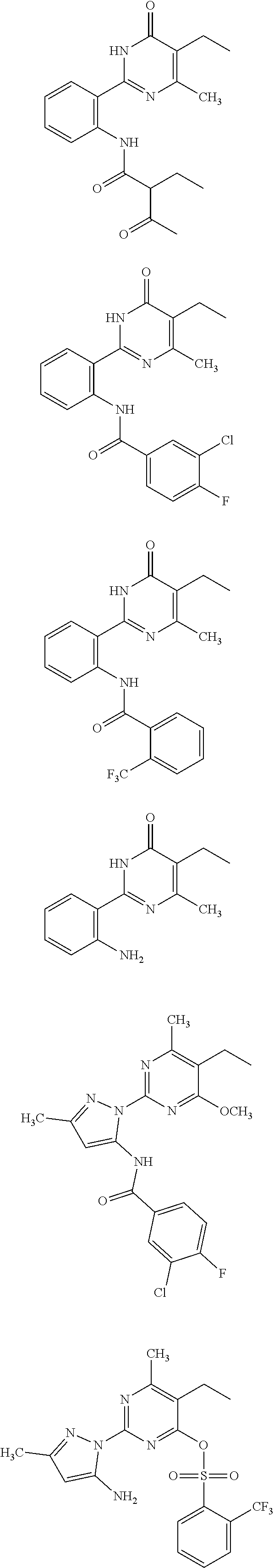

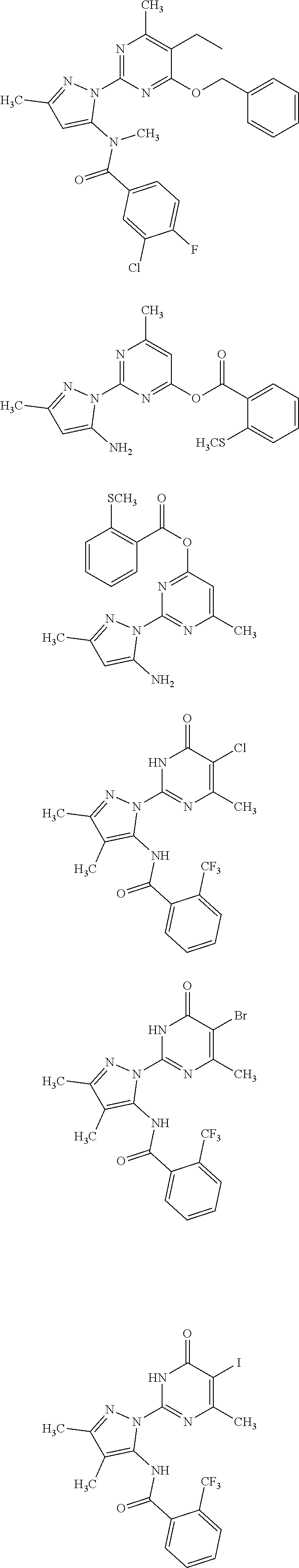

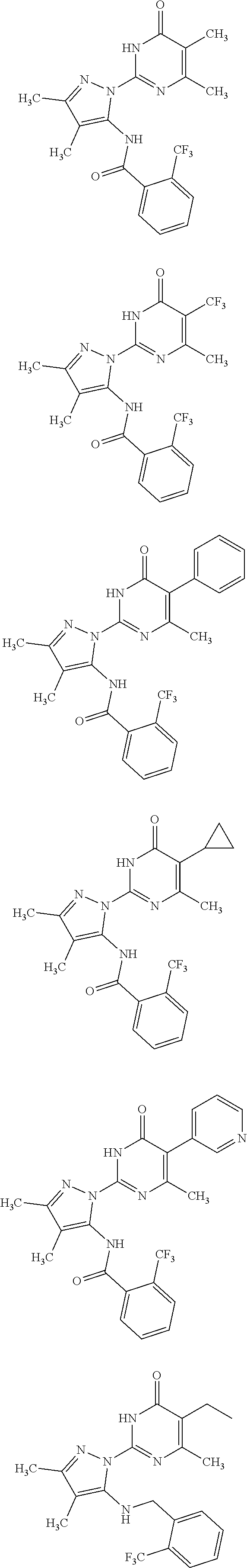

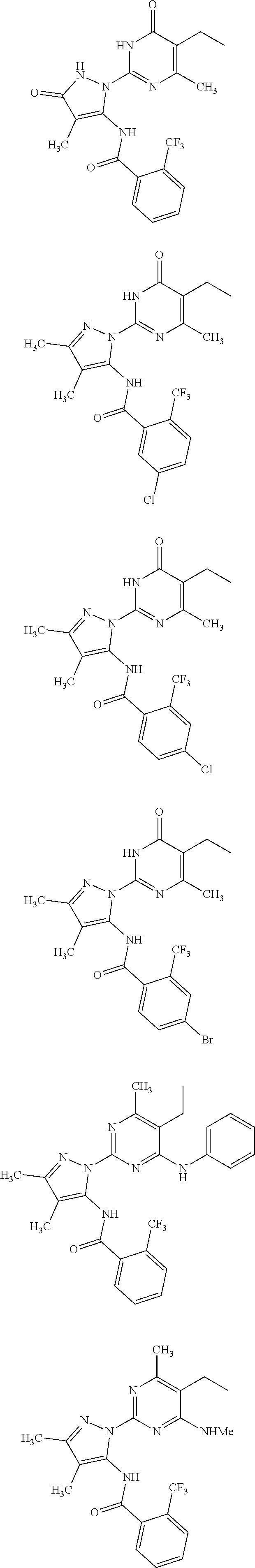

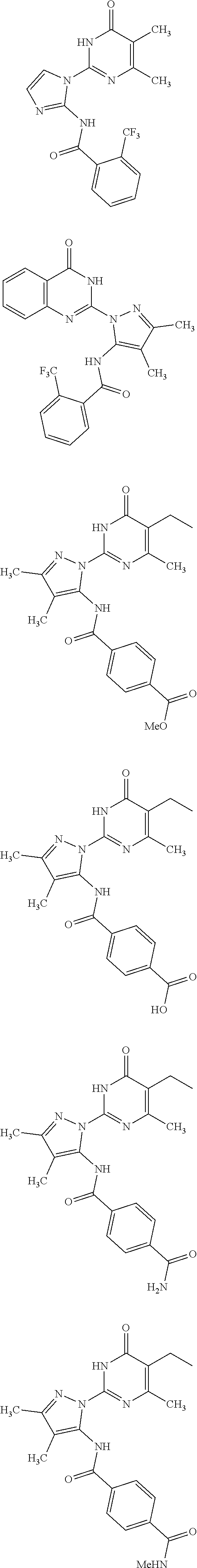

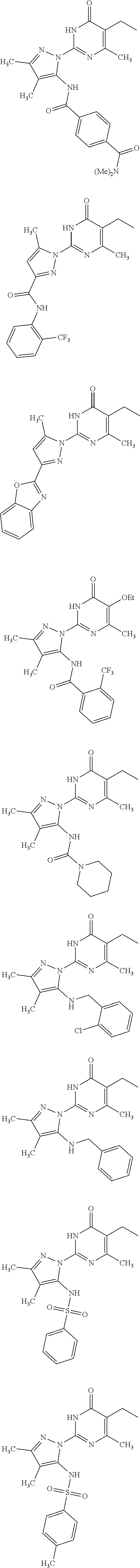

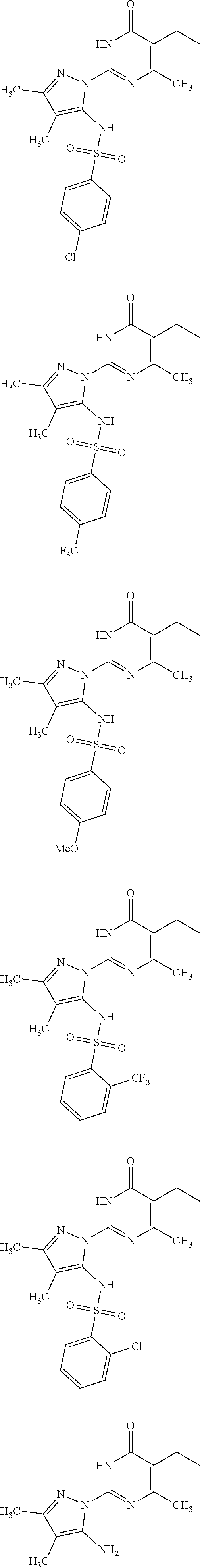

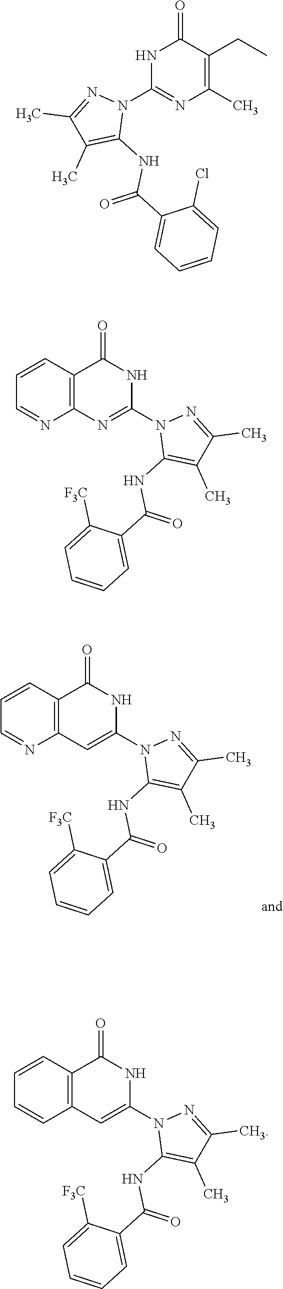 [SMILES: CCc1c(C)nc(-n2nc(C)c(C)c2NC(=O)c2ccccc2Cl)[nH]c1=O ; Cc1nn(-c2nc3ncccc3c(=O)[nH]2)c(NC(=O)c2ccccc2C(F)(F)F)c1C ; Cc1nn(-c2cc3ncccc3c(=O)[nH]2)c(NC(=O)c2ccccc2C(F)(F)F)c1C ; Cc1nn(-c2cc3ccccc3c(=O)[nH]2)c(NC(=O)c2ccccc2C(F)(F)F)c1C]

24. A composition comprising a compound of the formula below: 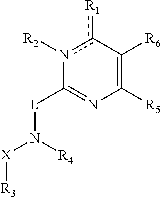
where:
R₁ is alkyl, oxygen, alkoxy, substituted or unsubstituted amine, or substituted or unsubstituted benzoate;
R₂ is H, alkyl, or substituted or unsubstituted benzyl;
L is a substituted or unsubstituted aryl or substituted or unsubstituted heteroaryl, wherein L optionally includes a carbonyl moiety linking L to the amine group;
X is H, CO, SO₂, or CONH;
when X is not H, R₃ is alkyl, substituted or unsubstituted cycloalkyl, substituted or unsubstituted heterocyclyl, substituted or unsubstituted aryl, or substituted or unsubstituted heteroaryl;
R₄ is H, alkyl, substituted or unsubstituted aryl, substituted or unsubstituted heteroaryl, substituted or unsubstituted heterocyclyl, substituted or unsubstituted cycloalkyl, or substituted or unsubstituted benzyl; and
R₅ and R₆ are independently selected from H, halide, linear, cyclic or branched alkyl, alkoxy, substituted or unsubstituted aryl, or substituted or unsubstituted heteroaryl, substituted or unsubstituted benzyloxy, substituted or unsubstituted benzoate, and substituted or unsubstituted arylsulfonate, or join to form a carbocyclic or heterocyclic ring;
wherein when R₁ is alkyl, alkoxy, substituted or unsubstituted amine, or benzoate, the bond between the R₁-bearing carbon and the vicinal amine is a double bond and there is no R₂ substituent, and when R₁ is oxygen, the bond between the R₁-bearing carbon and the vicinal amine is a single bond and the bond between the R₁-bearing carbon and oxygen is a double bond (carbonyl bond);
or a pharmaceutically-acceptable salt, or prodrug thereof.

25. The composition of claim 24, wherein R₁ is oxygen and R₂ is H.

26. The composition of claim 24, wherein X is CO and R₄ is substituted aromatic.

27. The composition of claim 24, wherein R₆ is methyl and R₇ is ethyl.

28. The composition of claim 24, wherein the compound of Formula I is further defined as one of: 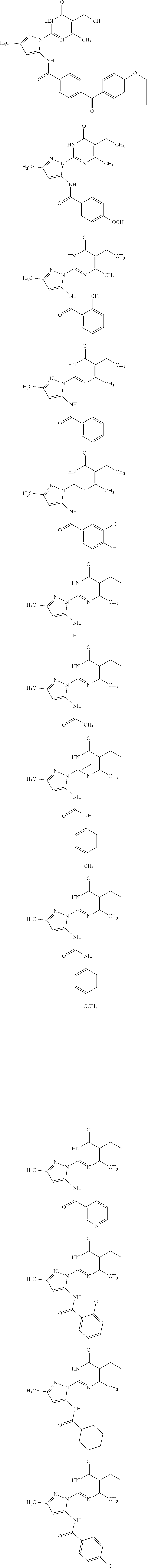 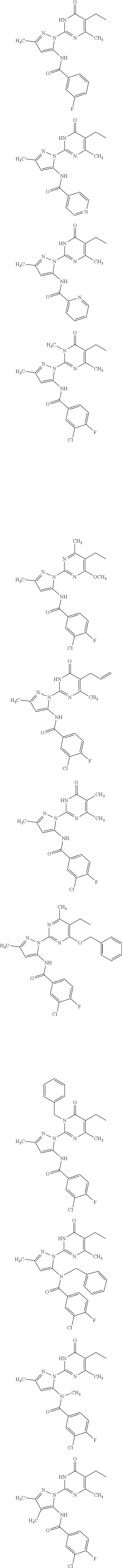 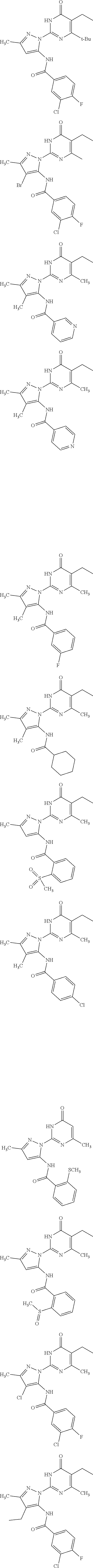 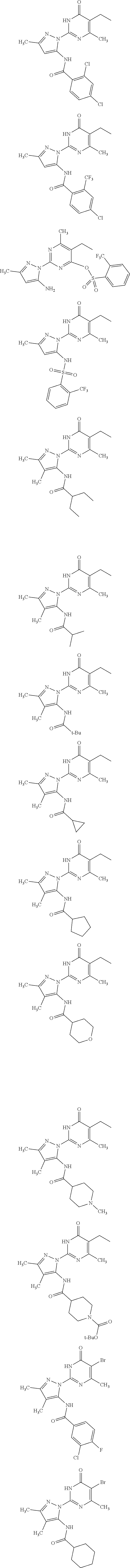 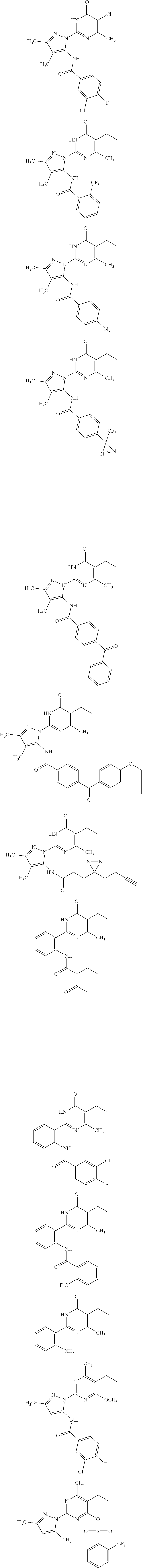 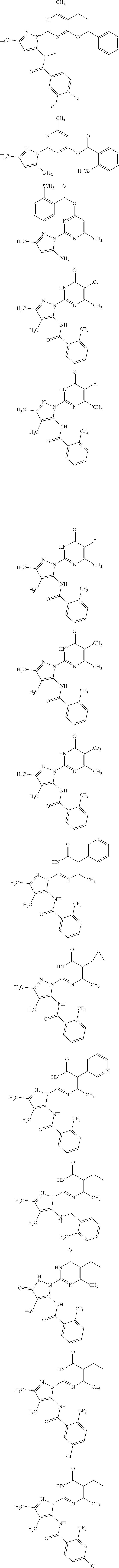 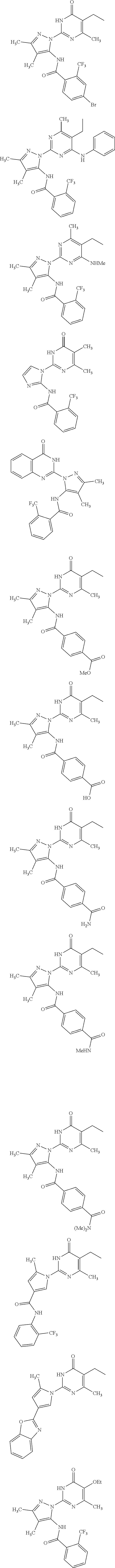 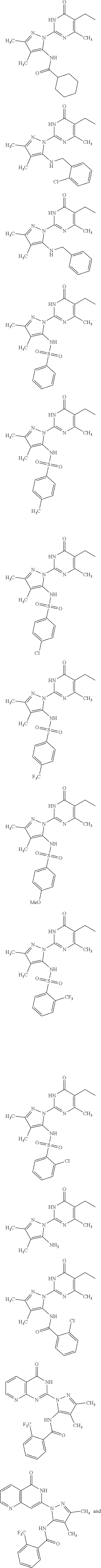 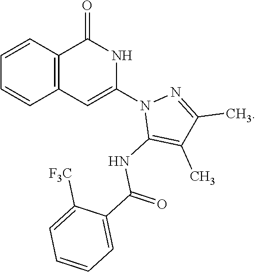 [SMILES: Cc1nn(-c2cc3ccccc3c(=O)[nH]2)c(NC(=O)c2ccccc2C(F)(F)F)c1C]
